(19) United States
(12) Patent Application Publication
(10) Pub. No.: US 2025/0144248 A1
(43) Pub. Date: May 8, 2025

(54) METHODS AND SYSTEMS FOR IMPROVED NUCLEIC ACID DELIVERY VIA ULTRASOUND

(71) Applicant: SonoThera, Inc., South San Francisco, CA (US)

(72) Inventors: Steven B. FEINSTEIN, Highland Park, IL (US); Kenneth GREENBERG, Berkeley, CA (US); Chuan FU, Sausalito, CA (US); Ivan KRIVEGA, Burlingame, CA (US); Maria KONOVALENKO, San Francisco, CA (US); Barry CAMPBELL, South San Francisco, CA (US); Zoya GLUZMAN-POLTORAK, South San Francisco, CA (US); David SATYADI, South San Francisco, CA (US); Margarita KRIVEGA, Burlingame, CA (US)

(21) Appl. No.: 18/937,809

(22) Filed: Nov. 5, 2024

Related U.S. Application Data
(60) Provisional application No. 63/711,635, filed on Oct. 24, 2024.
(60) Provisional application No. 63/656,080, filed on Jun. 4, 2024.
(60) Provisional application No. 63/547,672, filed on Nov. 7, 2023.

(51) Int. Cl. A61K 48/00; A61K 41/00; A61K 47/54

(52) U.S. Cl. CPC A61K 48/0058; A61K 41/0047; A61K 47/549

(57) ABSTRACT

Provided are methods, compositions, and kits for improving a delivery of a nucleic acid to a cell in an organ of a subject. Such methods, compositions, and kits may include a sonoactive agent and a nucleic acid comprising a cargo polynucleotide and a nuclear localization element and/or an innate immune response avoidance moiety, which may be an aptamer. Delivery may involve sonoporation.

### CROSS REFERENCE TO RELATED APPLICATIONS

This application claims the benefit of U.S. Provisional Patent Application No. 63/547,672 filed Nov. 7, 2023, and U.S. Provisional Patent Application No. 63/656,080 filed Jun. 4, 2024, and U.S. Provisional Patent Application No. 63/711,635 filed Oct. 24, 2024, each of which is incorporated herein by reference in its entirety and for all purposes.

### INCORPORATION BY REFERENCE OF SEQUENCE LISTING

The present application is being filed along with a Sequence Listing in electronic format. The Sequence Listing is provided as a file entitled 62668-729601.XML, created Oct. 31, 2024, which is 413,293 bytes in size. The information in the electronic format of the Sequence Listing is incorporated by reference in its entirety.

### BACKGROUND

Gene therapy, in which a functional copy of a gene is transfected into a cell, has been proposed as a possible method of treating genetic diseases. One proposed method of delivering a gene therapy to a subject is delivery of therapeutic agents using ultrasound and microbubbles, also referred to as sonoporation. However, prior art methods of gene therapy using ultrasound or sonoporation suffer from significant shortcomings such as low transfection rates, and insufficient gene expression, which have prevented the clinical development and commercialization of these methodologies. There remains a need in the art for more effective gene therapy techniques that can transfect a gene to a cell in an organ or a tissue in a subject in a safe, effective, and durable manner.

### SUMMARY OF THE INVENTION

While sonoporation is extensively studied, little remains known regarding the underlying causes of poor efficiency of nucleic acid delivery using the technique. One potential barrier to efficient gene expression proposed herein includes poor nuclear localization of genetic cargos which are transfected to the cell, for example, due to the low tendency of genetic cargos to move through the cytosol and translocate across the nuclear envelope, or due to the clearance of double stranded DNA payloads in the cytosol by the cell's innate immune system prior to nuclear localization. Disclosed and described herein are compositions and methods for overcoming such technical challenges which can increase nuclear localization of genetic payloads delivered to cells using sonoporation, and/or which can reduce the activation of the cell's innate immune system and resulting clearance of the genetic cargo.

Aspects disclosed herein provide a method for delivering a nucleic acid to a target cell of a subject, the method comprising: administering to the subject (1) a microbubble and (2) the nucleic acid comprising a cargo polynucleotide and an aptamer; and applying ultrasonic acoustic energy to the target cell of the subject. Aspects disclosed herein provide a method for delivering a nucleic acid to a target cell of a subject, the method comprising: administering to the subject (1) a sonoactive agent and (2) the nucleic acid, wherein the nucleic acid comprises a cargo polynucleotide and a nuclear localization element; and applying ultrasonic acoustic energy to the target cell of the subject, thereby producing expression of the nucleic acid cargo. Aspects disclosed herein provide a method for delivering a nucleic acid to a target cell of a subject, the method comprising: administering to the subject (1) a sonoactive agent (2) the nucleic acid, wherein the nucleic acid comprises a cargo polynucleotide and an innate immune response avoidance moiety; and applying ultrasonic acoustic energy to the target cell of the subject. Aspects disclosed herein provide a method for expressing a nucleic acid in a target cell of a subject, the method comprising administering to the subject a nucleic acid comprising a cargo polynucleotide and a nuclear localization element, wherein the nuclear localization element increases expression of the cargo polynucleotide in the target cell by at least 1.25 fold as compared to a nucleic acid lacking the nuclear localization element. Aspects disclosed herein provide a method for expressing a nucleic acid in a target cell of a subject, the method comprising administering to the subject a nucleic acid comprising a cargo polynucleotide and an innate immune response avoidance moiety, wherein the innate immune response avoidance moiety increases expression of the cargo polynucleotide in the target cell by at least 1.2 fold as compared to a nucleic acid lacking the immune response avoidance moiety Aspects disclosed herein provide a method for delivering a nucleic acid(s) to a target cell of a subject, the method comprising: administering to the subject (1) a microbubble and (2) the nucleic acid(s) comprising a cargo polynucleotide and an aptamer; and applying ultrasonic acoustic energy to the target cell of the subject. In some embodiments, the nuclear localization element comprises an aptamer. In some embodiments, the nucleic acid further comprises an innate immune response avoidance moiety. In some embodiments, the innate immune response avoidance moiety comprises an aptamer. In some embodiments, the nucleic acid further comprises a nuclear localization element. In some embodiments, the nuclear localization element comprises an aptamer. In some embodiments, the aptamer comprises a sequence configured to promote or perform an intracellular function. In some embodiments, the intracellular function is innate immune response avoidance. In some embodiments, the innate immune response avoidance is reduction of an innate immune response to extra-nuclear DNA. In some embodiments, the intracellular function comprises increasing nuclear localization. In some embodiments, the aptamer comprises a sequence having at least 80% sequence identity to any one of SEQ ID NO: 3-54, or 78-85. In some embodiments, the aptamer comprises a sequence of any one of SEQ ID NO: 3-54, or 78-85. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47. In some embodiments, the sequence configured to bind nucleolin comprises a nucleic acid sequence of any one of SEQ ID NO: 43-47, OR SEQ ID NO: 135-148. In some embodiments, the nuclear localization element or the aptamer comprises a sequence configured to bind importin. In some embodiments, the sequence configured to bind importin comprises a nucleic acid sequence of SEQ ID NO: 48, 129, OR 130. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some embodiments, the aptamer comprises a sequence configured to reduce an innate immune response to extra-nuclear DNA in a cell. In some embodiments, the innate immune response avoidance moiety comprises a cyclic GMP-AMP synthase (cGAS) antagonist, absent in melanoma 2 inflammasome (AIM2) antagonist, or toll-like receptor 9 (TLR9) antagonist. In some embodiments, the aptamer comprises a nucleic acid sequence configured to bind cGAS. In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises any one of SEQ ID NO: 51, 54. In some embodiments, the aptamer comprising the nucleic acid sequence configured to bind cGAS is a cGAS antagonist. In some embodiments, the aptamer comprises a nucleic acid sequence configured to bind an AIM2. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises any one of SEQ ID NO: 51, or 54. In some embodiments, the aptamer comprising the nucleic acid sequence configured to bind AIM2 is an AIM2 antagonist. In some embodiments, the aptamer comprises a nucleic acid sequence configured to bind TLR9. In some embodiments, the sequence configured to bind TLR9 comprises a CpG motif. In some embodiments, the CpG motif comprises any one of SEQ ID NO: 90-93. In some embodiments, the nucleic acid sequence configured to bind TLR9 comprises any one of SEQ ID NO: 51-54. In some embodiments, the aptamer comprising the nucleic acid sequence configured to bind TLR9 is a TLR9 antagonist. In some embodiments, the extra-nuclear DNA comprises DNA located in cytosol in the cell. In some embodiments, the intracellular function comprises increased resistance to one or more intracellular nucleases. In some embodiments, the intracellular function comprises improved transcription of the cargo polynucleotide. In some embodiments, the cargo polynucleotide is covalently coupled to the aptamer, the nuclear localization element, or the innate immune response avoidance moiety. In some embodiments, the cargo polynucleotide is 5′ of the aptamer, the nuclear localization element, or the innate immune response avoidance moiety. In some embodiments, the cargo polynucleotide is 3′ of the aptamer, the nuclear localization element, or the innate immune response avoidance moiety. The nucleic acid delivery vector can comprise a spacer sequence 2015 preceding or following (e.g., 5′ or 3′) of the expression cassette 2035 before the closed end. In some embodiments, the spacer sequence can be at least 3, at least 4, at least 5, at least 6, at least 7, at least 8, at least 9, at least 10, at least 11, at least 12, at least 15, at least 18, at least 21, at least 23, at least 27, or at least 30 nucleotides. In some embodiments, applying ultrasonic acoustic energy to the cell of the subject comprises applying the ultrasound at a first mechanical index (MI) that is less than or equal to 0.4 (e.g., 0<MI≤0.4). in some embodiments, the method includes applying ultrasonic acoustic energy to the cell of the subject at a second MI that is greater than the first MI. In some embodiments, the first MI is about 0.07. In some embodiments, the second MI is at least 1.5. In some embodiments, the second MI is at least 2.0. In some embodiments, the second MI is at least 2.9. In some embodiments, applying ultrasonic acoustic energy to the cell at the second MI comprises applying the ultrasonic acoustic energy in a pulse. In some embodiments, the pulse is less than 2 s. In some embodiments, the pulse is up to 500 microseconds. In some embodiments, the pulse is 1 microsecond to 500 microseconds. In some embodiments, the pulse is about 100-300 microseconds. In some embodiments, the pulse is about 200 microseconds. In some embodiments, the cargo polynucleotide comprises an expression cassette encoding a therapeutic transgene. In some embodiments, the therapeutic transgene is FVIII, COL4A5, or PKD1. In some embodiments, the nucleic acid is a linear DNA construct. In some embodiments, the nucleic acid is a closed linear DNA construct. In some embodiments, the nucleic acid is a closed linear DNA construct comprising at least 2 modified nucleotides. In some embodiments, the nucleic acid is a closed linear DNA construct comprising a stem region comprising a double stranded DNA sequence covalently closed at both ends by hairpin loops. In some embodiments, the hairpin loops are single-stranded. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the at least two modified nucleotides are in one or both single stranded end loops forming the stem region of the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, at least 4, at least 5, at least 6, at least 7, at least 8, at least 9, at least 10, at least 11, at least 12, at least 13, at least 14, 15, at least 16, at least 17, at least 18, at least 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeat(s) sequence (ITR). In some embodiments, the aptamer is positioned between two ITR sequences. In some embodiments, the microbubbles comprise an average diameter of at least 1, 1.5, 2, 2.5, or 3 micron(s). In some embodiments, the method further includes administering ultrasonic acoustic energy to the target cell of the subject. In some embodiments, the method further includes administering a sonoactive agent to the subject. In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or at least 5 fold as compared to a nucleic acid lacking the nuclear localization element. In some embodiments, the innate immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or at least 5 fold as compared to a nucleic acid lacking the nuclear localization element. In some embodiments, the aptamer is a DNA aptamer. In some embodiments, the subject is a mammalian subject. In some embodiments, the nucleic acid(s) is not comprised by or encapsulated within a viral capsid or a viral vector. In some embodiments, the aptamer is a separate nucleic acid construct from the nucleic acid. In some embodiments, the hairpin loops comprise the aptamer. In some embodiments, the nucleic acid(s) are administered simultaneously with the sonoactive agent or microbubble. In some embodiments, the nucleic acid(s) and the sonoactive agent or microbubble are administered in a same intravenous infusion. In some embodiments, the nucleic acid(s) and the sonoactive agent or microbubble are administered in a same pharmaceutical composition. In some embodiments, the nuclear localization element comprises a DNA aptamer, and the aptamer comprises a sequence configured to bind a nucleoporin protein; the cargo polynucleotide comprises an expression cassette encoding a therapeutic transgene; the cargo polynucleotide is covalently coupled to the aptamer; the nucleic acid is a closed linear DNA construct comprising covalently closed at both ends by hairpin loops comprising the aptamer. In some embodiments, the nuclear localization element increases expression of the cargo polynucleotide in the target cell by at least 2.0 fold as compared to a nucleic acid lacking the nuclear localization element. In some embodiments, the innate immune response avoidance moiety comprises a DNA aptamer, and the aptamer comprises a sequence which is a cGAS or a TLR9 antagonist; the cargo polynucleotide comprises an expression cassette encoding a therapeutic transgene; the cargo polynucleotide is covalently coupled to the aptamer; the nucleic acid is a closed linear DNA construct comprising covalently closed at both ends by hairpin loops comprising the aptamer. In some embodiments, the innate immune response avoidance moiety increases expression of the cargo polynucleotide in the target cell by at least 2.0 fold as compared to a nucleic acid lacking the innate immune response avoidance moiety. In some embodiments, the sonoactive agent is a microbubble. In some embodiments, the target cell is a liver cell. In some embodiments, the target cell is a hepatocyte. In some embodiments, the target cell is a LSEC. In some embodiments, the target cell is a kidney cell. In some embodiments, the target cell is a proximal tubular epithelial cell. In some embodiments, the target cell is a podocyte. In some embodiments, the target cell is a muscle cell. In some embodiments, the method is a method to treat a subject in need of a gene therapy or a protein replacement therapy. In some embodiments, the method is a method of treating a mammalian subject having a genetic disorder with a nucleic acid encoding a therapeutic transgene. In some embodiments, the cargo polynucleotide comprises an expression cassette encoding the therapeutic transgene, wherein the therapeutic transgene is configured for expression in the target cell of the subject. In some embodiments, the method is a method for use of the nucleic acid, the sonoactive agent or the microbubble, and the ultrasound in treatment of a subject having a genetic disorder requiring a gene therapy or a protein replacement therapy. In some embodiments, the subject is a subject having Hemophilia A or FVIII deficiency, the nucleic acid encodes FVIII, and the target cell is a liver cell. In some embodiments, the subject is a subject having Alport's Syndrome or COL4A5 deficiency, and the nucleic acid encodes alpha5(IV) chain of collagen IV, and the target cell is a podocyte. In some embodiments, the subject is a subject having PKD1 or polycystin-1 deficiency, and the nucleic acid encodes polycystin-1, and the target cell is a kidney cell. In some embodiments, the subject is a human subject. In some embodiments, the method further includes administering to the subject the sonoactive agent, the nucleic acid, and ultrasound acoustic energy at least a second time at least 24 hours after an initial administration of the sonoactive agent, the nucleic acid, and ultrasound acoustic energy.

Aspects disclosed herein provide a pharmaceutical composition comprising: a microbubble; and a nucleic acid comprising (1) a cargo polynucleotide comprising an expression cassette that comprises a therapeutic transgene and (2) an aptamer, wherein the aptamer comprises a sequence configured to increase nuclear localization. Aspects disclosed herein provide a pharmaceutical composition comprising: a sonoactive agent; a nucleic acid comprising (1) a cargo polynucleotide comprising an expression cassette that encodes a therapeutic transgene and (2) one or both of: (i) a nuclear localization element, and/or (ii) an innate immune response avoidance moiety. In some embodiments, one or both of: (i) the nuclear localization element, or (ii) the innate immune response avoidance moiety, comprise an aptamer. Aspects disclosed herein provide a pharmaceutical composition comprising an isolated nucleic acid comprising a cargo polynucleotide and a nuclear localization element, wherein the nuclear localization element increases expression of the cargo polynucleotide in the target cell by at least 1.25 fold as compared to a nucleic acid lacking the nuclear localization element. Aspects disclosed herein provide a pharmaceutical composition comprising an isolated nucleic acid comprising a cargo polynucleotide and an innate immune response avoidance moiety, wherein the innate immune response avoidance moiety increases expression of the cargo polynucleotide in the target cell by at least 1.25 fold as compared to a nucleic acid lacking the nuclear localization element. Aspects disclosed herein provide a pharmaceutical composition comprising: a sonoactive agent; and nucleic acids comprising (1) a cargo polynucleotide comprising an expression cassette that encodes a therapeutic transgene and (2) one or both of: (i) a nuclear localization element, and/or (ii) an innate immune response avoidance moiety. In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or at least 5 fold as compared to a nucleic acid lacking the nuclear localization element. In some embodiments, the immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or 5 fold as compared to a nucleic acid lacking the immune response avoidance moiety. In some embodiments, the isolated nucleic acid further comprises an innate immune response avoidance moiety. In some embodiments, the isolated nucleic acid further comprises an innate immune response avoidance moiety. In some embodiments, the nucleic acid is up to 40 nucleotides in length. In some embodiments, the nucleic acid is an isolated nucleic acid. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47. In some embodiments, the sequence configured to bind nucleolin comprises a nucleic acid sequence of any one of SEQ ID NO: 43-47, OR SEQ ID NO: 135-148. In some embodiments, the nuclear localization element or the aptamer comprises a sequence configured to bind importin. In some embodiments, the sequence configured to bind importin comprises a nucleic acid sequence of SEQ ID NO: 48, 129, OR 130. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some embodiments, the innate immune response avoidance moiety comprises a cyclic GMP-AMP synthase (cGAS), absent in melanoma 2 inflammasome (AIM2), or toll-like receptor 9 (TLR9) antagonist. In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a nucleic acid sequence configured to bind cGAS. In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises any one of SEQ ID NO: 51, 54. In some embodiments, the aptamer comprising the nucleic acid sequence configured to bind cGAS is a cGAS antagonist. In some embodiments, the aptamer comprises a nucleic acid sequence configured to bind an AIM2. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises any one of SEQ ID NO: 51, or 54. In some embodiments, the aptamer comprising the nucleic acid sequence configured to bind AIM2 is an AIM2 antagonist. In some embodiments, the aptamer comprises a nucleic acid sequence configured to bind TLR9. In some embodiments, the sequence configured to bind TLR9 comprises a CpG motif. In some embodiments, the CpG motif comprises any one of SEQ ID NO: 90-93. In some embodiments, the sequence configured to bind TLR9 comprises any one of SEQ ID NO: 51-54. In some embodiments, the aptamer comprising the nucleic acid sequence configured to bind TLR9 is a TLR9 antagonist. The nucleic acid delivery vector can comprise a spacer sequence 2015 preceding or following (e.g., 5′ or 3′) of the expression cassette 2035 before the closed end. In some embodiments, the spacer sequence can be at least 3, at least 4, at least 5, at least 6, at least 7, at least 8, at least 9, at least 10, at least 11, at least 12, at least 15, at least 18, at least 21, at least 23, at least 27, or at least 30 nucleotides. In some embodiments, the microbubble comprises a protein-stabilized shell. In some embodiments, the protein-stabilized shell comprises albumin. In some embodiments, the cargo polynucleotide comprises an expression cassette encoding a therapeutic transgene. In some embodiments, the nucleic acid is a linear DNA construct. In some embodiments, the nucleic acid is a closed linear DNA construct closed linear DNA construct. In some embodiments, the nucleic acid is a closed linear DNA construct comprising at least 2 modified nucleotides. In some embodiments, the nucleic acid is a closed linear DNA construct comprising a stem region comprising a double stranded DNA sequence covalently closed at both ends by hairpin loops. In some embodiments, the hairpin loops are single-stranded. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the at least two modified nucleotides are in one or both single stranded end loops forming the stem region of the aptamer. In some embodiments, the hairpin loops comprise the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, 4, at least 5, at least 6, at least 7, at least 8, at least 9, at least 10, at least 11, at least 12, at least 13, at least 14, 15, at least 16, at least 17, at least 18, at least 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeats (ITR). Aspects disclosed herein provide a pharmaceutical composition for use in treating a subject having a genetic disorder requiring a gene therapy or a protein replacement therapy. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1.

Aspects disclosed herein provide a nucleic acid comprising a sequence having at least 80% sequence identity to SEQ ID NO: 49 or 50. In some embodiments, the nucleic acid comprises at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to SEQ ID NO: 49 or 50. In some embodiments, the nucleic acid comprises sequence of SEQ ID NO: 49 or 50. In some embodiments, the nucleic acid is an aptamer configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleic acid is an aptamer configured to bind NUP 358. In some embodiments, the nucleic acid is DNA or a DNA aptamer. Aspects disclosed herein provide a nucleic acid for use in treating a subject having a genetic disorder requiring a gene therapy or a protein replacement therapy. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1.

Aspects disclosed herein provide a kit comprising: nucleic acids comprising (1) a cargo polynucleotide comprising an expression cassette that encodes a therapeutic transgene and (2) one or both of: (i) a nuclear localization element, and/or (ii) an innate immune response avoidance moiety; and a sonoactive agent. In some embodiments, the kit further includes instructions for applying ultrasound acoustic energy to a subject, wherein the ultrasound acoustic energy is configured to deliver the nucleic acids to the cell. In some embodiments, the ultrasound acoustic energy is configured to deliver the nucleic acids to the cell. In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or 5 fold as compared to a nucleic acid lacking the nuclear localization element. In some embodiments, the immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25, at least 2.5, at least 2.75. 3, at least 3.5, at least 4, at least 4.5, or 5 fold as compared to a nucleic acid lacking the immune response avoidance moiety. In some embodiments, one or both of: (i) the nuclear localization element, or (ii) the innate immune response avoidance moiety, comprise an aptamer. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47, OR SEQ ID NO: 135-148. In some embodiments, the aptamer comprises a sequence having at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to any one of SEQ ID NO: 3-54 or 78-85. In some embodiments, the nuclear localization element or the aptamer comprises a sequence configured to bind importin. In some embodiments, the sequence configured to bind importin comprises a nucleic acid sequence of SEQ ID NO: 48, 129, or 130. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some embodiments, the innate immune response avoidance moiety comprises a cyclic GMP-AMP synthase (cGAS), absent in melanoma 2 inflammasome (AIM2), or toll-like receptor 9 (TLR9) antagonist. In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a nucleic acid sequence configured to bind cGAS. In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises any one of SEQ ID NO: 51, 54. In some embodiments, the aptamer comprising the nucleic acid sequence configured to bind cGAS is a cGAS antagonist. In some embodiments, the aptamer comprises a nucleic acid sequence configured to bind an AIM2. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises any one of SEQ ID NO: 51, or 54. In some embodiments, the aptamer comprising the nucleic acid sequence configured to bind AIM2 is an AIM2 antagonist. In some embodiments, the aptamer comprises a nucleic acid sequence configured to bind TLR9. In some embodiments, the sequence configured to bind TLR9 comprises a CpG motif. In some embodiments, the CpG motif comprises any one of SEQ ID NO: 90-93. In some embodiments, the sequence configured to bind TLR9 comprises any one of SEQ ID NO: 51-54. In some embodiments, the aptamer comprising the nucleic acid sequence configured to bind TLR9 is a TLR9 antagonist. In some embodiments, the sonoactive agent comprises a microbubble. In some embodiments, the sonoactive agent comprises a protein-stabilized shell. In some embodiments, the sonoactive agent comprises a lipid stabilized shell. In some embodiments, the cargo polynucleotide is covalently coupled to one or both of: (i) a nuclear localization element, and/or (ii) an innate immune response avoidance moiety. In some embodiments, the nucleic acid comprising the cargo polynucleotide is a linear DNA construct. In some embodiments, the nucleic acid comprising the cargo polynucleotide is a closed linear DNA construct closed linear DNA construct. In some embodiments, the nucleic acid comprising the cargo polynucleotide is a closed linear DNA construct comprising at least 2 modified nucleotides. In some embodiments, the nucleic acid comprising the cargo polynucleotide is a closed linear DNA construct comprising a stem region comprising a double stranded DNA sequence covalently closed at both ends by hairpin loops. In some embodiments, the hairpin loops are single-stranded. In some embodiments, the hairpin loops comprise any one of SEQ ID NO: 55-62, 101-128, or 205-252. In some embodiments, a first hairpin loop of the hairpin loops comprise any one of SEQ ID NO: 55-62, 101-128, or 205-252, and wherein a second hairpin loop of the hairpin loops comprise a different sequence than the first hairpin loop of any one of SEQ ID NO: 55-62, 101-128, or 205-252. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the hairpin loops comprise the aptamer. In some embodiments, the at least two modified nucleotides are in one or both single stranded end loops forming the stem region of the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, 4, at least 5, at least 6, at least 7, at least 8, at least 9, at least 10, at least 11, at least 12, at least 13, at least 14, at least 15, at least 16, at least 17, at least 18, at least 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeat(s) (ITR) sequence.

Aspects disclosed herein provide an isolated nucleic acid comprising: a cargo polynucleotide comprising an expression cassette that encodes a therapeutic transgene configured for expression in a target cell of a subject, and a nuclear localization element configured to increase expression of the cargo polynucleotide in the target cell. In some embodiments, the nuclear localization element increases expression of the cargo polynucleotide in the target cell by at least 1.25 fold as compared to a nucleic acid lacking the nuclear localization element. In some embodiments, the nuclear localization element comprises an aptamer. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47. In some embodiments, the sequence configured to bind nucleolin comprises a nucleic acid sequence of any one of SEQ ID NO: 43-47, OR SEQ ID NO: 135-148. OR SEQ ID NO: 43-48, or SEQ ID NO: 48, 129, OR 130 In some embodiments, the nuclear localization element or the aptamer comprises a sequence configured to bind importin. In some embodiments, the sequence configured to bind importin comprises a nucleic acid sequence of SEQ ID NO: 48, 129, or 130. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some embodiments, the cargo polynucleotide is covalently coupled to the nuclear localization element. In some embodiments, the isolated nucleic acid comprising the cargo polynucleotide is a linear DNA construct. In some embodiments, the isolated nucleic acid comprising the cargo polynucleotide is a closed linear DNA construct closed linear DNA construct. In some embodiments, the isolated nucleic acid comprise rising the cargo polynucleotide is a closed linear DNA construct comprising at least 2 modified nucleotides. In some embodiments, the isolated nucleic acid comprise the cargo polynucleotide is a closed linear DNA construct comprising a stem region comprising a double stranded DNA sequence covalently closed at both ends by hairpin loops. In some embodiments, the hairpin loops are single-stranded. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the at least two modified nucleotides are in one or both single stranded end loops forming the stem region of the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, at least 4, at least 5, at least 6, at least 7, at least 8, at least 9, at least 10, at least 11, at least 12, at least 13, at least 14, 15, at least 16, at least 17, at least 18, at least 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeat(s) (ITR) sequence(s). In some embodiments, the ITR sequence(s) are located in the stem region of the aptamer. In some embodiments, the second aptamer comprises a different nucleic acid sequence than the nuclear localization element which comprises the aptamer. In some embodiments, the isolated nucleic acid comprises a spacer sequence preceding or following (e.g., 5′ or 3′) of the expression cassette before the hairpin loop(s). In some embodiments, the spacer sequence is at least 3, at least 4, at least 5, at least 6, at least 7, at least 8, at least 9, at least 10, at least 11, at least 12, at least 15, at least 18, at least 21, at least 23, at least 27, or at least 30 nucleotides. In some embodiments, the aptamer(s) are comprised within the hairpin loop(s). In some embodiments, the aptamer(s) are at least partially single stranded. In some embodiments, the aptamer(s) comprise any one of SEQ ID NO: 3-54, 129-130, or 135-148. In some embodiments, the isolated nucleic acid comprises a spacer sequences preceding and following (e.g., 5′ or 3′) of the expression cassette before the hairpin loop(s). In some embodiments, the hairpin loops comprise the aptamer. In some embodiments, the isolated nucleic acid is configured to form an episome in a nucleus of the cell. In some embodiments, the therapeutic transgene configured for expression in the target cell is an exogenous transgene to the subject. In some embodiments, the exogenous transgene provides a gain of function to the subject by expression of the therapeutic transgene. In some embodiments, the expression cassette is at least 4.5, 5, at least 6, at least 7, at least 8, at least 9, at least 10, at least 11, at least 12, at least 13, at least 14, or 15 kb long. In some embodiments, the isolated nucleic acid is at least 4.5, at least 5, at least 6, at least 7, at least 8, at least 9, at least 10, at least 11, at least 12, at least 13, at least 14, or 15 kb long. In some embodiments, the therapeutic transgene encodes FVIII, FIX, alpha5(IV) chain of collagen IV, alpha4(IV) chain of collagen IV, alpha3(IV) chain of collagen IV, protein polycystin-1 (PC1), or polycystin-2 protein (PC2). In some embodiments, the therapeutic transgene is FVIII, FIX, COL4A3, COL4A5, COL4A4, PKD1, or PKD2. Aspects disclosed herein provide an isolated nucleic acid for use in treating a subject having a genetic disorder requiring a gene therapy or a protein replacement therapy. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1. Aspects disclosed herein provide an isolated nucleic acid encoding any one of SEQ ID NO: 1-253.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a nucleic acid comprising a cargo polynucleotide and an aptamer modulating intracellular function;

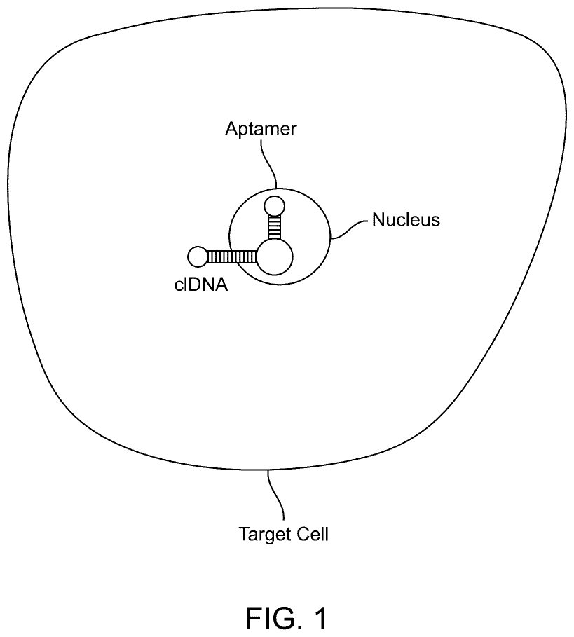

FIG. 2 illustrates an embodiment of a nucleic acid delivery vector of the present disclosure;

FIG. 3 illustrates an embodiment of a nucleic acid delivery vector of the present disclosure;

FIG. 4 illustrates the nuclear pore complex and import of cargo through the nuclear pore complex;

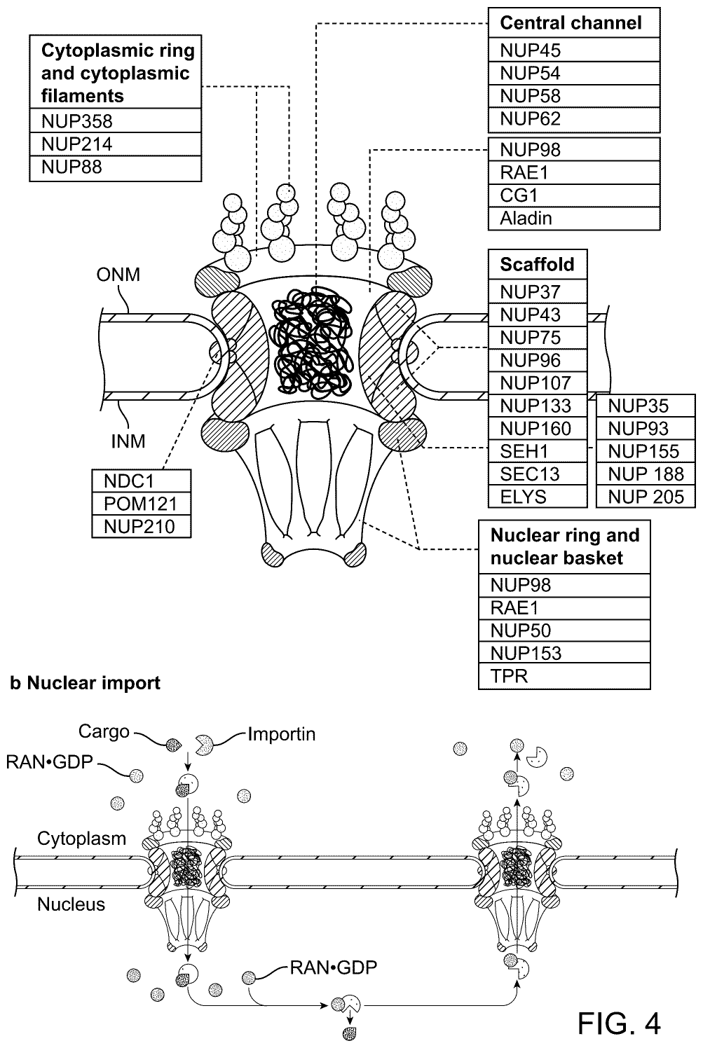

FIG. 5 provides data illustrating in-vitro gene expression resulting from transfection with nucleic acid delivery vectors of the present disclosure;

FIG. 6 illustrates a process in which embodiments of nucleic acid delivery vectors of the present disclosure are manufactured;

FIG. 7 illustrates a conformation of an aptamer of the present disclosure;

FIG. 8 illustrates a conformation of an aptamer of the present disclosure;

FIG. 9 illustrates a conformation of an aptamer of the present disclosure;

FIG. 10 provides data illustrating in-vivo gene expression resulting from sonoporation of a murine liver transfecting nucleic acid delivery vectors of the present disclosure;

FIG. 11 illustrates the cGAS-STING pathway of the innate immune system for sensing and clearance of cytosolic DNA; and

FIG. 12 provides data illustrating in-vivo gene expression resulting from sonoporation of a murine liver transfecting nucleic acid delivery vectors with aptamers of the present disclosure.

### DETAILED DESCRIPTION OF THE INVENTION

While sonoporation is extensively studied, little remains known regarding the underlying causes of poor efficiency of nucleic acid delivery using the technique. One potential barrier to efficient gene expression proposed herein includes poor nuclear localization of genetic cargos which are transfected to the cell, for example, due to the low tendency of genetic cargos to move through the cytosol and translocate across the nuclear envelope, or due to the clearance of double stranded DNA payloads in the cytosol by the cell's innate immune system prior to nuclear localization. Disclosed and described herein are compositions and methods for overcoming such technical challenges which can increase nuclear localization of genetic payloads delivered to cells using sonoporation, and/or which can reduce the activation of the cell's innate immune system and resulting clearance of the genetic cargo.

Translocating a DNA payload across the nuclear envelope presents numerous technical challenges due to the complex architecture of the nuclear envelope, the characteristics of DNA molecules, and the mechanisms involved in nuclear transport, many of which have evolved in mammalian cells as defense mechanisms against injection by viral, bacterial, and fungal injections. The nuclear envelope is a double lipid bilayer structure that is formed from the outer nuclear membrane, which is generally continuous with the endoplasmic reticulum, and the inner nuclear membrane, which interacts with the nuclear lamina and chromatin DNA within the nucleus. Embedded within the nuclear envelope are nuclear pore complexes and nucleoporin proteins, which regulate molecular traffic between the cytoplasm and the nucleus. While small molecules can pass through nuclear pore complex via passive diffusion, larger macromolecules such as DNA generally require active transport mechanisms. DNA, including encapsulated DNA, is much larger than the cargo typically allowed by nuclear pore complex, and the large size of unencapsulated DNA often impedes efficient translocation. DNA further carries a negative charge due to its phosphate backbone, which complicates its passage through the cellular environment and the nuclear pore complexes (NPCs), as nuclear import mechanisms are not optimized for such charged macromolecules. Nuclear import of large molecules generally requires nuclear localization signals that are recognized by transport proteins like importins, which facilitate their movement through nucleoporin complexes. However, unencapsulated DNA administered through sonoporation, as is known within the art, lacks these nuclear localization signals, necessitating improvements to the art to facilitate transportation into the nucleus.

Described herein are methods and compositions which are adapted for increasing nuclear localization of genetic payloads in cell. Aspects disclosed herein provide a method for delivering a nucleic acid to a target cell of a subject, the method comprising: administering to the subject (1) a sonoactive agent and (2) the nucleic acid, wherein the nucleic acid comprises a cargo polynucleotide and a nuclear localization element; and applying ultrasonic acoustic energy to the target cell of the subject, thereby producing expression of the nucleic acid cargo. Aspects disclosed herein provide a method for delivering a nucleic acid(s) to a target cell of a subject, the method comprising: administering to the subject (1) a microbubble and (2) the nucleic acid(s) comprising a cargo polynucleotide and an aptamer; and applying ultrasonic acoustic energy to the target cell of the subject. Aspects disclosed herein provide a pharmaceutical composition comprising: a sonoactive agent; a nucleic acid comprising (1) a cargo polynucleotide comprising an expression cassette that encodes a therapeutic transgene and (2) a nuclear localization element. In some embodiments, the nuclear localization element comprises an aptamer. Aspects disclosed herein provide a method for expressing a nucleic acid in a target cell of a subject, the method comprising administering to the subject a nucleic acid comprising a cargo polynucleotide and a nuclear localization element, wherein the nuclear localization element increases expression of the cargo polynucleotide in the target cell by at least 1.25 fold as compared to a nucleic acid lacking the nuclear localization element.

In some cases, the nuclear localization element is configured to bind an importin protein which facilitates transport of the cargo polynucleotide comprising an expression cassette that comprises a therapeutic transgene into nucleus. In some embodiments, the nuclear localization element or the aptamer comprises a sequence configured to bind importin. In some embodiments, the sequence configured to bind importin comprises a nucleic acid sequence of SEQ ID NO: 3-48, OR SEQ ID NO: 43-48, or SEQ ID NO: 48, 129, OR 130. In some embodiments, the aptamer comprises a sequence having at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to any one of SEQ ID NO: 3-54 or 78-85 OR SEQ ID NO: 43-48, or SEQ ID NO: 48, 129, OR 130. In some cases, the nuclear localization element or the aptamer comprising the sequence configured to bind importin provides a beneficial technical effect of increasing nuclear localization and resulting gene expression of the cargo polynucleotide encoding a therapeutic transgene.

In some embodiments, the nuclear localization element or the aptamer comprises a sequence configured to bind nucleolin. In some cases, the nuclear localization element is configured to bind a nucleolin protein primarily found in the dense fibrillar regions of the nucleus, which facilitates transport of the cargo polynucleotide comprising an expression cassette that comprises a therapeutic transgene into nucleus. In some embodiments, the sequence configured to bind nucleolin comprises a nucleic acid sequence of any one of SEQ ID NO: 3-47. In some embodiments, the aptamer comprises a sequence having at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to any one of SEQ ID NO: 3-54 or 78-85. In some embodiments, the sequence configured to bind nucleolin comprises a nucleic acid sequence of any one of SEQ ID NO: 43-47, OR SEQ ID NO: 135-148. In some cases, the nuclear localization element or the aptamer comprising the sequence configured to bind nucleolin provides a beneficial technical effect of increasing nuclear localization and resulting gene expression of the cargo polynucleotide encoding a therapeutic transgene.

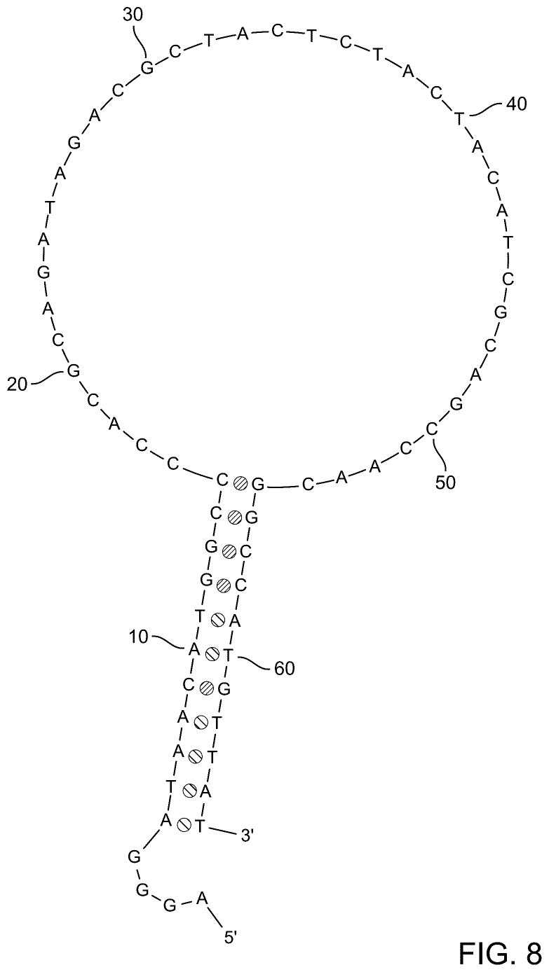

In some cases, the nuclear localization element is configured to bind a nuclear pore or a nucleoporin protein to facilitate transport of the cargo polynucleotide comprising an expression cassette that comprises a therapeutic transgene into nucleus. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. The aptamer encoded by SEQ ID NO: 50 is shown in conformational form in FIG. 8. In some cases, the nuclear localization element or the aptamer comprising the sequence configured to bind a nucleoporin protein provides a beneficial technical effect of increasing nuclear localization of the nucleic acid and resulting gene expression of the cargo polynucleotide encoding a therapeutic transgene. In some embodiments, the method further includes administering ultrasonic acoustic energy to the target cell of the subject. In some embodiments, the method further includes administering a sonoactive agent to the subject. In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or 5 fold as compared to a nucleic acid lacking the nuclear localization element. In some embodiments, the nuclear localization element comprises a DNA aptamer, and the aptamer comprises a sequence configured to bind a nucleoporin protein; the cargo polynucleotide comprises an expression cassette encoding a therapeutic transgene; the cargo polynucleotide is covalently coupled to the aptamer; the nucleic acid is a closed linear DNA construct comprising covalently closed at both ends by hairpin loops comprising the aptamer. In some embodiments, the nuclear localization element increases expression of the cargo polynucleotide in the target cell by at least 2.0 fold as compared to a nucleic acid lacking the nuclear localization element.

Aspects provided herein include sequences of nucleic acids and DNA aptamers which bind nucleoporin proteins, for example, NUP358. Aspects disclosed herein provide a nucleic acid comprising a sequence having at least 80% sequence identity to SEQ ID NO: 49 or 50. In some embodiments, the sequence of the nucleic acid has at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to SEQ ID NO: 49 or 50. In some embodiments, the sequence of the nucleic acid comprises the sequence of SEQ ID NO: 49 or 50. In some embodiments, the nucleic acid comprises an aptamer configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleic acid is an aptamer configured to bind NUP358. In some embodiments, the nucleic acid is DNA and/or comprises a DNA aptamer. Aspects disclosed herein provide a nucleic acid for use in treating a subject having a genetic disorder requiring a gene therapy or a protein replacement therapy. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1.

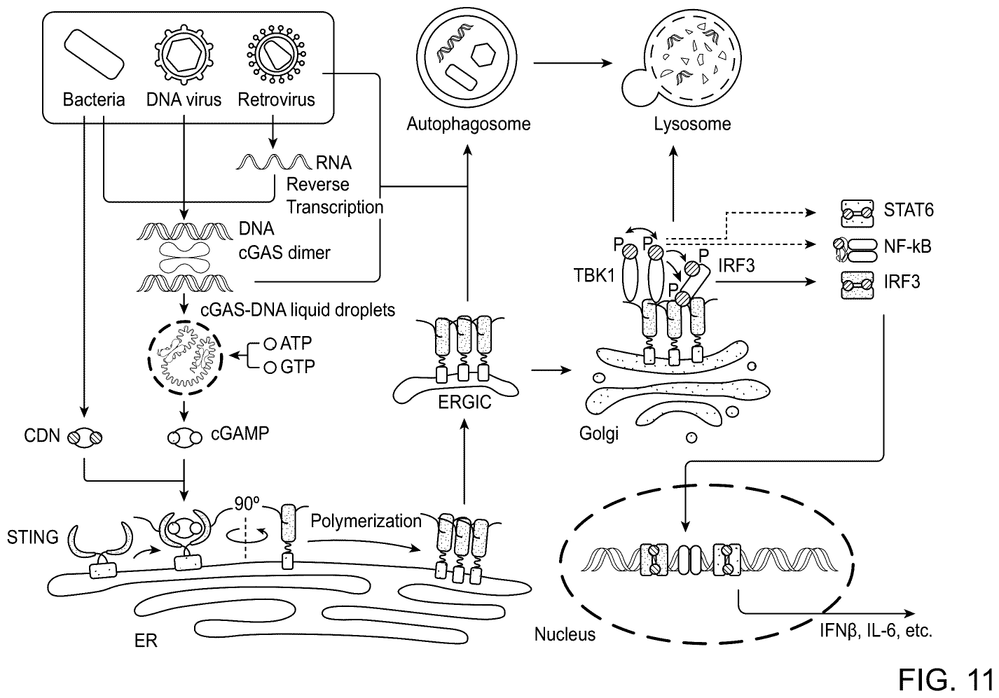

In addition to challenges associated with nuclear localization, the delivery of DNA to cells faces significant challenges due to the innate immune system's ability to recognize and clear foreign genetic material. The innate immune system is the body's first line of defense, and it has evolved to detect and eliminate potential threats, including exogenous DNA which biologically is delivered to the cell because of microbial infection. The innate immune system is equipped with pattern recognition receptors such as Toll-like receptors (TLRs) and cytosolic DNA sensors such as cyclic GMP-AMP synthase (cGAS), which detect foreign DNA. For example, sensing to cytosolic DNA can include binding of double stranded cytosolic DNA by cGAS, which can lead to a signaling cascade of an immune response including synthesis of a special asymmetric cyclic-dinucleotide, 2′3′-cGAMP, and activation of STING (endoplasmic reticulum (ER) membrane protein) for subsequent production of type I interferons and other immune-modulatory genes, as is illustrated in FIG. 11. In addition to type I IFN production, activation of the cGAS-STING pathway can also lead to the production of pro-inflammatory cytokines, such as tumor necrosis factor alpha (TNF-α) and interleukin-6 (IL-6), induction of cellular autophagy, activation of activation of caspase-1 and subsequent processing and secretion of pro-inflammatory cytokines such as interleukin-1 beta (IL-1β) and interleukin-18 (IL-18), and activation of apoptotic pathways. Other aspects of the innate immune system include TLR9 activation in endosomes which can recognize unmethylated CpG motifs in bacterial or synthetic DNA, triggering immune responses that activate inflammatory cytokines and interferons. Once exogenous cytosolic DNA is detected by any mechanism, the immune system launches a powerful inflammatory response, involving the production of type I interferons and other cytokines, working to activate immune cells such as natural killer (NK) cells, macrophages, and dendritic cells to clear the DNA and destroy transfected cells. In the context of a sonoporation-based gene therapy, this can result in clearance of DNA delivered to a cell and resulting gene expression.

Described herein are methods and compositions which are adapted for evading the cell's innate immune system for clearing exogenous genetic cargos. Aspects disclosed herein provide a method for delivering a cargo polynucleotide to a target cell of a subject. Aspects disclosed herein provide a method for delivering a nucleic acid to a target cell of a subject, the method comprising: administering to the subject (1) a sonoactive agent (2) the nucleic acid, wherein the nucleic acid comprises a cargo polynucleotide and an innate immune response avoidance moiety; and applying ultrasonic acoustic energy to the target cell of the subject. In some embodiments, the innate immune response avoidance moiety comprises an aptamer. Aspects disclosed herein provide a method for expressing a nucleic acid in a target cell of a subject, the method comprising administering to the subject a nucleic acid comprising a cargo polynucleotide and an innate immune response avoidance moiety, wherein the innate immune response avoidance moiety increases expression of the cargo polynucleotide in the target cell by at least 1.2 fold as compared to a nucleic acid lacking the immune response avoidance moiety. In some embodiments, the innate immune response avoidance moiety comprises a DNA aptamer, and the aptamer comprises a sequence which is a cGAS or a TLR9 antagonist; the cargo polynucleotide comprises an expression cassette encoding a therapeutic transgene; the cargo polynucleotide is covalently coupled to the aptamer; the nucleic acid is a closed linear DNA construct comprising covalently closed at both ends by hairpin loops comprising the aptamer. In some embodiments, the innate immune response avoidance moiety increases expression of the cargo polynucleotide in the target cell by at least 2.0 fold as compared to a nucleic acid lacking the innate immune response avoidance moiety. In some embodiments, the sonoactive agent is a microbubble.

In some embodiments, the aptamer comprises a sequence configured to reduce an innate immune response to extra-nuclear DNA in a cell. In some embodiments, the innate immune response avoidance moiety comprises a cyclic GMP-AMP synthase (cGAS) antagonist. In some embodiments, the aptamer comprises a nucleic acid sequence configured to bind cGAS. In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises SEQ ID NO: 51 or 54. In some embodiments, the aptamer comprising the nucleic acid sequence configured to bind cGAS is a cGAS antagonist. In some cases, the innate immune response avoidance moiety or the aptamer comprising the sequence configured to bind cGAS competes with cGAS binding by the exogenous DNA, and acts as a cGAS antagonist, prevents activation of cGAS by the exogenous DNA, and reduces or eliminates clearance of the nucleic acid from the cell. In some cases, the innate immune response avoidance moiety or the aptamer comprising the sequence configured to bind cGAS provides a beneficial technical effect of reducing clearance of the nucleic acid by the cell and increasing gene expression of the cargo polynucleotide encoding a therapeutic transgene.

In some embodiments, the aptamer comprises a sequence configured to reduce an innate immune response to extra-nuclear DNA in a cell. In some embodiments, the innate immune response avoidance moiety comprises an absent in melanoma 2 inflammasome (AIM2) antagonist. In some embodiments, the aptamer comprises a nucleic acid sequence configured to bind an AIM2. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises SEQ ID NO: 51 or 54. In some embodiments, the aptamer comprising the nucleic acid sequence configured to bind AIM2 is an AIM2 antagonist. In some cases, the innate immune response avoidance moiety or the aptamer comprising the sequence configured to bind AIM2 competes for binding to the cytosolic DNA sensor AIM2, and acts as an AIM2 antagonist, prevents activation of AIM2 by the exogenous DNA, and reduces or eliminates clearance of the nucleic acid from the cell. In some cases, the innate immune response avoidance moiety or the aptamer comprising the sequence configured to bind AIM2 provides a beneficial technical effect of reducing clearance of the nucleic acid by the cell and increasing gene expression of the cargo polynucleotide encoding a therapeutic transgene.

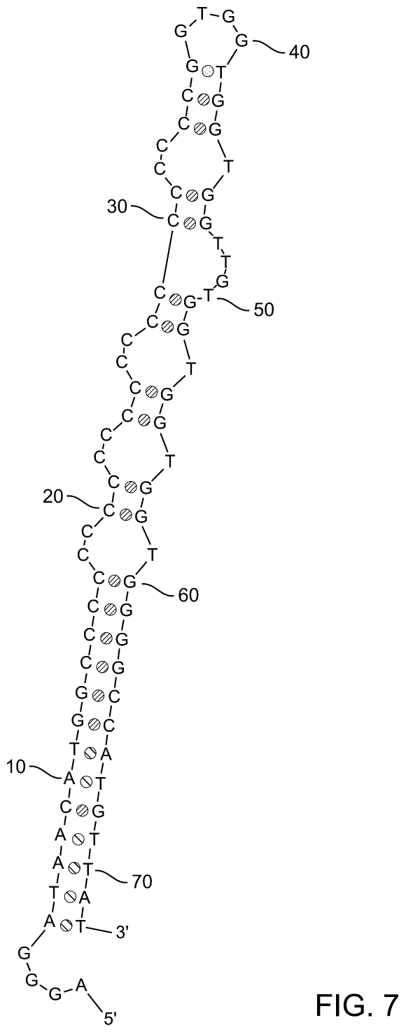

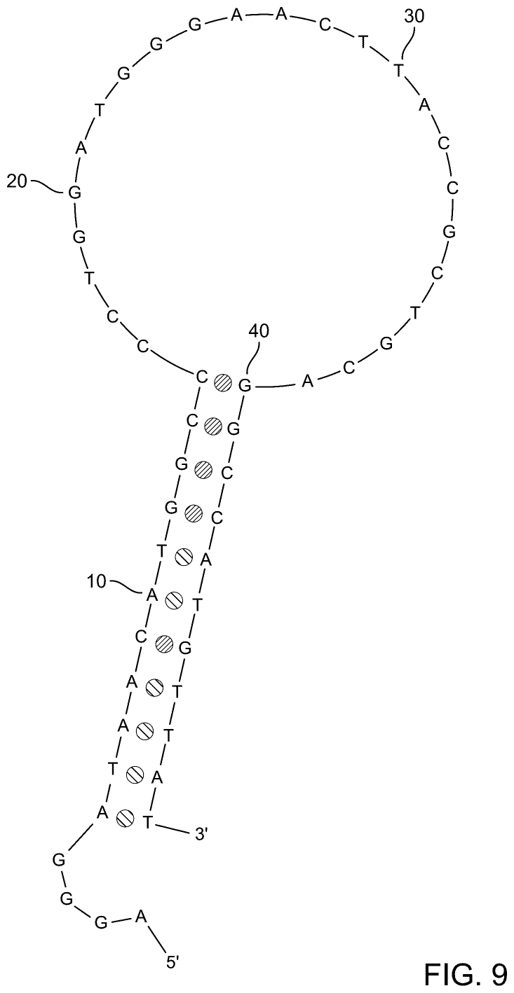

In some embodiments, the aptamer comprises a sequence configured to reduce an innate immune response to extra-nuclear DNA in a cell. In some embodiments, the aptamer comprises a nucleic acid sequence configured to bind TLR9. In some embodiments, the innate immune response avoidance moiety comprises a toll-like receptor 9 (TLR9) antagonist. In some embodiments, the sequence configured to bind TLR9 comprises a CpG motif. In some embodiments, the CpG motif comprises any one of SEQ ID NOS: 90-93. In some embodiments, the nucleic acid sequence configured to bind TLR9 comprises any one of SEQ ID NOS: 51-54. An exemplary aptamer encoded by SEQ ID NO: 53 configured to bind TLR9 is shown in FIG. 7. As is shown in FIGS. 7-9, the aptamers may comprise a double stranded stem region 10 and a single stranded region 20 which has a conformational form in which the aptamer will bind the target antigen. In some embodiments, the aptamer comprising the nucleic acid sequence configured to bind TLR9 is a TLR9 antagonist. In some cases, the innate immune response avoidance moiety or the aptamer comprising the sequence is an antagonist of TLR9 and prevents TLR9-mediated activation of B-cells and macrophages which would work to clear the exogenous DNA, and reduces or eliminates clearance of the nucleic acid from the cell. In some cases, the innate immune response avoidance moiety or the aptamer comprising the sequence configured to bind TLR9 provides a beneficial technical effect of reducing clearance of the nucleic acid by the cell and increasing gene expression of the cargo polynucleotide encoding a therapeutic transgene.

In some embodiments, the innate immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, or 5 fold as compared to a nucleic acid lacking the nuclear localization element. In some embodiments, the aptamer is a DNA aptamer. In some embodiments, the subject is a mammalian subject. In some embodiments, the nucleic acid(s) is not comprised by or encapsulated within a viral capsid or a viral vector. In some embodiments, the aptamer is a separate nucleic acid construct from the nucleic acid. In some embodiments, the hairpin loops comprise the aptamer. In some embodiments, the nucleic acid(s) are administered simultaneously with the sonoactive agent or microbubble. In some embodiments, the nucleic acid(s) and the sonoactive agent or microbubble are administered in a same intravenous infusion. In some embodiments, the nucleic acid(s) and the sonoactive agent or microbubble are administered in a same pharmaceutical composition.

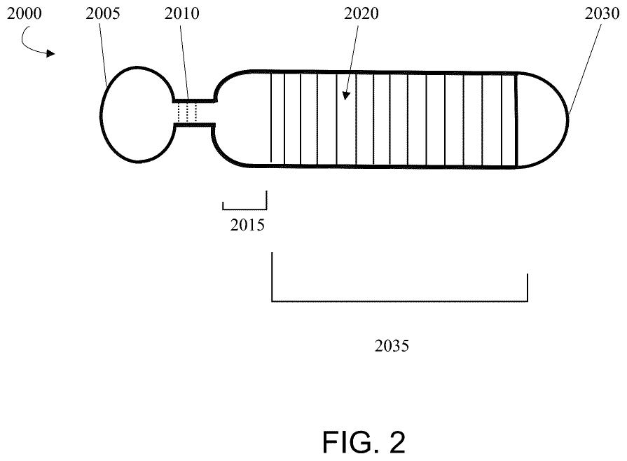

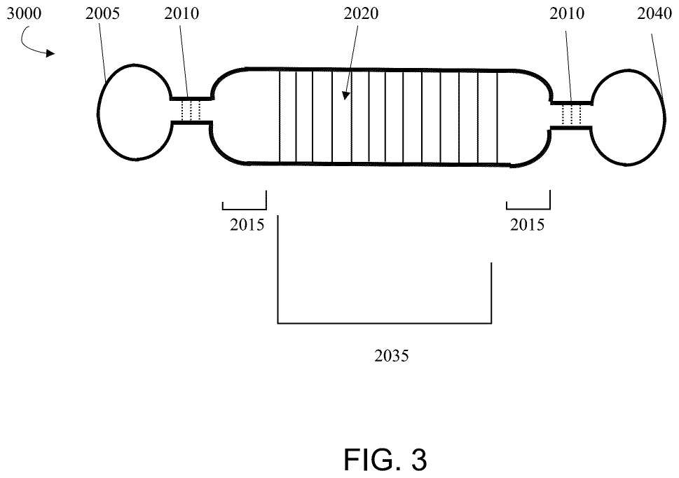

Illustrated in FIGS. 2-3 are nucleic acid delivery vectors of the present disclosure which comprise a (1) a cargo polynucleotide comprising an expression cassette that encodes a therapeutic transgene and (2) one or more of: i) a nuclear localization element, ii) a immune response avoidance moiety; or iii) an aptamer. Referring to FIG. 2, the nucleic acid delivery vector can be a linear nucleic acid delivery vector 2000 with a closed end(s) 2030. The nucleic acid delivery vector can comprise an expression cassette 2035 encoding a therapeutic transgene in the double stranded portion 2020 of the vector in which the sense strand is complementarily paired to the antisense strand. The nucleic acid delivery vector can comprise a spacer sequence 2015 preceding or following (e.g., 5′ or 3′) of the expression cassette 2035 before the closed end on a given strand (e.g., the sense strand). In some embodiments, the spacer sequence can be at least 3, at least 4, at least 5, at least 6, at least 7, at least 8, at least 9, at least 10, at least 11, at least 12, at least 15, at least 18, at least 21, at least 23, at least 27, or at least 30 nucleotides. In some cases, the spacer sequence provides a beneficial technical effect of reducing steric hindrance of the polynucleotide chain, permitting the proper base pairing of the aptamer 2005 to form its intended 3D conformation (see, e.g., FIGS. 7-9) to allow for the aptamer to bind its target antigen or moiety. In some cases, the nucleic acid delivery vector can be closed by an aptamer 2005. The aptamer may comprise a stem region 2010 which is formed from inverted terminal repeat sequences which are complementarily paired to another. Referring to FIG. 3, an alternative nucleic acid delivery vector 3000 can be closed by an aptamer 2005/2040 at each end. In some cases, a second aptamer 2040 which comprises a different nucleic acid sequence and which is configured to bind a different moiety or antigen can be on the second end of the nucleic acid delivery vector 3000. In some embodiments the aptamer 2005/2040 at a first end is the nuclear localization element, and the aptamer 2005/2040 at the second end is the innate immune response avoidance moiety. In some embodiments the aptamers 2005/2040 at a first end and the second end are the same.

In some embodiments, the cargo polynucleotide is covalently coupled to the aptamer, the nuclear localization element, or the innate immune response avoidance moiety. In some embodiments, the cargo polynucleotide is 5′ of the aptamer with reference to the sense strand, the nuclear localization element, or the innate immune response avoidance moiety. In some embodiments, the cargo polynucleotide is 3′ of the aptamer, the nuclear localization element, or the innate immune response avoidance moiety with reference to the sense strand.

In some embodiment, the aptamer is configured to promote an intracellular function. In some embodiments, the intracellular function comprises increased resistance to one or more intracellular nucleases. In some embodiments, the intracellular function comprises improved transcription of the cargo polynucleotide.

Aspects disclosed herein provide a nucleic acid for use in treating a subject having a genetic disorder requiring a gene therapy or a protein replacement therapy. In some embodiments, the subject is a subject having Hemophilia A or FVIII deficiency, and the nucleic acid encodes FVIII. In some embodiments, the subject is a subject having Alport's Syndrome or COL4A5 deficiency, and the nucleic acid encodes alpha5(IV) chain of collagen IV. In some embodiments, the subject is a subject having PKD1 or polycystin-1 deficiency, and the nucleic acid encodes polycystin-1.

In some embodiments, the method further includes administering to the subject the sonoactive agent, the nucleic acid, and ultrasound acoustic energy at least a second time at least 24 hours after an initial administration of the sonoactive agent, the nucleic acid, and ultrasound acoustic energy.

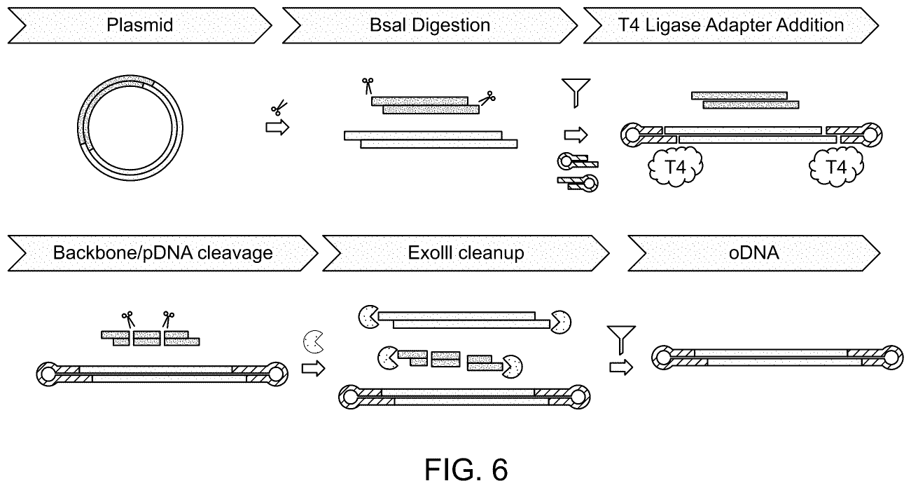

Referring to FIG. 6, the nucleic acid delivery vectors of the present disclosure can be synthesized in an exemplary process beginning from double-stranded circular DNA. For example, a plasmid encoding an expression cassette of interest can be chemically synthesized, while single stranded DNA encoding the aptamer sequence of interest can be chemically synthesized, using methods known within the art. The plasmid DNA can be linearized with endonuclease digestion, e.g., BasI digestion, which can cut endonuclease recognition sites within the pDNA. The aptamer adaptors can by synthesized to comprise complementary sequences to the overhang sequences which are left by the endonuclease digestion, and the hybridized linear DNA molecule can be ligated using a ligase, for example, T4 ligase. After ligation, remaining backbone, aptamer sequences, and pDNA can be digested using additional endonucleases (e.g., Nhel, Pcil), and then treated with yet additional endonuclease to digest any remaining DNA which is not closed at each end (e.g., ExoIII). The remaining product can then be purified, concentrated, and stored or sequenced as necessary.

Aptamers of the present disclosure can be identified using a SELEX (Systematic Evolution of Ligands by Exponential Enrichment) technique, to identify high-affinity aptamers that specifically bind to a target antigen, for example, the Nup358 protein. The process can begin with the synthesis and immobilization of the target antigen on a solid support, such as NHS-activated Sepharose beads or high-binding plates, ensuring proper protein folding and functionality. A randomized nucleic acid library, composed of single-stranded DNA (ssDNA) or RNA, can be prepared with random nucleotide sequences of 20-80 bases, along with primer-binding sites for later amplification. To promote correct structural formation, the library can be denatured by heating to 95° C. and then rapidly cooled. These nucleic acids may be then incubated with the immobilized target antigen in an appropriate buffer system that may include components like Tris-HCl, NaCl, KCl, and MgCl₂ to maintain aptamer-protein interactions.

Following incubation, the unbound or weakly bound nucleic acids may be washed away using buffers of increasing ionic strength to improve the specificity of binding. The tightly bound aptamers may be eluted using either a high-salt buffer or a low-pH buffer, depending on the nature of the binding interaction. For RNA aptamers, mild heating may be used during elution to avoid degradation. The eluted aptamers may be then amplified through PCR (for ssDNA) or reverse transcription followed by PCR (for RNA), ensuring that the enriched sequences may be available for the next round of SELEX. The SELEX process can be carried out iteratively, typically over 8-12 rounds, with each cycle involving incubation with the target protein, washing, elution, and amplification. Over successive rounds, the washing stringency can be increased to enrich for aptamers with higher affinity and specificity for the target antigen. The progress of enrichment can be tracked using techniques such as fluorescence anisotropy, surface plasmon resonance (SPR), or electrophoretic mobility shift assays (EMSA), which measure the binding affinity of the selected aptamer pool.

To further refine specificity, a counter-SELEX step can be introduced. This involves exposing the nucleic acid library to irrelevant proteins or beads without the target antigen to eliminate non-specific binders. After the final SELEX round, the enriched pool of aptamers can be cloned into a suitable vector for sequencing, and bioinformatic analysis can be used to identify unique aptamer sequences. These sequences may be synthesized or transcribed for further characterization, including determining their dissociation constants (Kd) and binding specificity to NUP 358. High-affinity aptamers may be expected to exhibit minimal cross-reactivity with other proteins. Throughout the SELEX process, various parameters such as washing stringency, immobilization techniques, and negative controls may be optimized to ensure high specificity and reduce non-specific binding.

Provided herein are methods for delivery or transfection of a nucleic acid to a cell, tissue, or organ of a subject in a targeted manner using sonoporation (e.g., a process comprising applying an ultrasonic acoustic energy to a cell, tissue, or organ, such as to provide increased porosity in the cell, tissue, or organ). Provided in certain embodiments herein are methods for delivering to a cell a nucleic acid comprising a cargo polynucleotide (e.g., an expression cassette comprising a transgene or therapeutic oligonucleotide) and an aptamer to a target cell using sonoporation. In certain embodiments the aptamer comprises a sequence configured to promote an intracellular function. In some cases, the intracellular function comprises increasing nuclear localization in the target cell, preventing degradation of the cargo polynucleotide from the one or more intracellular nucleases, or increasing transcription of the cargo polynucleotide. Provided in certain embodiments herein are methods for transfecting a nucleic acid into a target cell or tissue (e.g., of a subject) by applying an ultrasonic acoustic energy to a cell, tissue, or organ. In some cases, the applying the ultrasonic acoustic energy comprises applying a first ultrasonic acoustic energy to the cell, tissue, or organ, and applying a second ultrasonic acoustic energy to the cell, tissue, or organ. In specific embodiments herein are methods for transfecting a nucleic acid into a target cell or tissue by applying a first ultrasonic acoustic energy having a first mechanical index (MI) and applying a second ultrasonic acoustic energy having a second mechanical index (MI). The present disclosure provides methods for enhancing transfection of a nucleic acid into the target cell or tissue by applying alternating ultrasonic acoustic energy, the alternating acoustic energy alternating between a first mechanical index (MI) and a second MI. Application of ultrasonic acoustic energy can be repeated several times during sonoporation, and may increase the efficiency of nucleic acid transfection and/or delivery.

In some embodiments, a process provided herein provides sonoporation at two or more different ultrasonic acoustic energies (e.g., a first and second ultrasonic acoustic energy having a first and second MI, respectively). In certain embodiments, a process provided herein provides a process wherein an ultrasonic acoustic energy is continuously applied (e.g., ultrasonic acoustic energy transitions from the first ultrasonic acoustic energy to the second ultrasonic acoustic energy, without a period of no ultrasonic acoustic energy being applied). In certain embodiments, a transitory (e.g., third, fourth, etc.) ultrasonic acoustic energy is applied between application of the first and second ultrasonic acoustic energies.

In some embodiments, a sonoporation treatment (e.g., application of a first ultrasonic acoustic energy, a second ultrasonic acoustic energy, a single cycle of a first ultrasonic acoustic energy and a second ultrasonic acoustic energy, or series of cycles comprising a plurality of applications of a first ultrasonic acoustic energy and a plurality of applications of a second acoustic energy) can last for a few seconds (e.g., 1-100 seconds) or more, such as up to a few minutes (e.g., 1-3 minutes). In specific embodiments, a sonoporation treatment last for 1-30 seconds. In some specific embodiments, a sonoporation treatment lasts for 5-100 seconds. In certain embodiments, a sonoporation treatment lasts for at least 1 minute (e.g., 1-30 minutes).

In some embodiments, a first MI is a Low MI (e.g., less than 0.4). In certain embodiments, a second MI is a High MI (e.g., 0.4 or greater). In some embodiments, a first MI is a Low MI (e.g., less than 0.4) and a second MI is a High MI (e.g., 0.4 or greater). In some embodiments, a second MI is a Low MI (e.g., less than 0.4). In certain embodiments, a first MI is a High MI (e.g., 0.4 or greater). In specific embodiments, a second MI is a Low MI (e.g., less than 0.4) and a first MI is a High MI (e.g., 0.4 or greater).

In some embodiments, a Low MI is <0.3. In specific embodiments, a Low MI is <0.2. In more specific embodiments, a Low MI is <0.1. In still more specific embodiments, a Low MI is about 0.09. In still more specific embodiments, a Low MI is about 0.04. In still more specific embodiments, a Low MI is about 0.03.

In some embodiments, a second MI is a High MI (e.g., 0.4 or greater). In some embodiments, a High MI is >0.5. In specific embodiments, a High MI is 0.5 to 2.0 or is between 0.5 and 2.0. In more specific embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is greater than 0.4. In some embodiments, a High MI is >0.5. In specific embodiments, a High MI is 0.5 to 2.0 or is between 0.5 and 2.3. In more specific embodiments, a High MI is 0.5 to 1 or is between 0.5 and 2.0. In some embodiments, a High MI is >0.5. In specific embodiments, a High MI is 0.5 to 2.3 or is between 0.5 and 2.3. In more specific embodiments, a High MI is 0.5 to 1 or is between 0.5 and 2.3. In some embodiments, a High MI is 1.5. In some embodiments, a High MI is 1.8. In some embodiments, a High MI is 2.0. In some embodiments, a High MI is >0.5. In specific embodiments, a High MI is 0.5 to 2.9 or is between 0.5 and 2.9. In more specific embodiments, a High MI is 0.5 to 1 or is between 0.5 and 2.9. In some embodiments, a High MI is >0.5. In specific embodiments, a High MI is 0.5 to 2.0 or is between 0.5 and 2.0. In more specific embodiments, a High MI is 0.5 to 1 or is between 0.5 and 2.9. In some embodiments, a High MI is 1.5.

In certain embodiments, any process provided herein (e.g., a sonoporation treatment) comprises administering of a continuous ultrasonic acoustic energy (which may have varying energy levels) that alternates (e.g., in identical, similar, or variable periods) between Low MI and High MI. In some embodiments, a (e.g., continuous, such as continuous but for administration of a second ultrasonic acoustic energy) Low MI (e.g., <0.1) (e.g., first) ultrasonic acoustic energy (also referred to herein as a Low MI) is administered to the subject with a set number pulses (e.g., of less than 30 seconds) of High MI (e.g., second) ultrasonic acoustic energy (also referred to herein as a High MI). In some embodiments, a process provided herein comprises administration of a plurality of pulses of High MI (e.g., second) ultrasonic acoustic energy, e.g., during an otherwise continuous administration of a Low MI (e.g., first) ultrasonic acoustic energy. In specific embodiments, the number of High MI pulses is about 4 or more, such as up to about 12, or an unlimited number of pulses. In specific embodiments the number of High MI pulses is 6-30. In still more specific embodiments, the number of High MI pulses is between 8, 9, 12, 15, or 18, or any number therebetween.

In specific embodiments, a pulse length is any suitable length, such as less than 30 seconds. In more specific embodiments, a pulse length is less than 15 seconds. In still more specific embodiments, a pulse length is less than 10 seconds. In yet more specific embodiments, a pulse length is less than 5 seconds. In more specific embodiments, a pulse length is less than 2 seconds. In still more specific embodiments, a pulse length is less than 1 second and/or may be greater than or equal to 1 microsecond. In some embodiments, a pulse length ranges from 100 to 300 microseconds. In some embodiments, a pulse length is up to about 200 microseconds. In some embodiments, a pulse length is up to about 500 microseconds. In some embodiments, a pulse length ranges from 1 to 500 microseconds.

In various embodiments, a High MI ultrasonic acoustic energy is provided first temporally (e.g., first in order). In other embodiments, a Low MI ultrasonic acoustic energy is provided second temporally (e.g., second in order).

In some embodiments, any process provided herein further comprises administering (e.g., systemically administering, such as via infusion) a nucleic acid (e.g., any nucleic acid provided herein) to a subject (e.g., to whom the ultrasonic acoustic energies are applied).

In some embodiments, any process provided herein further comprises administering (e.g., systemically administering, such as via infusion) a sonoactive structure (e.g., any sonoactive structure or microbubble described herein) to a subject (e.g., to whom the ultrasonic acoustic energies are applied).

In certain embodiments, provided herein is a method of delivering a nucleic acid in a target cell (e.g., of a tissue or organ) of a subject, the method comprising: administering to the subject a nucleic acid comprising the cargo polynucleotide; administering to the subject a plurality of sonoactive microstructures; and administering a sonoporation treatment.

In some embodiments, the sonoporation treatment comprises applying an ultrasonic acoustic energy to the target cell (e.g., of a tissue or organ of the subject) (e.g., the ultrasonic acoustic energy having a mechanical index (MI)). In some embodiments, applying an ultrasonic acoustic energy to the target cell comprises applying a first ultrasonic acoustic energy to the target cell and applying a second ultrasonic acoustic energy to the target cell. In some embodiments, the (e.g., first or second) ultrasonic acoustic energy has a first mechanical index (MI). In certain embodiments, (e.g., the other of the first or second) ultrasonic energy has a second mechanical index (MI). In some embodiments, the (e.g., first or second) MI is less than 0.4. In certain embodiments (e.g., the other of the first or second) MI is greater than 0.4 (e.g., and less than 2.3). In certain embodiments (e.g., the other of the first or second) MI is greater than 0.4 (e.g., and less than 2.9). In certain embodiments (e.g., the other of the first or second) MI is greater than 0.4 (e.g., and less than 2.0).

In specific embodiments, a first ultrasonic acoustic energy and a second ultrasonic acoustic energy are applied sequentially in a repeated manner.

In certain embodiments, the first (either High MI or Low MI) ultrasonic acoustic energy is applied before or after administration of any other agent, such as the nucleic acid and/or sonoactive structure. In some embodiments, the first ultrasonic acoustic energy is applied after administration of the sonoactive structure to the subject. In certain embodiments, the first ultrasonic acoustic energy is applied after administration of the nucleic acid to the subject. In some embodiments, the first ultrasonic acoustic energy is applied after administration of both the nucleic acid and the sonoactive structure(s).

In some embodiments, the first ultrasonic acoustic energy is administered within 60 minutes of administration of the nucleic acid and/or sonoactive structure(s). In specific embodiments, the first ultrasonic acoustic energy is administered within 30 minutes of administration of the nucleic acid and/or sonoactive structure(s). In more specific embodiments, the first ultrasonic acoustic energy is administered within 5 minutes of administration of the nucleic acid and/or sonoactive structure(s). In still more specific embodiments, the first ultrasonic acoustic energy is administered within 2 minutes of administration of the nucleic acid and/or sonoactive structure(s). In still more specific embodiments, the first ultrasonic acoustic energy may be applied simultaneously with administration of the nucleic acid and/or sonoactive structure(s).

In specific embodiments, the first (e.g., High MI) ultrasonic acoustic energy is applied immediately upon administration (e.g., infusion) or a period of time after administration (e.g., infusion) of the sonoactive structure(s) and/or nucleic acid.

In some embodiments, either the first or second ultrasonic acoustic energy is an ultrasonic acoustic energy (e.g., Low MI) that when applied to a cell, tissue, or organ of a subject results in stable cavitation (or stable vibrational cavitation) of the sonoactive structure and/or a change in the average diameter of the sonoactive structure(s), for example, due to inherent resonance properties of the microbubbles.

In certain embodiments, the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In certain embodiments, either the first or second ultrasonic acoustic energy is an ultrasonic acoustic energy (e.g., Low MI) that when applied to a cell, tissue, or organ of a subject results in stable cavitation (or stable vibrational cavitation) and/or a change in the average diameter of the sonoactive structure(s), and the other of the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In some instances, disruption of cell membrane allows target cells to become permeable to circulating agents such as nucleic acids. In certain instances, such circulating agents can then enter the target cells, tissues or organs, such as in a more rapid manner (e.g., relative to either Low MI or High MI ultrasonic acoustic energy application alone, or in the absence of ultrasonic acoustic energy application).

In some embodiments, the methods herein comprise alternating the ultrasonic acoustic energy applied between a first ultrasonic acoustic energy having a first MI and a second ultrasonic acoustic energy having a second MI. In some embodiments, applying alternating ultrasonic acoustic energy administered to a subject between a first MI and a second MI is performed repeatedly over a number of times, such as to enhance gene transfection into the target cells, tissue or organ (e.g., relative to a similar process wherein a first and second ultrasonic acoustic energy are not used and/or are not alternately applied and/or are not alternately applied repeatedly).

In some embodiments, the method comprises administering ultrasound energy transcutaneously to the subject in proximity to one or more target cells. In some embodiments, the one or more target cells are hepatic cells. In some embodiments, the one or more target cells are renal cells. In some embodiments, the one or more target cells are pancreatic cells. In some embodiments, the one or more target cells are cardiac cells. In some embodiments, the one or more target cells are myocytes. In some embodiments, the one or more target cells are neuronal cells. In some embodiments, the one or more target cells are brain cells. In some embodiments, the target cells are cancerous cells.

In some embodiments, the one or more target cells are comprised in a tissue. In some embodiments, the tissue is skeletal muscle tissue. In some embodiments, the tissue is smooth muscle tissue. In some embodiments, the tissue is connective tissue. In some embodiments, the tissue is lymphatic tissue. In some embodiments, the tissue is nervous tissue. In some embodiments, the tissue is diseased tissue, e.g., cancerous tissue, fibrotic tissue, or tissue otherwise in need of gene therapy.

In some embodiments, the target tissue is comprised in an organ. In some embodiments, the organ is the liver. In some embodiments, the organ is a kidney. In some embodiments, the organ is the pancreas. In some embodiments, the organ is the heart. In some embodiments, the organ is the brain. In some cases, the target cell comprises a hepatocyte, an LSEC, a podocyte, a cardiac cell, a cardiac myocyte, a pancreatic cell, a neural cell, or a muscle cell.

In some embodiments, the one or more target cells are comprised in a tumor. In some embodiments, the tumor is a solid tumor. In some embodiments, the tumor is a liquid tumor.

In some embodiments, cells, tissue or organ are those of the liver. In some embodiments, cells, tissue or organ are those of the kidney.

In certain embodiments, a subject herein is a mammal. In some embodiments, the mammal is, by way of non-limiting example, a human, rat, mouse, monkey, and other non-human primates.

In certain embodiments, changing parameters of the ultrasound acoustic energy or MI can be performed to induce and/or enhance an expression of a transgene in a cell or an organ of a subject. In one aspect, provided herein are methods of transfection by alternating the ultrasonic acoustic energy using a first MI and a second MI. In some embodiments, the first MI that results in stable vibrational cavitation is applied prior to the second MI, which results in inertial cavitation. In some embodiments, the ultrasonic acoustic energy using the first MI and the second MI are reapplied for a number of times to increase transfection efficiency at the target cell. In some embodiments, during the application of sonoporation, the ultrasonic acoustic energy is applied at the first MI continuously except for when the ultrasonic acoustic energy is applied at the second MI. For example, applying an ultrasonic acoustic energy to the target cell at the first MI then applying an ultrasonic acoustic energy to the target cell at the second MI are repeated between 4 to 18 times. In some embodiments, applying an ultrasonic acoustic energy to the target cell at the first MI then applying an ultrasonic acoustic energy to the target cell at the second MI are repeated an unlimited number of times. In one aspect, during this time, the ultrasonic acoustic energy of the first MI is applied continuously except for when the ultrasonic acoustic energy of the second MI is applied.

In some embodiments, the first MI ranges from about 0.05 to about 0.4. In some embodiments, the first MI ranges from about 0.1 to about 0.4. In some embodiments, the first MI ranges from about 0.15 to about 0.4. In some embodiments, the first MI ranges from about 0.2 to about 0.4. In some embodiments, the first MI ranges from about 0.25 to about 0.4. In some embodiments, the first MI ranges from about 0.3 to about 0.4. In some embodiments, the first MI ranges from about 0.05 to about 0.3. In some embodiments, the first MI ranges from about 0.1 to about 0.3. In some embodiments, the first MI ranges from about 0.15 to about 0.3. In some embodiments, the first MI ranges from about 0.2 to about 0.3. In some embodiments, the first MI ranges from about 0.25 to about 0.3. In some embodiments, the first MI is about 0.05. In some embodiments, the first MI is about 0.07. In some embodiments, the first MI is about 0.09. In some embodiments, the first MI is about 0.11.

In some embodiments, the second MI is greater than the first MI. In some embodiments, the second MI ranges from about 0.5 to about 2.3. In some embodiments, the second MI ranges from about 1.0 to about 1.8. In some embodiments, the second MI ranges from about 1.0 to about 2.0. In some embodiments, the second MI ranges from about 1.0 to about 2.9.

In some embodiments, the second MI is at least about 1. In some embodiments, the second MI is at least about 1.5. In some embodiments, the second MI is at least about 2.0. In some embodiments, the second MI is at least about 2.5. In some embodiments, the second MI is at least about 2.9. In some embodiments, the second MI is at least about 3.0. In some embodiments, the second MI is at least about 3.5.

In some embodiments, the applying the ultrasound acoustic energy comprises applying the ultrasound acoustic energy at the first MI or the second MI with an ultrasound probe applying the ultrasonic acoustic energy is in constant contact with the surface of the subject's skin at the location of application (e.g., abdomen, chest wall, skull, etc.). In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In certain embodiments, a transitory (e.g., third, fourth, etc.) ultrasonic acoustic energy is applied between application of the first and second ultrasonic acoustic energies. In certain embodiments, applying the ultrasound acoustic energy comprises applying the ultrasonic acoustic energy without regard to an EKG gating signal regulating the application of the ultrasound acoustic energy. In certain embodiments, applying the ultrasound acoustic energy comprises applying the ultrasonic acoustic energy without turning off power to the ultrasound transducer off. In some embodiments, applying the ultrasound acoustic energy comprises an ultrasound transducer sending ultrasound acoustic energy or receiving reflected ultrasound acoustic energy at least 95% of a period of time in which an ultrasound transducer continuously is contacting the subject.

In some instances, the ultrasonic acoustic energy of the second MI is applied using a pulse. In some instances, a pulse comprises applying the ultrasonic acoustic energy in a short pulse (e.g., microsecond length pulse). In some cases, the high MI is applied with the pulse, results in induces inertial cavitation and destruction of the sonoactive microstructure, resulting in the disruption of cell membrane and vascular endothelial integrity, transducing the cargo polynucleotide to the cell. In some instances, the pulse is applied with a duration of from about 1 μs to about 2 s. In some instances, the pulse is applied with a duration of from about 1 μs to about 1 s. In some instances, the pulse is applied with a duration of from about 1 μs to about 0.5 s. In some instances, the pulse is applied with a duration of from about 1 μs to about 5000 μs. In some instances, the pulse is applied with a duration of from about 1 μs to about 1000 μs. In some instances, the pulse is applied with a duration of from about 1 μs to about 500 μs. In some instances, the pulse is applied with a duration of from about 1 μs to about 300 μs. In some instances, the pulse is applied with a duration of from about 1 μs to about 200 μs. In some instances, the pulse is applied with a duration of from about 1 μs to about 100 μs. In some instances, the pulse is applied with a duration of from about 1 μs to about 50 μs. In some instances, the pulse is applied with a duration of from about 100 μs to about 2 s. In some instances, the pulse is applied with a duration of from about 100 μs to about 1 s. In some instances, the pulse is applied with a duration of from about 100 μs to about 0.5 s. In some instances, the pulse is applied with a duration of from about 100 μs to about 5000 μs. In some instances, the pulse is applied with a duration of from about 100 μs to about 1000 μs. In some instances, the pulse is applied with a duration of from about 100 μs to about 500 μs. In some instances, the pulse is applied with a duration of from about 100 μs to about 300 μs. In some instances, the pulse is applied with a duration of from about 100 μs to about 200 μs. In some instances, the pulse is applied with a duration of from about 200 μs to about 2 s. In some instances, the pulse is applied with a duration of from about 200 μs to about 1 s. In some instances, the pulse is applied with a duration of from about 200 μs to about 0.5 s. In some instances, the pulse is applied with a duration of from about 200 μs to about 5000 μs. In some instances, the pulse is applied with a duration of from about 200 μs to about 1000 μs. In some instances, the pulse is applied with a duration of from about 200 μs to about 500 μs. In some instances, the pulse is applied with a duration of from about 200 μs to about 300 μs. In some instances, the pulse is applied with a duration of from about 300 μs to about 2 s. In some instances, the pulse is applied with a duration of from about 300 μs to about 1 s. In some instances, the pulse is applied with a duration of from about 300 μs to about 0.5 s. In some instances, the pulse is applied with a duration of from about 300 μs to about 5000 μs. In some instances, the pulse is applied with a duration of from about 300 μs to about 1000 μs. In some instances, the pulse is applied with a duration of from about 300 μs to about 500 μs. In some instances, the pulse is applied with a duration of from about 500 μs to about 2 s. In some instances, the pulse is applied with a duration of from about 500 μs to about 1 s. In some instances, the pulse is applied with a duration of from about 500 μs to about 0.5 s. In some instances, the pulse is applied with a duration of from about 500 μs to about 5000 μs. In some instances, the pulse is applied with a duration of from about 500 μs to about 1000 μs. In some instances, the pulse is applied with a duration of from about 1000 μs to about 2 s. In some instances, the pulse is applied with a duration of from about 1000 μs to about 1 s. In some instances, the pulse is applied with a duration of from about 1000 μs to about 0.5 s. In some instances, the pulse is applied with a duration of from about 1000 μs to about 5000 μs. In some instances, the pulse is applied with a duration of from about 200 μs.

In some cases, alternating the ultrasonic acoustic energy between the first MI and the second MI for a number of times also allows reperfusion of the sonoactive microstructures and the nucleic acids to the target cell, tissue, or organ, following disruption of the sonoactive microstructures within or proximal to the target cell, tissue, or organ.

In some embodiments, applying ultrasonic acoustic energy at the first MI or the low MI induces stable vibration cavitation of the sonoactive microstructures. In some embodiments, applying ultrasonic acoustic energy at the first MI or the low MI does not induce substantial disruption of the sonoactive microstructures. In some embodiments, applying ultrasonic acoustic energy at the first MI or the low MI does not induce substantial disruption of the sonoactive microstructures in a vasculature space and an extravascular space, or induces stable vibration cavitation of the sonoactive microstructures in a vasculature space and an extravascular space.

In some embodiments, applying ultrasonic acoustic energy at the first MI or the low MI induces formation of an intercellular gap or an interendothelial gap or endocytosis. In some embodiments, the intercellular gap or the interendothelial gap ranges from about 10 nm to about 10 um. In some embodiments, the stable vibration cavitation of the sonoactive microstructures moves the nucleic acid from an intravenous space into an interstitial space or into the cytoplasm.

In some embodiments, applying ultrasonic acoustic energy in at the second MI or the high MI induces inertial cavitation of the sonoactive microstructures to disrupt the sonoactive microstructures. In some embodiments, applying ultrasonic acoustic energy at the second MI or the high MI induces inertial cavitation of the sonoactive microstructures to disrupt the sonoactive microstructures in a vasculature space and an extravascular space. In some embodiments, the extravascular spaces comprise an interstitial space, a subcutaneous space, intramuscular or a lymphatic space. In some embodiments, the extravascular spaces comprise an extravascular tissue. In some embodiments, the extravascular tissue comprises an interstitial space, a cytoplasmic space, a subcutaneous, a lymph tissues, muscular or combinations thereof.

In some embodiments, applying the ultrasonic acoustic energy at the second MI or the high MI induces formation of a pore in a membrane of the cell. In some embodiments, the formation of a pore in a membrane of the cell ranges from about 10 nm to about 10 um.

In some embodiments, administration of the sonoactive microstructures and nucleic acids occurs simultaneously in that the sonoactive microstructures are mixed with a solution comprising the nucleic acids prior to delivery to the subject. Such mixtures can comprise of 50% v/v of the sonoactive microstructures (e.g., Optison) and 50% v/v of a solution comprising a nucleic acid. Such mixtures can comprise varying percentages 5-90% v/v of the sonoactive microstructures.

In some embodiments, the nucleic acid comprises a miniplasmid backbone in the closed linear DNA construct. As used herein, the term “miniplasmid (mpDNA) backbone” refers to nucleic acids that are smaller in size (i.e., contain fewer base pairs (bp)) than conventional plasmids or pDNA in non-coding and non-regulatory portions of the vector. In some embodiments, the miniplasmid backbone comprises a backbone smaller than 1 kb. In some embodiments, the miniplasmid backbone is smaller than 1000 bp excluding an expression cassette. In some embodiments, the miniplasmid backbone comprises a backbone smaller than 0.5 kb. In some embodiments, the miniplasmid backbone comprises are smaller than 500 bp excluding an expression cassette. In some embodiments, the miniplasmid backbone comprises not comprise a bacterial origin of replication. As used herein, the term “Nanoplasmid™” (e.g., Nanopasmid sourced from Aldevron, Fargo, South Dakota.) refers to a small mpDNA construct that has a plasmid backbone that is less than 500 bp and does not contain an antibiotic resistance gene.

The miniplasmid backbone comprises can be utilized to deliver an expression cassette, a transgene, or a nonendogenous gene to cells in target cell-types, tissues or organs. In some embodiments, the miniplasmid backbone comprises less than 1000 base pairs excluding an expression cassette. In some embodiments, the miniplasmid backbone comprises less than 500 base pairs excluding an expression cassette. In some embodiments, the miniplasmid backbone does not comprise antibiotic resistant genes. In some embodiments, the miniplasmid backbone does not comprise a bacterial genome. In some embodiments, miniplasmid backbone enhances the expression of the nonendogenous gene or a therapeutic transgene when used in conjunction with the claimed methods and ultrasound acoustic profiles. In some embodiments, the cargo polynucleotide comprises an expression cassette. In some embodiments, the expression cassette comprises a transgene. In some embodiments, the cargo polynucleotide comprises a transgene (endogenous or non-endogenous). In some embodiments, the transgene comprises a therapeutic transgene. In some embodiments, inducing expression of the cargo polynucleotide comprises inducing expression of the therapeutic transgene. In some embodiments, the transgene comprises a detectible marker. In some embodiments, the transgene comprises luciferase. In some embodiments, inducing expression of the cargo polynucleotide comprises inducing expression of luciferase.

In some embodiments, a cargo polynucleotide comprises a regulatory element such as a promoter, (e.g., APOE-ATT). In some embodiments, a total amount (e.g., dose) of the nucleic acid (e.g., DNA) administered to a subject for purposes of sonoporation can range from 100 micrograms to 200 mg.

In some embodiments, the cargo polynucleotide is covalently coupled to the aptamer. In some embodiments, the cargo polynucleotide is 5′ of the aptamer. In some embodiments, the cargo polynucleotide is 3′ of the aptamer.

In some embodiments, the therapeutic payload is a nonendogenous gene. In some embodiments, the cargo polynucleotide is configured to perform gene augmentation, gene replacement, gene editing, gene knockdown, or gene knockout.

In some embodiments, the nucleic acid comprises one or more regulatory elements, such as a promoter, enhancer, ribosome binding site, or transcription termination signal. In some embodiments, the nucleic acid comprises a constitutively active promoter. In some embodiments, the nucleic acid comprises an organ specific promoter. In some embodiments, the nucleic acid comprises a tissue specific promoter. In some embodiments, the nucleic acid comprises a cell specific promoter. Examples of promoters contemplated herein include, but are not limited to, e.g., CMV promoter, UbC promoter, CAG promoter, EF-1α promoter, ApoE promoter, ApoE-AAT1 promoter, 3XSERP promoter, or P3-hybrid promoter. In some embodiments, the nucleic acid comprises a promoter sequence comprising CAG. In some embodiments, the nucleic acid comprises a promoter sequence comprising ApoE. In some embodiments, the nucleic acid comprises a promoter sequence comprising SERP. In some embodiments, the nucleic acid comprises a promoter sequence comprising P3.

In some embodiments, the nucleic acid is a linear DNA construct. In some embodiments, a linear DNA construct is a DNA molecule which is not a circular double stranded construct. In some embodiments, the nucleic acid is a closed linear DNA construct. In some embodiments, a linear DNA construct is formed from a circular DNA that has been linearized using a restriction enzyme or a CRISPR nuclease to create a double stranded break prior to closing the linearized ends with an aptamer. In some embodiments, the nucleic acid is a closed linear DNA construct comprising a stem region comprising a double stranded DNA sequence covalently closed at both ends by hairpin loops. In some embodiments, the hairpin loops are single-stranded. In some embodiments, the hairpin loops form a stem region of the aptamer.

In some embodiments, the nucleic acid is a closed linear DNA construct comprising at least two modified nucleotides. In some embodiments, the nucleic acid is a closed linear DNA construct comprising at least three modified nucleotides. In some embodiments, the nucleic acid is a closed linear DNA construct comprising at least four modified nucleotides. In some embodiments, the nucleic acid is a closed linear DNA construct comprising at least five modified nucleotides. In some embodiments, the at least two modified nucleotides are located in one single-stranded end loop of the closed linear DNA construct. In some embodiments, the at least two modified nucleotides are located in both single-stranded end loops of the closed linear DNA construct. In some embodiments, at least one modified nucleotide is located in one of the single stranded end loops. In some embodiments, the at least three modified nucleotides are located in both single-stranded end loops of the closed linear DNA construct. In some embodiments, the at least four modified nucleotides are located in one single-stranded end loop of the closed linear DNA construct. In some embodiments, the at least four modified nucleotides are located in both single-stranded end loops of the closed linear DNA construct. In some embodiments, the at least five modified nucleotides are located in one single-stranded end loop of the closed linear DNA construct. In some embodiments, the at least five modified nucleotides are located in both single-stranded end loops of the closed linear DNA construct. In some embodiments, at least one modified nucleotide is located in one of the single stranded end loops. In some embodiments, at least two modified nucleotides are located in one of the single stranded end loops. In some embodiments, at least three modified nucleotides are located in one of the single stranded end loops. In some embodiments, at least four modified nucleotides are located in one of the single stranded end loops. In some embodiments, at least five modified nucleotides are located in one of the single stranded end loops. In some embodiments, at least one modified nucleotide is located in one of the single stranded end loops forming the stem region of the aptamer. In some embodiments, at least two modified nucleotides are located in one of the single stranded end loops forming the stem region of the aptamer. In some embodiments, at least three modified nucleotides are located in one of the single stranded end loops forming the stem region of the aptamer. In some embodiments, at least four modified nucleotides are located in one of the single stranded end loops forming the stem region of the aptamer. In some embodiments, at least five modified nucleotides are located in one of the single stranded end loops forming the stem region of the aptamer. In some embodiments, at least one modified nucleotide is located in one of the single stranded end loops and at least one modified nucleotide is located in one of the single stranded end loops forming the stem region of the aptamer. In some embodiments, at least one modified nucleotide is located in one of the single stranded end loops and at least two modified nucleotides are located in one of the single stranded end loops forming the stem region of the aptamer. In some embodiments, at least one modified nucleotide is located in one of the single stranded end loops and at least three modified nucleotides are located in one of the single stranded end loops forming the stem region of the aptamer. In some embodiments, at least one modified nucleotide is located in one of the single stranded end loops and at least four modified nucleotides are located in one of the single stranded end loops forming the stem region of the aptamer. In some embodiments, at least two modified nucleotides are located in one of the single stranded end loops and at least one modified nucleotide is located in one of the single stranded end loops forming the stem region of the aptamer. In some embodiments, at least two modified nucleotides are located in one of the single stranded end loops and at least two modified nucleotides are located in one of the single stranded end loops forming the stem region of the aptamer. In some embodiments, at least two modified nucleotides are located in one of the single stranded end loops and at least three modified nucleotides are located in one of the single stranded end loops forming the stem region of the aptamer. In some embodiments, at least three modified nucleotides are located in one of the single stranded end loops and at least two modified nucleotides are located in one of the single stranded end loops forming the stem region of the aptamer. In some embodiments, at least three modified nucleotides are located in one of the single stranded end loops and at least one modified nucleotide is located in one of the single stranded end loops forming the stem region of the aptamer. In some embodiments, at least four modified nucleotides are located in one of the single stranded end loops and at least one modified nucleotide is located in one of the single stranded end loops forming the stem region of the aptamer.

In some embodiments, at least two modified nucleotides are in one or both single stranded end loops forming the stem region of the aptamer. In some embodiments, at least three modified nucleotides are in one or both single stranded end loops forming the stem region of the aptamer. In some embodiments, at least four modified nucleotides are in one or both single stranded end loops forming the stem region of the aptamer. In some embodiments, at least five modified nucleotides are in one or both single stranded end loops forming the stem region of the aptamer.

In some embodiments, the aptamer comprises a sequence configured to increase nuclear localization of the cargo polynucleotide. In some embodiments, the aptamer comprises a sequence configured to bind nucleolin. In some embodiments, the aptamer comprises a sequence having at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99% sequence identity to any one of SEQ ID NO: 3-54 or 78-85. In some embodiments, the aptamer comprises a sequence of any one of SEQ ID NO: 3-54, 129-130, or 135-148. In some embodiments, the aptamer comprises a sequence configured to bind importin. In some embodiments, the aptamer comprises the sequence of SEQ ID NO: 48, 129, OR 130. In some embodiments, the aptamer comprises a sequence configured to bind a nucleoporin protein. In some embodiments, the aptamer comprises a sequence of any one of SEQ ID NO: 49-50. In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a sequence of any one of SEQ ID NO: 51-54.

In some embodiments, nucleic acid comprising the cargo polynucleotide described herein may be administered with an aptamer. In some cases, the aptamer is a separate nucleic acid construct from the nucleic acid. In some embodiments, the nucleic acid may be co-formulated with an aptamer. In some embodiments, the nucleic acid may be co-formulated with an aptamer by using a polymer, and/or a polymeric nanoparticle.

In some embodiments, at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiment, at least two modified nucleotides are 2-amino-deoxyadenosine. In some embodiment, at least two modified nucleotides are 5-methyl-deoxycytidine. In some embodiment, at least two modified nucleotides are thiophosphate nucleotide. In some embodiment, at least two modified nucleotides are inosine nucleotide. In some embodiment, at least two modified nucleotides are locked nucleic acid (LNA) nucleotide. In some embodiment, at least two modified nucleotides are L-DNA nucleotide. In some embodiment, at least two modified nucleotides are 8-oxo-deoxyadenosine nucleotide. In some embodiment, at least two modified nucleotides are 5-Fluoro-deoxyuracil nucleotide.

In some embodiments, the closed linear DNA construct comprises from about 3 to about 20 modified nucleotides. In some embodiments, the closed linear DNA construct comprises about 1 modified nucleotide. In some embodiments, the closed linear DNA construct comprises about 2 modified nucleotides. In some embodiments, the closed linear DNA construct comprises about 3 modified nucleotides. In some embodiments, the closed linear DNA construct comprises about 4 modified nucleotides. In some embodiments, the closed linear DNA construct comprises about 5 modified nucleotides. In some embodiments, the closed linear DNA construct comprises about 6 modified nucleotides. In some embodiments, the closed linear DNA construct comprises about 7 modified nucleotides. In some embodiments, the closed linear DNA construct comprises about 8 modified nucleotides. In some embodiments, the closed linear DNA construct comprises about 9 modified nucleotides. In some embodiments, the closed linear DNA construct comprises about 10 modified nucleotides. In some embodiments, the closed linear DNA construct comprises about 11 modified nucleotides. In some embodiments, the closed linear DNA construct comprises about 12 modified nucleotides. In some embodiments, the closed linear DNA construct comprises about 13 modified nucleotides. In some embodiments, the closed linear DNA construct comprises about 14 modified nucleotides. In some embodiments, the closed linear DNA construct comprises about 15 modified nucleotides. In some embodiments, the closed linear DNA construct comprises about 16 modified nucleotides. In some embodiments, the closed linear DNA construct comprises about 17 modified nucleotides. In some embodiments, the closed linear DNA construct comprises about 18 modified nucleotides. In some embodiments, the closed linear DNA construct comprises about 19 modified nucleotides. In some embodiments, the closed linear DNA construct comprises about 20 modified nucleotides. In some embodiments, the closed linear DNA construct comprises about 21 modified nucleotides. In some embodiments, the closed linear DNA construct comprises about 22 modified nucleotides.

In some embodiments, the closed linear DNA construct comprises at least two LNA nucleotides. In some embodiments, the closed linear DNA construct comprises at least three LNA nucleotides. In some embodiments, the closed linear DNA construct comprises at least four LNA nucleotides. In some embodiments, the closed linear DNA construct comprises at least five LNA nucleotides. In some embodiments, the closed linear DNA construct comprises at least two thiophosphate nucleotides. In some embodiments, the closed linear DNA construct comprises at least three thiophosphate nucleotides. In some embodiments, the closed linear DNA construct comprises at least four thiophosphate nucleotides. In some embodiments, the closed linear DNA construct comprises at least five thiophosphate nucleotides. In some embodiments, the closed linear DNA construct comprises at least two restriction sites flanking the expression cassette. In some embodiments, the closed linear DNA construct comprises at least three restriction sites flanking the expression cassette. In some embodiments, the closed linear DNA construct comprises at least four restriction sites flanking the expression cassette. In some embodiments, the closed linear DNA construct comprises at least five restriction sites flanking the expression cassette. In some embodiment, the closed linear DNA construct comprises a primase recognition site. In some embodiments, the closed linear DNA construct comprises two primase recognition sites. In some embodiments, the closed linear DNA construct comprises three primase recognition sites. In some embodiments, the closed linear DNA construct comprises an inverted terminal repeats (ITR). In some embodiments, the closed linear DNA construct comprises two ITRs. In some embodiments, the closed linear DNA construct comprises three ITRs.

In some embodiments, inducing expression of the cargo polynucleotide comprises inducing production of RNA encoded by the payload. In some embodiments, inducing expression of the cargo polynucleotide comprises inducing production of protein encoded by the payload.

Aspects disclosed herein provide an isolated nucleic acid encoding any one of SEQ ID NO: 1-253.

Mammalian somatic cells generally exhibit innate immune DNA sensing to cytosolic DNA, providing a pathological immune response cytosolic DNA. The present of an innate immune response to cytosolic DNA provides several benefits to cells such as defense against various pathogens, for example, viruses, in addition to detection and response to cellular damage or aberrant cellular processes. For example, cytosolic DNA sensing allows for: detection of pathogens when viral, bacterial, or parasitic DNA genomes are released into the cytosol during the pathogen replication cycle, allowing for an inflammatory immune response to occur; detection of cellular damage resulting in DNA fragmentation, for example, during apoptosis or necrosis; and maintenance of genome integrity, by detection and response to aberrant DNA which may be associated with development of cancer. However, when delivering DNA to cells as part of a gene therapy treatment, an immune response from the host cell is not ideal, and may reduce the delivery of the nucleic acid payload to the cell, and resulting gene expression.

Mechanisms of innate immune DNA sensing to cytosolic DNA can include binding of double stranded cytosolic DNA by cyclic GMP-AMP synthase (cGAS), leading to a signaling cascade driving a further immune response including synthesis of a special asymmetric cyclic-dinucleotide, 2′3′-cGAMP, and activation of STING (endoplasmic reticulum (ER) membrane protein) for subsequent production of type I interferons and other immune-modulatory genes, as is illustrated in FIG. 11. In addition to type I IFN production, activation of the cGAS-STING pathway can also lead to the production of pro-inflammatory cytokines, such as tumor necrosis factor alpha (TNF-α) and interleukin-6 (IL-6), induction of cellular autophagy, activation of activation of caspase-1 and subsequent processing and secretion of pro-inflammatory cytokines such as interleukin-1 beta (IL-1β) and interleukin-18 (IL-18), and activation of apoptotic pathways. The design of nucleic acid delivery vectors which can successfully avoid mechanisms of innate immune DNA sensing and resulting immune responses has the potential to improve the delivery of nucleic acids to target cells in gene therapy treatments, resulting transgene expression, and subject outcomes with reduced inflammatory responses. In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a sequence of any one of SEQ ID NO: 51-54.

In some embodiments, the payload comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides). In some embodiments, the nucleic acid is configured to perform gene augmentation, gene replacement, base editing, base knockdown, gene editing gene knockdown, or gene knockout. In some embodiments, delivering the cargo polynucleotide to the target cell of the subject increases or decreases expression of a gene in the target cell.

In some embodiments, the payload comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease a regulatory protein or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR). In some embodiments, the payload may comprise payload may further comprise a template DNA molecule which is a transposase, an ARCUS, a TPRT enzyme, or a CAS transposases, or a nucleic acid which encodes a transposase, an ARCUS, a TPRT enzyme, or a CAS transposases.

In some embodiments, the payload comprises a nucleic acid that exceeds the size limitation of conventional gene therapy vectors. In some embodiments, the payload exceeds the size limitation of an adeno-associated viral vector (AAV). In some embodiments, the payload is greater than about 4.7 kb. In some embodiments, the payload is greater than about 5, about 5.5, about 6, about 6.5, about 7, about 7.5, about 8, about 8.5, about 9, about 9.5, about 10, about 10.5, about 11, about 11.5, about 12, about 12.5, or about 13 kb.

Aptamers are short sequences of artificial DNA, or RNA sequences that bind to one or more target molecules. In some embodiments, the aptamer comprises a sequence configured to promote an intracellular function. In some embodiments, the intracellular function comprises nuclear localization. In some embodiments, the intracellular function comprises increasing nuclear localization in the target cell. In some embodiments, the intracellular function comprises increased resistance to one or more intracellular nucleases. In some embodiments, the intracellular function comprises preventing degradation of the cargo polynucleotide by preventing degradation of the cargo polynucleotide from the one or more intracellular nucleases. In some embodiments, the intracellular function comprises improved transcription of the cargo polynucleotide. In some embodiments, the intracellular function comprises increasing transcription of the cargo polynucleotide. In some embodiments, the aptamer comprises a sequence configured to increase nuclear localization of the cargo polynucleotide. In some embodiments, the aptamer comprises a sequence configured to bind nucleolin. In some embodiments, the aptamer comprises a sequence of any one of SEQ ID NOS: 1-47. In some embodiments, the aptamer comprises a sequence configured to bind importin. In some embodiments, the aptamer comprises the sequence of SEQ ID NO: 48, 129, OR 130. In some embodiments, the aptamer comprises a sequence configured to bind a nucleoporin protein. In some embodiments, the aptamer comprises a sequence of any one of SEQ ID NOS: 48-49.

Sonoactive agents (also referred to as sonoactive microstructures acoustic microspheres or “microbubbles”) contemplated herein include, but are not limited to, those used as ultrasonic imaging contrast agents. In some embodiments, the sonoactive agent comprises a phospholipid stabilized microstructure. In some embodiments, the phospholipid stabilized microstructure comprises a high molecular weight gas core, or a perflutran core. Examples of sonoactive agents include, but are not limited to, OPTISON (GE Healthcare), Sonazoid (GE Healthcare), or DEFINITY and Definity RT (Lantheus Medical Imaging, Inc). In some embodiments, the sonoactive agents are LUMASON (Bracco) (sulfur hexafluoride lipid-type A microspheres). In some embodiments, the sonoactive agents are SonoVue (sulfur hexafluoride microbubbles). In some embodiments, the sonoactive agents comprise a protein stabilized microstructure. In some embodiments, the sonoactive agents are Optison microbubbles.

The sonoactive agent can be administered prior to, after, or simultaneous (e.g., coadministered) with the administration of the nucleic acid (or cargo polynucleotide). In some embodiments, the nucleic acid and the sonoactive agent are coadministered. In some embodiments, the administering of the nucleic acid and the sonoactive agent occurs serially, concurrently, sequentially, or continuously. In some embodiments, the administering of the nucleic acid and the sonoactive agent occurs serially. In some embodiments, the administering of the nucleic acid and the sonoactive agent occurs concurrently. In some embodiments, the administering of the nucleic acid and the sonoactive agent occurs sequentially. In some embodiments, the administering of the nucleic acid and the sonoactive agent occurs continuously.

In some embodiments, the nucleic acid is administered at a dosage of about 0.5 mg/kg to about 500 mg/kg. In some embodiments, about 2×10\hat{ }13 to about 3×10\hat{ }13 copies of the nucleic acid are administered to the subject.

In some embodiments, the sonoactive microstructures are administered at a dosage of about 1-50 mL, for example 1 mL of Optison. The sonoactive microstructures may be administered at a concentration of about 5M to about 8M microstructures per mL. In some embodiments, the sonoactive microstructures are administered at a concentration of about 5×10\hat{ }8 to about 1.2×10\hat{ }9 microstructures/mL, for example 1×10\hat{ }9 of Definity RT. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of about 5×10\hat{ }8 to about 8×10\hat{ }8 microstructures/mL.

In some embodiments, the nucleic acid and the sonoactive microstructures are mixed prior to being coadministered. In some instances, the sonoactive microstructures are mixed with the nucleic acids before administering to the subject. In some instances, the sonoactive microstructures are mixed with the nucleic acids along with additional buffers or agents such as saline or other biocompatible solutions with varying electrostatic charges and surface chemistries and ligands before administering to the subject. For example, Optison sonoactive microstructures can be mixed with a miniplasmid construct, e.g., a Nanoplasmid, comprising a promoter coupled to a transgene, e.g., APOE-Fluc, and saline, and administered together.

In some embodiments, the administering of the nucleic acid and the sonoactive agent is by intravenous administration or subcutaneous or intramuscular or intra-arterial or inter-osseus or direct organ puncture.

In some embodiments, after administering of the nucleic acid and sonoactive agent, the ultrasound acoustic energy is applied at the target cell, tissue, or organ.

Once the nucleic acids are inside the target cell, expression of the cargo polynucleotide is induced. In some embodiments, the cargo polynucleotide comprises luciferase.

In some embodiments, inducing expression of the cargo polynucleotide comprises inducing expression within about 3 to about 12 hours of administering the payload. In some embodiments, inducing expression of the cargo polynucleotide comprises inducing expressing within about 3 hours of administration. In some embodiments, inducing expression of the cargo polynucleotide comprises inducing expressing within about 6 hours of administration. In some embodiments, inducing expression of the cargo polynucleotide comprises inducing expression within about 12 hours of administration.

Undesirable effects on living cells or tissues can occur due to ultrasound applications. In some embodiments, the present disclosure provides methods for improvement of gene transfection and not result in substantial DNA or cell damage in the target cells, tissues, or organs, using sonoporation by alternating ultrasonic acoustic energy between the first MI and the second MI. In some embodiments, the method does not result in substantial cellular damage to the target cell. In some embodiments, the method results in less than 1%, 5%, or 10% of target cells undergoing apoptosis.

A sonoporation treatment using the methods described herein can be used to induce expression of a cargo polynucleotide in a cell in a liver or a cell in a kidney.

A sonoporation treatment using the methods described herein can be used to treat a subject in need for gene therapy or protein replacement treatment. In another aspect, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher's (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

In some embodiments, the present disclosure provides methods of treating a subject having a liver condition with a therapeutic transgene. In some embodiments, the therapeutic transgene encodes one or more of: ATP7B; ABCB11; ABCB4; ATP8B1; TJP2; VWF; FVIII; FIX; F5; MAN2B1; GBA; SMPD1; CPS1; GDE/AGL; CTNS; SERPINA1; ASS1, and/or SLC25A13.

In some embodiments, the present disclosure provides methods of treating a subject having a liver condition with a therapeutic transgene. In some embodiments, the liver condition is Wilson's Disease, and the therapeutic transgene encodes ATP7B. In some embodiments, the liver condition is Cholestasis, progressive familial intrahepatic (PFIC1-4) and the therapeutic transgene encodes one or more of ABCB11, ABCB4, ATP8B1 and/or TJP2. In some embodiments, the liver condition is Von Willebrand Disease and the therapeutic transgene encodes VWF. In some embodiments, the liver condition is Hemophilia A, and the therapeutic transgene encodes FVIII. In some embodiments, the liver condition is Hemophilia B, and the therapeutic transgene encodes FIX. In some embodiments, the liver condition is Factor V Deficiency, and the therapeutic transgene encodes F5. In some embodiments, the liver condition is Alpha-Mannosidosis, and the therapeutic transgene encodes MAN2B1. In some embodiments, the liver condition is Gaucher's (glucocerebrosidase deficiency, glucocerebrosidosis), and the therapeutic transgene encodes GBA. In some embodiments, the liver condition is Niemann Pick Disease A/B, and the therapeutic transgene encodes SMPD1. In some embodiments, the liver condition is Carbamoylphosphate Synthetase I Deficiency, and the therapeutic transgene encodes CPS1. In some embodiments, the liver condition is Glycogen Storage Disease Type III, and the therapeutic transgene encodes GDE/AGL. In some embodiments, the liver condition is Cystinosis, and the therapeutic transgene encodes CTNS. In some embodiments, the liver condition is A1AT deficiency, and the therapeutic transgene encodes SERPINA1. In some embodiments, the liver condition is Citrullinemia Type I & II, and the therapeutic transgene encodes one or more of ASS1 and/or SLC25A13. In some embodiments, the methods comprise administering to the subject a nucleic acid comprising the cargo polynucleotide (e.g., a therapeutic transgene); administering to the subject a plurality of sonoactive microstructures; and administering a sonoporation treatment. In some embodiments, the sonoporation treatment comprises applying an ultrasonic acoustic energy to a liver at a first mechanical index (MI) that is less than 0.4; applying an ultrasonic acoustic energy to the liver at a second MI that is greater than 0.4 and less than 2.0; In some embodiments, the method comprises repeating application of the low MI and the high MI a number of times. In some embodiments, the method comprises delivering the cargo polynucleotide and the plurality of sonoactive microstructures systemically (e.g., by intravenous administration). In some embodiments, the sonoporation treatment comprises applying an ultrasonic acoustic energy to a liver at a first mechanical index (MI) that is less than 0.4; applying an ultrasonic acoustic energy to the liver at a second MI that is greater than 0.4 and less than 2.3; In some embodiments, the method comprises repeating application of the low MI and the high MI a number of times. In some embodiments, the sonoporation treatment comprises applying an ultrasonic acoustic energy to a liver at a first mechanical index (MI) that is less than 0.4; applying an ultrasonic acoustic energy to the liver at a second MI that is greater than 0.4 and less than 2.9. In some embodiments, the method comprises repeating application of the low MI and the high MI a number of times. In some embodiments, the method comprises delivering the cargo polynucleotide and the plurality of sonoactive microstructures systemically (e.g., by intravenous administration).

In some embodiments, provided herein is a method of treating a subject having Hemophilia A comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, provided herein is a method of treating a subject having Hemophilia A comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.3 (e.g., 0.4<MI≤2.3). In some embodiments, provided herein is a method of treating a subject having Hemophilia A comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.9 (e.g., 0.4<MI≤2.9). In some embodiments, the therapeutic transgene is operably linked to a liver specific promoter. In some embodiments, the therapeutic transgene comprises a nucleic acid sequence encoding Factor VIII. In some embodiments, the nucleic acid and the plurality of sonoactive microstructures are administered systemically (e.g., by intravenous administration).

In some embodiments, provided herein is a method of treating a subject having Wilson's Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, provided herein is a method of treating a subject having Wilson's Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.3 (e.g., 0.4<MI≤2.3). In some embodiments, provided herein is a method of treating a subject having Wilson's Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.9 (e.g., 0.4<MI≤2.9). In some embodiments, the therapeutic transgene is operably linked to a liver specific promoter. In some embodiments, the therapeutic transgene comprises a nucleic acid sequence encoding ATP7B. In some embodiments, the nucleic acid and the plurality of sonoactive microstructures are administered systemically (e.g., by intravenous administration).

In one aspect, using the methods described herein, the present disclosure provides methods of treating a subject having a kidney condition. In some embodiments, the kidney condition treated is: Alport Syndrome, or Autosomal Dominant Polycystic Kidney Disease.

In some embodiments, the present disclosure provides methods of treating a subject having a kidney condition with a therapeutic transgene. In some embodiments, the therapeutic transgene encodes one or more of COL4A3, COL4A4, COL4A5, PKD1 and/or PKD2.

In some embodiments, the present disclosure provides methods of treating a subject having a kidney condition with a therapeutic transgene. In some embodiments, the kidney condition is Alport Syndrome, and the therapeutic transgene encodes one or more of COL4A3, COL4A4, and/or COL4A5. In some embodiments, the kidney condition is Autosomal Dominant Polycystic Kidney Disease, and the therapeutic transgene encodes one or more of PKD1 and/or PKD2. In some embodiments, the methods comprise administering to the subject a nucleic acid comprising the cargo polynucleotide; administering to the subject a plurality of sonoactive microstructures; and administering a sonoporation treatment. In some embodiments, the sonoporation treatment comprises applying an ultrasonic acoustic energy to a kidney at a first mechanical index (MI) that is less than 0.4; applying an ultrasonic acoustic energy to the kidney at a second MI that is greater than 0.4 and less than 2.0. In some embodiments, the sonoporation treatment comprises applying an ultrasonic acoustic energy to a kidney at a first mechanical index (MI) that is less than 0.4; applying an ultrasonic acoustic energy to the kidney at a second MI that is greater than 0.4 and less than 2.3. In some embodiments, the method comprises repeating application of the low MI and the high MI a number of times. In some embodiments, the sonoporation treatment comprises applying an ultrasonic acoustic energy to a kidney at a first mechanical index (MI) that is less than 0.4; applying an ultrasonic acoustic energy to the kidney at a second MI that is greater than 0.4 and less than 2.9. In some embodiments, the method comprises delivering the cargo polynucleotide and the plurality of sonoactive microstructures systemically (e.g., by intravenous administration).

In some embodiments, provided herein is a method of treating a subject having Alport Syndrome comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, provided herein is a method of treating a subject having Alport Syndrome comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.3 (e.g., 0.4<MI≤2.3). In some embodiments, provided herein is a method of treating a subject having Alport Syndrome comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.9 (e.g., 0.4<MI≤2.9). In some embodiments, the therapeutic transgene is operably linked to a kidney specific promoter. In some embodiments, the therapeutic transgene comprises a nucleic acid sequence encoding COL4A3. In some embodiments, the therapeutic transgene comprises a nucleic acid sequence encoding COL4A4. In some embodiments, the therapeutic transgene comprises a nucleic acid sequence encoding COL4A5. In some embodiments, the nucleic acid and the plurality of sonoactive microstructures are administered systemically (e.g., by intravenous administration).

In some embodiments, provided herein is a method of treating a subject having Autosomal Polycystic Kidney Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, provided herein is a method of treating a subject having Autosomal Polycystic Kidney Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.3 (e.g., 0.4<MI≤2.3). In some embodiments, provided herein is a method of treating a subject having Autosomal Polycystic Kidney Disease comprising administering to the subject a nucleic acid comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.9 (e.g., 0.4<MI≤2.9). In some embodiments, the therapeutic transgene is operably linked to a kidney specific promoter. In some embodiments, the therapeutic transgene comprises a nucleic acid sequence encoding PKD1. In some embodiments, the therapeutic transgene comprises a nucleic acid sequence encoding PKD2. In some embodiments, the nucleic acid and the plurality of sonoactive microstructures are administered systemically (e.g., by intravenous administration).

Aspects disclosed herein provide a pharmaceutical composition comprising: a microbubble; and a nucleic acid comprising (1) a cargo polynucleotide comprising an expression cassette that comprises a therapeutic transgene and (2) an aptamer, wherein the aptamer comprises a sequence configured to increase nuclear localization. Aspects disclosed herein provide a pharmaceutical composition comprising: a sonoactive agent; a nucleic acid comprising (1) a cargo polynucleotide comprising an expression cassette that encodes a therapeutic transgene and (2) one or both of: (i) a nuclear localization element, and/or (ii) an innate immune response avoidance moiety.

Aspects disclosed herein provide a pharmaceutical composition comprising an isolated nucleic acid comprising a cargo polynucleotide and a nuclear localization element, wherein the nuclear localization element increases expression of the cargo polynucleotide in the target cell by at least 1.25 fold as compared to a nucleic acid lacking the nuclear localization element. Aspects disclosed herein provide a pharmaceutical composition comprising an isolated nucleic acid comprising a cargo polynucleotide and an innate immune response avoidance moiety, wherein the innate immune response avoidance moiety increases expression of the cargo polynucleotide in the target cell by at least 1.25 fold as compared to a nucleic acid lacking the nuclear localization element. Aspects disclosed herein provide a pharmaceutical composition comprising: a sonoactive agent; and nucleic acids comprising (1) a cargo polynucleotide comprising an expression cassette that encodes a therapeutic transgene and (2) one or both of: (i) a nuclear localization element, and/or (ii) an innate immune response avoidance moiety.

In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, at least or 5 fold as compared to a nucleic acid lacking the nuclear localization element. In some embodiments, the immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, at least or 5 fold as compared to a nucleic acid lacking the immune response avoidance moiety. In some embodiments, the isolated nucleic acid further comprises an innate immune response avoidance moiety. In some embodiments, the isolated nucleic acid further comprises an innate immune response avoidance moiety. In some embodiments, the nucleic acid is up to 40 nucleotides in length. In some embodiments, the nucleic acid is an isolated nucleic acid. In some embodiments, the target cell is a liver cell. In some embodiments, the target cell is a hepatocyte. In some embodiments, the target cell is a LSEC. In some embodiments, the target cell is a kidney cell. In some embodiments, the target cell is a proximal tubular epithelial cell. In some embodiments, the target cell is a podocyte. In some embodiments, the target cell is a muscle cell. In some embodiments, the method is a method to treat a subject in need of a gene therapy or a protein replacement therapy. In some embodiments, the method is a method of treating a mammalian subject having a genetic disorder with a nucleic acid encoding a therapeutic transgene. In some embodiments, the cargo polynucleotide comprises an expression cassette encoding the therapeutic transgene, wherein the therapeutic transgene is configured for expression in the target cell of the subject. In some embodiments, the method is a method for use of the nucleic acid, the sonoactive agent or the microbubble, and the ultrasound in treatment of a subject having a genetic disorder requiring a gene therapy or a protein replacement therapy. In some embodiments, the subject is a subject having Hemophilia A or FVIII deficiency, the nucleic acid encodes FVIII, and the target cell is a liver cell. In some embodiments, the subject is a subject having Alport's Syndrome or COL4A5 deficiency, and the nucleic acid encodes alpha5(IV) chain of collagen IV, and the target cell is a podocyte. In some embodiments, the subject is a subject having PKD1 or polycystin-1 deficiency, and the nucleic acid encodes polycystin-1, and the target cell is a kidney cell. In some embodiments, the subject is a human subject.

In some embodiments, one or both of: (i) the nuclear localization element, or (ii) the innate immune response avoidance moiety, comprise an aptamer. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47. In some embodiments, the sequence configured to bind nucleolin comprises a nucleic acid sequence of any one of SEQ ID NO: 43-47, OR SEQ ID NO: 135-148. In some cases, the nuclear localization element or the aptamer comprising the sequence configured to bind nucleolin provides a beneficial technical effect of increasing nuclear localization and resulting gene expression of the cargo polynucleotide encoding a therapeutic transgene.

In some embodiments, the nuclear localization element or the aptamer comprises a sequence configured to bind importin. In some embodiments, the sequence configured to bind importin comprises a nucleic acid sequence of SEQ ID NO: 48, OR SEQ ID NO: 129-130. In some cases, the nuclear localization element or the aptamer comprising the sequence configured to bind importin provides a beneficial technical effect of increasing nuclear localization and resulting gene expression of the cargo polynucleotide encoding a therapeutic transgene.

In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NOS: 49-50. In some cases, the nuclear localization element or the aptamer comprising the sequence configured to bind a nucleoporin protein provides a beneficial technical effect of increasing nuclear localization and resulting gene expression of the cargo polynucleotide encoding a therapeutic transgene.

In some embodiments, the innate immune response avoidance moiety comprises a cyclic GMP-AMP synthase (cGAS) antagonist, absent in melanoma 2 inflammasome (AIM2) antagonist, or toll-like receptor 9 (TLR9) antagonist.

In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a nucleic acid sequence configured to bind cGAS.

In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises any SEQ ID NO: 51 or 54. In some embodiments, the aptamer comprising the nucleic acid sequence configured to bind cGAS is a cGAS antagonist. In some cases, the innate immune response avoidance moiety or the aptamer comprising the sequence configured to bind cGAS provides a beneficial technical effect of reducing clearance of the nucleic acid by the cell and increasing gene expression of the cargo polynucleotide encoding a therapeutic transgene.

In some embodiments, the aptamer comprises a nucleic acid sequence configured to bind an AIM2 inflammasome. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises SEQ ID NO: 51 or 54. In some embodiments, the aptamer comprising the nucleic acid sequence configured to bind AIM2 is an AIM2 antagonist. In some cases, the innate immune response avoidance moiety or the aptamer comprising the sequence configured to bind AIM2 provides a beneficial technical effect of reducing clearance of the nucleic acid by the cell and increasing gene expression of the cargo polynucleotide encoding a therapeutic transgene.

In some embodiments, the aptamer comprises a nucleic acid sequence configured to bind TLR9. In some embodiments, the sequence configured to bind TLR9 comprises a CpG motif. In some embodiments, the CpG motif comprises any one of SEQ ID NO: 90-93. In some embodiments, the sequence configured to bind TLR9 comprises any one of SEQ ID NO: 51-54. In some embodiments, the aptamer comprising the nucleic acid sequence configured to bind TLR9 is a TLR9 antagonist. In some cases, the innate immune response avoidance moiety or the aptamer comprising the sequence configured to bind TLR9 provides a beneficial technical effect of reducing clearance of the nucleic acid by the cell and increasing gene expression of the cargo polynucleotide encoding a therapeutic transgene.

In another aspect, the present disclosure provides a kit to perform the methods described herein. In some embodiments, the kit comprises: (a) a first container comprising microbubbles for sonoporation; and (b) a second container comprising nucleic acids comprising a cargo polynucleotide encoding a transgene and an aptamer; and (c) instructions for administration of ultrasound acoustic energy. In some embodiments, the kit comprises: (a) a first container comprising sonoactive agents; and (b) a second container comprising nucleic acids comprising a cargo polynucleotide encoding a transgene and an aptamer; and (c) instructions for administration of ultrasound acoustic energy.

Aspects disclosed herein provide a kit comprising: nucleic acids comprising (1) a cargo polynucleotide comprising an expression cassette that encodes a therapeutic transgene and (2) one or both of: (i) a nuclear localization element, and/or (ii) an innate immune response avoidance moiety; and a sonoactive agent. In some embodiments, the kit further includes instructions for applying ultrasound acoustic energy to a subject, wherein the ultrasound acoustic energy is configured to deliver the nucleic acids to the cell. In some embodiments, the ultrasound acoustic energy is configured to deliver the nucleic acids to the cell. In some embodiments, the nuclear localization element increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, at least or 5 fold as compared to a nucleic acid lacking the nuclear localization element. In some embodiments, the immune response avoidance moiety increases expression of the cargo polynucleotide in the target cell by at least 1.35, at least 1.40, at least 1.45, at least 1.50, at least 1.55, at least 1.60, at least 1.65, at least 1.75, at least 1.80, at least 1.85, at least 1.90, at least 1.95, at least 2.0, at least 2.25. 2.5, at least 2.75. 3, at least 3.5, at least 4, at least 4.5, at least or 5 fold as compared to a nucleic acid lacking the immune response avoidance moiety.

In some embodiments, one or both of: (i) the nuclear localization element, or (ii) the innate immune response avoidance moiety, comprise an aptamer. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47, OR SEQ ID NO: 135-148. In some cases, the nuclear localization element or the aptamer comprising the sequence configured to bind nucleolin provides a beneficial technical effect of increasing nuclear localization and resulting gene expression of the cargo polynucleotide encoding a therapeutic transgene.

In some embodiments, the nuclear localization element or the aptamer comprises a sequence configured to bind importin. In some embodiments, the sequence configured to bind importin comprises a nucleic acid sequence of SEQ ID NO: 48, 129, or 130. In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some cases, the nuclear localization element or the aptamer comprising the sequence configured to bind the nucleoporin protein provides a beneficial technical effect of increasing nuclear localization and resulting gene expression of the cargo polynucleotide encoding a therapeutic transgene.

In some embodiments, the innate immune response avoidance moiety comprises a cyclic GMP-AMP synthase (cGAS), absent in melanoma 2 inflammasome (AIM2), or toll-like receptor 9 (TLR9) antagonist. In some embodiments, the aptamer comprises a sequence configured to reduce an innate immune response to extra-nuclear DNA in a cell. In some embodiments, the extra-nuclear DNA comprises DNA located in cytosol in the cell. In some embodiments, the aptamer comprises a nucleic acid sequence configured to bind cGAS. In some embodiments, the nucleic acid sequence configured to bind a cGAS comprises a telomeric motif. In some embodiments, the telomeric motif comprises SEQ ID NO: 89. In some embodiments, the nucleic acid sequence configured to bind the cGAS comprises any one of SEQ ID NO: 51, 54. In some embodiments, the aptamer comprising the nucleic acid sequence configured to bind cGAS is a cGAS antagonist. In some cases, the innate immune response avoidance moiety or the aptamer comprising the sequence configured to bind cGAS provides a beneficial technical effect of reducing clearance of the nucleic acid by the cell and increasing gene expression of the cargo polynucleotide encoding a therapeutic transgene.

In some embodiments, the aptamer comprises a nucleic acid sequence configured to bind an AIM2. In some embodiments, the nucleic acid sequence configured to bind the AIM2 comprises a telomeric motif. In some embodiments, the telomeric motif comprises SEQ ID NO: 89. In some embodiments, the nucleic acid sequence configured to bind the AIM2 comprises any one of SEQ ID NO: 51, or 54. In some embodiments, the aptamer comprising the nucleic acid sequence configured to bind AIM2 is an AIM2 antagonist. In some cases, the innate immune response avoidance moiety or the aptamer comprising the sequence configured to bind AIM2 provides a beneficial technical effect of reducing clearance of the nucleic acid by the cell and increasing gene expression of the cargo polynucleotide encoding a therapeutic transgene.

In some embodiments, the aptamer comprises a nucleic acid sequence configured to bind TLR9. In some embodiments, the sequence configured to bind TLR9 comprises a CpG motif. In some embodiments, the CpG motif comprises any one of SEQ ID NO: 90-93. In some embodiments, the sequence configured to bind TLR9 comprises any one of SEQ ID NO: 51-54. In some embodiments, the aptamer comprising the nucleic acid sequence configured to bind TLR9 is a TLR9 antagonist. In some cases, the innate immune response avoidance moiety or the aptamer comprising the sequence configured to bind TLR9 provides a beneficial technical effect of reducing clearance of the nucleic acid by the cell and increasing gene expression of the cargo polynucleotide encoding a therapeutic transgene.

In some embodiments, the sonoactive agent comprises a microbubble. In some embodiments, the sonoactive agent comprises a protein-stabilized shell. In some embodiments, the sonoactive agent comprises a lipid stabilized shell. In some embodiments, the cargo polynucleotide is covalently coupled to one or both of: (i) a nuclear localization element, and/or (ii) an innate immune response avoidance moiety. In some embodiments, the nucleic acid comprising the cargo polynucleotide is a linear DNA construct. In some embodiments, the nucleic acid comprising the cargo polynucleotide is a closed linear DNA construct closed linear DNA construct. In some embodiments, the nucleic acid comprising the cargo polynucleotide is a closed linear DNA construct comprising at least 2 modified nucleotides. In some embodiments, the nucleic acid comprising the cargo polynucleotide is a closed linear DNA construct comprising a stem region comprising a double stranded DNA sequence covalently closed at both ends by hairpin loops. In some embodiments, the hairpin loops are single-stranded. In some embodiments, the hairpin loops comprise any one of SEQ ID NO: 55-62, 101-128, or 205-252. In some embodiments, a first hairpin loop of the hairpin loops comprise any one of SEQ ID NO: 55-62, 101-128, or 205-252, and wherein a second hairpin loop of the hairpin loops comprise a different sequence than the first hairpin loop of any one of SEQ ID NO: 55-62, 101-128, or 205-252. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the at least two modified nucleotides are in one or both single stranded end loops forming the stem region of the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, 4, at least 5, at least 6, at least 7, at least 8, at least 9, at least 10, at least 11, at least 12, at least 13, at least 14, at least 15, at least 16, at least 17, at least 18, at least 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeat(s) (ITR) sequence.

Aspects disclosed herein provide an isolated nucleic acid comprising: a cargo polynucleotide comprising an expression cassette that encodes a therapeutic transgene configured for expression in a target cell of a subject, and a nuclear localization element configured to increase expression of the cargo polynucleotide in the target cell. In some embodiments, the nuclear localization element increases expression of the cargo polynucleotide in the target cell by at least 1.25 fold as compared to a nucleic acid lacking the nuclear localization element. In some embodiments, the nuclear localization element comprises an aptamer. In some embodiments, the nuclear localization element or the aptamer comprises a sequence configured to bind nucleolin. In some embodiments, the sequence configured to bind nucleolin comprises a nucleic acid sequence of any one of SEQ ID NO: 3-47. In some embodiments, the sequence configured to bind nucleolin comprises a nucleic acid sequence of any one of SEQ ID NO: 43-47, OR SEQ ID NO: 135-148. In some cases, the nuclear localization element or the aptamer comprising the sequence configured to bind nucleolin provides a beneficial technical effect of increasing nuclear localization and resulting gene expression of the cargo polynucleotide encoding a therapeutic transgene. In some embodiments, the target cell is a liver cell. In some embodiments, the target cell is a hepatocyte. In some embodiments, the target cell is a LSEC. In some embodiments, the target cell is a kidney cell. In some embodiments, the target cell is a proximal tubular epithelial cell. In some embodiments, the target cell is a podocyte. In some embodiments, the target cell is a muscle cell. In some embodiments, the method is a method to treat a subject in need of a gene therapy or a protein replacement therapy. In some embodiments, the method is a method of treating a mammalian subject having a genetic disorder with a nucleic acid encoding a therapeutic transgene. In some embodiments, the cargo polynucleotide comprises an expression cassette encoding the therapeutic transgene, wherein the therapeutic transgene is configured for expression in the target cell of the subject. In some embodiments, the method is a method for use of the nucleic acid, the sonoactive agent or the microbubble, and the ultrasound in treatment of a subject having a genetic disorder requiring a gene therapy or a protein replacement therapy. In some embodiments, the subject is a subject having Hemophilia A or FVIII deficiency, the nucleic acid encodes FVIII, and the target cell is a liver cell. In some embodiments, the subject is a subject having Alport's Syndrome or COL4A5 deficiency, and the nucleic acid encodes alpha5(IV) chain of collagen IV, and the target cell is a podocyte. In some embodiments, the subject is a subject having PKD1 or polycystin-1 deficiency, and the nucleic acid encodes polycystin-1, and the target cell is a kidney cell. In some embodiments, the subject is a human subject.

In some embodiments, the nuclear localization element or the aptamer comprises a sequence configured to bind importin. In some embodiments, the sequence configured to bind importin comprises a nucleic acid sequence of SEQ ID NO: 48, 129, or 130. In some cases, the nuclear localization element or the aptamer comprising the sequence configured to bind importin provides a beneficial technical effect of increasing nuclear localization and resulting gene expression of the cargo polynucleotide encoding a therapeutic transgene.

In some embodiments, the nuclear localization element or the aptamer comprises a sequence configured to bind a nucleoporin protein. In some embodiments, the nucleoporin protein is positioned on a cytoplasmic ring or a cytoplasmic filament of the nuclear pore complex. In some embodiments, the nucleoporin protein comprises NUP 214, NUP 88, or NUP 358. In some embodiments, the nucleoporin protein comprises NUP 358. In some embodiments, the sequence configured to bind the nucleoporin protein comprises a nucleic acid sequence of any one of SEQ ID NO: 49-50. In some cases, the nuclear localization element or the aptamer comprising the sequence configured to bind a nucleoporin protein provides a beneficial technical effect of increasing nuclear localization and resulting gene expression of the cargo polynucleotide encoding a therapeutic transgene.

In some embodiments, the cargo polynucleotide is covalently coupled to the nuclear localization element. In some cases, the cargo polynucleotide being covalently coupled to the nuclear localization element provides a beneficial technical effect of increasing nuclear localization and resulting gene expression of the cargo polynucleotide encoding a therapeutic transgene. In some embodiments, the isolated nucleic acid comprising the cargo polynucleotide is a linear DNA construct. In some embodiments, the isolated nucleic acid comprising the cargo polynucleotide is a closed linear DNA construct. In some embodiments, the isolated nucleic acid comprising the cargo polynucleotide is a closed linear DNA construct comprising at least 2 modified nucleotides. In some embodiments, the isolated nucleic acid comprising the cargo polynucleotide is a closed linear DNA construct comprising a stem region comprising a double stranded DNA sequence covalently closed at both ends by hairpin loops. In some embodiments, the hairpin loops are single-stranded. In some embodiments, the hairpin loops form a stem region of the aptamer. In some embodiments, the at least two modified nucleotides are located in one or both single-stranded end loops of the closed linear DNA construct; at least one modified nucleotide is located in one of the single stranded end loops and at least another modified nucleotide is located in one of the single stranded end loops forming the stem region of the aptamer. In some embodiments, the at least two modified nucleotides are in one or both single stranded end loops forming the stem region of the aptamer. In some embodiments, the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide. In some embodiments, the closed linear DNA construct comprises from 3 to 20 modified nucleotides, for example, 4, 5, 6, 7, 8, 9, 10, 11, 12, 13, 14, 15, 16, 17, 18, 19 or 20 modified nucleotides. In some embodiments, the closed linear DNA construct comprises at least two LNA nucleotides. In some embodiments, the closed linear DNA construct comprises at least two thiophosphate nucleotides. In some embodiments, the closed linear DNA construct comprises at least two restriction sites flanking the expression cassette. In some embodiments, the closed linear DNA construct comprises a primase recognition site. In some embodiments, the closed linear DNA construct comprises an inverted terminal repeat(s) (ITR) sequence(s). In some embodiments, the ITR sequence(s) are located in the stem region of the aptamer. In some embodiments, the second aptamer comprises a different nucleic acid sequence than the nuclear localization element which comprises the aptamer. In some embodiments, the isolated nucleic acid comprises a spacer sequence preceding or following (e.g., 5′ or 3′) of the expression cassette before the hairpin loop(s). In some embodiments, the spacer sequence is at least 3, 4, 5, 6, 7, 8, 9, 10, 11, 12, 15, 18, 21, 23, 27, or 30 nucleotides. In some cases, the spacer sequence provides a beneficial technical effect of allowing for proper aptamer secondary conformation forms (see, e.g., FIGS. 7-9) and improves resulting gene expression of the cargo polynucleotide encoding a therapeutic transgene In some embodiments, the aptamer(s) are comprised within the hairpin loop(s). In some embodiments, the aptamer(s) are at least partially single stranded. In some embodiments, the aptamer(s) comprise any one of SEQ ID NO: 3-54, 129-130, or 135-148. In some embodiments, the isolated nucleic acid comprises a spacer sequences preceding and following (e.g., 5′ or 3′) of the expression cassette before the hairpin loop(s). In some embodiments, the isolated nucleic acid is configured to form an episome in a nucleus of the cell. In some embodiments, the therapeutic transgene configured for expression in the target cell is an exogenous transgene to the subject. In some embodiments, the exogenous transgene provides a gain of function to the subject by expression of the therapeutic transgene. In some embodiments, the expression cassette is at least 4.5, at least 5, at least 6, at least 7, at least 8, at least 9, at least 10, at least 11, at least 12, at least 13, at least 14, or at least 15 kb long. In some embodiments, the isolated nucleic acid is at least 4.5, at least 5, at least 6, at least 7, at least 8, at least 9, at least 10, at least 11, at least 12, at least 13, at least 14, at least or 15 kb long. In some embodiments, the therapeutic transgene encodes FVIII, FIX, alpha5(IV) chain of collagen IV, alpha4(IV) chain of collagen IV, alpha3(IV) chain of collagen IV, protein polycystin-1 (PC1), or polycystin-2 protein (PC2). In some embodiments, the therapeutic transgene is FVIII, FIX, COL4A3, COL4A5, COL4A4, PKD1, or PKD2.

In some embodiments, the nucleic acids comprises an expression cassette. As used herein, an expression cassette comprises a coding nucleic acid sequences, e.g., an expression cassette encoding a transgene. In some cases, an expression cassette can comprise a regulatory element such as a promoter, enhancer, ribosome binding site, or transcription termination signal.

In some embodiments, the first container and second container are configured to induce the expression of the transgene in the target cell of the subject within 20 hours after the transfection.

In some embodiments, the method further includes inducing expression of the cargo polynucleotide and maintaining expression of a protein encoded by the cargo polynucleotide for at least 1, at least 2, at least 3, at least 4, at least 5, at least 6, at least or 7 days following administration of the microbubble and the nucleic acid, and application of the ultrasonic acoustic energy. In some embodiments, the method further includes inducing expression of the cargo polynucleotide and maintaining expression of a protein encoded by the cargo polynucleotide for at least 1, at least 2, at least 3, at least 4, at least 5, at least 6, at least or 7 days following administration of the microbubble and the nucleic acid, and application of the ultrasonic acoustic energy.

In some embodiments, the method further includes increasing expression of the cargo polynucleotide by increasing the dosage of the cargo polynucleotide administered to the subject. In some embodiments, the method further includes increasing expression of the cargo polynucleotide by increasing the dosage of the nucleic acid administered to the subject in a linear manner.

In some embodiments, the kit further comprises instructions for software and hardware directions for the safe and effective operation of an ultrasound machine sufficient to disrupt the sonoactive microstructures or sonoactive agents to generate the sonoporation processes which include but are not limited to the following: disrupting the microstructures, inducing inertial and stable cavitation, promoting endocytosis and inter-endothelial gap formation, microstreaming at cell surfaces, thereby increasing transfection of a cargo polynucleotide to a cell. In some embodiments, the instructions described methods for improvement of gene transfection using sonoporation by applying alternating ultrasonic acoustic energy between a first MI then a second MI. In some embodiments, the kit further comprises instructions for administration of the first container and the second container.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

As used herein, the term “aptamer” refers to an oligonucleotide sequence which is at least partially single stranded comprising a sequence of nucleic acids which bind a target antigen.

As used herein the term “sequence identity” refers to the percentage identity calculated as the matching residues divided by the total number of residues in the total alignment when performing a consensus alignment of two sequences, with gaps in the alignment scored as a mismatching residue.

The terms “subject,” “individual,” or “patient” are often used interchangeably herein. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### EXAMPLES

The following examples are included for illustrative purposes only and are not intended to limit the scope of the invention.

### Example 1: Sonoporation of Liver Cells with Closed Linear DNA Construct Linked to a Nucleolin Targeted Aptamer

The following embodiment illustrates the sonoporation mediated delivery of a closed ended linear DNAclosed linear DNA construct genetic payload that is covalently linked to a DNA aptamer targeting nucleolin.

In this experiment, gene expression levels and kinetics of the reporter gene luciferase are investigated in a mouse liver.

### Experimental Conditions and Protocols:

There are four experimental groups, each of which includes 4 BALB/c mice. Prior to the experiment, each mouse is implanted with a jugular vein catheter (JVC), through which the sonoactive microstructure and nucleotide constructs are administered. All animals have the abdomen shaved, and a depilatory agent is applied.

The ApoE-AAT/luciferase plasmid which encodes wildtype firefly luciferase is used as a reporter gene in this experiment under a promoter sequence. The cargo polynucleotide includes: ApoE-AAT-Fluc.

The ApoE-AAT/luciferase closed end DNA (luc-closed linear DNA construct) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA (AGGGATAACATGGCC/I/CTC/I/GGCCATGTTAT) SEQ ID NO: 2. This luc-closed linear DNA construct is not targeted to any specific moiety.

The ApoE-AAT/luciferase closed end DNA targeting nucleolin (luc-closed linear DNA construct-Nuc) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA is a nucleolin binding (e.g., facilitating nuclear entry) aptamer generated using SELEX method against nucleolin. An exemplary anti-nucleolin aptamer may comprise a sequence of any one of SEQ ID NO: 3-47.

### Optison Injectate Preparation

Mice in this experiment are randomized into the following experimental groups:

Group 1. Naïve control animals. No ultrasound is applied or any material injected.

Group 2. Animals receive an injection of Optison microbubble and ApoE-AAT/luciferase nanoplasmid, and ultrasound energy is applied.

Group 3. Animals receive an injection of Optison microbubble and luc-closed linear DNA construct, and ultrasound energy is applied.

Group 4. Animals receive an injection of Optison microbubble and luc-closed linear DNA construct-Nuc, and ultrasound energy is applied.

Following administration of the microbubbles and nucleic acid payload, ultrasound acoustic energy is delivered to the liver area of mice in these experiments using a L6-24 probe positioned perpendicular to the mouse to locate the lateral view of liver using B-mode ultrasound imaging at the low mechanical index (MI) value of 0.07. The depth setting is set to 2 cm, and the zoom to 0. Ultrasound is delivered continuously and alternated between a low mechanical index (MI) value of 0.07 and a high MI value of 1.5, without ceasing application of the ultrasound energy at any point during the treatment session. Nine flashes of high MI ultrasound at 1.5 are delivered with an interval of 4 seconds between each flash, and the administration of the 9 pulses is repeated three times. The high MI pulse duration is about 0.82 microseconds. The administration of the ultrasound is less than 110 seconds.

IVIS fluorescence radiance imaging for mice in all groups provides maximum average fluorescence radiance values for mice at the 24 h, 72 h, and 1 week time points. The mean of fluorescence radiance values for mice in each group are considered.

### Results:

The Group 1 mice do not reveal any recorded bioluminescence. The bioluminescence signal levels are comparable for Groups 2 and 3. The signal is substantially higher in the animals from Group 4 that receive the ApoE-AAT/luciferase closed ended DNA with nucleolin (e.g., nuclear entry facilitating) targeted aptamers.

### Example 2: Sonoporation of Liver Cells with Closed Linear DNA Construct Linked to a Nucleolin or Nucleoporin Targeted Aptamer

The following embodiment illustrates the sonoporation mediated delivery of a closed ended linear DNAclosed linear DNA construct genetic payload that is covalently linked to a DNA aptamer targeting nucleolin.

In this experiment, gene expression levels and kinetics of the reporter gene luciferase are investigated in a mouse liver.

### Experimental Conditions and Protocols:

There are four experimental groups, each of which includes 4 BALB/c mice. Prior to the experiment, each mouse is implanted with a jugular vein catheter (JVC), through which the sonoactive microstructure and nucleotide constructs are administered. All animals have the abdomen shaved, and a depilatory agent is applied.

The ApoE-AAT/luciferase plasmid which encodes wildtype firefly luciferase is used as a reporter gene in this experiment under a promoter sequence. The cargo polynucleotide included: ApoE-AAT-Fluc.

The ApoE-AAT/luciferase closed end DNA (luc-closed linear DNA construct) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA (AGGGATAACATGGCC/I/CTC/I/GGCCATGTTAT) SEQ ID NO: 2. This luc-closed linear DNA construct is not targeted to any specific moiety.

The ApoE-AAT/luciferase closed end DNA targeting nucleolin (luc-closed linear DNA construct-Nuc) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA is a nucleolin binding (e.g., facilitating nuclear entry) aptamer generated using SELEX method against nucleolin. An exemplary anti-nucleolin aptamer may comprise a sequence of any one of SEQ ID NO: 3-47.

### Sonazoid Injectate Preparation

Mice in this experiment are randomized into the following experimental groups:

Group 1. Naïve control animals. No ultrasound is applied or any material injected.

Group 2. Animals receive an injection of Sonazoid microbubble and ApoE-AAT/luciferase nanoplasmid, and ultrasound energy is applied.

Group 3. Animals receive an injection of Sonazoid microbubble and luc-closed linear DNA construct, and ultrasound energy is applied.

Group 4. Animals receive an injection of Sonazoid microbubble and luc-closed linear DNA construct-Nuc, and ultrasound energy is applied.

Following administration of the microbubbles and nucleic acid payload, ultrasound acoustic energy is delivered to the liver area of mice in these experiments using a L6-24 probe positioned perpendicular to the mouse to locate the lateral view of liver using B-mode ultrasound imaging at the low mechanical index (MI) value of 0.07. The depth setting is set to 2 cm, and the zoom to 0. Ultrasound is delivered continuously and alternated between a low mechanical index (MI) value of 0.07 and a high MI value of 1.5, without ceasing application of the ultrasound energy at any point during the treatment session. Nine flashes of high MI ultrasound at 1.5 are delivered with an interval of 4 seconds between each flash, and the administration of the 9 pulses is repeated three times. The high MI pulse duration is about 0.82 microseconds. The administration of the ultrasound is less than 110 seconds.

IVIS fluorescence radiance imaging for mice in all groups provides maximum average fluorescence radiance values for mice at the 24 h, 72 h, and 1 week time points. The mean of fluorescence radiance values for mice in each group are considered.

### Results:

The Group 1 mice do not reveal any recorded bioluminescence. The bioluminescence signal levels are comparable for Groups 2 and 3. The signal is substantially higher in the animals from Group 4 that received the ApoE-AAT/luciferase closed ended DNA with nucleolin (e.g., nuclear entry facilitating) targeted aptamers.

### Example 3: Sonoporation of Liver Cells with Closed Linear DNA Construct Linked to an Importin-Targeted Aptamer

The following embodiment illustrates the sonoporation mediated delivery of a closed ended linear DNAclosed linear DNA construct genetic payload that is covalently linked to a DNA aptamer targeting importin.

In this experiment, gene expression levels and kinetics of the reporter gene luciferase are investigated in a mouse liver.

### Experimental Conditions and Protocols:

There are four experimental groups, each of which includes 4 BALB/c mice. Prior to the experiment, each mouse is implanted with a jugular vein catheter (JVC), through which the sonoactive microstructure and nucleotide constructs are administered. All animals have the abdomen shaved, and a depilatory agent is applied.

The ApoE-AAT/luciferase plasmid which encodes wildtype firefly luciferase is used as a reporter gene in this experiment under a promoter sequence. The cargo polynucleotide includes: ApoE-AAT-Fluc.

The ApoE-AAT/luciferase closed end DNA (luc-closed linear DNA construct) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA (AGGGATAACATGGCC/I/CTC/I/GGCCATGTTAT) SEQ ID NO: 2. This luc-closed linear DNA construct is not targeted to any specific moiety.

The ApoE-AAT/luciferase closed end DNA targeting importin (luc-closed linear DNA construct-Imp) is generated as described in US20230075380A1, the disclosure of which is hereby incorporated by reference. In this case instead of an eGFP sequence the ApoE-AAT/luciferase sequence is used as the promoter and transgene. The hairpin DNA adaptor used to close the ends of the double-stranded DNA is an importin binding (e.g., facilitating nuclear entry) aptamer generated using SELEX method against importin. An exemplary anti-importin aptamer may comprise a sequence of SEQ ID NO: 48.

### Optison Injectate Preparation

Mice in this experiment are randomized into the following experimental groups:

Group 1. Naïve control animals. No ultrasound is applied or any material injected.

Group 2. Animals receive an injection of Optison microbubble and ApoE-AAT/luciferase nanoplasmid, and ultrasound energy is applied.

Group 3. Animals receive an injection of Optison microbubble and luc-closed linear DNA construct, and ultrasound energy is applied.

Group 4. Animals receive an injection of Optison microbubble and luc-closed linear DNA construct-Imp, and ultrasound energy is applied.

Following administration of the microbubbles and nucleic acid payload, ultrasound acoustic energy is delivered to the liver area of mice in these experiments using a L6-24 probe positioned perpendicular to the mouse to locate the lateral view of liver using B-mode ultrasound imaging at the low mechanical index (MI) value of 0.07. The depth setting is set to 2 cm, and the zoom to 0. Ultrasound is delivered continuously and alternated between a low mechanical index (MI) value of 0.07 and a high MI value of 1.5, without ceasing application of the ultrasound energy at any point during the treatment session. Nine flashes of high MI ultrasound at 1.5 are delivered with an interval of 4 seconds between each flash, and the administration of the 9 pulses is repeated three times. The high MI pulse duration is about 0.82 microseconds. The administration of the ultrasound is less than 110 seconds.

IVIS fluorescence radiance imaging for mice in all groups provides maximum average fluorescence radiance values for mice at the 24 h, 72 h, and 1 week time points. The mean of fluorescence radiance values for mice in each group are considered.

### Results:

The Group 1 mice do not reveal any recorded bioluminescence. The bioluminescence signal levels are comparable for Groups 2 and 3. The signal is substantially higher in the animals from Group 4 that receive the ApoE-AAT/luciferase closed ended DNA with importin (e.g., nuclear entry facilitating) targeted aptamers.

### Example 4: Protein SELEX Method for Screening of Nucleic Acid Aptamer

In this example, nucleic acids are screened to target recombinant proteins to produce aptamers.

Recombinant NUP 358 protein is prepared and immobilized onto a solid support, such as NHS-activated Sepharose beads or high-binding 96-well plates, ensuring proper folding and functional activity of the target protein. A randomized nucleic acid library, consisting of either single-stranded DNA (ssDNA) or RNA, is used to initiate the SELEX process. This library contains random sequence regions of 20-80 nucleotides and flanking primer-binding sites for amplification. The nucleic acid library is denatured by heating to 95° C. and rapidly cooled to promote proper folding, followed by incubation with the immobilized NUP 358 in a suitable binding buffer. The buffer may include Tris-HCl, NaCl, KCl, and MgCl₂, with optional stabilizers for RNA aptamers.

Following incubation, unbound or weakly bound nucleic acids are removed through a series of washes using a washing buffer with gradually increasing salt concentrations to enhance specificity. The tightly bound aptamers are eluted using either a high-salt buffer or low-pH elution buffer, depending on the nature of the interaction. For RNA aptamers, elution may require gentle heating in an RNase-free environment to preserve integrity. The eluted aptamers are then amplified by PCR (for ssDNA) or reverse transcription followed by PCR (for RNA aptamers), ensuring recovery of the selected sequences for subsequent rounds.

The SELEX process is iterative, with 8-12 rounds of selection, each round involving incubation, washing, elution, and amplification. In each successive round, the washing stringency is increased to enrich the pool for high-affinity aptamers that specifically bind NUP 358. Enrichment is monitored by techniques such as fluorescence anisotropy, surface plasmon resonance (SPR), or electrophoretic mobility shift assays (EMSA), to track the increasing binding affinity of the enriched aptamer pool. To enhance the specificity of aptamers, a counter-SELEX step is introduced, wherein the library is incubated with irrelevant proteins and beads without NUP 358 to remove non-specific binders.

After the final SELEX round, the enriched pool is cloned into a suitable vector for sequencing, and bioinformatic analysis is conducted to identify unique aptamer sequences. Individual sequences are synthesized or transcribed for further characterization, including determining their dissociation constants (K_{d}) and binding specificity to NUP 358. High-affinity aptamers are expected to exhibit minimal cross-reactivity with other proteins. Optimization of the SELEX process may include fine-tuning washing stringency, adjusting target protein immobilization, and employing negative controls to assess non-specific background binding. The resulting aptamers are found to bind the nuclear pore complex at NUP 358.

### Example 5: Synthesis of Closed End Linear DNA Constructs with Single Stranded Aptamers

The following protocol describes formation of linear nucleic acid vectors closed at each end with single-stranded aptamers which target nucleolin, a nuclear pore protein, or immune proteins active in innate immune system activation toward double-stranded DNA in the cell.

Preliminarily, double-stranded plasmid DNA (pDNA) encoding the expression cassette of interest was chemically synthesized (e.g., SEQ ID NO: 75). Separately, single stranded DNA (ssDNA) encoding the aptamer sequence of interest (e.g., any one of SEQ ID NO: 43-54) preceded by a 5′ ITR upstream of the aptamer sequence and flanked by a 3′ ITR sequence downstream of the aptamer sequence (e.g., SEQ ID NO: 76 and 77, respectively), for example see SEQ ID NO: 78 (5′ ITR-NUP 358 Apt-3′ ITR) was chemically synthesized.

The ssDNA was resuspended in water to a concentration of about 100 uM in 20× saline sodium citrate buffer. The ssDNA was denatured by heating to 95 C for 10 min and allowed to anneal naturally at room temperature for 30 min, forming a double-stranded region by hybridization of the ITR regions and leaving the aptamer sequence as a single stranded region.

The dsDNA was linearized with Bsal digestion in water in 10× rCutSmart buffer, incubated at 37 C for 2 hours, and heat inactivated by heating to 75 C for 10 min. The linearized dsDNA was purified and concentrated to a concentration of 92 ug/uL.

Following linearization, solutions of ssDNA and dsDNA were combined, and the ssDNA was covalently bonded to the linearized dsDNA by ligation with 10×T4 ligase buffer and T4 DNA ligase at 16 C, followed by heat inactivation at 75 C for 10 minute to form the dsDNA product closed at each end by single stranded DNA comprising the aptamers. The ligated product was then purified and concentrated.

Following ligation, the product was treated with endonucleases Nhel (New England Biolabs) and Pcil (New England Biolabs) to cut residual pDNA, followed by incubation in a thermal cycler for 1 hr at 37 C, addition of 1 μL of ExoIII endonuclease, incubation in a thermal cycler for another 1 hr at 37 C, and heat inactivation by heating to 75 C for 10 min. The product I then purified using DNA Clean Up and Concentrator 25 (Zymo) and eluted in water. Concentration was then determined using Qubit dsDNA BR Assay Kit. Linearization was validated by staining with nucleic acid stain on an agarose gel using gel electrophoresis.

### Example 6: In-Vitro Evaluation of Vectors with Nuclear Localization Elements and Innate Immune Response Avoidance Moieties in Transfection of iPSC-Derived Hepatocytes

The following experiment evaluates expression of a fluorescent reporter transgene following transfection of iPSC-derived hepatocytes with linear nucleic acid vectors closed at each end with single stranded aptamers which target nucleolin, a nuclear pore protein, or an innate immune system sensor of extranuclear doubles stranded DNA in the cell.

An expression cassette comprising a fluorescent reporter transgene (tdTomato) under the influence of a CAG promoter was utilized. The expression cassette was positioned in the center double-stranded region of the linear nucleic acid vectors, which were closed at each end by aptamer sequences. In brief, linear nucleic acid vectors having the expression cassette in double-stranded portion of the vector with closed single stranded ends comprising aptamers were chemically synthesized as described in Example 5. Each nucleic acid delivery vector utilized in the evaluation included an expression cassette having the sequence of SEQ ID NO: 75. The closed end linear nucleic acids comprised the sequences of SEQ ID NO: 64-74, and 86-87. The closed end linear nucleic acids vectors tested comprised the sequences shown in SEQ ID NO: 64-74. A closed end linear nucleic acid vector with a single stranded adaptor non-comprising an aptamer sequence was utilized as a control (Adaptor-19, SEQ ID NO: 87), and results were also compared against circular DNA formats including miniplasmid DNA comprising the same expression cassette (Nanoplasmid, SEQ ID NO: 88) and a standard plasmid format comprising the same expression cassette (PUC57, SEQ ID NO: 86).

The cell line utilized in this experiment was iCell Hepatocytes 2.0 (Fujifilm Cellular Dynamics, #01434). Cells were plated on collagen I-coated 96-well plates after thaw and were differentiated for 5 days in plating media with a daily media change. After plating for 5 days, cells were cultured in cell plating media until the day of transfection. Cells were transfected 8 days post-differentiation using 300 ng (300 uL) of the closed end linear nucleic acids vectors (SEQ ID NO: 64-74) using 0.45 uL of Lipofectamine 3000 (Invitrogen, Cat: L3000008). Upon harvest, cells were analyzed using Steady-Glo assay system for evaluation of fluorescence reported as relative light units (RLU) to evaluate the efficiency of the transfection.

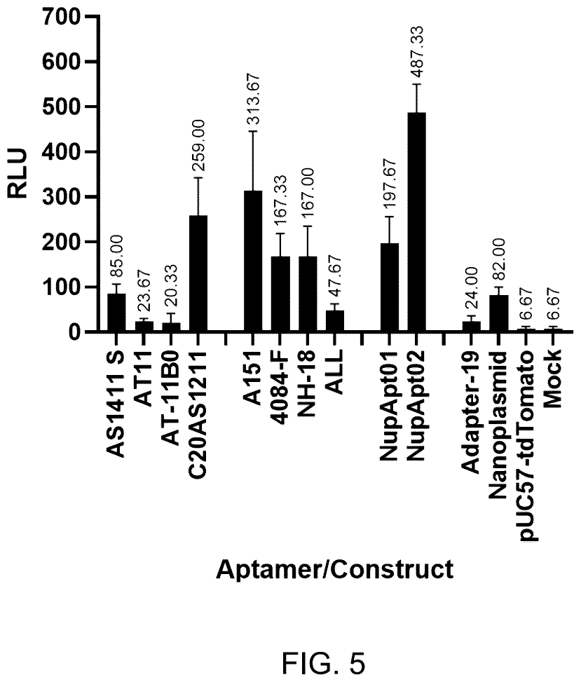

Results are illustrated in FIG. 5, in which it is shown that cells transfected with the closed end linear nucleic acids vectors comprising the nuclear localization element with a sequence configured to bind importin (SEQ ID NOS: 43, 46) exhibited approximately 3.5-fold and 10.8-fold increases in fluorescence, respectively, as compared to the closed end linear nucleic acid lacking the nuclear localization element (Adaptor 19, FIG. 5).

Results are further illustrated in FIG. 5, in which it is shown that cells transfected with the closed end linear nucleic acids vectors comprising the nuclear localization element with a sequence configured to bind a nucleoporin protein (SEQ ID NOS: 49, 50) exhibited approximately 8.2-fold and 20.3-fold increases in fluorescence, respectively, as compared to the closed end linear nucleic acid lacking the nuclear localization element (Adaptor 19, FIG. 5).

Results are further illustrated in FIG. 5, in which it is shown that cells transfected with the closed end linear nucleic acids vectors comprising the innate immune response avoidance moiety with a sequence configured to bind TLR9, AIM2, and cGAS (SEQ ID NOS: 51, 54) exhibited approximately 13-fold and 2-fold increases in fluorescence, respectively, as compared to the closed end linear nucleic acid lacking the innate immune response avoidance moiety (Adaptor 19, FIG. 5).

Results are further illustrated in FIG. 5, in which it is shown that cells transfected with the closed end linear nucleic acids vectors comprising the innate immune response avoidance moiety with a sequence configured to bind TLR9 (SEQ ID NOS: 52, 53) exhibited approximately 3-fold and 3-fold increases in fluorescence, respectively, as compared to the closed end linear nucleic acid lacking the innate immune response avoidance moiety (Adaptor 19, FIG. 5).

### Example 7: In-Vivo Evaluation of Vectors with Nuclear Localization Elements in a Murine Model of Sonoporation in the Liver

The following recites the evaluation the sonoporation-mediated delivery of a closed ended linear DNAclosed linear DNA construct genetic payload that is covalently linked to a DNA aptamer targeting nucleoporin proteins. In this experiment, gene expression levels and kinetics of the reporter gene luciferase were investigated in a mouse liver.

### Experimental Conditions and Protocols:

2 experimental groups were evaluated: a first control group evaluated a closed end linear nucleic acid vector with a single stranded adaptor lacking an aptamer sequence (Adaptor-19, SEQ ID NO: 87); a second experimental group evaluated a closed end linear nucleic acid vector comprising the nuclear localization element with a sequence configured to bind a nucleoporin protein (aptamer; SEQ ID NO: 50) (total vector sequence; SEQ ID NO: 72).

Prior to the experiment, each mouse was implanted with a jugular vein catheter (JVC), through which the sonoactive microstructure and nucleotide constructs were administered. All animals had their abdomen shaved, and a depilatory agent was applied. An acoustic contact agent (Aqua gel) was directly applied to the abdominal surface and then was applied to the upper abdominal skin surface of each mouse.

Sonazoid was reconstituted with 2 mL of sterile water for injection by injecting sterile water into the vial, inverting the vial gently 10 to 20 times to thoroughly mix the microbubbles, avoiding shaking the vial vigorously to avoid damaging the microbubbles. The final suspension was a uniform, milky-white suspension without large visible particles. 190 μL of the reconstituted microbubble suspension was drawn into a syringe, a solution of approximately 50 μg of the closed end linear nucleic acid vector under evaluation was drawn into the same syringe, and then nucleic acids were then mixed with the microbubble suspension by rolling the syringe between the fingers until the suspension appears homogenous. The DNA+microbubble suspension was then drawn out of the needle dead space (about 50 microliters), and the 18 G needle was exchanged for a 25 G blunt needle for injection into the JVC, and the suspension was intravenously administered into the JVC.

Following administration of the microbubbles and nucleic acid payload, ultrasound acoustic energy was delivered to the liver area of mice in these experiments using a GE LOGIC E9 equipped with a C1-6 probe positioned perpendicular to the mouse to locate the lateral view of liver using B-mode ultrasound at an MI of about 0.07, and the presence of microbubbles was confirmed in the liver. Following confirmation of microbubbles, the ultrasound focal depth setting was set to 2 cm, and the zoom to 0, and ultrasound was delivered at a mechanical index of 1.4 at a frequency of 2.28 MHz, alternating between 10 seconds of ultrasound application, and 20 seconds of rest in which the transducer was removed from the skin of the subject, for a total application of 30 seconds of ultrasound application.

IVIS fluorescence radiance imaging for mice in all groups provides maximum average fluorescence radiance values for mice at the 24 h, 72 h, and 1 week time points. The mean of fluorescence radiance values for mice in each group were considered.

### Results:

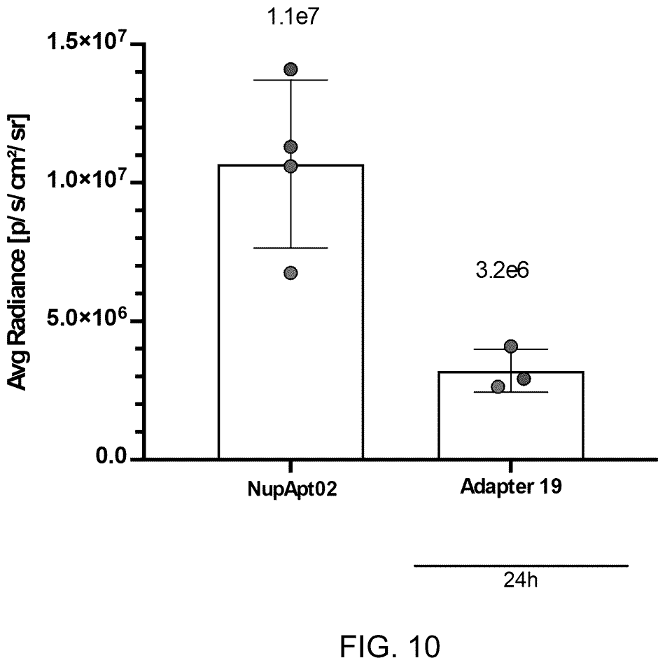

IVIS fluorescence radiance imaging of all groups was performed 24 h after the delivery of the first dose, the fluorescence measured at this time indicated the expression level of the luciferase payload. Results are illustrated in FIG. 10 in which, the Group 1 mice administered the closed end linear nucleic acid vector with a single stranded adaptor not comprising an aptamer sequence (Adaptor-19, SEQ ID NO: 87) exhibited an average radiance of 3.2 e6 p/s/cm2/sr, while Group 2 mice administered the closed end linear nucleic acids vector comprising the nuclear localization element with a sequence configured to bind a nucleoporin protein (aptamer; SEQ ID NO: 50) (total vector sequence SEQ ID NO: 72) exhibit an average radiance of 1.1 e7 p/s/cm2/sr-representing approximately a 3-fold increase over the Group 1 control.

### Example 8: In-Vivo Evaluation of Vectors and Immune Inhibitor Aptamers in a Murine Model of Sonoporation in the Liver

The following recites the evaluation the sonoporation-mediated delivery of a nucleic acids encoding genetic payloads coadministered with immune inhibitor aptamers. In this experiment, gene expression levels and kinetics of FVIII were investigated in a mouse liver.

### Experimental Conditions and Protocols:

3 experimental groups each of three RAG2 mice were evaluated: a first control group mice administered a miniplasmid vector encoding FVIII with no additional aptamers in phosphate buffered saline; a second experimental group evaluated a miniplasmid vector encoding FVIII with the administration of 50 μg of A151 innate immune suppressor aptamer (SEQ ID NO: 51) (an antagonist inhibitor of AIM2, TLR9, TLR7, and cGAS); and a third experimental group was administered a miniplasmid vector encoding FVIII with the administration of 50 μg of INH-18 innate immune suppressor aptamer (SEQ ID NO: 53) (an antagonist inhibitor of TLR9 and TLR7). Each group was administered identical 50 ug doses of the miniplasmid vector encoding FVIII, and were administered Optison microbubbles as the sonoactive agent.

Prior to the experiment, each mouse was implanted with a jugular vein catheter (JVC), through which the sonoactive microstructure and nucleotide constructs were administered. All animals had their abdomen shaved, and a depilatory agent was applied. An acoustic contact agent (Aqua gel) was directly applied to the abdominal surface and then was applied to the upper abdominal skin surface of each mouse.

A dose of sonoactive microstructure and DNA solution was readied by first preparing the sonoactive microstructures (Optison) as instructed on the label: remove from 4 C storage and roll between the palms for 20 seconds; removing protective plastic and aluminum covering from Optison vial; placing 25 G needle through the rubber gasket to provide a pressure vent; and using 1.5 inch 18 G needle to draw up 180 μL of Optison into a syringe. With the same needle and syringe, 10 μL of solution comprising 50 μg of DNA payload and 10 μL of either PBS or the aptamer under evaluation suspended in PBS was drawn into the syringe to combine the DNA and Optison and aptamer. The payload were mixed in the syringe by rolling the syringe between the fingers until the solution was homogenous. The DNA+Optison solution was drawn out of the needle dead space. Then the 18 G needle was exchanged for a 25 G blunt needle for injection into a subject JVC.

Following administration of the microbubbles and nucleic acid payload and aptamer (where applicable), ultrasound acoustic energy was delivered to the liver area of mice in these experiments using a GE LOGIC E9 equipped with a C1-6 probe positioned perpendicular to the mouse to locate the lateral view of liver using B-mode ultrasound at an MI of about 0.07, and the presence of microbubbles was confirmed in the liver. Following confirmation of microbubbles, the ultrasound focal depth setting was set to 2 cm, and the zoom to 0, and ultrasound was delivered at a mechanical index of 1.4 at a frequency of 2.28 MHz, alternating between 10 seconds of ultrasound application, and 20 seconds of rest in which the transducer was removed from the skin of the subject, for a total application of 30 seconds of ultrasound application.

The above procedure of administration of the microbubbles and nucleic acid payload and aptamer (where applicable), and delivery of ultrasound acoustic energy was repeated twice for a total of three treatments, each treatment 24 hours apart.

### Results

5-days following the last treatment, plasma samples were collected from each subject and transgenic FVIII level in mouse plasma was measured by MSD assay. Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921 U/ml to 0.01 IU/ml. 2-fold diluted samples and standards were added to the wells in 96-well plate. Incubated 2 hours at room temperature and washed 3 times. The detection was performed by incubating samples with GMA-8023 antibody during 2 hours following triple wash. Signal was developed by Sulfo-TAG and detected by MSD machine.

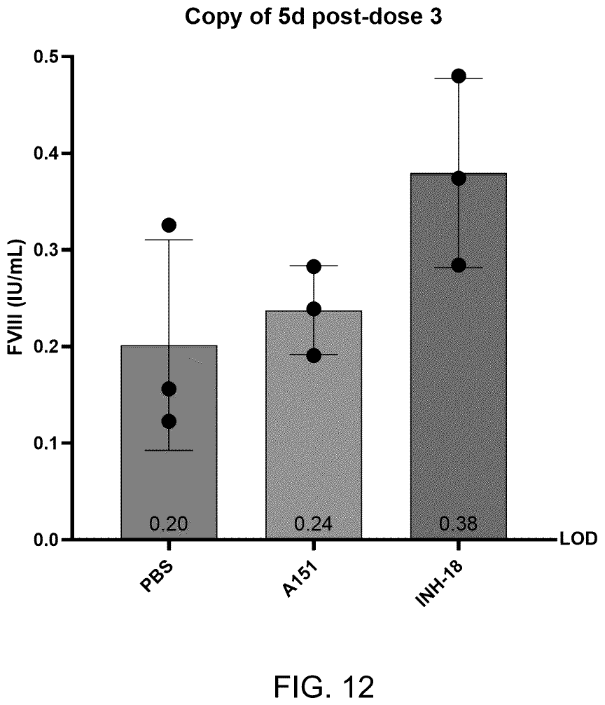

Results are shown in FIG. 12 in which control Group 1 exhibited an average FVIII level of about 0.2 IU/mL, Group 2 administered the A151 (SEQ ID NO: 51) (an antagonist inhibitor of AIM2, TLR9, TLR7, and cGAS) exhibited an average FVIII level of about 0.24 IU/mL representing approximately a 1.2 fold increase over the control Group 1, and Group 3 administered the INH-18 innate immune suppressor aptamer (SEQ ID NO: 53) (an antagonist inhibitor of TLR9 and TLR7) exhibited an average FVIII level of about 0.38 IU/mL representing approximately a 2-fold increase over the control Group 1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

| # | Desc. | Sequence |
|---|---|---|
| 1. | Nucleolin | GGTGGTGGTGGTTGTGGTGGTGGTGGT |
| | SELEX | |
| | Seq. | |
| 2. | Hairpin | AGGGATAACATGGCC/I/CTC/I/GGCCATGTTAT |
| | DNA | |
| | adaptor | |
| 3. | Anti- | GTTGTTTGGGGTGG |
| | Nucleolin | |
| | Aptamer | |
| 4. | Anti- | GTTGTTTGGGGTGGT |
| | Nucleolin | |
| | Aptamer | |
| 5. | Anti- | GGTTGGGGTGGGTGGGGTGGGTGGG |
| | Nucleolin | |
| | Aptamer | |
| 6. | Anti- | TTTGGTGGTGGTGGTTGTGGTGGTGGTG |
| | Nucleolin | |
| | Aptamer | |
| 7. | Anti- | TTTGGTGGTGGTGGTGGTGGTGGTGGTGG |
| | Nucleolin | |
| | Aptamer | |
| 8. | Anti- | TTTGGTGGTGGTGGTTTGGGTGGTGGTGG |
| | Nucleolin | |
| | Aptamer | |
| 9. | Anti- | TGGTGGTGGTGGT |
| | Nucleolin | |
| | Aptamer | |
| 10. | Anti- | GGTGGTTGTGGTGG |
| | Nucleolin | |
| | Aptamer | |
| 11. | Anti- | GGTGGTGGTGGTTGTGGTGGTGGTGGTTGTGGTGGTGGTGGTTGTGGTGGTGGT |
| | Nucleolin | GG |
| | Aptamer | |
| 12. | Anti- | GGTGGTTGTGGTGGTTGTGGTGGTTGTGGTGG |
| | Nucleolin | |
| | Aptamer | |
| 13. | Anti- | TTTGGTGGTGGTGGTTGTGGTGGTGGTGGTTT |
| | Nucleolin | |
| | Aptamer | |
| 14. | Anti- | GGTGGTGGTGGTTGTGGTGGTGGTGGTTT |
| | Nucleolin | |
| | Aptamer | |
| 15. | Anti- | TGGTGGTGGT |
| | Nucleolin | |
| | Aptamer | |
| 16. | Anti- | CCAUCUAGAUCUCCGUAGAUUCCCCCGGCUCUUUCUCGC |
| | Nucleolin | |
| | Aptamer | |
| 17. | Anti- | AGCCAGCUUUGCAUACCACGUGCAAUUCACUCCACCCGUCA |
| | Nucleolin | |
| | Aptamer | |
| 18. | Anti- | AAGAUCUGCUAAGUGCACGCACAAUCACCAUCGAGCGUCU |
| | Nucleolin | |
| | Aptamer | |
| 19. | Anti- | CACAUGGUACGCCCAAAGCGAGGCCCGCUGCGUAGUGC |
| | Nucleolin | |
| | Aptamer | |
| 20. | Anti- | CACGGUCCAGCGCUAACUGUACCUGCUGUGCCACCCACCG |
| | Nucleolin | |
| | Aptamer | |
| 21. | Anti- | ACCACGCGCCAACGUGUCAGCUACACGCCGUGUUCCCCGG |
| | Nucleolin | |
| | Aptamer | |
| 22. | Anti- | AAGAUCCUCGCGCAUCUGCCGAGCAAUCACCAUCGGACG |
| | Nucleolin | |
| | Aptamer | |
| 23. | Anti- | CCAAAUGCCAAGCCGUAGCCCGGCCAGUAGCCCACACGUC |
| | Nucleolin | |
| | Aptamer | |
| 24. | Anti- | UGCCAAGCCGAGGCCCGGCCACCAUCCACUGAUAGUGGGC |
| | Nucleolin | |
| | Aptamer | |
| 25. | Anti- | CCAUCUAGAUCUCCGUAGAUUCCCCCGGCUCUUUCUCGC |
| | Nucleolin | |
| | Aptamer | |
| 26. | Anti- | AGCCAGCUUUGCAUACCACGUGCAAUUCACUCCACCCGUCA |
| | Nucleolin | |
| | Aptamer | |
| 27. | Anti- | AAGAUCUGCUAAGUGCACGCACAAUCACCAUCGAGCGUCU |
| | Nucleolin | |
| | Aptamer | |
| 28. | Anti- | CACAUGGUACGCCCAAAGCGAGGCCCGCUGCGUAGUGC |
| | Nucleolin | |
| | Aptamer | |
| 29. | Anti- | CACGGUCCAGCGCUAACUGUACCUGCUGUGCCACCCACCG |
| | Nucleolin | |
| | Aptamer | |
| 30. | Anti- | ACCACGCGCCAACGUGUCAGCUACACGCCGUGUUCCCCGG |
| | Nucleolin | |
| | Aptamer | |
| 31. | Anti- | AAGAUCCUCGCGCAUCUGCCGAGCAAUCACCAUCGGACG |
| | Nucleolin | |
| | Aptamer | |
| 32. | Anti- | CCAAAUGCCAAGCCGUAGCCCGGCCAGUAGCCCACACGUC |
| | Nucleolin | |
| | Aptamer | |
| 33. | Anti- | UGCCAAGCCGAGGCCCGGCCACCAUCCACUGAUAGUGGGC |
| | Nucleolin | |
| | Aptamer | |
| 34. | Anti- | GGAAGAGGGAUGGGUGCCAGCUUUGCAUACCACGUGCAAUUCACUCCACCC |
| | Nucleolin | GUCAC |
| | Aptamer | |
| 35. | Anti- | GGGAGAGAGGAAGAGGGAUGGGCACGGUCCAGCGCUAACUGUACCUGCUGU |
| | Nucleolin | GCCACCCACCG |
| | Aptamer | |
| 36. | Anti- | GGAAGAGGGAUGGGCACGGUCCAGCGCUAACUGUACCUGCUGUGCCACCCAC |
| | Nucleolin | C |
| | Aptamer | |
| 37. | Anti- | GGGAGAGAGGAAGAGGGAUGGGACCACGCGCCAACGUGUCAGCUACACGCC |
| | Nucleolin | GUGUUCCCCGG |
| | Aptamer | |
| 38. | Anti- | GGGAGGAAGAGGGAUGGGCACGGUCCAGCGCUAACUGUACCUGCUGUGCCA |
| | Nucleolin | CCC |
| | Aptamer | |
| 39. | Anti- | GGGAGGAAGAGGAUGGGCACGGUCCAGCGCUAACUGUACCUGCUGUGCCAC |
| | Nucleolin | C |
| | Aptamer | |
| 40. | Anti- | GGGAGGAAGAGGGAUGGGCACGGUCCAGCGCACUGUACCUGCUGUGCCACC |
| | Nucleolin | C |
| | Aptamer | |
| 41. | Anti- | GGGAGAGAGGAAGAGGGAUGGGCACGGUCCAGCGCUAACUGUACCUGCUGU |
| | Nucleolin | GCCACCC |
| | Aptamer | |
| 42. | Anti- | GGGAGAGAGGAAGAGGGAUGGGCACGGUCCAGCGCACUGUACCUGCUGUGC |
| | Nucleolin | CACCCACCG |
| | Aptamer | |
| 43. | Anti- | GGTGGTGGTGGTTGTGGTGGTGGTGG |
| | Nucleolin | |
| | Aptamer | |
| | AS1411 | |
| 44. | Anti- | TGGTGGTGGTTGTTGTGGTGGTGGTGGT |
| | Nucleolin | |
| | Aptamer | |
| | AT11 | |
| 45. | Anti- | TGGTGGTGGTTGGTGGTGGTGGTGGT |
| | Nucleolin | |
| | Aptamer | |
| | AT-11B0 | |
| 46. | Anti- | CCCCCCCCCCCCCCCCCCCCGGTGGTGGTGGTTGTGGTGGTGGTGG |
| | Nucleolin | |
| | Aptamer | |
| | C20AS1211 | |
| 47. | Anti- | GGTGGTGGTGGZZGTGGTGGTGGTGG |
| | Nucleolin | |
| | Aptamer | |
| | AS1411Z | |
| 48. | Anti- | TGGGGACTTTCCGCTGGGGACTTTCCGCTGGGGACTTTCCGC |
| | Importin | |
| | Aptamer | |
| 49. | Anti- | TGACAGTGGCGGCAGTCACTGAGAAAAACGAAACCGGAGC |
| | Nucleoporin | |
| | Aptamer | |
| | (NUP | |
| | 358)* | |
| 50. | Anti- | CCACGCAGATAGACGCTACTCTACTACATCGCAGCCAAC |
| | Nucleoporin | |
| | Aptamer* | |
| 51. | Innate | TTAGGGTTAGGGTTAGGGTTAGGG |
| | Immune | |
| | Suppressor | |
| | Aptamer | |
| | A151 | |
| 52. | Innate | CCTGGATGGGAA |
| | Immune | |
| | Suppressor | |
| | Aptamer | |
| | 4084-F | |
| 53. | Innate | CCTGGATGGGAACTTACCGCTGCA |
| | Immune | |
| | Suppressor | |
| | Aptamer | |
| | INH-18 | |
| 54. | Innate | CCTGGATGGGAATTAGGGTTAGGGTTAGGGTTAGGGCCTGGATGGGAACTTAC |
| | Immune | CGCTGCA |
| | Suppressor | |
| | Aptamer | |
| | ALL | |
| 55. | Hairpin | AGGGATAACA+T+G+G+C+CACTCAGGCCATGTTAT |
| | DNA | |
| | Adaptor | |
| 56. | Hairpin | AGGGATCCACTCAGGAT |
| | DNA | |
| | Adaptor | |
| 57. | Hairpin | AGGGATCC+A+C+T+C+AGGAT |
| | DNA | |
| | Adaptor | |
| 58. | Hairpin | AGGGCTAACCACTCAGGTTAG |
| | DNA | |
| | Adaptor | |
| 59. | Hairpin | AGGGCTAACCXCTCXGGTTAG |
| | DNA | |
| | Adaptor | |
| 60. | Hairpin | AGGGCTAACCA/i5F-dU/T/i5FdU/AGGTTAG |
| | DNA | |
| | Adaptor | |
| 61. | Hairpin | AGGGATAACATGGCCACTCAGGCCATGTTAT |
| | DNA | |
| | Adaptor | |
| 62. | Hairpin | AGGGATAACATGGCC/18-oxo-dA/CTC/18-oxo-dA/GGCCATGTTAT |
| | DNA | |
| | Adaptor | |
| 63. | Hairpin | AGGGCTTACG+C+G+C+GTAAG |
| | DNA | |
| | Adaptor | |
| 64. | oGOI- | AGGGATAACATGGCCACTCAGGCCATGTTATCCCTCGAGACCTGCATCTAG |
| | CAG- | ATCCGACGTTACATAACTTACGGTAAATGGCCCGCCTGGCTGACCGCCCAACG |
| | H2B- | ACCCCCGCCCATTGACGTCAATAATGACGTATGTTCCCATAGTAACGCCAATA |
| | tdTomato- | GGGACTTTCCATTGACGTCAATGGGTGGAGTATTTACGGTAAACTGCCCACTTG |
| | noV5- | GCAGTACATCAAGTGTATCATATGCCAAGTACGCCCCCTATTGACGTCAATGA |
| | P2A-Fluc- | CGGTAAATGGCCCGCCTGGCATTATGCCCAGTACATGACCTTATGGGACTTTCC |
| | WPRE3- | TACTTGGCAGTACATCTACGTATTAGTCATCGCTATTACCATGGTCGAGGTGAG |
| | DTS- | CCCCACGTTCTGCTTCACTCTCCCCATCTCCCCCCCCTCCCCACCCCCAATTTTG |
| | Adapter- | TATTTATTTATTTTTTAATTATTTTGTGCAGCGATGGGGGCGGGGGGGGGGGGG |
| | 19 | GCGCGCGCCAGGCGGGGCGGGGCGGGGCGAGGGGCGGGGGGGGCGAGGCG |
| | | GAGAGGTGCGGCGGCAGCCAATCAGAGCGGCGCGCTCCGAAAGTTTCCTTTTA |
| | | TGGCGAGGCGGCGGCGGCGGCGGCCCTATAAAAAGCGAAGCGCGCGGCGGGC |
| | | GGGAGTCGCTGCGCGCTGCCTTCGCCCCGTGCCCCGCTCCGCCGCCGCCTCGCG |
| | | CCGCCCGCCCCGGCTCTGACTGACCGCGTTACTCCCACAGGTGAGCGGGCGGG |
| | | ACGGCCCTTCTCCTCCGGGCTGTAATTAGCGCTTGGTTTAATGACGGCTTGTTT |
| | | CTTTTCTGTGGCTGCGTGAAAGCCTTGAGGGGCTCCGGGAGGGCCCTTTGTGCG |
| | | GGGGGAGCGGCTCGGGGGGTGCGTGCGTGTGTGTGTGCGTGGGGAGCGCCGC |
| | | GTGCGGCTCCGCGCTGCCCGGCGGCTGTGAGCGCTGCGGGCGCGGCGCGGGGC |
| | | TTTGTGCGCTCCGCAGTGTGCGCGAGGGGAGCGCGGCCGGGGGCGGTGCCCCG |
| | | CGGTGCGGGGGGGGCTGCGAGGGGAACAAAGGCTGCGTGCGGGGTGTGTGCG |
| | | TGGGGGGGTGAGCAGGGGGTGTGGGCGCGTCGGTCGGGCTGCAACCCCCCCTG |
| | | CACCCCCCTCCCCGAGTTGCTGAGCACGGCCCGGCTTCGGGTGCGGGGCTCCG |
| | | TACGGGGCGTGGCGCGGGGCTCGCCGTGCCGGGCGGGGGGTGGCGGCAGGTG |
| | | GGGGTGCCGGGCGGGGCGGGGCCGCCTCGGGCCGGGGAGGGCTCGGGGGAGG |
| | | GGCGCGGCGGCCCCCGGAGCGCCGGCGGCTGTCGAGGCGCGGCGAGCCGCAG |
| | | CCATTGCCTTTTATGGTAATCGTGCGAGAGGGCGCAGGGACTTCCTTTGTCCCA |
| | | AATCTGTGCGGAGCCGAAATCTGGGAGGCGCCGCCGCACCCCCTCTAGCGGGC |
| | | GCGGGGCGAAGCGGTGCGGCGCCGGCAGGAAGGAAATGGGCGGGGAGGGCCT |
| | | TCGTGCGTCGCCGCGCCGCCGTCCCCTTCTCCCTCTCCAGCCTCGGGGCTGTCC |
| | | GCGGGGGGACGGCTGCCTTCGGGGGGGACGGGGCAGGGCGGGGTTCGGCTTC |
| | | TGGCGTGTGACCGGCGGCTCTAGAGCCTCTGCTAACCATGTTCATGCCTTCTTC |
| | | TTTTTCCTACAGCTCCTGGGCAACGTGCTGGTTATTGTGCTGTCTCATCATTTTG |
| | | GCAAAGAACGCGCGATCGCCACCATGCCAGAGCCAGCGAAGTCTGCTCCCGCC |
| | | CCGAAAAAGGGCTCCAAGAAGGCGGTGACTAAGGCGCAGAAGAAAGGCGGCA |
| | | AGAAGCGCAAGCGCAGCCGCAAGGAGAGCTATTCCATCTATGTGTACAAGGTT |
| | | CTGAAGCAGGTCCACCCTGACACCGGCATTTCGTCCAAGGCCATGGGCATCAT |
| | | GAATTCGTTTGTGAACGACATTTTCGAGCGCATCGCAGGTGAGGCTTCCCGCCT |
| | | GGCGCATTACAACAAGCGCTCGACCATCACCTCCAGGGAGATCCAGACGGCCG |
| | | TGCGCCTGCTGCTGCCTGGGGAGTTGGCCAAGCACGCCGTGTCCGAGGGTACT |
| | | AAGGCCATCACCAAGTACACCAGCGCTAAGGATCCACCGGTCGCCACCATGGT |
| | | GAGCAAGGGCGAGGAGGTCATCAAAGAGTTCATGCGCTTCAAGGTGCGCATG |
| | | GAGGGCTCCATGAACGGCCACGAGTTCGAGATCGAGGGCGAGGGCGAGGGCC |
| | | GCCCCTACGAGGGCACCCAGACCGCCAAGCTGAAGGTGACCAAGGGCGGCCC |
| | | CCTGCCCTTCGCCTGGGACATCCTGTCCCCCCAGTTCATGTACGGCTCCAAGGC |
| | | GTACGTGAAGCACCCCGCCGACATCCCCGATTACAAGAAGCTGTCCTTCCCCG |
| | | AGGGCTTCAAGTGGGAGCGCGTGATGAACTTCGAGGACGGCGGTCTGGTGACC |
| | | GTGACCCAGGACTCCTCCCTGCAGGACGGCACGCTGATCTACAAGGTGAAGAT |
| | | GCGCGGCACCAACTTCCCCCCCGACGGCCCCGTAATGCAGAAGAAGACCATGG |
| | | GCTGGGAGGCCTCCACCGAGCGCCTGTACCCCCGCGACGGCGTGCTGAAGGGC |
| | | GAGATCCACCAGGCCCTGAAGCTGAAGGACGGCGGCCACTACCTGGTGGAGTT |
| | | CAAGACCATCTACATGGCCAAGAAGCCCGTGCAACTGCCCGGCTACTACTACG |
| | | TGGACACCAAGCTGGACATCACCTCCCACAACGAGGACTACACCATCGTGGAA |
| | | CAGTACGAGCGCTCCGAGGGCCGCCACCACCTGTTCCTGGGGCATGGCACCGG |
| | | CAGCACCGGCAGCGGCAGCTCCGGCACCGCCTCCTCCGAGGACAACAACATGG |
| | | CCGTCATCAAAGAGTTCATGCGCTTCAAGGTGCGCATGGAGGGCTCCATGAAC |
| | | GGCCACGAGTTCGAGATCGAGGGCGAGGGCGAGGGCCGCCCCTACGAGGGCA |
| | | CCCAGACCGCCAAGCTGAAGGTGACCAAGGGCGGCCCCCTGCCCTTCGCCTGG |
| | | GACATCCTGTCCCCCCAGTTCATGTACGGCTCCAAGGCGTACGTGAAGCACCC |
| | | CGCCGACATCCCCGATTACAAGAAGCTGTCCTTCCCCGAGGGCTTCAAGTGGG |
| | | AGCGCGTGATGAACTTCGAGGACGGCGGTCTGGTGACCGTGACCCAGGACTCC |
| | | TCCCTGCAGGACGGCACGCTGATCTACAAGGTGAAGATGCGCGGCACCAACTT |
| | | CCCCCCCGACGGCCCCGTAATGCAGAAGAAGACCATGGGCTGGGAGGCCTCCA |
| | | CCGAGCGCCTGTACCCCCGCGACGGCGTGCTGAAGGGCGAGATCCACCAGGCC |
| | | CTGAAGCTGAAGGACGGCGGCCACTACCTGGTGGAGTTCAAGACCATCTACAT |
| | | GGCCAAGAAGCCCGTGCAACTGCCCGGCTACTACTACGTGGACACCAAGCTGG |
| | | ACATCACCTCCCACAACGAGGACTACACCATCGTGGAACAGTACGAGCGCTCC |
| | | GAGGGCCGCCACCACCTGTTCCTGTACGGCATGGACGAGCTGTACAAGGCTAC |
| | | TAACTTCAGCCTGCTGAAGCAGGCTGGAGACGTGGAGGAGAACCCTGGACCTA |
| | | TGGAAGACGCCAAAAACATAAAGAAAGGCCCGGCGCCATTCTATCCGCTGGA |
| | | AGATGGAACCGCTGGAGAGCAACTGCATAAGGCTATGAAGAGATACGCCCTG |
| | | GTTCCTGGAACAATTGCTTTTACAGATGCACATATCGAGGTGGACATCACTTAC |
| | | GCTGAGTACTTCGAAATGTCCGTTCGGTTGGCAGAAGCTATGAAACGATATGG |
| | | GCTGAATACAAATCACAGAATCGTCGTATGCAGTGAAAACTCTCTTCAATTCTT |
| | | TATGCCGGTGTTGGGCGCGTTATTTATCGGAGTTGCAGTTGCGCCCGCGAACGA |
| | | CATTTATAATGAACGTGAATTGCTCAACAGTATGGGCATTTCGCAGCCTACCGT |
| | | GGTGTTCGTTTCCAAAAAGGGGTTGCAAAAAATTTTGAACGTGCAAAAAAAGC |
| | | TCCCAATCATCCAAAAAATTATTATCATGGATTCTAAAACGGATTACCAGGGA |
| | | TTTCAGTCGATGTACACGTTCGTCACATCTCATCTACCTCCCGGTTTTAATGAAT |
| | | ACGATTTTGTGCCAGAGTCCTTCGATAGGGACAAGACAATTGCACTGATCATG |
| | | AACTCCTCTGGATCTACTGGTCTGCCTAAAGGTGTCGCTCTGCCTCATAGAACT |
| | | GCCTGCGTGAGATTCTCGCATGCCAGAGATCCTATTTTTGGCAATCAAATCATT |
| | | CCGGATACTGCGATTTTAAGTGTTGTTCCATTCCATCACGGTTTTGGAATGTTTA |
| | | CTACACTCGGATATTTGATATGTGGATTTCGAGTCGTCTTAATGTATAGATTTG |
| | | AAGAAGAGCTGTTTCTGAGGAGCCTTCAGGATTACAAGATTCAAAGTGCGCTG |
| | | CTGGTGCCAACCCTATTCTCCTTCTTCGCCAAAAGCACTCTGATTGACAAATAC |
| | | GATTTATCTAATTTACACGAAATTGCTTCTGGTGGCGCTCCCCTCTCTAAGGAA |
| | | GTCGGGGAAGCGGTTGCCAAGAGGTTCCATCTGCCAGGTATCAGGCAAGGATA |
| | | TGGGCTCACTGAGACTACATCAGCTATTCTGATTACACCCGAGGGGGATGATA |
| | | AACCGGGCGCGGTCGGTAAAGTTGTTCCATTTTTTGAAGCGAAGGTTGTGGATC |
| | | TGGATACCGGGAAAACGCTGGGCGTTAATCAAAGAGGCGAACTGTGTGTGAG |
| | | AGGTCCTATGATTATGTCCGGTTATGTAAACAATCCGGAAGCGACCAACGCCT |
| | | TGATTGACAAGGATGGATGGCTACATTCTGGAGACATAGCTTACTGGGACGAA |
| | | GACGAACACTTCTTCATCGTTGACCGCCTGAAGTCTCTGATTAAGTACAAAGGC |
| | | TATCAGGTGGCTCCCGCTGAATTGGAATCCATCTTGCTCCAACACCCCAACATC |
| | | TTCGACGCAGGTGTCGCAGGTCTTCCCGACGATGACGCCGGTGAACTTCCCGC |
| | | CGCCGTTGTTGTTTTGGAGCACGGAAAGACGATGACGGAAAAAGAGATCGTGG |
| | | ATTACGTCGCCAGTCAAGTAACAACCGCGAAAAAGTTGCGCGGAGGAGTTGTG |
| | | TTTGTGGACGAAGTACCGAAAGGTCTTACCGGAAAACTCGACGCAAGAAAAAT |
| | | CAGAGAGATCCTCATAAAGGCCAAGAAGGGCGGAAAGATCGCCGTGTAACTC |
| | | GAGAATCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTGGTGTGCAC |
| | | TGTGTTTGCTGACGCAACCCCCACTGGTTGGGGCATTGCCACCACCTGTCAGCT |
| | | CCTTTCCGGGACTTTCGCTTTCCCCCTCCCTATTGCCACGGCGGAACTCATCGC |
| | | CGCCTGCCTTGCCCGCTGCTGGACAGGGGCTCGGCTGTTGGGCACTGACAATTC |
| | | CGTGGGGTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGG |
| | | GGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGG |
| | | ACTTTCCGCTGGGGACTTTCCGCTGGGGACTTTCCGCCTTCAGCTAAGGAAGCT |
| | | ACCAATATTTAGAGGTACATTTTGTTCTAGAACAAAATGTACCGGTACATTTTG |
| | | TTCTGGTACATTTTGTTCTATTAAGGACAACGTAAGTATAGCGCATAGACACGG |
| | | TCTCGAGGGATAACATGGCCACTCAGGCCATGTTATCCCT |
| 65. | oGOI- | AGGGATAACATGGCCCCTGGATGGGAATTAGGGTTAGGGTTAGGGTTAGGGC |
| | CAG- | CTGGATGGGAACTTACCGCTGCAGGCCATGTTATCCCTCGAGACCTGCATCTA |
| | H2B- | GATCCGACGTTACATAACTTACGGTAAATGGCCCGCCTGGCTGACCGCCCAAC |
| | tdTomato- | GACCCCCGCCCATTGACGTCAATAATGACGTATGTTCCCATAGTAACGCCAAT |
| | noV5- | AGGGACTTTCCATTGACGTCAATGGGTGGAGTATTTACGGTAAACTGCCCACTT |
| | P2A-Fluc- | GGCAGTACATCAAGTGTATCATATGCCAAGTACGCCCCCTATTGACGTCAATG |
| | WPRE3- | ACGGTAAATGGCCCGCCTGGCATTATGCCCAGTACATGACCTTATGGGACTTTC |
| | DTS-ALL | CTACTTGGCAGTACATCTACGTATTAGTCATCGCTATTACCATGGTCGAGGTGA |
| | | GCCCCACGTTCTGCTTCACTCTCCCCATCTCCCCCCCCTCCCCACCCCCAATTTT |
| | | GTATTTATTTATTTTTTAATTATTTTGTGCAGCGATGGGGGCGGGGGGGGGGGG |
| | | GGCGCGCGCCAGGCGGGGGGGGCGGGGCGAGGGGCGGGGGGGGCGAGGC |
| | | GGAGAGGTGCGGCGGCAGCCAATCAGAGCGGCGCGCTCCGAAAGTTTCCTTTT |
| | | ATGGCGAGGCGGCGGCGGCGGCGGCCCTATAAAAAGCGAAGCGCGCGGCGGG |
| | | CGGGAGTCGCTGCGCGCTGCCTTCGCCCCGTGCCCCGCTCCGCCGCCGCCTCGC |
| | | GCCGCCCGCCCCGGCTCTGACTGACCGCGTTACTCCCACAGGTGAGCGGGCGG |
| | | GACGGCCCTTCTCCTCCGGGCTGTAATTAGCGCTTGGTTTAATGACGGCTTGTT |
| | | TCTTTTCTGTGGCTGCGTGAAAGCCTTGAGGGGCTCCGGGAGGGCCCTTTGTGC |
| | | GGGGGGAGCGGCTCGGGGGGTGCGTGCGTGTGTGTGTGCGTGGGGAGCGCCG |
| | | CGTGCGGCTCCGCGCTGCCCGGCGGCTGTGAGCGCTGCGGGCGCGGCGCGGGG |
| | | CTTTGTGCGCTCCGCAGTGTGCGCGAGGGGAGCGCGGCCGGGGGCGGTGCCCC |
| | | GCGGTGCGGGGGGGGCTGCGAGGGGAACAAAGGCTGCGTGCGGGGTGTGTGC |
| | | GTGGGGGGGTGAGCAGGGGGTGTGGGCGCGTCGGTCGGGCTGCAACCCCCCCT |
| | | GCACCCCCCTCCCCGAGTTGCTGAGCACGGCCCGGCTTCGGGTGCGGGGCTCC |
| | | GTACGGGGCGTGGCGCGGGGCTCGCCGTGCCGGGCGGGGGGTGGCGGCAGGT |
| | | GGGGGTGCCGGGCGGGGGGGGGCCGCCTCGGGCCGGGGAGGGCTCGGGGGAG |
| | | GGGCGCGGCGGCCCCCGGAGCGCCGGCGGCTGTCGAGGCGCGGCGAGCCGCA |
| | | GCCATTGCCTTTTATGGTAATCGTGCGAGAGGGCGCAGGGACTTCCTTTGTCCC |
| | | AAATCTGTGCGGAGCCGAAATCTGGGAGGCGCCGCCGCACCCCCTCTAGCGGG |
| | | CGCGGGGCGAAGCGGTGCGGCGCCGGCAGGAAGGAAATGGGCGGGGAGGGCC |
| | | TTCGTGCGTCGCCGCGCCGCCGTCCCCTTCTCCCTCTCCAGCCTCGGGGCTGTC |
| | | CGCGGGGGGACGGCTGCCTTCGGGGGGGACGGGGCAGGGCGGGGTTCGGCTT |
| | | CTGGCGTGTGACCGGCGGCTCTAGAGCCTCTGCTAACCATGTTCATGCCTTCTT |
| | | CTTTTTCCTACAGCTCCTGGGCAACGTGCTGGTTATTGTGCTGTCTCATCATTTT |
| | | GGCAAAGAACGCGCGATCGCCACCATGCCAGAGCCAGCGAAGTCTGCTCCCGC |
| | | CCCGAAAAAGGGCTCCAAGAAGGCGGTGACTAAGGCGCAGAAGAAAGGCGGC |
| | | AAGAAGCGCAAGCGCAGCCGCAAGGAGAGCTATTCCATCTATGTGTACAAGGT |
| | | TCTGAAGCAGGTCCACCCTGACACCGGCATTTCGTCCAAGGCCATGGGCATCA |
| | | TGAATTCGTTTGTGAACGACATTTTCGAGCGCATCGCAGGTGAGGCTTCCCGCC |
| | | TGGCGCATTACAACAAGCGCTCGACCATCACCTCCAGGGAGATCCAGACGGCC |
| | | GTGCGCCTGCTGCTGCCTGGGGAGTTGGCCAAGCACGCCGTGTCCGAGGGTAC |
| | | TAAGGCCATCACCAAGTACACCAGCGCTAAGGATCCACCGGTCGCCACCATGG |
| | | TGAGCAAGGGCGAGGAGGTCATCAAAGAGTTCATGCGCTTCAAGGTGCGCATG |
| | | GAGGGCTCCATGAACGGCCACGAGTTCGAGATCGAGGGCGAGGGCGAGGGCC |
| | | GCCCCTACGAGGGCACCCAGACCGCCAAGCTGAAGGTGACCAAGGGCGGCCC |
| | | CCTGCCCTTCGCCTGGGACATCCTGTCCCCCCAGTTCATGTACGGCTCCAAGGC |
| | | GTACGTGAAGCACCCCGCCGACATCCCCGATTACAAGAAGCTGTCCTTCCCCG |
| | | AGGGCTTCAAGTGGGAGCGCGTGATGAACTTCGAGGACGGCGGTCTGGTGACC |
| | | GTGACCCAGGACTCCTCCCTGCAGGACGGCACGCTGATCTACAAGGTGAAGAT |
| | | GCGCGGCACCAACTTCCCCCCCGACGGCCCCGTAATGCAGAAGAAGACCATGG |
| | | GCTGGGAGGCCTCCACCGAGCGCCTGTACCCCCGCGACGGCGTGCTGAAGGGC |
| | | GAGATCCACCAGGCCCTGAAGCTGAAGGACGGCGGCCACTACCTGGTGGAGTT |
| | | CAAGACCATCTACATGGCCAAGAAGCCCGTGCAACTGCCCGGCTACTACTACG |
| | | TGGACACCAAGCTGGACATCACCTCCCACAACGAGGACTACACCATCGTGGAA |
| | | CAGTACGAGCGCTCCGAGGGCCGCCACCACCTGTTCCTGGGGCATGGCACCGG |
| | | CAGCACCGGCAGCGGCAGCTCCGGCACCGCCTCCTCCGAGGACAACAACATGG |
| | | CCGTCATCAAAGAGTTCATGCGCTTCAAGGTGCGCATGGAGGGCTCCATGAAC |
| | | GGCCACGAGTTCGAGATCGAGGGCGAGGGCGAGGGCCGCCCCTACGAGGGCA |
| | | CCCAGACCGCCAAGCTGAAGGTGACCAAGGGCGGCCCCCTGCCCTTCGCCTGG |
| | | GACATCCTGTCCCCCCAGTTCATGTACGGCTCCAAGGCGTACGTGAAGCACCC |
| | | CGCCGACATCCCCGATTACAAGAAGCTGTCCTTCCCCGAGGGCTTCAAGTGGG |
| | | AGCGCGTGATGAACTTCGAGGACGGCGGTCTGGTGACCGTGACCCAGGACTCC |
| | | TCCCTGCAGGACGGCACGCTGATCTACAAGGTGAAGATGCGCGGCACCAACTT |
| | | CCCCCCCGACGGCCCCGTAATGCAGAAGAAGACCATGGGCTGGGAGGCCTCCA |
| | | CCGAGCGCCTGTACCCCCGCGACGGCGTGCTGAAGGGCGAGATCCACCAGGCC |
| | | CTGAAGCTGAAGGACGGCGGCCACTACCTGGTGGAGTTCAAGACCATCTACAT |
| | | GGCCAAGAAGCCCGTGCAACTGCCCGGCTACTACTACGTGGACACCAAGCTGG |
| | | ACATCACCTCCCACAACGAGGACTACACCATCGTGGAACAGTACGAGCGCTCC |
| | | GAGGGCCGCCACCACCTGTTCCTGTACGGCATGGACGAGCTGTACAAGGCTAC |
| | | TAACTTCAGCCTGCTGAAGCAGGCTGGAGACGTGGAGGAGAACCCTGGACCTA |
| | | TGGAAGACGCCAAAAACATAAAGAAAGGCCCGGCGCCATTCTATCCGCTGGA |
| | | AGATGGAACCGCTGGAGAGCAACTGCATAAGGCTATGAAGAGATACGCCCTG |
| | | GTTCCTGGAACAATTGCTTTTACAGATGCACATATCGAGGTGGACATCACTTAC |
| | | GCTGAGTACTTCGAAATGTCCGTTCGGTTGGCAGAAGCTATGAAACGATATGG |
| | | GCTGAATACAAATCACAGAATCGTCGTATGCAGTGAAAACTCTCTTCAATTCTT |
| | | TATGCCGGTGTTGGGCGCGTTATTTATCGGAGTTGCAGTTGCGCCCGCGAACGA |
| | | CATTTATAATGAACGTGAATTGCTCAACAGTATGGGCATTTCGCAGCCTACCGT |
| | | GGTGTTCGTTTCCAAAAAGGGGTTGCAAAAAATTTTGAACGTGCAAAAAAAGC |
| | | TCCCAATCATCCAAAAAATTATTATCATGGATTCTAAAACGGATTACCAGGGA |
| | | TTTCAGTCGATGTACACGTTCGTCACATCTCATCTACCTCCCGGTTTTAATGAAT |
| | | ACGATTTTGTGCCAGAGTCCTTCGATAGGGACAAGACAATTGCACTGATCATG |
| | | AACTCCTCTGGATCTACTGGTCTGCCTAAAGGTGTCGCTCTGCCTCATAGAACT |
| | | GCCTGCGTGAGATTCTCGCATGCCAGAGATCCTATTTTTGGCAATCAAATCATT |
| | | CCGGATACTGCGATTTTAAGTGTTGTTCCATTCCATCACGGTTTTGGAATGTTTA |
| | | CTACACTCGGATATTTGATATGTGGATTTCGAGTCGTCTTAATGTATAGATTTG |
| | | AAGAAGAGCTGTTTCTGAGGAGCCTTCAGGATTACAAGATTCAAAGTGCGCTG |
| | | CTGGTGCCAACCCTATTCTCCTTCTTCGCCAAAAGCACTCTGATTGACAAATAC |
| | | GATTTATCTAATTTACACGAAATTGCTTCTGGTGGCGCTCCCCTCTCTAAGGAA |
| | | GTCGGGGAAGCGGTTGCCAAGAGGTTCCATCTGCCAGGTATCAGGCAAGGATA |
| | | TGGGCTCACTGAGACTACATCAGCTATTCTGATTACACCCGAGGGGGATGATA |
| | | AACCGGGCGCGGTCGGTAAAGTTGTTCCATTTTTTGAAGCGAAGGTTGTGGATC |
| | | TGGATACCGGGAAAACGCTGGGCGTTAATCAAAGAGGCGAACTGTGTGTGAG |
| | | AGGTCCTATGATTATGTCCGGTTATGTAAACAATCCGGAAGCGACCAACGCCT |
| | | TGATTGACAAGGATGGATGGCTACATTCTGGAGACATAGCTTACTGGGACGAA |
| | | GACGAACACTTCTTCATCGTTGACCGCCTGAAGTCTCTGATTAAGTACAAAGGC |
| | | TATCAGGTGGCTCCCGCTGAATTGGAATCCATCTTGCTCCAACACCCCAACATC |
| | | TTCGACGCAGGTGTCGCAGGTCTTCCCGACGATGACGCCGGTGAACTTCCCGC |
| | | CGCCGTTGTTGTTTTGGAGCACGGAAAGACGATGACGGAAAAAGAGATCGTGG |
| | | ATTACGTCGCCAGTCAAGTAACAACCGCGAAAAAGTTGCGCGGAGGAGTTGTG |
| | | TTTGTGGACGAAGTACCGAAAGGTCTTACCGGAAAACTCGACGCAAGAAAAAT |
| | | CAGAGAGATCCTCATAAAGGCCAAGAAGGGCGGAAAGATCGCCGTGTAACTC |
| | | GAGAATCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTGGTGTGCAC |
| | | TGTGTTTGCTGACGCAACCCCCACTGGTTGGGGCATTGCCACCACCTGTCAGCT |
| | | CCTTTCCGGGACTTTCGCTTTCCCCCTCCCTATTGCCACGGCGGAACTCATCGC |
| | | CGCCTGCCTTGCCCGCTGCTGGACAGGGGCTCGGCTGTTGGGCACTGACAATTC |
| | | CGTGGGGTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGG |
| | | GGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGG |
| | | ACTTTCCGCTGGGGACTTTCCGCTGGGGACTTTCCGCCTTCAGCTAAGGAAGCT |
| | | ACCAATATTTAGAGGTACATTTTGTTCTAGAACAAAATGTACCGGTACATTTTG |
| | | TTCTGGTACATTTTGTTCTATTAAGGACAACGTAAGTATAGCGCATAGACACGG |
| | | TCTCGAGGGATAACATGGCCCCTGGATGGGAATTAGGGTTAGGGTTAGGGTT |
| | | AGGGCCTGGATGGGAACTTACCGCTGCAGGCCATGTTATCCCT |
| 66. | oGOI- | AGGGATAACATGGCCGGTGGTGGTGGTTGTGGTGGTGGTGGGGCCATGTTAT |
| | CAG- | CCCTCGAGACCTGCATCTAGATCCGACGTTACATAACTTACGGTAAATGGCCC |
| | H2B- | GCCTGGCTGACCGCCCAACGACCCCCGCCCATTGACGTCAATAATGACGTATG |
| | tdTomato- | TTCCCATAGTAACGCCAATAGGGACTTTCCATTGACGTCAATGGGTGGAGTATT |
| | noV5- | TACGGTAAACTGCCCACTTGGCAGTACATCAAGTGTATCATATGCCAAGTACG |
| | P2A-Fluc- | CCCCCTATTGACGTCAATGACGGTAAATGGCCCGCCTGGCATTATGCCCAGTAC |
| | WPRE3- | ATGACCTTATGGGACTTTCCTACTTGGCAGTACATCTACGTATTAGTCATCGCT |
| | DTS- | ATTACCATGGTCGAGGTGAGCCCCACGTTCTGCTTCACTCTCCCCATCTCCCCC |
| | AS1411 | CCCTCCCCACCCCCAATTTTGTATTTATTTATTTTTTAATTATTTTGTGCAGCGA |
| | | TGGGGGCGGGGGGGGGGGGGGCGCGCGCCAGGCGGGGCGGGGCGGGGCGAG |
| | | GGGCGGGGCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGCCCTATAAA |
| | | AAGCGAAGCGCGCGGCGGGCGGGAGTCGCTGCGCGCTGCCTTCGCCCCGTGCC |
| | | CCGCTCCGCCGCCGCCTCGCGCCGCCCGCCCCGGCTCTGACTGACCGCGTTACT |
| | | CCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGC |
| | | TCCGGGAGGGCCCTTTGTGCGGGGGGAGCGGCTCGGGGGGTGCGTGCGTGTGT |
| | | GTGTGCGTGGGGAGCGCCGCGTGCGGCTCCGCGCTGCCCGGCGGCTGTGAGCG |
| | | CTGCGGGCGCGGCGCGGGGCTTTGTGCGCTCCGCAGTGTGCGCGAGGGGAGCG |
| | | CGGCCGGGGGCGGTGCCCCGCGGTGCGGGGGGGGCTGCGAGGGGAACAAAGG |
| | | CTGCGTGCGGGGTGTGTGCGTGGGGGGGTGAGCAGGGGGTGTGGGCGCGTCG |
| | | GTCGGGCTGCAACCCCCCCTGCACCCCCCTCCCCGAGTTGCTGAGCACGGCCC |
| | | GGCTTCGGGTGCGGGGCTCCGTACGGGGCGTGGCGCGGGGCTCGCCGTGCCGG |
| | | GCGGGGGGTGGCGGCAGGTGGGGGTGCCGGGCGGGGCGGGGCCGCCTCGGGC |
| | | CGGGGAGGGCTCGGGGGAGGGGCGCGGCGGCCCCCGGAGCGCCGGCGGCTGT |
| | | CGAGGCGCGGCGAGCCGCAGCCATTGCCTTTTATGGTAATCGTGCGAGAGGGC |
| | | GCAGGGACTTCCTTTGTCCCAAATCTGTGCGGAGCCGAAATCTGGGAGGCGCC |
| | | GCCGCACCCCCTCTAGCGGGCGCGGGGCGAAGCGGTGCGGCGCCGGCAGGAA |
| | | GGAAATGGGCGGGGAGGGCCTTCGTGCGTCGCCGCGCCGCCGTCCCCTTCTCC |
| | | CTCTCCAGCCTCGGGGCTGTCCGCGGGGGGACGGCTGCCTTCGGGGGGGACGG |
| | | GGCAGGGCGGGGTTCGGCTTCTGGCGTGTGACCGGCGGCTCTAGAGCCTCTGC |
| | | TAACCATGTTCATGCCTTCTTCTTTTTCCTACAGCTCCTGGGCAACGTGCTGGTT |
| | | ATTGTGCTGTCTCATCATTTTGGCAAAGAACGCGCGATCGCCACCATGCCAGA |
| | | GCCAGCGAAGTCTGCTCCCGCCCCGAAAAAGGGCTCCAAGAAGGCGGTGACTA |
| | | AGGCGCAGAAGAAAGGCGGCAAGAAGCGCAAGCGCAGCCGCAAGGAGAGCT |
| | | ATTCCATCTATGTGTACAAGGTTCTGAAGCAGGTCCACCCTGACACCGGCATTT |
| | | CGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGGTCTCGAGGGATAACATGGCCGGTGGTG |
| | | GTGGTTGTGGTGGTGGTGGGGCCATGTTATCCCT |
| 67. | oGOI- | AGGGATAACATGGCCTGGTGGTGGTTGTTGTGGTGGTGGTGGTGGCCATGTT |
| | CAG- | ATCCCTCGAGACCTGCATCTAGATCCGACGTTACATAACTTACGGTAAATGGC |
| | H2B- | CCGCCTGGCTGACCGCCCAACGACCCCCGCCCATTGACGTCAATAATGACGTA |
| | tdTomato- | TGTTCCCATAGTAACGCCAATAGGGACTTTCCATTGACGTCAATGGGTGGAGT |
| | noV5- | ATTTACGGTAAACTGCCCACTTGGCAGTACATCAAGTGTATCATATGCCAAGTA |
| | P2A-Fluc- | CGCCCCCTATTGACGTCAATGACGGTAAATGGCCCGCCTGGCATTATGCCCAGT |
| | WPRE3- | ACATGACCTTATGGGACTTTCCTACTTGGCAGTACATCTACGTATTAGTCATCG |
| | DTS- | CTATTACCATGGTCGAGGTGAGCCCCACGTTCTGCTTCACTCTCCCCATCTCCC |
| | AT11 | CCCCCTCCCCACCCCCAATTTTGTATTTATTTATTTTTTAATTATTTTGTGCAGC |
| | | GATGGGGGCGGGGGGGGGGGGGGCGCGCGCCAGGCGGGGCGGGGCGGGGCG |
| | | AGGGGCGGGGCGGGGCGAGGCGGAGAGGTGCGGCGGCAGCCAATCAGAGCG |
| | | GCGCGCTCCGAAAGTTTCCTTTTATGGCGAGGCGGCGGCGGCGGCGGCCCTAT |
| | | AAAAAGCGAAGCGCGCGGCGGGCGGGAGTCGCTGCGCGCTGCCTTCGCCCCGT |
| | | GCCCCGCTCCGCCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAATTAGC |
| | | GCTTGGTTTAATGACGGCTTGTTTCTTTTCTGTGGCTGCGTGAAAGCCTTGAGG |
| | | GGCTCCGGGAGGGCCCTTTGTGCGGGGGGAGCGGCTCGGGGGGTGCGTGCGTG |
| | | TGTGTGTGCGTGGGGAGCGCCGCGTGCGGCTCCGCGCTGCCCGGCGGCTGTGA |
| | | GCGCTGCGGGCGCGGCGCGGGGCTTTGTGCGCTCCGCAGTGTGCGCGAGGGGA |
| | | GCGCGGCCGGGGGCGGTGCCCCGCGGTGCGGGGGGGGCTGCGAGGGGAACAA |
| | | AGGCTGCGTGCGGGGTGTGTGCGTGGGGGGGTGAGCAGGGGGTGTGGGCGCG |
| | | TCGGTCGGGCTGCAACCCCCCCTGCACCCCCCTCCCCGAGTTGCTGAGCACGGC |
| | | CCGGCTTCGGGTGCGGGGCTCCGTACGGGGCGTGGCGCGGGGCTCGCCGTGCC |
| | | GGGCGGGGGGTGGCGGCAGGTGGGGGTGCCGGGCGGGGCGGGGCCGCCTCGG |
| | | GCCGGGGAGGGCTCGGGGGAGGGGCGCGGCGGCCCCCGGAGCGCCGGCGGCT |
| | | GTCGAGGCGCGGCGAGCCGCAGCCATTGCCTTTTATGGTAATCGTGCGAGAGG |
| | | GCGCAGGGACTTCCTTTGTCCCAAATCTGTGCGGAGCCGAAATCTGGGAGGCG |
| | | CCGCCGCACCCCCTCTAGCGGGCGCGGGGCGAAGCGGTGCGGCGCCGGCAGG |
| | | AAGGAAATGGGCGGGGAGGGCCTTCGTGCGTCGCCGCGCCGCCGTCCCCTTCT |
| | | CCCTCTCCAGCCTCGGGGCTGTCCGCGGGGGGACGGCTGCCTTCGGGGGGGAC |
| | | GGGGCAGGGCGGGGTTCGGCTTCTGGCGTGTGACCGGCGGCTCTAGAGCCTCT |
| | | GCTAACCATGTTCATGCCTTCTTCTTTTTCCTACAGCTCCTGGGCAACGTGCTGG |
| | | TTATTGTGCTGTCTCATCATTTTGGCAAAGAACGCGCGATCGCCACCATGCCAG |
| | | AGCCAGCGAAGTCTGCTCCCGCCCCGAAAAAGGGCTCCAAGAAGGCGGTGACT |
| | | AAGGCGCAGAAGAAAGGCGGCAAGAAGCGCAAGCGCAGCCGCAAGGAGAGC |
| | | TATTCCATCTATGTGTACAAGGTTCTGAAGCAGGTCCACCCTGACACCGGCATT |
| | | TCGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGGTCTCGAGGGATAACATGGCCTGGTGGT |
| | | GGTTGTTGTGGTGGTGGTGGTGGCCATGTTATCCCT |
| 68. | oGOI- | AGGGATAACATGGCCTGGTGGTGGTTGGTGGTGGTGGTGGTGGCCATGTTAT |
| | CAG- | CCCTCGAGACCTGCATCTAGATCCGACGTTACATAACTTACGGTAAATGGCCC |
| | H2B- | GCCTGGCTGACCGCCCAACGACCCCCGCCCATTGACGTCAATAATGACGTATG |
| | tdTomato- | TTCCCATAGTAACGCCAATAGGGACTTTCCATTGACGTCAATGGGTGGAGTATT |
| | noV5- | TACGGTAAACTGCCCACTTGGCAGTACATCAAGTGTATCATATGCCAAGTACG |
| | P2A-Fluc- | CCCCCTATTGACGTCAATGACGGTAAATGGCCCGCCTGGCATTATGCCCAGTAC |
| | WPRE3- | ATGACCTTATGGGACTTTCCTACTTGGCAGTACATCTACGTATTAGTCATCGCT |
| | DTS-AT- | ATTACCATGGTCGAGGTGAGCCCCACGTTCTGCTTCACTCTCCCCATCTCCCCC |
| | 11B0 | CCCTCCCCACCCCCAATTTTGTATTTATTTATTTTTTAATTATTTTGTGCAGCGA |
| | | TGGGGGCGGGGGGGGGGGGGGCGCGCGCCAGGCGGGGCGGGGCGGGGCGAG |
| | | GGGCGGGGCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGCCCTATAAA |
| | | AAGCGAAGCGCGCGGCGGGCGGGAGTCGCTGCGCGCTGCCTTCGCCCCGTGCC |
| | | CCGCTCCGCCGCCGCCTCGCGCCGCCCGCCCCGGCTCTGACTGACCGCGTTACT |
| | | CCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGC |
| | | TCCGGGAGGGCCCTTTGTGCGGGGGGAGCGGCTCGGGGGGTGCGTGCGTGTGT |
| | | GTGTGCGTGGGGAGCGCCGCGTGCGGCTCCGCGCTGCCCGGCGGCTGTGAGCG |
| | | CTGCGGGCGCGGCGCGGGGCTTTGTGCGCTCCGCAGTGTGCGCGAGGGGAGCG |
| | | CGGCCGGGGGCGGTGCCCCGCGGTGCGGGGGGGGCTGCGAGGGGAACAAAGG |
| | | CTGCGTGCGGGGTGTGTGCGTGGGGGGGTGAGCAGGGGGTGTGGGCGCGTCG |
| | | GTCGGGCTGCAACCCCCCCTGCACCCCCCTCCCCGAGTTGCTGAGCACGGCCC |
| | | GGCTTCGGGTGCGGGGCTCCGTACGGGGCGTGGCGCGGGGCTCGCCGTGCCGG |
| | | GCGGGGGGTGGCGGCAGGTGGGGGTGCCGGGCGGGGCGGGGCCGCCTCGGGC |
| | | CGGGGAGGGCTCGGGGGAGGGGCGCGGCGGCCCCCGGAGCGCCGGCGGCTGT |
| | | CGAGGCGCGGCGAGCCGCAGCCATTGCCTTTTATGGTAATCGTGCGAGAGGGC |
| | | GCAGGGACTTCCTTTGTCCCAAATCTGTGCGGAGCCGAAATCTGGGAGGCGCC |
| | | GCCGCACCCCCTCTAGCGGGCGCGGGGCGAAGCGGTGCGGCGCCGGCAGGAA |
| | | GGAAATGGGCGGGGAGGGCCTTCGTGCGTCGCCGCGCCGCCGTCCCCTTCTCC |
| | | CTCTCCAGCCTCGGGGCTGTCCGCGGGGGGACGGCTGCCTTCGGGGGGGACGG |
| | | GGCAGGGCGGGGTTCGGCTTCTGGCGTGTGACCGGCGGCTCTAGAGCCTCTGC |
| | | TAACCATGTTCATGCCTTCTTCTTTTTCCTACAGCTCCTGGGCAACGTGCTGGTT |
| | | ATTGTGCTGTCTCATCATTTTGGCAAAGAACGCGCGATCGCCACCATGCCAGA |
| | | GCCAGCGAAGTCTGCTCCCGCCCCGAAAAAGGGCTCCAAGAAGGCGGTGACTA |
| | | AGGCGCAGAAGAAAGGCGGCAAGAAGCGCAAGCGCAGCCGCAAGGAGAGCT |
| | | ATTCCATCTATGTGTACAAGGTTCTGAAGCAGGTCCACCCTGACACCGGCATTT |
| | | CGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGGTCTCGAGGGATAACATGGCCTGGTGGT |
| | | GGTTGGTGGTGGTGGTGGTGGCCATGTTATCCCT |
| 69. | oGOI- | AGGGATAACATGGCCCCCCCCCCCCCCCCCCCCCCGGTGGTGGTGGTTGTGGT |
| | CAG | GGTGGTGGGGCCATGTTATCCCTCGAGACCTGCATCTAGATCCGACGTTACAT |
| | H2B- | AACTTACGGTAAATGGCCCGCCTGGCTGACCGCCCAACGACCCCCGCCCATTG |
| | tdTomato- | ACGTCAATAATGACGTATGTTCCCATAGTAACGCCAATAGGGACTTTCCATTGA |
| | noV5- | CGTCAATGGGTGGAGTATTTACGGTAAACTGCCCACTTGGCAGTACATCAAGT |
| | P2A-Fluc- | GTATCATATGCCAAGTACGCCCCCTATTGACGTCAATGACGGTAAATGGCCCG |
| | WPRE3- | CCTGGCATTATGCCCAGTACATGACCTTATGGGACTTTCCTACTTGGCAGTACA |
| | DTS- | TCTACGTATTAGTCATCGCTATTACCATGGTCGAGGTGAGCCCCACGTTCTGCT |
| | C20AS1211 | TCACTCTCCCCATCTCCCCCCCCTCCCCACCCCCAATTTTGTATTTATTTATTTTT |
| | | TAATTATTTTGTGCAGCGATGGGGGCGGGGGGGGGGGGGGCGCGCGCCAGGC |
| | | GGGGCGGGGCGGGGCGAGGGGCGGGGCGGGGCGAGGCGGAGAGGTGCGGCG |
| | | GCAGCCAATCAGAGCGGCGCGCTCCGAAAGTTTCCTTTTATGGCGAGGCGGCG |
| | | GCGGCGGCGGCCCTATAAAAAGCGAAGCGCGCGGCGGGCGGGAGTCGCTGCG |
| | | CGCTGCCTTCGCCCCGTGCCCCGCTCCGCCGCCGCCTCGCGCCGCCCGCCCCGG |
| | | CTCTGACTGACCGCGTTACTCCCACAGGTGAGCGGGCGGGACGGCCCTTCTCCT |
| | | CCGGGCTGTAATTAGCGCTTGGTTTAATGACGGCTTGTTTCTTTTCTGTGGCTGC |
| | | GTGAAAGCCTTGAGGGGCTCCGGGAGGGCCCTTTGTGCGGGGGGAGCGGCTCG |
| | | GGGGGTGCGTGCGTGTGTGTGTGCGTGGGGAGCGCCGCGTGCGGCTCCGCGCT |
| | | GCCCGGCGGCTGTGAGCGCTGCGGGCGCGGCGCGGGGCTTTGTGCGCTCCGCA |
| | | GTGTGCGCGAGGGGAGCGCGGCCGGGGGCGGTGCCCCGCGGTGCGGGGGGGG |
| | | CTGCGAGGGGAACAAAGGCTGCGTGCGGGGTGTGTGCGTGGGGGGGTGAGCA |
| | | GGGGGTGTGGGCGCGTCGGTCGGGCTGCAACCCCCCCTGCACCCCCCTCCCCG |
| | | AGTTGCTGAGCACGGCCCGGCTTCGGGTGCGGGGCTCCGTACGGGGCGTGGCG |
| | | CGGGGCTCGCCGTGCCGGGCGGGGGGTGGCGGCAGGTGGGGGTGCCGGGCGG |
| | | GGCGGGGCCGCCTCGGGCCGGGGAGGGCTCGGGGGAGGGGCGCGGCGGCCCC |
| | | CGGAGCGCCGGCGGCTGTCGAGGCGCGGCGAGCCGCAGCCATTGCCTTTTATG |
| | | GTAATCGTGCGAGAGGGCGCAGGGACTTCCTTTGTCCCAAATCTGTGCGGAGC |
| | | CGAAATCTGGGAGGCGCCGCCGCACCCCCTCTAGCGGGCGCGGGGCGAAGCG |
| | | GTGCGGCGCCGGCAGGAAGGAAATGGGCGGGGAGGGCCTTCGTGCGTCGCCG |
| | | CGCCGCCGTCCCCTTCTCCCTCTCCAGCCTCGGGGCTGTCCGCGGGGGGACGGC |
| | | TGCCTTCGGGGGGGACGGGGCAGGGCGGGGTTCGGCTTCTGGCGTGTGACCGG |
| | | CGGCTCTAGAGCCTCTGCTAACCATGTTCATGCCTTCTTCTTTTTCCTACAGCTC |
| | | CTGGGCAACGTGCTGGTTATTGTGCTGTCTCATCATTTTGGCAAAGAACGCGCG |
| | | ATCGCCACCATGCCAGAGCCAGCGAAGTCTGCTCCCGCCCCGAAAAAGGGCTC |
| | | CAAGAAGGCGGTGACTAAGGCGCAGAAGAAAGGCGGCAAGAAGCGCAAGCG |
| | | CAGCCGCAAGGAGAGCTATTCCATCTATGTGTACAAGGTTCTGAAGCAGGTCC |
| | | ACCCTGACACCGGCATTTCGTCCAAGGCCATGGGCATCATGAATTCGTTTGTGA |
| | | ACGACATTTTCGAGCGCATCGCAGGTGAGGCTTCCCGCCTGGCGCATTACAAC |
| | | AAGCGCTCGACCATCACCTCCAGGGAGATCCAGACGGCCGTGCGCCTGCTGCT |
| | | GCCTGGGGAGTTGGCCAAGCACGCCGTGTCCGAGGGTACTAAGGCCATCACCA |
| | | AGTACACCAGCGCTAAGGATCCACCGGTCGCCACCATGGTGAGCAAGGGCGA |
| | | GGAGGTCATCAAAGAGTTCATGCGCTTCAAGGTGCGCATGGAGGGCTCCATGA |
| | | ACGGCCACGAGTTCGAGATCGAGGGCGAGGGCGAGGGCCGCCCCTACGAGGG |
| | | CACCCAGACCGCCAAGCTGAAGGTGACCAAGGGCGGCCCCCTGCCCTTCGCCT |
| | | GGGACATCCTGTCCCCCCAGTTCATGTACGGCTCCAAGGCGTACGTGAAGCAC |
| | | CCCGCCGACATCCCCGATTACAAGAAGCTGTCCTTCCCCGAGGGCTTCAAGTG |
| | | GGAGCGCGTGATGAACTTCGAGGACGGCGGTCTGGTGACCGTGACCCAGGACT |
| | | CCTCCCTGCAGGACGGCACGCTGATCTACAAGGTGAAGATGCGCGGCACCAAC |
| | | TTCCCCCCCGACGGCCCCGTAATGCAGAAGAAGACCATGGGCTGGGAGGCCTC |
| | | CACCGAGCGCCTGTACCCCCGCGACGGCGTGCTGAAGGGCGAGATCCACCAGG |
| | | CCCTGAAGCTGAAGGACGGCGGCCACTACCTGGTGGAGTTCAAGACCATCTAC |
| | | ATGGCCAAGAAGCCCGTGCAACTGCCCGGCTACTACTACGTGGACACCAAGCT |
| | | GGACATCACCTCCCACAACGAGGACTACACCATCGTGGAACAGTACGAGCGCT |
| | | CCGAGGGCCGCCACCACCTGTTCCTGGGGCATGGCACCGGCAGCACCGGCAGC |
| | | GGCAGCTCCGGCACCGCCTCCTCCGAGGACAACAACATGGCCGTCATCAAAGA |
| | | GTTCATGCGCTTCAAGGTGCGCATGGAGGGCTCCATGAACGGCCACGAGTTCG |
| | | AGATCGAGGGCGAGGGCGAGGGCCGCCCCTACGAGGGCACCCAGACCGCCAA |
| | | GCTGAAGGTGACCAAGGGCGGCCCCCTGCCCTTCGCCTGGGACATCCTGTCCC |
| | | CCCAGTTCATGTACGGCTCCAAGGCGTACGTGAAGCACCCCGCCGACATCCCC |
| | | GATTACAAGAAGCTGTCCTTCCCCGAGGGCTTCAAGTGGGAGCGCGTGATGAA |
| | | CTTCGAGGACGGCGGTCTGGTGACCGTGACCCAGGACTCCTCCCTGCAGGACG |
| | | GCACGCTGATCTACAAGGTGAAGATGCGCGGCACCAACTTCCCCCCCGACGGC |
| | | CCCGTAATGCAGAAGAAGACCATGGGCTGGGAGGCCTCCACCGAGCGCCTGTA |
| | | CCCCCGCGACGGCGTGCTGAAGGGCGAGATCCACCAGGCCCTGAAGCTGAAG |
| | | GACGGCGGCCACTACCTGGTGGAGTTCAAGACCATCTACATGGCCAAGAAGCC |
| | | CGTGCAACTGCCCGGCTACTACTACGTGGACACCAAGCTGGACATCACCTCCC |
| | | ACAACGAGGACTACACCATCGTGGAACAGTACGAGCGCTCCGAGGGCCGCCA |
| | | CCACCTGTTCCTGTACGGCATGGACGAGCTGTACAAGGCTACTAACTTCAGCCT |
| | | GCTGAAGCAGGCTGGAGACGTGGAGGAGAACCCTGGACCTATGGAAGACGCC |
| | | AAAAACATAAAGAAAGGCCCGGCGCCATTCTATCCGCTGGAAGATGGAACCG |
| | | CTGGAGAGCAACTGCATAAGGCTATGAAGAGATACGCCCTGGTTCCTGGAACA |
| | | ATTGCTTTTACAGATGCACATATCGAGGTGGACATCACTTACGCTGAGTACTTC |
| | | GAAATGTCCGTTCGGTTGGCAGAAGCTATGAAACGATATGGGCTGAATACAAA |
| | | TCACAGAATCGTCGTATGCAGTGAAAACTCTCTTCAATTCTTTATGCCGGTGTT |
| | | GGGCGCGTTATTTATCGGAGTTGCAGTTGCGCCCGCGAACGACATTTATAATG |
| | | AACGTGAATTGCTCAACAGTATGGGCATTTCGCAGCCTACCGTGGTGTTCGTTT |
| | | CCAAAAAGGGGTTGCAAAAAATTTTGAACGTGCAAAAAAAGCTCCCAATCATC |
| | | CAAAAAATTATTATCATGGATTCTAAAACGGATTACCAGGGATTTCAGTCGAT |
| | | GTACACGTTCGTCACATCTCATCTACCTCCCGGTTTTAATGAATACGATTTTGT |
| | | GCCAGAGTCCTTCGATAGGGACAAGACAATTGCACTGATCATGAACTCCTCTG |
| | | GATCTACTGGTCTGCCTAAAGGTGTCGCTCTGCCTCATAGAACTGCCTGCGTGA |
| | | GATTCTCGCATGCCAGAGATCCTATTTTTGGCAATCAAATCATTCCGGATACTG |
| | | CGATTTTAAGTGTTGTTCCATTCCATCACGGTTTTGGAATGTTTACTACACTCGG |
| | | ATATTTGATATGTGGATTTCGAGTCGTCTTAATGTATAGATTTGAAGAAGAGCT |
| | | GTTTCTGAGGAGCCTTCAGGATTACAAGATTCAAAGTGCGCTGCTGGTGCCAA |
| | | CCCTATTCTCCTTCTTCGCCAAAAGCACTCTGATTGACAAATACGATTTATCTA |
| | | ATTTACACGAAATTGCTTCTGGTGGCGCTCCCCTCTCTAAGGAAGTCGGGGAA |
| | | GCGGTTGCCAAGAGGTTCCATCTGCCAGGTATCAGGCAAGGATATGGGCTCAC |
| | | TGAGACTACATCAGCTATTCTGATTACACCCGAGGGGGATGATAAACCGGGCG |
| | | CGGTCGGTAAAGTTGTTCCATTTTTTGAAGCGAAGGTTGTGGATCTGGATACCG |
| | | GGAAAACGCTGGGCGTTAATCAAAGAGGCGAACTGTGTGTGAGAGGTCCTATG |
| | | ATTATGTCCGGTTATGTAAACAATCCGGAAGCGACCAACGCCTTGATTGACAA |
| | | GGATGGATGGCTACATTCTGGAGACATAGCTTACTGGGACGAAGACGAACACT |
| | | TCTTCATCGTTGACCGCCTGAAGTCTCTGATTAAGTACAAAGGCTATCAGGTGG |
| | | CTCCCGCTGAATTGGAATCCATCTTGCTCCAACACCCCAACATCTTCGACGCAG |
| | | GTGTCGCAGGTCTTCCCGACGATGACGCCGGTGAACTTCCCGCCGCCGTTGTTG |
| | | TTTTGGAGCACGGAAAGACGATGACGGAAAAAGAGATCGTGGATTACGTCGCC |
| | | AGTCAAGTAACAACCGCGAAAAAGTTGCGCGGAGGAGTTGTGTTTGTGGACGA |
| | | AGTACCGAAAGGTCTTACCGGAAAACTCGACGCAAGAAAAATCAGAGAGATC |
| | | CTCATAAAGGCCAAGAAGGGCGGAAAGATCGCCGTGTAACTCGAGAATCAAC |
| | | CTCTGGATTACAAAATTTGTGAAAGATTGACTGGTATTCTTAACTATGTTGCTC |
| | | CTTTTACGCTATGTGGATACGCTGCTTTAATGCCTTTGTATCATGCTATTGCTTC |
| | | CCGTATGGCTTTCATTTTCTCCTCCTTGTATAAATCCTGGTTGCTGTCTCTTTAT |
| | | GAGGAGTTGTGGCCCGTTGTCAGGCAACGTGGCGTGGTGTGCACTGTGTTTGCT |
| | | GACGCAACCCCCACTGGTTGGGGCATTGCCACCACCTGTCAGCTCCTTTCCGGG |
| | | ACTTTCGCTTTCCCCCTCCCTATTGCCACGGCGGAACTCATCGCCGCCTGCCTT |
| | | GCCCGCTGCTGGACAGGGGCTCGGCTGTTGGGCACTGACAATTCCGTGGGGTG |
| | | TGCCTTCTAGTTGCCAGCCATCTGTTGTTTGCCCCTCCCCCGTGCCTTCCTTGAC |
| | | CCTGGAAGGTGCCACTCCCACTGTCCTTTCCTAATAAAATGAGGAAATTGCATC |
| | | GCATTGTCTGAGTAGGTGTCATTCTATTCTGGGGGGTGGGGTGGGGCAGGACA |
| | | GCAAGGGGGAGGATTGGGAAGACAATAGCAGGCATGCTGGGGATGCGGTGGG |
| | | CTCTATGGGAAGATGTCTACTGAGCTGTGCGATCCCTGCTGGGGACTTTCCGCT |
| | | GGGGACTTTCCGCTGGGGACTTTCCGCCTTCAGCTAAGGAAGCTACCAATATTT |
| | | AGAGGTACATTTTGTTCTAGAACAAAATGTACCGGTACATTTTGTTCTGGTACA |
| | | TTTTGTTCTATTAAGGACAACGTAAGTATAGCGCATAGACACGGTCTCGAGGG |
| | | ATAACATGGCCCCCCCCCCCCCCCCCCCCCCGGTGGTGGTGGTTGTGGTGGTG |
| | | GTGGGGCCATGTTATCCCT |
| 70. | oGOI- | AGGGATAACATGGCCCCTGGATGGGAACTTACCGCTGCAGGCCATGTTATC |
| | CAG- | CCTCGAGACCTGCATCTAGATCCGACGTTACATAACTTACGGTAAATGGCCCG |
| | H2B- | CCTGGCTGACCGCCCAACGACCCCCGCCCATTGACGTCAATAATGACGTATGTT |
| | tdTomato- | CCCATAGTAACGCCAATAGGGACTTTCCATTGACGTCAATGGGTGGAGTATTT |
| | noV5- | ACGGTAAACTGCCCACTTGGCAGTACATCAAGTGTATCATATGCCAAGTACGC |
| | P2A-Fluc- | CCCCTATTGACGTCAATGACGGTAAATGGCCCGCCTGGCATTATGCCCAGTAC |
| | WPRE3- | ATGACCTTATGGGACTTTCCTACTTGGCAGTACATCTACGTATTAGTCATCGCT |
| | DTS-NH- | ATTACCATGGTCGAGGTGAGCCCCACGTTCTGCTTCACTCTCCCCATCTCCCCC |
| | 18 | CCCTCCCCACCCCCAATTTTGTATTTATTTATTTTTTAATTATTTTGTGCAGCGA |
| | | TGGGGGCGGGGGGGGGGGGGGCGCGCGCCAGGCGGGGCGGGGCGGGGCGAG |
| | | GGGCGGGGCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGCCCTATAAA |
| | | AAGCGAAGCGCGCGGCGGGCGGGAGTCGCTGCGCGCTGCCTTCGCCCCGTGCC |
| | | CCGCTCCGCCGCCGCCTCGCGCCGCCCGCCCCGGCTCTGACTGACCGCGTTACT |
| | | CCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGC |
| | | TCCGGGAGGGCCCTTTGTGCGGGGGGAGCGGCTCGGGGGGTGCGTGCGTGTGT |
| | | GTGTGCGTGGGGAGCGCCGCGTGCGGCTCCGCGCTGCCCGGCGGCTGTGAGCG |
| | | CTGCGGGCGCGGCGCGGGGCTTTGTGCGCTCCGCAGTGTGCGCGAGGGGAGCG |
| | | CGGCCGGGGGCGGTGCCCCGCGGTGCGGGGGGGGCTGCGAGGGGAACAAAGG |
| | | CTGCGTGCGGGGTGTGTGCGTGGGGGGGTGAGCAGGGGGTGTGGGCGCGTCG |
| | | GTCGGGCTGCAACCCCCCCTGCACCCCCCTCCCCGAGTTGCTGAGCACGGCCC |
| | | GGCTTCGGGTGCGGGGCTCCGTACGGGGCGTGGCGCGGGGCTCGCCGTGCCGG |
| | | GCGGGGGGTGGCGGCAGGTGGGGGTGCCGGGCGGGGCGGGGCCGCCTCGGGC |
| | | CGGGGAGGGCTCGGGGGAGGGGCGCGGCGGCCCCCGGAGCGCCGGCGGCTGT |
| | | CGAGGCGCGGCGAGCCGCAGCCATTGCCTTTTATGGTAATCGTGCGAGAGGGC |
| | | GCAGGGACTTCCTTTGTCCCAAATCTGTGCGGAGCCGAAATCTGGGAGGCGCC |
| | | GCCGCACCCCCTCTAGCGGGCGCGGGGCGAAGCGGTGCGGCGCCGGCAGGAA |
| | | GGAAATGGGCGGGGAGGGCCTTCGTGCGTCGCCGCGCCGCCGTCCCCTTCTCC |
| | | CTCTCCAGCCTCGGGGCTGTCCGCGGGGGGACGGCTGCCTTCGGGGGGGACGG |
| | | GGCAGGGCGGGGTTCGGCTTCTGGCGTGTGACCGGCGGCTCTAGAGCCTCTGC |
| | | TAACCATGTTCATGCCTTCTTCTTTTTCCTACAGCTCCTGGGCAACGTGCTGGTT |
| | | ATTGTGCTGTCTCATCATTTTGGCAAAGAACGCGCGATCGCCACCATGCCAGA |
| | | GCCAGCGAAGTCTGCTCCCGCCCCGAAAAAGGGCTCCAAGAAGGCGGTGACTA |
| | | AGGCGCAGAAGAAAGGCGGCAAGAAGCGCAAGCGCAGCCGCAAGGAGAGCT |
| | | ATTCCATCTATGTGTACAAGGTTCTGAAGCAGGTCCACCCTGACACCGGCATTT |
| | | CGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGGTCTCGAGGGATAACATGGCCCCTGGAT |
| | | GGGAACTTACCGCTGCAGGCCATGTTATCCCT |
| 71. | oGOI- | AGGGATAACATGGCCTGACAGTGGCGGCAGTCACTGAGAAAAACGAAACCG |
| | CAG- | GAGCGGCCATGTTATCCCTCGAGACCTGCATCTAGATCCGACGTTACATAACT |
| | H2B- | TACGGTAAATGGCCCGCCTGGCTGACCGCCCAACGACCCCCGCCCATTGACGT |
| | tdTomato- | CAATAATGACGTATGTTCCCATAGTAACGCCAATAGGGACTTTCCATTGACGTC |
| | noV5- | AATGGGTGGAGTATTTACGGTAAACTGCCCACTTGGCAGTACATCAAGTGTAT |
| | P2A-Fluc- | CATATGCCAAGTACGCCCCCTATTGACGTCAATGACGGTAAATGGCCCGCCTG |
| | WPRE3- | GCATTATGCCCAGTACATGACCTTATGGGACTTTCCTACTTGGCAGTACATCTA |
| | DTS- | CGTATTAGTCATCGCTATTACCATGGTCGAGGTGAGCCCCACGTTCTGCTTCAC |
| | NupApt01 | TCTCCCCATCTCCCCCCCCTCCCCACCCCCAATTTTGTATTTATTTATTTTTTAAT |
| | | TATTTTGTGCAGCGATGGGGGCGGGGGGGGGGGGGGCGCGCGCCAGGCGGGG |
| | | CGGGGGGGGCGAGGGGCGGGGCGGGGCGAGGCGGAGAGGTGCGGCGGCAG |
| | | CCAATCAGAGCGGCGCGCTCCGAAAGTTTCCTTTTATGGCGAGGCGGCGGCGG |
| | | CGGCGGCCCTATAAAAAGCGAAGCGCGCGGCGGGCGGGAGTCGCTGCGCGCT |
| | | GCCTTCGCCCCGTGCCCCGCTCCGCCGCCGCCTCGCGCCGCCCGCCCCGGCTCT |
| | | GACTGACCGCGTTACTCCCACAGGTGAGCGGGCGGGACGGCCCTTCTCCTCCG |
| | | GGCTGTAATTAGCGCTTGGTTTAATGACGGCTTGTTTCTTTTCTGTGGCTGCGTG |
| | | AAAGCCTTGAGGGGCTCCGGGAGGGCCCTTTGTGCGGGGGGAGCGGCTCGGG |
| | | GGGTGCGTGCGTGTGTGTGTGCGTGGGGAGCGCCGCGTGCGGCTCCGCGCTGC |
| | | CCGGCGGCTGTGAGCGCTGCGGGCGCGGCGCGGGGCTTTGTGCGCTCCGCAGT |
| | | GTGCGCGAGGGGAGCGCGGCCGGGGGCGGTGCCCCGCGGTGCGGGGGGGGCT |
| | | GCGAGGGGAACAAAGGCTGCGTGCGGGGTGTGTGCGTGGGGGGGTGAGCAGG |
| | | GGGTGTGGGCGCGTCGGTCGGGCTGCAACCCCCCCTGCACCCCCCTCCCCGAG |
| | | TTGCTGAGCACGGCCCGGCTTCGGGTGCGGGGCTCCGTACGGGGCGTGGCGCG |
| | | GGGCTCGCCGTGCCGGGCGGGGGGTGGCGGCAGGTGGGGGTGCCGGGCGGGG |
| | | CGGGGCCGCCTCGGGCCGGGGAGGGCTCGGGGGAGGGGCGCGGCGGCCCCCG |
| | | GAGCGCCGGCGGCTGTCGAGGCGCGGCGAGCCGCAGCCATTGCCTTTTATGGT |
| | | AATCGTGCGAGAGGGCGCAGGGACTTCCTTTGTCCCAAATCTGTGCGGAGCCG |
| | | AAATCTGGGAGGCGCCGCCGCACCCCCTCTAGCGGGCGCGGGGCGAAGCGGT |
| | | GCGGCGCCGGCAGGAAGGAAATGGGCGGGGAGGGCCTTCGTGCGTCGCCGCG |
| | | CCGCCGTCCCCTTCTCCCTCTCCAGCCTCGGGGCTGTCCGCGGGGGGACGGCTG |
| | | CCTTCGGGGGGGACGGGGCAGGGCGGGGTTCGGCTTCTGGCGTGTGACCGGCG |
| | | GCTCTAGAGCCTCTGCTAACCATGTTCATGCCTTCTTCTTTTTCCTACAGCTCCT |
| | | GGGCAACGTGCTGGTTATTGTGCTGTCTCATCATTTTGGCAAAGAACGCGCGAT |
| | | CGCCACCATGCCAGAGCCAGCGAAGTCTGCTCCCGCCCCGAAAAAGGGCTCCA |
| | | AGAAGGCGGTGACTAAGGCGCAGAAGAAAGGCGGCAAGAAGCGCAAGCGCA |
| | | GCCGCAAGGAGAGCTATTCCATCTATGTGTACAAGGTTCTGAAGCAGGTCCAC |
| | | CCTGACACCGGCATTTCGTCCAAGGCCATGGGCATCATGAATTCGTTTGTGAAC |
| | | GACATTTTCGAGCGCATCGCAGGTGAGGCTTCCCGCCTGGCGCATTACAACAA |
| | | GCGCTCGACCATCACCTCCAGGGAGATCCAGACGGCCGTGCGCCTGCTGCTGC |
| | | CTGGGGAGTTGGCCAAGCACGCCGTGTCCGAGGGTACTAAGGCCATCACCAAG |
| | | TACACCAGCGCTAAGGATCCACCGGTCGCCACCATGGTGAGCAAGGGCGAGG |
| | | AGGTCATCAAAGAGTTCATGCGCTTCAAGGTGCGCATGGAGGGCTCCATGAAC |
| | | GGCCACGAGTTCGAGATCGAGGGCGAGGGCGAGGGCCGCCCCTACGAGGGCA |
| | | CCCAGACCGCCAAGCTGAAGGTGACCAAGGGCGGCCCCCTGCCCTTCGCCTGG |
| | | GACATCCTGTCCCCCCAGTTCATGTACGGCTCCAAGGCGTACGTGAAGCACCC |
| | | CGCCGACATCCCCGATTACAAGAAGCTGTCCTTCCCCGAGGGCTTCAAGTGGG |
| | | AGCGCGTGATGAACTTCGAGGACGGCGGTCTGGTGACCGTGACCCAGGACTCC |
| | | TCCCTGCAGGACGGCACGCTGATCTACAAGGTGAAGATGCGCGGCACCAACTT |
| | | CCCCCCCGACGGCCCCGTAATGCAGAAGAAGACCATGGGCTGGGAGGCCTCCA |
| | | CCGAGCGCCTGTACCCCCGCGACGGCGTGCTGAAGGGCGAGATCCACCAGGCC |
| | | CTGAAGCTGAAGGACGGCGGCCACTACCTGGTGGAGTTCAAGACCATCTACAT |
| | | GGCCAAGAAGCCCGTGCAACTGCCCGGCTACTACTACGTGGACACCAAGCTGG |
| | | ACATCACCTCCCACAACGAGGACTACACCATCGTGGAACAGTACGAGCGCTCC |
| | | GAGGGCCGCCACCACCTGTTCCTGGGGCATGGCACCGGCAGCACCGGCAGCGG |
| | | CAGCTCCGGCACCGCCTCCTCCGAGGACAACAACATGGCCGTCATCAAAGAGT |
| | | TCATGCGCTTCAAGGTGCGCATGGAGGGCTCCATGAACGGCCACGAGTTCGAG |
| | | ATCGAGGGCGAGGGCGAGGGCCGCCCCTACGAGGGCACCCAGACCGCCAAGC |
| | | TGAAGGTGACCAAGGGCGGCCCCCTGCCCTTCGCCTGGGACATCCTGTCCCCC |
| | | CAGTTCATGTACGGCTCCAAGGCGTACGTGAAGCACCCCGCCGACATCCCCGA |
| | | TTACAAGAAGCTGTCCTTCCCCGAGGGCTTCAAGTGGGAGCGCGTGATGAACT |
| | | TCGAGGACGGCGGTCTGGTGACCGTGACCCAGGACTCCTCCCTGCAGGACGGC |
| | | ACGCTGATCTACAAGGTGAAGATGCGCGGCACCAACTTCCCCCCCGACGGCCC |
| | | CGTAATGCAGAAGAAGACCATGGGCTGGGAGGCCTCCACCGAGCGCCTGTACC |
| | | CCCGCGACGGCGTGCTGAAGGGCGAGATCCACCAGGCCCTGAAGCTGAAGGA |
| | | CGGCGGCCACTACCTGGTGGAGTTCAAGACCATCTACATGGCCAAGAAGCCCG |
| | | TGCAACTGCCCGGCTACTACTACGTGGACACCAAGCTGGACATCACCTCCCAC |
| | | AACGAGGACTACACCATCGTGGAACAGTACGAGCGCTCCGAGGGCCGCCACC |
| | | ACCTGTTCCTGTACGGCATGGACGAGCTGTACAAGGCTACTAACTTCAGCCTGC |
| | | TGAAGCAGGCTGGAGACGTGGAGGAGAACCCTGGACCTATGGAAGACGCCAA |
| | | AAACATAAAGAAAGGCCCGGCGCCATTCTATCCGCTGGAAGATGGAACCGCTG |
| | | GAGAGCAACTGCATAAGGCTATGAAGAGATACGCCCTGGTTCCTGGAACAATT |
| | | GCTTTTACAGATGCACATATCGAGGTGGACATCACTTACGCTGAGTACTTCGAA |
| | | ATGTCCGTTCGGTTGGCAGAAGCTATGAAACGATATGGGCTGAATACAAATCA |
| | | CAGAATCGTCGTATGCAGTGAAAACTCTCTTCAATTCTTTATGCCGGTGTTGGG |
| | | CGCGTTATTTATCGGAGTTGCAGTTGCGCCCGCGAACGACATTTATAATGAACG |
| | | TGAATTGCTCAACAGTATGGGCATTTCGCAGCCTACCGTGGTGTTCGTTTCCAA |
| | | AAAGGGGTTGCAAAAAATTTTGAACGTGCAAAAAAAGCTCCCAATCATCCAAA |
| | | AAATTATTATCATGGATTCTAAAACGGATTACCAGGGATTTCAGTCGATGTACA |
| | | CGTTCGTCACATCTCATCTACCTCCCGGTTTTAATGAATACGATTTTGTGCCAG |
| | | AGTCCTTCGATAGGGACAAGACAATTGCACTGATCATGAACTCCTCTGGATCT |
| | | ACTGGTCTGCCTAAAGGTGTCGCTCTGCCTCATAGAACTGCCTGCGTGAGATTC |
| | | TCGCATGCCAGAGATCCTATTTTTGGCAATCAAATCATTCCGGATACTGCGATT |
| | | TTAAGTGTTGTTCCATTCCATCACGGTTTTGGAATGTTTACTACACTCGGATATT |
| | | TGATATGTGGATTTCGAGTCGTCTTAATGTATAGATTTGAAGAAGAGCTGTTTC |
| | | TGAGGAGCCTTCAGGATTACAAGATTCAAAGTGCGCTGCTGGTGCCAACCCTA |
| | | TTCTCCTTCTTCGCCAAAAGCACTCTGATTGACAAATACGATTTATCTAATTTA |
| | | CACGAAATTGCTTCTGGTGGCGCTCCCCTCTCTAAGGAAGTCGGGGAAGCGGT |
| | | TGCCAAGAGGTTCCATCTGCCAGGTATCAGGCAAGGATATGGGCTCACTGAGA |
| | | CTACATCAGCTATTCTGATTACACCCGAGGGGGATGATAAACCGGGCGCGGTC |
| | | GGTAAAGTTGTTCCATTTTTTGAAGCGAAGGTTGTGGATCTGGATACCGGGAA |
| | | AACGCTGGGCGTTAATCAAAGAGGCGAACTGTGTGTGAGAGGTCCTATGATTA |
| | | TGTCCGGTTATGTAAACAATCCGGAAGCGACCAACGCCTTGATTGACAAGGAT |
| | | GGATGGCTACATTCTGGAGACATAGCTTACTGGGACGAAGACGAACACTTCTT |
| | | CATCGTTGACCGCCTGAAGTCTCTGATTAAGTACAAAGGCTATCAGGTGGCTCC |
| | | CGCTGAATTGGAATCCATCTTGCTCCAACACCCCAACATCTTCGACGCAGGTGT |
| | | CGCAGGTCTTCCCGACGATGACGCCGGTGAACTTCCCGCCGCCGTTGTTGTTTT |
| | | GGAGCACGGAAAGACGATGACGGAAAAAGAGATCGTGGATTACGTCGCCAGT |
| | | CAAGTAACAACCGCGAAAAAGTTGCGCGGAGGAGTTGTGTTTGTGGACGAAGT |
| | | ACCGAAAGGTCTTACCGGAAAACTCGACGCAAGAAAAATCAGAGAGATCCTC |
| | | ATAAAGGCCAAGAAGGGCGGAAAGATCGCCGTGTAACTCGAGAATCAACCTC |
| | | TGGATTACAAAATTTGTGAAAGATTGACTGGTATTCTTAACTATGTTGCTCCTT |
| | | TTACGCTATGTGGATACGCTGCTTTAATGCCTTTGTATCATGCTATTGCTTCCCG |
| | | TATGGCTTTCATTTTCTCCTCCTTGTATAAATCCTGGTTGCTGTCTCTTTATGAG |
| | | GAGTTGTGGCCCGTTGTCAGGCAACGTGGCGTGGTGTGCACTGTGTTTGCTGAC |
| | | GCAACCCCCACTGGTTGGGGCATTGCCACCACCTGTCAGCTCCTTTCCGGGACT |
| | | TTCGCTTTCCCCCTCCCTATTGCCACGGCGGAACTCATCGCCGCCTGCCTTGCC |
| | | CGCTGCTGGACAGGGGCTCGGCTGTTGGGCACTGACAATTCCGTGGGGTGTGC |
| | | CTTCTAGTTGCCAGCCATCTGTTGTTTGCCCCTCCCCCGTGCCTTCCTTGACCCT |
| | | GGAAGGTGCCACTCCCACTGTCCTTTCCTAATAAAATGAGGAAATTGCATCGC |
| | | ATTGTCTGAGTAGGTGTCATTCTATTCTGGGGGGTGGGGTGGGGCAGGACAGC |
| | | AAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGG |
| | | GGACTTTCCGCTGGGGACTTTCCGCCTTCAGCTAAGGAAGCTACCAATATTTAG |
| | | AGGTACATTTTGTTCTAGAACAAAATGTACCGGTACATTTTGTTCTGGTACATT |
| | | TTGTTCTATTAAGGACAACGTAAGTATAGCGCATAGACACGGTCTCGAGGGAT |
| | | AACATGGCCTGACAGTGGCGGCAGTCACTGAGAAAAACGAAACCGGAGCGG |
| | | CCATGTTATCCCT |
| 72. | oGOI- | AGGGATAACATGGCCCCACGCAGATAGACGCTACTCTACTACATCGCAGCCA |
| | CAG- | ACGGCCATGTTATCCCTCGAGACCTGCATCTAGATCCGACGTTACATAACTTA |
| | H2B- | CGGTAAATGGCCCGCCTGGCTGACCGCCCAACGACCCCCGCCCATTGACGTCA |
| | tdTomato- | ATAATGACGTATGTTCCCATAGTAACGCCAATAGGGACTTTCCATTGACGTCAA |
| | noV5- | TGGGTGGAGTATTTACGGTAAACTGCCCACTTGGCAGTACATCAAGTGTATCAT |
| | P2A-Fluc- | ATGCCAAGTACGCCCCCTATTGACGTCAATGACGGTAAATGGCCCGCCTGGCA |
| | WPRE3- | TTATGCCCAGTACATGACCTTATGGGACTTTCCTACTTGGCAGTACATCTACGT |
| | DTS- | ATTAGTCATCGCTATTACCATGGTCGAGGTGAGCCCCACGTTCTGCTTCACTCT |
| | NupApt02 | CCCCATCTCCCCCCCCTCCCCACCCCCAATTTTGTATTTATTTATTTTTTAATTAT |
| | | TTTGTGCAGCGATGGGGGCGGGGGGGGGGGGGGCGCGCGCCAGGCGGGGCGG |
| | | GGCGGGGCGAGGGGCGGGGCGGGGCGAGGCGGAGAGGTGCGGCGGCAGCCA |
| | | ATCAGAGCGGCGCGCTCCGAAAGTTTCCTTTTATGGCGAGGCGGCGGCGGCGG |
| | | CGGCCCTATAAAAAGCGAAGCGCGCGGCGGGCGGGAGTCGCTGCGCGCTGCCT |
| | | TCGCCCCGTGCCCCGCTCCGCCGCCGCCTCGCGCCGCCCGCCCCGGCTCTGACT |
| | | GACCGCGTTACTCCCACAGGTGAGCGGGCGGGACGGCCCTTCTCCTCCGGGCT |
| | | GTAATTAGCGCTTGGTTTAATGACGGCTTGTTTCTTTTCTGTGGCTGCGTGAAA |
| | | GCCTTGAGGGGCTCCGGGAGGGCCCTTTGTGCGGGGGGAGCGGCTCGGGGGGT |
| | | GCGTGCGTGTGTGTGTGCGTGGGGAGCGCCGCGTGCGGCTCCGCGCTGCCCGG |
| | | CGGCTGTGAGCGCTGCGGGCGCGGCGCGGGGCTTTGTGCGCTCCGCAGTGTGC |
| | | GCGAGGGGAGCGCGGCCGGGGGCGGTGCCCCGCGGTGCGGGGGGGGCTGCGA |
| | | GGGGAACAAAGGCTGCGTGCGGGGTGTGTGCGTGGGGGGGTGAGCAGGGGGT |
| | | GTGGGCGCGTCGGTCGGGCTGCAACCCCCCCTGCACCCCCCTCCCCGAGTTGCT |
| | | GAGCACGGCCCGGCTTCGGGTGCGGGGCTCCGTACGGGGCGTGGCGCGGGGCT |
| | | CGCCGTGCCGGGCGGGGGGTGGCGGCAGGTGGGGGTGCCGGGCGGGGCGGGG |
| | | CCGCCTCGGGCCGGGGAGGGCTCGGGGGAGGGGCGCGGCGGCCCCCGGAGCG |
| | | CCGGCGGCTGTCGAGGCGCGGCGAGCCGCAGCCATTGCCTTTTATGGTAATCG |
| | | TGCGAGAGGGCGCAGGGACTTCCTTTGTCCCAAATCTGTGCGGAGCCGAAATC |
| | | TGGGAGGCGCCGCCGCACCCCCTCTAGCGGGCGCGGGGCGAAGCGGTGCGGC |
| | | GCCGGCAGGAAGGAAATGGGCGGGGAGGGCCTTCGTGCGTCGCCGCGCCGCC |
| | | GTCCCCTTCTCCCTCTCCAGCCTCGGGGCTGTCCGCGGGGGGACGGCTGCCTTC |
| | | GGGGGGGACGGGGCAGGGCGGGGTTCGGCTTCTGGCGTGTGACCGGCGGCTCT |
| | | AGAGCCTCTGCTAACCATGTTCATGCCTTCTTCTTTTTCCTACAGCTCCTGGGCA |
| | | ACGTGCTGGTTATTGTGCTGTCTCATCATTTTGGCAAAGAACGCGCGATCGCCA |
| | | CCATGCCAGAGCCAGCGAAGTCTGCTCCCGCCCCGAAAAAGGGCTCCAAGAAG |
| | | GCGGTGACTAAGGCGCAGAAGAAAGGCGGCAAGAAGCGCAAGCGCAGCCGCA |
| | | AGGAGAGCTATTCCATCTATGTGTACAAGGTTCTGAAGCAGGTCCACCCTGAC |
| | | ACCGGCATTTCGTCCAAGGCCATGGGCATCATGAATTCGTTTGTGAACGACATT |
| | | TTCGAGCGCATCGCAGGTGAGGCTTCCCGCCTGGCGCATTACAACAAGCGCTC |
| | | GACCATCACCTCCAGGGAGATCCAGACGGCCGTGCGCCTGCTGCTGCCTGGGG |
| | | AGTTGGCCAAGCACGCCGTGTCCGAGGGTACTAAGGCCATCACCAAGTACACC |
| | | AGCGCTAAGGATCCACCGGTCGCCACCATGGTGAGCAAGGGCGAGGAGGTCA |
| | | TCAAAGAGTTCATGCGCTTCAAGGTGCGCATGGAGGGCTCCATGAACGGCCAC |
| | | GAGTTCGAGATCGAGGGCGAGGGCGAGGGCCGCCCCTACGAGGGCACCCAGA |
| | | CCGCCAAGCTGAAGGTGACCAAGGGCGGCCCCCTGCCCTTCGCCTGGGACATC |
| | | CTGTCCCCCCAGTTCATGTACGGCTCCAAGGCGTACGTGAAGCACCCCGCCGA |
| | | CATCCCCGATTACAAGAAGCTGTCCTTCCCCGAGGGCTTCAAGTGGGAGCGCG |
| | | TGATGAACTTCGAGGACGGCGGTCTGGTGACCGTGACCCAGGACTCCTCCCTG |
| | | CAGGACGGCACGCTGATCTACAAGGTGAAGATGCGCGGCACCAACTTCCCCCC |
| | | CGACGGCCCCGTAATGCAGAAGAAGACCATGGGCTGGGAGGCCTCCACCGAG |
| | | CGCCTGTACCCCCGCGACGGCGTGCTGAAGGGCGAGATCCACCAGGCCCTGAA |
| | | GCTGAAGGACGGCGGCCACTACCTGGTGGAGTTCAAGACCATCTACATGGCCA |
| | | AGAAGCCCGTGCAACTGCCCGGCTACTACTACGTGGACACCAAGCTGGACATC |
| | | ACCTCCCACAACGAGGACTACACCATCGTGGAACAGTACGAGCGCTCCGAGGG |
| | | CCGCCACCACCTGTTCCTGGGGCATGGCACCGGCAGCACCGGCAGCGGCAGCT |
| | | CCGGCACCGCCTCCTCCGAGGACAACAACATGGCCGTCATCAAAGAGTTCATG |
| | | CGCTTCAAGGTGCGCATGGAGGGCTCCATGAACGGCCACGAGTTCGAGATCGA |
| | | GGGCGAGGGCGAGGGCCGCCCCTACGAGGGCACCCAGACCGCCAAGCTGAAG |
| | | GTGACCAAGGGCGGCCCCCTGCCCTTCGCCTGGGACATCCTGTCCCCCCAGTTC |
| | | ATGTACGGCTCCAAGGCGTACGTGAAGCACCCCGCCGACATCCCCGATTACAA |
| | | GAAGCTGTCCTTCCCCGAGGGCTTCAAGTGGGAGCGCGTGATGAACTTCGAGG |
| | | ACGGCGGTCTGGTGACCGTGACCCAGGACTCCTCCCTGCAGGACGGCACGCTG |
| | | ATCTACAAGGTGAAGATGCGCGGCACCAACTTCCCCCCCGACGGCCCCGTAAT |
| | | GCAGAAGAAGACCATGGGCTGGGAGGCCTCCACCGAGCGCCTGTACCCCCGCG |
| | | ACGGCGTGCTGAAGGGCGAGATCCACCAGGCCCTGAAGCTGAAGGACGGCGG |
| | | CCACTACCTGGTGGAGTTCAAGACCATCTACATGGCCAAGAAGCCCGTGCAAC |
| | | TGCCCGGCTACTACTACGTGGACACCAAGCTGGACATCACCTCCCACAACGAG |
| | | GACTACACCATCGTGGAACAGTACGAGCGCTCCGAGGGCCGCCACCACCTGTT |
| | | CCTGTACGGCATGGACGAGCTGTACAAGGCTACTAACTTCAGCCTGCTGAAGC |
| | | AGGCTGGAGACGTGGAGGAGAACCCTGGACCTATGGAAGACGCCAAAAACAT |
| | | AAAGAAAGGCCCGGCGCCATTCTATCCGCTGGAAGATGGAACCGCTGGAGAG |
| | | CAACTGCATAAGGCTATGAAGAGATACGCCCTGGTTCCTGGAACAATTGCTTTT |
| | | ACAGATGCACATATCGAGGTGGACATCACTTACGCTGAGTACTTCGAAATGTC |
| | | CGTTCGGTTGGCAGAAGCTATGAAACGATATGGGCTGAATACAAATCACAGAA |
| | | TCGTCGTATGCAGTGAAAACTCTCTTCAATTCTTTATGCCGGTGTTGGGCGCGT |
| | | TATTTATCGGAGTTGCAGTTGCGCCCGCGAACGACATTTATAATGAACGTGAAT |
| | | TGCTCAACAGTATGGGCATTTCGCAGCCTACCGTGGTGTTCGTTTCCAAAAAGG |
| | | GGTTGCAAAAAATTTTGAACGTGCAAAAAAAGCTCCCAATCATCCAAAAAATT |
| | | ATTATCATGGATTCTAAAACGGATTACCAGGGATTTCAGTCGATGTACACGTTC |
| | | GTCACATCTCATCTACCTCCCGGTTTTAATGAATACGATTTTGTGCCAGAGTCC |
| | | TTCGATAGGGACAAGACAATTGCACTGATCATGAACTCCTCTGGATCTACTGGT |
| | | CTGCCTAAAGGTGTCGCTCTGCCTCATAGAACTGCCTGCGTGAGATTCTCGCAT |
| | | GCCAGAGATCCTATTTTTGGCAATCAAATCATTCCGGATACTGCGATTTTAAGT |
| | | GTTGTTCCATTCCATCACGGTTTTGGAATGTTTACTACACTCGGATATTTGATAT |
| | | GTGGATTTCGAGTCGTCTTAATGTATAGATTTGAAGAAGAGCTGTTTCTGAGGA |
| | | GCCTTCAGGATTACAAGATTCAAAGTGCGCTGCTGGTGCCAACCCTATTCTCCT |
| | | TCTTCGCCAAAAGCACTCTGATTGACAAATACGATTTATCTAATTTACACGAAA |
| | | TTGCTTCTGGTGGCGCTCCCCTCTCTAAGGAAGTCGGGGAAGCGGTTGCCAAG |
| | | AGGTTCCATCTGCCAGGTATCAGGCAAGGATATGGGCTCACTGAGACTACATC |
| | | AGCTATTCTGATTACACCCGAGGGGGATGATAAACCGGGCGCGGTCGGTAAAG |
| | | TTGTTCCATTTTTTGAAGCGAAGGTTGTGGATCTGGATACCGGGAAAACGCTGG |
| | | GCGTTAATCAAAGAGGCGAACTGTGTGTGAGAGGTCCTATGATTATGTCCGGT |
| | | TATGTAAACAATCCGGAAGCGACCAACGCCTTGATTGACAAGGATGGATGGCT |
| | | ACATTCTGGAGACATAGCTTACTGGGACGAAGACGAACACTTCTTCATCGTTG |
| | | ACCGCCTGAAGTCTCTGATTAAGTACAAAGGCTATCAGGTGGCTCCCGCTGAA |
| | | TTGGAATCCATCTTGCTCCAACACCCCAACATCTTCGACGCAGGTGTCGCAGGT |
| | | CTTCCCGACGATGACGCCGGTGAACTTCCCGCCGCCGTTGTTGTTTTGGAGCAC |
| | | GGAAAGACGATGACGGAAAAAGAGATCGTGGATTACGTCGCCAGTCAAGTAA |
| | | CAACCGCGAAAAAGTTGCGCGGAGGAGTTGTGTTTGTGGACGAAGTACCGAAA |
| | | GGTCTTACCGGAAAACTCGACGCAAGAAAAATCAGAGAGATCCTCATAAAGG |
| | | CCAAGAAGGGCGGAAAGATCGCCGTGTAACTCGAGAATCAACCTCTGGATTAC |
| | | AAAATTTGTGAAAGATTGACTGGTATTCTTAACTATGTTGCTCCTTTTACGCTAT |
| | | GTGGATACGCTGCTTTAATGCCTTTGTATCATGCTATTGCTTCCCGTATGGCTTT |
| | | CATTTTCTCCTCCTTGTATAAATCCTGGTTGCTGTCTCTTTATGAGGAGTTGTGG |
| | | CCCGTTGTCAGGCAACGTGGCGTGGTGTGCACTGTGTTTGCTGACGCAACCCCC |
| | | ACTGGTTGGGGCATTGCCACCACCTGTCAGCTCCTTTCCGGGACTTTCGCTTTC |
| | | CCCCTCCCTATTGCCACGGCGGAACTCATCGCCGCCTGCCTTGCCCGCTGCTGG |
| | | ACAGGGGCTCGGCTGTTGGGCACTGACAATTCCGTGGGGTGTGCCTTCTAGTTG |
| | | CCAGCCATCTGTTGTTTGCCCCTCCCCCGTGCCTTCCTTGACCCTGGAAGGTGC |
| | | CACTCCCACTGTCCTTTCCTAATAAAATGAGGAAATTGCATCGCATTGTCTGAG |
| | | TAGGTGTCATTCTATTCTGGGGGGTGGGGTGGGGCAGGACAGCAAGGGGGAGG |
| | | ATTGGGAAGACAATAGCAGGCATGCTGGGGATGCGGTGGGCTCTATGGGAAG |
| | | ATGTCTACTGAGCTGTGCGATCCCTGCTGGGGACTTTCCGCTGGGGACTTTCCG |
| | | CTGGGGACTTTCCGCCTTCAGCTAAGGAAGCTACCAATATTTAGAGGTACATTT |
| | | TGTTCTAGAACAAAATGTACCGGTACATTTTGTTCTGGTACATTTTGTTCTATTA |
| | | AGGACAACGTAAGTATAGCGCATAGACACGGTCTCGAGGGATAACATGGCCC |
| | | CACGCAGATAGACGCTACTCTACTACATCGCAGCCAACGGCCATGTTATCCC |
| | | T |
| 73. | oGOI- | AGGGATAACATGGCCTTAGGGTTAGGGTTAGGGTTAGGGGGCCATGTTATC |
| | CAG- | CCTCGAGACCTGCATCTAGATCCGACGTTACATAACTTACGGTAAATGGCCCG |
| | H2B- | CCTGGCTGACCGCCCAACGACCCCCGCCCATTGACGTCAATAATGACGTATGTT |
| | tdTomato- | CCCATAGTAACGCCAATAGGGACTTTCCATTGACGTCAATGGGTGGAGTATTT |
| | noV5- | ACGGTAAACTGCCCACTTGGCAGTACATCAAGTGTATCATATGCCAAGTACGC |
| | P2A-Fluc- | CCCCTATTGACGTCAATGACGGTAAATGGCCCGCCTGGCATTATGCCCAGTAC |
| | WPRE3- | ATGACCTTATGGGACTTTCCTACTTGGCAGTACATCTACGTATTAGTCATCGCT |
| | DTS- | ATTACCATGGTCGAGGTGAGCCCCACGTTCTGCTTCACTCTCCCCATCTCCCCC |
| | A151 | CCCTCCCCACCCCCAATTTTGTATTTATTTATTTTTTAATTATTTTGTGCAGCGA |
| | | TGGGGGCGGGGGGGGGGGGGGCGCGCGCCAGGCGGGGCGGGGCGGGGCGAG |
| | | GGGCGGGGCGGGGCGAGGCGGAGAGGTGCGGCGGCAGCCAATCAGAGCGGCG |
| | | CGCTCCGAAAGTTTCCTTTTATGGCGAGGCGGCGGCGGCGGCGGCCCTATAAA |
| | | AAGCGAAGCGCGCGGCGGGCGGGAGTCGCTGCGCGCTGCCTTCGCCCCGTGCC |
| | | CCGCTCCGCCGCCGCCTCGCGCCGCCCGCCCCGGCTCTGACTGACCGCGTTACT |
| | | CCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGC |
| | | TCCGGGAGGGCCCTTTGTGCGGGGGGAGCGGCTCGGGGGGTGCGTGCGTGTGT |
| | | GTGTGCGTGGGGAGCGCCGCGTGCGGCTCCGCGCTGCCCGGCGGCTGTGAGCG |
| | | CTGCGGGCGCGGCGCGGGGCTTTGTGCGCTCCGCAGTGTGCGCGAGGGGAGCG |
| | | CGGCCGGGGGCGGTGCCCCGCGGTGCGGGGGGGGCTGCGAGGGGAACAAAGG |
| | | CTGCGTGCGGGGTGTGTGCGTGGGGGGGTGAGCAGGGGGTGTGGGCGCGTCG |
| | | GTCGGGCTGCAACCCCCCCTGCACCCCCCTCCCCGAGTTGCTGAGCACGGCCC |
| | | GGCTTCGGGTGCGGGGCTCCGTACGGGGCGTGGCGCGGGGCTCGCCGTGCCGG |
| | | GCGGGGGGTGGCGGCAGGTGGGGGTGCCGGGCGGGGCGGGGCCGCCTCGGGC |
| | | CGGGGAGGGCTCGGGGGAGGGGCGCGGCGGCCCCCGGAGCGCCGGCGGCTGT |
| | | CGAGGCGCGGCGAGCCGCAGCCATTGCCTTTTATGGTAATCGTGCGAGAGGGC |
| | | GCAGGGACTTCCTTTGTCCCAAATCTGTGCGGAGCCGAAATCTGGGAGGCGCC |
| | | GCCGCACCCCCTCTAGCGGGCGCGGGGCGAAGCGGTGCGGCGCCGGCAGGAA |
| | | GGAAATGGGCGGGGAGGGCCTTCGTGCGTCGCCGCGCCGCCGTCCCCTTCTCC |
| | | CTCTCCAGCCTCGGGGCTGTCCGCGGGGGGACGGCTGCCTTCGGGGGGGACGG |
| | | GGCAGGGCGGGGTTCGGCTTCTGGCGTGTGACCGGCGGCTCTAGAGCCTCTGC |
| | | TAACCATGTTCATGCCTTCTTCTTTTTCCTACAGCTCCTGGGCAACGTGCTGGTT |
| | | ATTGTGCTGTCTCATCATTTTGGCAAAGAACGCGCGATCGCCACCATGCCAGA |
| | | GCCAGCGAAGTCTGCTCCCGCCCCGAAAAAGGGCTCCAAGAAGGCGGTGACTA |
| | | AGGCGCAGAAGAAAGGCGGCAAGAAGCGCAAGCGCAGCCGCAAGGAGAGCT |
| | | ATTCCATCTATGTGTACAAGGTTCTGAAGCAGGTCCACCCTGACACCGGCATTT |
| | | CGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGGTCTCGAGGGATAACATGGCCTTAGGGT |
| | | TAGGGTTAGGGTTAGGGGGCCATGTTATCCCT |
| 74. | oGOI- | GAGGGATAACATGGCCCCTGGATGGGAAGGCCATGTTATCCCTCGAGACCT |
| | CAG | GCATCTAGATCCGACGTTACATAACTTACGGTAAATGGCCCGCCTGGCTGACC |
| | H2B | GCCCAACGACCCCCGCCCATTGACGTCAATAATGACGTATGTTCCCATAGTAA |
| | tdTomato- | CGCCAATAGGGACTTTCCATTGACGTCAATGGGTGGAGTATTTACGGTAAACT |
| | no V5- | GCCCACTTGGCAGTACATCAAGTGTATCATATGCCAAGTACGCCCCCTATTGAC |
| | P2A-Fluc- | GTCAATGACGGTAAATGGCCCGCCTGGCATTATGCCCAGTACATGACCTTATG |
| | WPRE3- | GGACTTTCCTACTTGGCAGTACATCTACGTATTAGTCATCGCTATTACCATGGT |
| | DTS- | CGAGGTGAGCCCCACGTTCTGCTTCACTCTCCCCATCTCCCCCCCCTCCCCACC |
| | 4084-F | CCCAATTTTGTATTTATTTATTTTTTAATTATTTTGTGCAGCGATGGGGGCGGGG |
| | | GGGGGGGGGGGGCGCGCCAGGCGGGGCGGGGCGGGGCGAGGGGCGGGGCGG |
| | | GGCGAGGCGGAGAGGTGCGGCGGCAGCCAATCAGAGCGGCGCGCTCCGAAAG |
| | | TTTCCTTTTATGGCGAGGCGGCGGCGGCGGCGGCCCTATAAAAAGCGAAGCGC |
| | | GCGGCGGGCGGGAGTCGCTGCGCGCTGCCTTCGCCCCGTGCCCCGCTCCGCCG |
| | | CCGCCTCGCGCCGCCCGCCCCGGCTCTGACTGACCGCGTTACTCCCACAGGTGA |
| | | GCGGGCGGGACGGCCCTTCTCCTCCGGGCTGTAATTAGCGCTTGGTTTAATGAC |
| | | GGCTTGTTTCTTTTCTGTGGCTGCGTGAAAGCCTTGAGGGGCTCCGGGAGGGCC |
| | | CTTTGTGCGGGGGGAGCGGCTCGGGGGGTGCGTGCGTGTGTGTGTGCGTGGGG |
| | | AGCGCCGCGTGCGGCTCCGCGCTGCCCGGCGGCTGTGAGCGCTGCGGGCGCGG |
| | | CGCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGG |
| | | TGTGTGCGTGGGGGGGTGAGCAGGGGGTGTGGGCGCGTCGGTCGGGCTGCAAC |
| | | CCCCCCTGCACCCCCCTCCCCGAGTTGCTGAGCACGGCCCGGCTTCGGGTGCGG |
| | | GGCTCCGTACGGGGCGTGGCGCGGGGCTCGCCGTGCCGGGCGGGGGGTGGCG |
| | | GCAGGTGGGGGTGCCGGGCGGGGCGGGGCCGCCTCGGGCCGGGGAGGGCTCG |
| | | GGGGAGGGGCGCGGCGGCCCCCGGAGCGCCGGCGGCTGTCGAGGCGCGGCGA |
| | | GCCGCAGCCATTGCCTTTTATGGTAATCGTGCGAGAGGGCGCAGGGACTTCCTT |
| | | TGTCCCAAATCTGTGCGGAGCCGAAATCTGGGAGGCGCCGCCGCACCCCCTCT |
| | | AGCGGGCGCGGGGCGAAGCGGTGCGGCGCCGGCAGGAAGGAAATGGGCGGG |
| | | GAGGGCCTTCGTGCGTCGCCGCGCCGCCGTCCCCTTCTCCCTCTCCAGCCTCGG |
| | | GGCTGTCCGCGGGGGGACGGCTGCCTTCGGGGGGGACGGGGCAGGGCGGGGT |
| | | TCGGCTTCTGGCGTGTGACCGGCGGCTCTAGAGCCTCTGCTAACCATGTTCATG |
| | | CCTTCTTCTTTTTCCTACAGCTCCTGGGCAACGTGCTGGTTATTGTGCTGTCTCA |
| | | TCATTTTGGCAAAGAACGCGCGATCGCCACCATGCCAGAGCCAGCGAAGTCTG |
| | | CTCCCGCCCCGAAAAAGGGCTCCAAGAAGGCGGTGACTAAGGCGCAGAAGAA |
| | | AGGCGGCAAGAAGCGCAAGCGCAGCCGCAAGGAGAGCTATTCCATCTATGTGT |
| | | ACAAGGTTCTGAAGCAGGTCCACCCTGACACCGGCATTTCGTCCAAGGCCATG |
| | | GGCATCATGAATTCGTTTGTGAACGACATTTTCGAGCGCATCGCAGGTGAGGC |
| | | TTCCCGCCTGGCGCATTACAACAAGCGCTCGACCATCACCTCCAGGGAGATCC |
| | | AGACGGCCGTGCGCCTGCTGCTGCCTGGGGAGTTGGCCAAGCACGCCGTGTCC |
| | | GAGGGTACTAAGGCCATCACCAAGTACACCAGCGCTAAGGATCCACCGGTCGC |
| | | CACCATGGTGAGCAAGGGCGAGGAGGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGGGGCA |
| | | TGGCACCGGCAGCACCGGCAGCGGCAGCTCCGGCACCGCCTCCTCCGAGGACA |
| | | ACAACATGGCCGTCATCAAAGAGTTCATGCGCTTCAAGGTGCGCATGGAGGGC |
| | | TCCATGAACGGCCACGAGTTCGAGATCGAGGGCGAGGGCGAGGGCCGCCCCT |
| | | ACGAGGGCACCCAGACCGCCAAGCTGAAGGTGACCAAGGGCGGCCCCCTGCC |
| | | CTTCGCCTGGGACATCCTGTCCCCCCAGTTCATGTACGGCTCCAAGGCGTACGT |
| | | GAAGCACCCCGCCGACATCCCCGATTACAAGAAGCTGTCCTTCCCCGAGGGCT |
| | | TCAAGTGGGAGCGCGTGATGAACTTCGAGGACGGCGGTCTGGTGACCGTGACC |
| | | CAGGACTCCTCCCTGCAGGACGGCACGCTGATCTACAAGGTGAAGATGCGCGG |
| | | CACCAACTTCCCCCCCGACGGCCCCGTAATGCAGAAGAAGACCATGGGCTGGG |
| | | AGGCCTCCACCGAGCGCCTGTACCCCCGCGACGGCGTGCTGAAGGGCGAGATC |
| | | CACCAGGCCCTGAAGCTGAAGGACGGCGGCCACTACCTGGTGGAGTTCAAGAC |
| | | CATCTACATGGCCAAGAAGCCCGTGCAACTGCCCGGCTACTACTACGTGGACA |
| | | CCAAGCTGGACATCACCTCCCACAACGAGGACTACACCATCGTGGAACAGTAC |
| | | GAGCGCTCCGAGGGCCGCCACCACCTGTTCCTGTACGGCATGGACGAGCTGTA |
| | | CAAGGCTACTAACTTCAGCCTGCTGAAGCAGGCTGGAGACGTGGAGGAGAACC |
| | | CTGGACCTATGGAAGACGCCAAAAACATAAAGAAAGGCCCGGCGCCATTCTAT |
| | | CCGCTGGAAGATGGAACCGCTGGAGAGCAACTGCATAAGGCTATGAAGAGAT |
| | | ACGCCCTGGTTCCTGGAACAATTGCTTTTACAGATGCACATATCGAGGTGGAC |
| | | ATCACTTACGCTGAGTACTTCGAAATGTCCGTTCGGTTGGCAGAAGCTATGAA |
| | | ACGATATGGGCTGAATACAAATCACAGAATCGTCGTATGCAGTGAAAACTCTC |
| | | TTCAATTCTTTATGCCGGTGTTGGGCGCGTTATTTATCGGAGTTGCAGTTGCGC |
| | | CCGCGAACGACATTTATAATGAACGTGAATTGCTCAACAGTATGGGCATTTCG |
| | | CAGCCTACCGTGGTGTTCGTTTCCAAAAAGGGGTTGCAAAAAATTTTGAACGT |
| | | GCAAAAAAAGCTCCCAATCATCCAAAAAATTATTATCATGGATTCTAAAACGG |
| | | ATTACCAGGGATTTCAGTCGATGTACACGTTCGTCACATCTCATCTACCTCCCG |
| | | GTTTTAATGAATACGATTTTGTGCCAGAGTCCTTCGATAGGGACAAGACAATTG |
| | | CACTGATCATGAACTCCTCTGGATCTACTGGTCTGCCTAAAGGTGTCGCTCTGC |
| | | CTCATAGAACTGCCTGCGTGAGATTCTCGCATGCCAGAGATCCTATTTTTGGCA |
| | | ATCAAATCATTCCGGATACTGCGATTTTAAGTGTTGTTCCATTCCATCACGGTTT |
| | | TGGAATGTTTACTACACTCGGATATTTGATATGTGGATTTCGAGTCGTCTTAAT |
| | | GTATAGATTTGAAGAAGAGCTGTTTCTGAGGAGCCTTCAGGATTACAAGATTC |
| | | AAAGTGCGCTGCTGGTGCCAACCCTATTCTCCTTCTTCGCCAAAAGCACTCTGA |
| | | TTGACAAATACGATTTATCTAATTTACACGAAATTGCTTCTGGTGGCGCTCCCC |
| | | TCTCTAAGGAAGTCGGGGAAGCGGTTGCCAAGAGGTTCCATCTGCCAGGTATC |
| | | AGGCAAGGATATGGGCTCACTGAGACTACATCAGCTATTCTGATTACACCCGA |
| | | GGGGGATGATAAACCGGGCGCGGTCGGTAAAGTTGTTCCATTTTTTGAAGCGA |
| | | AGGTTGTGGATCTGGATACCGGGAAAACGCTGGGCGTTAATCAAAGAGGCGA |
| | | ACTGTGTGTGAGAGGTCCTATGATTATGTCCGGTTATGTAAACAATCCGGAAGC |
| | | GACCAACGCCTTGATTGACAAGGATGGATGGCTACATTCTGGAGACATAGCTT |
| | | ACTGGGACGAAGACGAACACTTCTTCATCGTTGACCGCCTGAAGTCTCTGATTA |
| | | AGTACAAAGGCTATCAGGTGGCTCCCGCTGAATTGGAATCCATCTTGCTCCAA |
| | | CACCCCAACATCTTCGACGCAGGTGTCGCAGGTCTTCCCGACGATGACGCCGG |
| | | TGAACTTCCCGCCGCCGTTGTTGTTTTGGAGCACGGAAAGACGATGACGGAAA |
| | | AAGAGATCGTGGATTACGTCGCCAGTCAAGTAACAACCGCGAAAAAGTTGCGC |
| | | GGAGGAGTTGTGTTTGTGGACGAAGTACCGAAAGGTCTTACCGGAAAACTCGA |
| | | CGCAAGAAAAATCAGAGAGATCCTCATAAAGGCCAAGAAGGGCGGAAAGATC |
| | | GCCGTGTAACTCGAGAATCAACCTCTGGATTACAAAATTTGTGAAAGATTGAC |
| | | TGGTATTCTTAACTATGTTGCTCCTTTTACGCTATGTGGATACGCTGCTTTAATG |
| | | CCTTTGTATCATGCTATTGCTTCCCGTATGGCTTTCATTTTCTCCTCCTTGTATAA |
| | | ATCCTGGTTGCTGTCTCTTTATGAGGAGTTGTGGCCCGTTGTCAGGCAACGTGG |
| | | CGTGGTGTGCACTGTGTTTGCTGACGCAACCCCCACTGGTTGGGGCATTGCCAC |
| | | CACCTGTCAGCTCCTTTCCGGGACTTTCGCTTTCCCCCTCCCTATTGCCACGGCG |
| | | GAACTCATCGCCGCCTGCCTTGCCCGCTGCTGGACAGGGGCTCGGCTGTTGGG |
| | | CACTGACAATTCCGTGGGGTGTGCCTTCTAGTTGCCAGCCATCTGTTGTTTGCC |
| | | CCTCCCCCGTGCCTTCCTTGACCCTGGAAGGTGCCACTCCCACTGTCCTTTCCTA |
| | | ATAAAATGAGGAAATTGCATCGCATTGTCTGAGTAGGTGTCATTCTATTCTGGG |
| | | GGGTGGGGTGGGGCAGGACAGCAAGGGGGAGGATTGGGAAGACAATAGCAG |
| | | GCATGCTGGGGATGCGGTGGGCTCTATGGGAAGATGTCTACTGAGCTGTGCGA |
| | | TCCCTGCTGGGGACTTTCCGCTGGGGACTTTCCGCTGGGGACTTTCCGCCTTCA |
| | | GCTAAGGAAGCTACCAATATTTAGAGGTACATTTTGTTCTAGAACAAAATGTA |
| | | CCGGTACATTTTGTTCTGGTACATTTTGTTCTATTAAGGACAACGTAAGTATAG |
| | | CGCATAGACACGGTCTCGAGGGATAACATGGCCCCTGGATGGGAAGGCCAT |
| | | GTTATCCCT |
| 75. | CAG- | CGTTACATAACTTACGGTAAATGGCCCGCCTGGCTGACCGCCCAACGACCCCC |
| | H2B- | GCCCATTGACGTCAATAATGACGTATGTTCCCATAGTAACGCCAATAGGGACT |
| | tdTomato- | TTCCATTGACGTCAATGGGTGGAGTATTTACGGTAAACTGCCCACTTGGCAGTA |
| | noV5- | CATCAAGTGTATCATATGCCAAGTACGCCCCCTATTGACGTCAATGACGGTAA |
| | P2A-Fluc- | ATGGCCCGCCTGGCATTATGCCCAGTACATGACCTTATGGGACTTTCCTACTTG |
| | WPRE3- | GCAGTACATCTACGTATTAGTCATCGCTATTACCATGGTCGAGGTGAGCCCCAC |
| | DTS | GTTCTGCTTCACTCTCCCCATCTCCCCCCCCTCCCCACCCCCAATTTTGTATTTA |
| | | TTTATTTTTTAATTATTTTGTGCAGCGATGGGGGCGGGGGGGGGGGGGGCGCG |
| | | CGCCAGGCGGGGCGGGGCGGGGCGAGGGGCGGGGCGGGGCGAGGCGGAGAG |
| | | GTGCGGCGGCAGCCAATCAGAGCGGCGCGCTCCGAAAGTTTCCTTTTATGGCG |
| | | AGGCGGCGGCGGCGGCGGCCCTATAAAAAGCGAAGCGCGCGGCGGGCGGGAG |
| | | TCGCTGCGCGCTGCCTTCGCCCCGTGCCCCGCTCCGCCGCCGCCTCGCGCCGCC |
| | | CGCCCCGGCTCTGACTGACCGCGTTACTCCCACAGGTGAGCGGGCGGGACGGC |
| | | CCTTCTCCTCCGGGCTGTAATTAGCGCTTGGTTTAATGACGGCTTGTTTCTTTTC |
| | | TGTGGCTGCGTGAAAGCCTTGAGGGGCTCCGGGAGGGCCCTTTGTGCGGGGGG |
| | | AGCGGCTCGGGGGGTGCGTGCGTGTGTGTGTGCGTGGGGAGCGCCGCGTGCGG |
| | | CTCCGCGCTGCCCGGCGGCTGTGAGCGCTGCGGGCGCGGCGCGGGGCTTTGTG |
| | | CGCTCCGCAGTGTGCGCGAGGGGAGCGCGGCCGGGGGCGGTGCCCCGCGGTG |
| | | CGGGGGGGGCTGCGAGGGGAACAAAGGCTGCGTGCGGGGTGTGTGCGTGGGG |
| | | GGGTGAGCAGGGGGTGTGGGCGCGTCGGTCGGGCTGCAACCCCCCCTGCACCC |
| | | CCCTCCCCGAGTTGCTGAGCACGGCCCGGCTTCGGGTGCGGGGCTCCGTACGG |
| | | GGCGTGGCGCGGGGCTCGCCGTGCCGGGCGGGGGGTGGCGGCAGGTGGGGGT |
| | | GCCGGGCGGGGCGGGGCCGCCTCGGGCCGGGGAGGGCTCGGGGGAGGGGCGC |
| | | GGCGGCCCCCGGAGCGCCGGCGGCTGTCGAGGCGCGGCGAGCCGCAGCCATT |
| | | GCCTTTTATGGTAATCGTGCGAGAGGGCGCAGGGACTTCCTTTGTCCCAAATCT |
| | | GTGCGGAGCCGAAATCTGGGAGGCGCCGCCGCACCCCCTCTAGCGGGCGCGGG |
| | | GCGAAGCGGTGCGGCGCCGGCAGGAAGGAAATGGGCGGGGAGGGCCTTCGTG |
| | | CGTCGCCGCGCCGCCGTCCCCTTCTCCCTCTCCAGCCTCGGGGCTGTCCGCGGG |
| | | GGGACGGCTGCCTTCGGGGGGGACGGGGCAGGGCGGGGTTCGGCTTCTGGCGT |
| | | GTGACCGGCGGCTCTAGAGCCTCTGCTAACCATGTTCATGCCTTCTTCTTTTTCC |
| | | TACAGCTCCTGGGCAACGTGCTGGTTATTGTGCTGTCTCATCATTTTGGCAAAG |
| | | AACGCGCGATCGCCACCATGCCAGAGCCAGCGAAGTCTGCTCCCGCCCCGAAA |
| | | AAGGGCTCCAAGAAGGCGGTGACTAAGGCGCAGAAGAAAGGCGGCAAGAAGC |
| | | GCAAGCGCAGCCGCAAGGAGAGCTATTCCATCTATGTGTACAAGGTTCTGAAG |
| | | CAGGTCCACCCTGACACCGGCATTTCGTCCAAGGCCATGGGCATCATGAATTC |
| | | GTTTGTGAACGACATTTTCGAGCGCATCGCAGGTGAGGCTTCCCGCCTGGCGC |
| | | ATTACAACAAGCGCTCGACCATCACCTCCAGGGAGATCCAGACGGCCGTGCGC |
| | | CTGCTGCTGCCTGGGGAGTTGGCCAAGCACGCCGTGTCCGAGGGTACTAAGGC |
| | | CATCACCAAGTACACCAGCGCTAAGGATCCACCGGTCGCCACCATGGTGAGCA |
| | | AGGGCGAGGAGGTCATCAAAGAGTTCATGCGCTTCAAGGTGCGCATGGAGGG |
| | | CTCCATGAACGGCCACGAGTTCGAGATCGAGGGCGAGGGCGAGGGCCGCCCCT |
| | | ACGAGGGCACCCAGACCGCCAAGCTGAAGGTGACCAAGGGCGGCCCCCTGCC |
| | | CTTCGCCTGGGACATCCTGTCCCCCCAGTTCATGTACGGCTCCAAGGCGTACGT |
| | | GAAGCACCCCGCCGACATCCCCGATTACAAGAAGCTGTCCTTCCCCGAGGGCT |
| | | TCAAGTGGGAGCGCGTGATGAACTTCGAGGACGGCGGTCTGGTGACCGTGACC |
| | | CAGGACTCCTCCCTGCAGGACGGCACGCTGATCTACAAGGTGAAGATGCGCGG |
| | | CACCAACTTCCCCCCCGACGGCCCCGTAATGCAGAAGAAGACCATGGGCTGGG |
| | | AGGCCTCCACCGAGCGCCTGTACCCCCGCGACGGCGTGCTGAAGGGCGAGATC |
| | | CACCAGGCCCTGAAGCTGAAGGACGGCGGCCACTACCTGGTGGAGTTCAAGAC |
| | | CATCTACATGGCCAAGAAGCCCGTGCAACTGCCCGGCTACTACTACGTGGACA |
| | | CCAAGCTGGACATCACCTCCCACAACGAGGACTACACCATCGTGGAACAGTAC |
| | | GAGCGCTCCGAGGGCCGCCACCACCTGTTCCTGGGGCATGGCACCGGCAGCAC |
| | | CGGCAGCGGCAGCTCCGGCACCGCCTCCTCCGAGGACAACAACATGGCCGTCA |
| | | TCAAAGAGTTCATGCGCTTCAAGGTGCGCATGGAGGGCTCCATGAACGGCCAC |
| | | GAGTTCGAGATCGAGGGCGAGGGCGAGGGCCGCCCCTACGAGGGCACCCAGA |
| | | CCGCCAAGCTGAAGGTGACCAAGGGCGGCCCCCTGCCCTTCGCCTGGGACATC |
| | | CTGTCCCCCCAGTTCATGTACGGCTCCAAGGCGTACGTGAAGCACCCCGCCGA |
| | | CATCCCCGATTACAAGAAGCTGTCCTTCCCCGAGGGCTTCAAGTGGGAGCGCG |
| | | TGATGAACTTCGAGGACGGCGGTCTGGTGACCGTGACCCAGGACTCCTCCCTG |
| | | CAGGACGGCACGCTGATCTACAAGGTGAAGATGCGCGGCACCAACTTCCCCCC |
| | | CGACGGCCCCGTAATGCAGAAGAAGACCATGGGCTGGGAGGCCTCCACCGAG |
| | | CGCCTGTACCCCCGCGACGGCGTGCTGAAGGGCGAGATCCACCAGGCCCTGAA |
| | | GCTGAAGGACGGCGGCCACTACCTGGTGGAGTTCAAGACCATCTACATGGCCA |
| | | AGAAGCCCGTGCAACTGCCCGGCTACTACTACGTGGACACCAAGCTGGACATC |
| | | ACCTCCCACAACGAGGACTACACCATCGTGGAACAGTACGAGCGCTCCGAGGG |
| | | CCGCCACCACCTGTTCCTGTACGGCATGGACGAGCTGTACAAGGCTACTAACTT |
| | | CAGCCTGCTGAAGCAGGCTGGAGACGTGGAGGAGAACCCTGGACCTATGGAA |
| | | GACGCCAAAAACATAAAGAAAGGCCCGGCGCCATTCTATCCGCTGGAAGATG |
| | | GAACCGCTGGAGAGCAACTGCATAAGGCTATGAAGAGATACGCCCTGGTTCCT |
| | | GGAACAATTGCTTTTACAGATGCACATATCGAGGTGGACATCACTTACGCTGA |
| | | GTACTTCGAAATGTCCGTTCGGTTGGCAGAAGCTATGAAACGATATGGGCTGA |
| | | ATACAAATCACAGAATCGTCGTATGCAGTGAAAACTCTCTTCAATTCTTTATGC |
| | | CGGTGTTGGGCGCGTTATTTATCGGAGTTGCAGTTGCGCCCGCGAACGACATTT |
| | | ATAATGAACGTGAATTGCTCAACAGTATGGGCATTTCGCAGCCTACCGTGGTG |
| | | TTCGTTTCCAAAAAGGGGTTGCAAAAAATTTTGAACGTGCAAAAAAAGCTCCC |
| | | AATCATCCAAAAAATTATTATCATGGATTCTAAAACGGATTACCAGGGATTTCA |
| | | GTCGATGTACACGTTCGTCACATCTCATCTACCTCCCGGTTTTAATGAATACGA |
| | | TTTTGTGCCAGAGTCCTTCGATAGGGACAAGACAATTGCACTGATCATGAACTC |
| | | CTCTGGATCTACTGGTCTGCCTAAAGGTGTCGCTCTGCCTCATAGAACTGCCTG |
| | | CGTGAGATTCTCGCATGCCAGAGATCCTATTTTTGGCAATCAAATCATTCCGGA |
| | | TACTGCGATTTTAAGTGTTGTTCCATTCCATCACGGTTTTGGAATGTTTACTACA |
| | | CTCGGATATTTGATATGTGGATTTCGAGTCGTCTTAATGTATAGATTTGAAGAA |
| | | GAGCTGTTTCTGAGGAGCCTTCAGGATTACAAGATTCAAAGTGCGCTGCTGGT |
| | | GCCAACCCTATTCTCCTTCTTCGCCAAAAGCACTCTGATTGACAAATACGATTT |
| | | ATCTAATTTACACGAAATTGCTTCTGGTGGCGCTCCCCTCTCTAAGGAAGTCGG |
| | | GGAAGCGGTTGCCAAGAGGTTCCATCTGCCAGGTATCAGGCAAGGATATGGGC |
| | | TCACTGAGACTACATCAGCTATTCTGATTACACCCGAGGGGGATGATAAACCG |
| | | GGCGCGGTCGGTAAAGTTGTTCCATTTTTTGAAGCGAAGGTTGTGGATCTGGAT |
| | | ACCGGGAAAACGCTGGGCGTTAATCAAAGAGGCGAACTGTGTGTGAGAGGTC |
| | | CTATGATTATGTCCGGTTATGTAAACAATCCGGAAGCGACCAACGCCTTGATTG |
| | | ACAAGGATGGATGGCTACATTCTGGAGACATAGCTTACTGGGACGAAGACGAA |
| | | CACTTCTTCATCGTTGACCGCCTGAAGTCTCTGATTAAGTACAAAGGCTATCAG |
| | | GTGGCTCCCGCTGAATTGGAATCCATCTTGCTCCAACACCCCAACATCTTCGAC |
| | | GCAGGTGTCGCAGGTCTTCCCGACGATGACGCCGGTGAACTTCCCGCCGCCGT |
| | | TGTTGTTTTGGAGCACGGAAAGACGATGACGGAAAAAGAGATCGTGGATTACG |
| | | TCGCCAGTCAAGTAACAACCGCGAAAAAGTTGCGCGGAGGAGTTGTGTTTGTG |
| | | GACGAAGTACCGAAAGGTCTTACCGGAAAACTCGACGCAAGAAAAATCAGAG |
| | | AGATCCTCATAAAGGCCAAGAAGGGCGGAAAGATCGCCGTGTAACTCGAGAA |
| | | TCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATT |
| | | GCTTCCCGTATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTGCACTGTGT |
| | | TTGCTGACGCAACCCCCACTGGTTGGGGCATTGCCACCACCTGTCAGCTCCTTT |
| | | CCGGGACTTTCGCTTTCCCCCTCCCTATTGCCACGGCGGAACTCATCGCCGCCT |
| | | GCCTTGCCCGCTGCTGGACAGGGGCTCGGCTGTTGGGCACTGACAATTCCGTG |
| | | GGGTGTGCCTTCTAGTTGCCAGCCATCTGTTGTTTGCCCCTCCCCCGTGCCTTCC |
| | | TTGACCCTGGAAGGTGCCACTCCCACTGTCCTTTCCTAATAAAATGAGGAAATT |
| | | GCATCGCATTGTCTGAGTAGGTGTCATTCTATTCTGGGGGGTGGGGTGGGGCA |
| | | GGACAGCAAGGGGGAGGATTGGGAAGACAATAGCAGGCATGCTGGGGATGCG |
| | | GTGGGCTCTATGGGAAGATGTCTACTGAGCTGTGCGATCCCTGCTGGGGACTTT |
| | | CCGCTGGGGACTTTCCGCTGGGGACTTTCCGCCTTCAGCTAAGGAAGCTACCA |
| | | ATATTTAGAGGTACATTTTGTTCTAGAACAAAATGTACCGGTACATTTTGTTCT |
| | | GGTACATTTTGTTCT |
| 76. | 5′ ITR | AGGGATAACATGGCC |
| 77. | 3′ ITR | GGCCATGTTATCCCT |
| 78. | 5′ ITR- | AGGGATAACATGGCCTGACAGTGGCGGCAGTCACTGAGAAAAACGAAACCG |
| | NUP 358 | GAGCGGCCATGTTATCCCT |
| | Apt-3′ | |
| | ITR | |
| 79. | 5′ ITR- | AGGGATAACATGGCCCCACGCAGATAGACGCTACTCTACTACATCGCAGCCA |
| | NUP Apt- | ACGGCCATGTTATCCCT |
| | 3′ ITR | |
| 80. | Adapter- | AGGGTATGGCACGGCCCACGCAGATAGACGCTACTCTACTACATCGCAGCCAA |
| | ST-1- | CGCCGTGCCATA |
| | NupApt02 | |
| 81. | Adapter- | AGGGCATGGCACGGCCCACGCAGATAGACGCTACTCTACTACATCGCAGCCAA |
| | ST-2- | CGCCGTGCCATG |
| | NupApt02 | |
| 82. | Adapter- | AGGGCATGACACGGCCCACGCAGATAGACGCTACTCTACTACATCGCAGCCAA |
| | ST-3- | CGCCGTGTCATG |
| | NupApt02 | |
| 83. | Adapter- | AGGGCTAACATGCGCCCACGCAGATAGACGCTACTCTACTACATCGCAGCCAA |
| | ST-4- | CGCGCATGTTAG |
| | NupApt02 | |
| 84. | Adapter- | AGGGCCCGAATATGACCACGCAGATAGACGCTACTCTACTACATCGCAGCCAA |
| | ST-5- | CTCATATTCGGG |
| | NupApt02 | |
| 85. | Adapter- | AGGGTCCTGACAGAACCACGCAGATAGACGCTACTCTACTACATCGCAGCCAA |
| | ST-6- | CTTCTGTCAGGA |
| | NupApt02 | |
| 86. | PUC57- | TCGCGCGTTTCGGTGATGACGGTGAAAACCTCTGACACATGCAGCTCCCGGAG |
| | tdTomato- | ACGGTCACAGCTTGTCTGTAAGCGGATGCCGGGAGCAGACAAGCCCGTCAGGG |
| | CAG- | CGCGTCAGCGGGTGTTGGCGGGTGTCGGGGCTGGCTTAACTATGCGGCATCAG |
| | H2B- | AGCAGATTGTACTGAGAGTGCACCATATGCGGTGTGAAATACCGCACAGATGC |
| | tdTomato- | GTAAGGAGAAAATACCGCATCAGGCGCCATTCGCCATTCAGGCTGCGCAACTG |
| | noV5- | TTGGGAAGGGCGATCGGTGCGGGCCTCTTCGCTATTACGCCAGCTGGCGAAAG |
| | P2A-Fluc- | GGGGATGTGCTGCAAGGCGATTAAGTTGGGTAACGCCAGGGTTTTCCCAGTCA |
| | WPRE3- | CGACGTTGTAAAACGACGGCCAGTGAATTCGAGCTCGGTACCTCGCGAATGCA |
| | DTS | TCTAGATGCGGCCGCTAGTTATTAATAGTAATCAATTACGGGGTCATTAGTTCA |
| | | TAGCCCATATATGGAGTTCCGCGTTACATAACTTACGGTAAATGGCCCGCCTGG |
| | | CTGACCGCCCAACGACCCCCGCCCATTGACGTCAATAATGACGTATGTTCCCAT |
| | | AGTAACGCCAATAGGGACTTTCCATTGACGTCAATGGGTGGAGTATTTACGGT |
| | | AAACTGCCCACTTGGCAGTACATCAAGTGTATCATATGCCAAGTACGCCCCCT |
| | | ATTGACGTCAATGACGGTAAATGGCCCGCCTGGCATTATGCCCAGTACATGAC |
| | | CTTATGGGACTTTCCTACTTGGCAGTACATCTACGTATTAGTCATCGCTATTAC |
| | | CATGGTCGAGGTGAGCCCCACGTTCTGCTTCACTCTCCCCATCTCCCCCCCCTC |
| | | CCCACCCCCAATTTTGTATTTATTTATTTTTTAATTATTTTGTGCAGCGATGGGG |
| | | GCGGGGGGGGGGGGGGCGCGCGCCAGGCGGGGCGGGGCGGGGCGAGGGGCG |
| | | GGGCGGGGCGAGGCGGAGAGGTGCGGCGGCAGCCAATCAGAGCGGCGCGCTC |
| | | CGAAAGTTTCCTTTTATGGCGAGGCGGCGGCGGCGGCGGCCCTATAAAAAGCG |
| | | AAGCGCGCGGCGGGCGGGAGTCGCTGCGCGCTGCCTTCGCCCCGTGCCCCGCT |
| | | CCGCCGCCGCCTCGCGCCGCCCGCCCCGGCTCTGACTGACCGCGTTACTCCCAC |
| | | AGGTGAGCGGGCGGGACGGCCCTTCTCCTCCGGGCTGTAATTAGCGCTTGGTTT |
| | | AATGACGGCTTGTTTCTTTTCTGTGGCTGCGTGAAAGCCTTGAGGGGCTCCGGG |
| | | AGGGCCCTTTGTGCGGGGGGAGCGGCTCGGGGGGTGCGTGCGTGTGTGTGTGC |
| | | GTGGGGAGCGCCGCGTGCGGCTCCGCGCTGCCCGGCGGCTGTGAGCGCTGCGG |
| | | GCGCGGCGCGGGGCTTTGTGCGCTCCGCAGTGTGCGCGAGGGGAGCGCGGCCG |
| | | GGGGCGGTGCCCCGCGGTGCGGGGGGGGCTGCGAGGGGAACAAAGGCTGCGT |
| | | GCGGGGTGTGTGCGTGGGGGGGTGAGCAGGGGGTGTGGGCGCGTCGGTCGGG |
| | | CTGCAACCCCCCCTGCACCCCCCTCCCCGAGTTGCTGAGCACGGCCCGGCTTCG |
| | | GGTGCGGGGCTCCGTACGGGGCGTGGCGCGGGGCTCGCCGTGCCGGGCGGGG |
| | | GGTGGCGGCAGGTGGGGGTGCCGGGCGGGGCGGGGCCGCCTCGGGCCGGGGA |
| | | GGGCTCGGGGGAGGGGCGCGGCGGCCCCCGGAGCGCCGGCGGCTGTCGAGGC |
| | | GCGGCGAGCCGCAGCCATTGCCTTTTATGGTAATCGTGCGAGAGGGCGCAGGG |
| | | ACTTCCTTTGTCCCAAATCTGTGCGGAGCCGAAATCTGGGAGGCGCCGCCGCA |
| | | CCCCCTCTAGCGGGCGCGGGGCGAAGCGGTGCGGCGCCGGCAGGAAGGAAAT |
| | | GGGCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGG |
| | | GCGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGTGC |
| | | TGTCTCATCATTTTGGCAAAGAACGCGTCGGCTAGCATGGCGGAAGGATCCGT |
| | | CGCCAGGCAGCCTGACCTCTTGACCTGCGACGATGAGCCGATCCATATCCCCG |
| | | GTGCCATCCAACCGCATGGACTGCTGCTCGCCCTCGCCGCCGACATGACGATC |
| | | GTTGCCGGCAGCGACAACCTTCCCGAACTCACCGGACTGGCGATCGGCGCCCT |
| | | GATCGGCCGCTCTGCGGCCGATGTCTTCGACTCGGAGACGCACAACCGTCTGA |
| | | CGATCGCCTTGGCCGAGCCCGGGGCGGCCGTCGGAGCACCGATCACTGTCGGC |
| | | TTCACGATGCGAAAGGACGCAGGCTTCATCGGCTCCTGGCATCGCCATGATCA |
| | | GCTCATCTTCCTCGAGCTCGAGCCTCCCCAGCGGGACGTCGCCGAGCCGCAGG |
| | | CGTTCTTCCGCCGCACCAACAGCGCCATCCGCCGCCTGCAGGCCGCCGAAACC |
| | | TTGGAAAGCGCCTGCGCCGCCGCGGCGCAAGAGGTGCGGAAGATTACCGGCTT |
| | | CGATCGGGTGATGATCTATCGCTTCGCCTCCGACTTCAGCGGGTCCGTGATCGC |
| | | AGAGGATCGGTGCGCCGAGGTCGAGTCAAAACTAGGCCTGCACTATCCTGCCT |
| | | CATTCATCCCGGCGCAGGCCCGTCGGCTCTATACCATCAACCCGGTACGGATC |
| | | ATTCCCGATATCAATTATCGGCCGGTGCCGGTCACCCCAGACCTCAATCCGGTC |
| | | ACCGGGCGGCCGATTGATCTTAGCTTCGCCATCCTGCGCAGCGTCTCGCCCAAC |
| | | CATCTGGAGTTCATGCGCAACATAGGCATGCACGGCACGATGTCGATCTCGAT |
| | | TTTGCGCGGCGAGCGACTGTGGGGATTGATCGTTTGCCATCACCGAACGCCGT |
| | | ACTACGTCGATCTCGATGGCCGCCAAGCCTGCGAGCTAGTCGCCCAGGTTCTG |
| | | GCCTGGCAGATCGGCGTGATGGAAGAGGGATCCGGAGCTACTAACTTCAGCCT |
| | | GCTGAAGCAGGCTGGAGACGTGGAGGAGAACCCTGGACCTATGGTGAGCAAG |
| | | GGCGAGGAGGTCATCAAAGAGTTCATGCGCTTCAAGGTGCGCATGGAGGGCTC |
| | | CATGAACGGCCACGAGTTCGAGATCGAGGGCGAGGGCGAGGGCCGCCCCTAC |
| | | GAGGGCACCCAGACCGCCAAGCTGAAGGTGACCAAGGGCGGCCCCCTGCCCTT |
| | | CGCCTGGGACATCCTGTCCCCCCAGTTCATGTACGGCTCCAAGGCGTACGTGA |
| | | AGCACCCCGCCGACATCCCCGATTACAAGAAGCTGTCCTTCCCCGAGGGCTTC |
| | | AAGTGGGAGCGCGTGATGAACTTCGAGGACGGCGGTCTGGTGACCGTGACCCA |
| | | GGACTCCTCCCTGCAGGACGGCACGCTGATCTACAAGGTGAAGATGCGCGGCA |
| | | CCAACTTCCCCCCCGACGGCCCCGTAATGCAGAAGAAGACCATGGGCTGGGAG |
| | | GCCTCCACCGAGCGCCTGTACCCCCGCGACGGCGTGCTGAAGGGCGAGATCCA |
| | | CCAGGCCCTGAAGCTGAAGGACGGCGGCCACTACCTGGTGGAGTTCAAGACCA |
| | | TCTACATGGCCAAGAAGCCCGTGCAACTGCCCGGCTACTACTACGTGGACACC |
| | | AAGCTGGACATCACCTCCCACAACGAGGACTACACCATCGTGGAACAGTACGA |
| | | GCGCTCCGAGGGCCGCCACCACCTGTTCCTGGGGCATGGCACCGGCAGCACCG |
| | | GCAGCGGCAGCTCCGGCACCGCCTCCTCCGAGGACAACAACATGGCCGTCATC |
| | | AAAGAGTTCATGCGCTTCAAGGTGCGCATGGAGGGCTCCATGAACGGCCACGA |
| | | GTTCGAGATCGAGGGCGAGGGCGAGGGCCGCCCCTACGAGGGCACCCAGACC |
| | | GCCAAGCTGAAGGTGACCAAGGGCGGCCCCCTGCCCTTCGCCTGGGACATCCT |
| | | GTCCCCCCAGTTCATGTACGGCTCCAAGGCGTACGTGAAGCACCCCGCCGACA |
| | | TCCCCGATTACAAGAAGCTGTCCTTCCCCGAGGGCTTCAAGTGGGAGCGCGTG |
| | | ATGAACTTCGAGGACGGCGGTCTGGTGACCGTGACCCAGGACTCCTCCCTGCA |
| | | GGACGGCACGCTGATCTACAAGGTGAAGATGCGCGGCACCAACTTCCCCCCCG |
| | | ACGGCCCCGTAATGCAGAAGAAGACCATGGGCTGGGAGGCCTCCACCGAGCG |
| | | CCTGTACCCCCGCGACGGCGTGCTGAAGGGCGAGATCCACCAGGCCCTGAAGC |
| | | TGAAGGACGGCGGCCACTACCTGGTGGAGTTCAAGACCATCTACATGGCCAAG |
| | | AAGCCCGTGCAACTGCCCGGCTACTACTACGTGGACACCAAGCTGGACATCAC |
| | | CTCCCACAACGAGGACTACACCATCGTGGAACAGTACGAGCGCTCCGAGGGCC |
| | | GCCACCACCTGTTCCTGTACGGCATGGACGAGCTGTACAAGGAGGGCCCGCGG |
| | | TTCGAAGGTAAGCCTATCCCTAACCCTCTCCTCGGTCTCGATTCTACGCGTACC |
| | | GGTTAGTGAATGCATGAATTCCTGCAGCCACTCGAGGGGATCAGCCTCTACTG |
| | | TGCCTTCTAGTTGCCAGCCATCTGTTGTTTGCCCCTCCCCCTTGCCTTCCTTGAC |
| | | CCTGGAAGGTGCCACTCCCACTGTCCTTTCCTAATAAAATGAGGAAATTGCATC |
| | | ACATTGTCTGAGTAGGTGTCATTCTATTCTGGGGGGTGGGGTGGGGCAGGACA |
| | | GCAAGGGGGAGGATTGGGAAGACAATAGCAGGCATGCTGGGGATGCAGTGGG |
| | | CTCTATGGCGGCCGCATCGGATCCCCGGGCCCGTCGACTGCAGAGGCCTGCAT |
| | | GCAAGCTTGGCGTAATCATGGTCATAGCTGTTTCCTGTGTGAAATTGTTATCCG |
| | | CTCACAATTCCACACAACATACGAGCCGGAAGCATAAAGTGTAAAGCCTGGGG |
| | | TGCCTAATGAGTGAGCTAACTCACATTAATTGCGTTGCGCTCACTGCCCGCTTT |
| | | CCAGTCGGGAAACCTGTCGTGCCAGCTGCATTAATGAATCGGCCAACGCGCGG |
| | | GGAGAGGCGGTTTGCGTATTGGGCGCTCTTCCGCTTCCTCGCTCACTGACTCGC |
| | | TGCGCTCGGTCGTTCGGCTGCGGCGAGCGGTATCAGCTCACTCAAAGGCGGTA |
| | | ATACGGTTATCCACAGAATCAGGGGATAACGCAGGAAAGAACATGTGAGCAA |
| | | AAGGCCAGCAAAAGGCCAGGAACCGTAAAAAGGCCGCGTTGCTGGCGTTTTTC |
| | | CATAGGCTCCGCCCCCCTGACGAGCATCACAAAAATCGACGCTCAAGTCAGAG |
| | | GTGGCGAAACCCGACAGGACTATAAAGATACCAGGCGTTTCCCCCTGGAAGCT |
| | | CCCTCGTGCGCTCTCCTGTTCCGACCCTGCCGCTTACCGGATACCTGTCCGCCTT |
| | | TCTCCCTTCGGGAAGCGTGGCGCTTTCTCATAGCTCACGCTGTAGGTATCTCAG |
| | | TTCGGTGTAGGTCGTTCGCTCCAAGCTGGGCTGTGTGCACGAACCCCCCGTTCA |
| | | GCCCGACCGCTGCGCCTTATCCGGTAACTATCGTCTTGAGTCCAACCCGGTAAG |
| | | ACACGACTTATCGCCACTGGCAGCAGCCACTGGTAACAGGATTAGCAGAGCGA |
| | | GGTATGTAGGCGGTGCTACAGAGTTCTTGAAGTGGTGGCCTAACTACGGCTAC |
| | | ACTAGAAGAACAGTATTTGGTATCTGCGCTCTGCTGAAGCCAGTTACCTTCGGA |
| | | AAAAGAGTTGGTAGCTCTTGATCCGGCAAACAAACCACCGCTGGTAGCGGTGG |
| | | TTTTTTTGTTTGCAAGCAGCAGATTACGCGCAGAAAAAAAGGATCTCAAGAAG |
| | | ATCCTTTGATCTTTTCTACGGGGTCTGACGCTCAGTGGAACGAAAACTCACGTT |
| | | AAGGGATTTTGGTCATGAGATTATCAAAAAGGATCTTCACCTAGATCCTTTTAA |
| | | ATTAAAAATGAAGTTTTAAATCAATCTAAAGTATATATGAGTAAACTTGGTCTG |
| | | ACAGTTACCAATGCTTAATCAGTGAGGCACCTATCTCAGCGATCTGTCTATTTC |
| | | GTTCATCCATAGTTGCCTGACTCCCCGTCGTGTAGATAACTACGATACGGGAGG |
| | | GCTTACCATCTGGCCCCAGTGCTGCAATGATACCGCGAGACCCACGCTCACCG |
| | | GCTCCAGATTTATCAGCAATAAACCAGCCAGCCGGAAGGGCCGAGCGCAGAA |
| | | GTGGTCCTGCAACTTTATCCGCCTCCATCCAGTCTATTAATTGTTGCCGGGAAG |
| | | CTAGAGTAAGTAGTTCGCCAGTTAATAGTTTGCGCAACGTTGTTGCCATTGCTA |
| | | CAGGCATCGTGGTGTCACGCTCGTCGTTTGGTATGGCTTCATTCAGCTCCGGTT |
| | | CCCAACGATCAAGGCGAGTTACATGATCCCCCATGTTGTGCAAAAAAGCGGTT |
| | | AGCTCCTTCGGTCCTCCGATCGTTGTCAGAAGTAAGTTGGCCGCAGTGTTATCA |
| | | CTCATGGTTATGGCAGCACTGCATAATTCTCTTACTGTCATGCCATCCGTAAGA |
| | | TGCTTTTCTGTGACTGGTGAGTACTCAACCAAGTCATTCTGAGAATAGTGTATG |
| | | CGGCGACCGAGTTGCTCTTGCCCGGCGTCAATACGGGATAATACCGCGCCACA |
| | | TAGCAGAACTTTAAAAGTGCTCATCATTGGAAAACGTTCTTCGGGGCGAAAAC |
| | | TCTCAAGGATCTTACCGCTGTTGAGATCCAGTTCGATGTAACCCACTCGTGCAC |
| | | CCAACTGATCTTCAGCATCTTTTACTTTCACCAGCGTTTCTGGGTGAGCAAAAA |
| | | CAGGAAGGCAAAATGCCGCAAAAAAGGGAATAAGGGCGACACGGAAATGTTG |
| | | AATACTCATACTCTTCCTTTTTCAATATTATTGAAGCATTTATCAGGGTTATTGT |
| | | CTCATGAGCGGATACATATTTGAATGTATTTAGAAAAATAAACAAATAGGGGT |
| | | TCCGCGCACATTTCCCCGAAAAGTGCCACCTGACGTCTAAGAAACCATTATTAT |
| | | CATGACATTAACCTATAAAAATAGGCGTATCACGAGGCCCTTTCGTC |
| 87. | oDNA | GTACCTCGCGAATGCATCTAGATCCGACGTTACATAACTTACGGTAAATGGCC |
| | Adaptor | CGCCTGGCTGACCGCCCAACGACCCCCGCCCATTGACGTCAATAATGACGTAT |
| | 19-CAG- | GTTCCCATAGTAACGCCAATAGGGACTTTCCATTGACGTCAATGGGTGGAGTA |
| | H2B- | TTTACGGTAAACTGCCCACTTGGCAGTACATCAAGTGTATCATATGCCAAGTAC |
| | tdTomato- | GCCCCCTATTGACGTCAATGACGGTAAATGGCCCGCCTGGCATTATGCCCAGT |
| | noV5- | ACATGACCTTATGGGACTTTCCTACTTGGCAGTACATCTACGTATTAGTCATCG |
| | P2A-Fluc- | CTATTACCATGGTCGAGGTGAGCCCCACGTTCTGCTTCACTCTCCCCATCTCCC |
| | WPRE3- | CCCCCTCCCCACCCCCAATTTTGTATTTATTTATTTTTTAATTATTTTGTGCAGC |
| | DTS | GATGGGGGCGGGGGGGGGGGGGGCGCGCGCCAGGCGGGGCGGGGCGGGGCG |
| | | AGGGGCGGGGCGGGGCGAGGCGGAGAGGTGCGGCGGCAGCCAATCAGAGCG |
| | | GCGCGCTCCGAAAGTTTCCTTTTATGGCGAGGCGGCGGCGGCGGCGGCCCTAT |
| | | AAAAAGCGAAGCGCGCGGCGGGCGGGAGTCGCTGCGCGCTGCCTTCGCCCCGT |
| | | GCCCCGCTCCGCCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAATTAGC |
| | | GCTTGGTTTAATGACGGCTTGTTTCTTTTCTGTGGCTGCGTGAAAGCCTTGAGG |
| | | GGCTCCGGGAGGGCCCTTTGTGCGGGGGGAGCGGCTCGGGGGGTGCGTGCGTG |
| | | TGTGTGTGCGTGGGGAGCGCCGCGTGCGGCTCCGCGCTGCCCGGCGGCTGTGA |
| | | GCGCTGCGGGCGCGGCGCGGGGCTTTGTGCGCTCCGCAGTGTGCGCGAGGGGA |
| | | GCGCGGCCGGGGGCGGTGCCCCGCGGTGCGGGGGGGGCTGCGAGGGGAACAA |
| | | AGGCTGCGTGCGGGGTGTGTGCGTGGGGGGGTGAGCAGGGGGTGTGGGCGCG |
| | | TCGGTCGGGCTGCAACCCCCCCTGCACCCCCCTCCCCGAGTTGCTGAGCACGGC |
| | | CCGGCTTCGGGTGCGGGGCTCCGTACGGGGCGTGGCGCGGGGCTCGCCGTGCC |
| | | GGGCGGGGGGTGGCGGCAGGTGGGGGTGCCGGGCGGGGCGGGGCCGCCTCGG |
| | | GCCGGGGAGGGCTCGGGGGAGGGGCGCGGCGGCCCCCGGAGCGCCGGCGGCT |
| | | GTCGAGGCGCGGCGAGCCGCAGCCATTGCCTTTTATGGTAATCGTGCGAGAGG |
| | | GCGCAGGGACTTCCTTTGTCCCAAATCTGTGCGGAGCCGAAATCTGGGAGGCG |
| | | CCGCCGCACCCCCTCTAGCGGGCGCGGGGCGAAGCGGTGCGGCGCCGGCAGG |
| | | AAGGAAATGGGCGGGGAGGGCCTTCGTGCGTCGCCGCGCCGCCGTCCCCTTCT |
| | | CCCTCTCCAGCCTCGGGGCTGTCCGCGGGGGGACGGCTGCCTTCGGGGGGGAC |
| | | GGGGCAGGGCGGGGTTCGGCTTCTGGCGTGTGACCGGCGGCTCTAGAGCCTCT |
| | | GCTAACCATGTTCATGCCTTCTTCTTTTTCCTACAGCTCCTGGGCAACGTGCTGG |
| | | TTATTGTGCTGTCTCATCATTTTGGCAAAGAACGCGCGATCGCCACCATGCCAG |
| | | AGCCAGCGAAGTCTGCTCCCGCCCCGAAAAAGGGCTCCAAGAAGGCGGTGACT |
| | | AAGGCGCAGAAGAAAGGCGGCAAGAAGCGCAAGCGCAGCCGCAAGGAGAGC |
| | | TATTCCATCTATGTGTACAAGGTTCTGAAGCAGGTCCACCCTGACACCGGCATT |
| | | TCGTCCAAGGCCATGGGCATCATGAATTCGTTTGTGAACGACATTTTCGAGCGC |
| | | ATCGCAGGTGAGGCTTCCCGCCTGGCGCATTACAACAAGCGCTCGACCATCAC |
| | | CTCCAGGGAGATCCAGACGGCCGTGCGCCTGCTGCTGCCTGGGGAGTTGGCCA |
| | | AGCACGCCGTGTCCGAGGGTACTAAGGCCATCACCAAGTACACCAGCGCTAAG |
| | | GATCCACCGGTCGCCACCATGGTGAGCAAGGGCGAGGAGGTCATCAAAGAGTT |
| | | CATGCGCTTCAAGGTGCGCATGGAGGGCTCCATGAACGGCCACGAGTTCGAGA |
| | | TCGAGGGCGAGGGCGAGGGCCGCCCCTACGAGGGCACCCAGACCGCCAAGCT |
| | | GAAGGTGACCAAGGGCGGCCCCCTGCCCTTCGCCTGGGACATCCTGTCCCCCC |
| | | AGTTCATGTACGGCTCCAAGGCGTACGTGAAGCACCCCGCCGACATCCCCGAT |
| | | TACAAGAAGCTGTCCTTCCCCGAGGGCTTCAAGTGGGAGCGCGTGATGAACTT |
| | | CGAGGACGGCGGTCTGGTGACCGTGACCCAGGACTCCTCCCTGCAGGACGGCA |
| | | CGCTGATCTACAAGGTGAAGATGCGCGGCACCAACTTCCCCCCCGACGGCCCC |
| | | GTAATGCAGAAGAAGACCATGGGCTGGGAGGCCTCCACCGAGCGCCTGTACCC |
| | | CCGCGACGGCGTGCTGAAGGGCGAGATCCACCAGGCCCTGAAGCTGAAGGAC |
| | | GGCGGCCACTACCTGGTGGAGTTCAAGACCATCTACATGGCCAAGAAGCCCGT |
| | | GCAACTGCCCGGCTACTACTACGTGGACACCAAGCTGGACATCACCTCCCACA |
| | | ACGAGGACTACACCATCGTGGAACAGTACGAGCGCTCCGAGGGCCGCCACCAC |
| | | CTGTTCCTGGGGCATGGCACCGGCAGCACCGGCAGCGGCAGCTCCGGCACCGC |
| | | CTCCTCCGAGGACAACAACATGGCCGTCATCAAAGAGTTCATGCGCTTCAAGG |
| | | TGCGCATGGAGGGCTCCATGAACGGCCACGAGTTCGAGATCGAGGGCGAGGG |
| | | CGAGGGCCGCCCCTACGAGGGCACCCAGACCGCCAAGCTGAAGGTGACCAAG |
| | | GGCGGCCCCCTGCCCTTCGCCTGGGACATCCTGTCCCCCCAGTTCATGTACGGC |
| | | TCCAAGGCGTACGTGAAGCACCCCGCCGACATCCCCGATTACAAGAAGCTGTC |
| | | CTTCCCCGAGGGCTTCAAGTGGGAGCGCGTGATGAACTTCGAGGACGGCGGTC |
| | | TGGTGACCGTGACCCAGGACTCCTCCCTGCAGGACGGCACGCTGATCTACAAG |
| | | GTGAAGATGCGCGGCACCAACTTCCCCCCCGACGGCCCCGTAATGCAGAAGAA |
| | | GACCATGGGCTGGGAGGCCTCCACCGAGCGCCTGTACCCCCGCGACGGCGTGC |
| | | TGAAGGGCGAGATCCACCAGGCCCTGAAGCTGAAGGACGGCGGCCACTACCT |
| | | GGTGGAGTTCAAGACCATCTACATGGCCAAGAAGCCCGTGCAACTGCCCGGCT |
| | | ACTACTACGTGGACACCAAGCTGGACATCACCTCCCACAACGAGGACTACACC |
| | | ATCGTGGAACAGTACGAGCGCTCCGAGGGCCGCCACCACCTGTTCCTGTACGG |
| | | CATGGACGAGCTGTACAAGGCTACTAACTTCAGCCTGCTGAAGCAGGCTGGAG |
| | | ACGTGGAGGAGAACCCTGGACCTATGGAAGACGCCAAAAACATAAAGAAAGG |
| | | CCCGGCGCCATTCTATCCGCTGGAAGATGGAACCGCTGGAGAGCAACTGCATA |
| | | AGGCTATGAAGAGATACGCCCTGGTTCCTGGAACAATTGCTTTTACAGATGCA |
| | | CATATCGAGGTGGACATCACTTACGCTGAGTACTTCGAAATGTCCGTTCGGTTG |
| | | GCAGAAGCTATGAAACGATATGGGCTGAATACAAATCACAGAATCGTCGTATG |
| | | CAGTGAAAACTCTCTTCAATTCTTTATGCCGGTGTTGGGCGCGTTATTTATCGG |
| | | AGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCAT |
| | | GGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAG |
| | | GGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAG |
| | | ATCCTATTTTTGGCAATCAAATCATTCCGGATACTGCGATTTTAAGTGTTGTTCC |
| | | ATTCCATCACGGTTTTGGAATGTTTACTACACTCGGATATTTGATATGTGGATTT |
| | | CGAGTCGTCTTAATGTATAGATTTGAAGAAGAGCTGTTTCTGAGGAGCCTTCAG |
| | | GATTACAAGATTCAAAGTGCGCTGCTGGTGCCAACCCTATTCTCCTTCTTCGCC |
| | | AAAAGCACTCTGATTGACAAATACGATTTATCTAATTTACACGAAATTGCTTCT |
| | | GGTGGCGCTCCCCTCTCTAAGGAAGTCGGGGAAGCGGTTGCCAAGAGGTTCCA |
| | | TCTGCCAGGTATCAGGCAAGGATATGGGCTCACTGAGACTACATCAGCTATTC |
| | | TGATTACACCCGAGGGGGATGATAAACCGGGCGCGGTCGGTAAAGTTGTTCCA |
| | | TTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAA |
| | | ACAATCCGGAAGCGACCAACGCCTTGATTGACAAGGATGGATGGCTACATTCT |
| | | GGAGACATAGCTTACTGGGACGAAGACGAACACTTCTTCATCGTTGACCGCCT |
| | | GAAGTCTCTGATTAAGTACAAAGGCTATCAGGTGGCTCCCGCTGAATTGGAAT |
| | | CCATCTTGCTCCAACACCCCAACATCTTCGACGCAGGTGTCGCAGGTCTTCCCG |
| | | ACGATGACGCCGGTGAACTTCCCGCCGCCGTTGTTGTTTTGGAGCACGGAAAG |
| | | ACGATGACGGAAAAAGAGATCGTGGATTACGTCGCCAGTCAAGTAACAACCG |
| | | CGAAAAAGTTGCGCGGAGGAGTTGTGTTTGTGGACGAAGTACCGAAAGGTCTT |
| | | ACCGGAAAACTCGACGCAAGAAAAATCAGAGAGATCCTCATAAAGGCCAAGA |
| | | AGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGG |
| | | TTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCTATTAAGGACA |
| | | ACGTAAGTATAGCGCATAGACACGTGGCGCCATCGGATCCCGGGTGGCTTGTT |
| | | GTCCACAACCATTAAACCTTAAAAGCTTTAAAAGCCTTATATATTCTTTTTTTTC |
| | | TTATAAAACTTAAAACCTTAGAGGCTATTTAAGTTGCTGATTTATATTAATTTT |
| | | ATTGTTCAAACATGAGAGCTTAGTACGTGAAACATGAGAGCTTAGTACATTAG |
| | | CCATGAGAGCTTAGTACATTAGCCATGAGGGTTTAGTTCATTAAACATGAGAG |
| | | CTTAGTACATTAAACATGAGAGCTTAGTACATTAAACATGAGAGCTTAGTACA |
| | | TACTATCAACAGGTTGAACTGCTGATCTGTACAGTAGAATTGGTAAAGAGAGT |
| | | TGTGTAAAATATTGAGTTCGCACATCTTGTTGTCTGATTATTGATTTTTGGCGAA |
| | | ACCATTTGATCATATGACAAGATGTGTATCTACCTTAACTTAATGATTTTGATA |
| | | AAAATCATTAG |
| 88. | Np- | CGAGACCTGCATCTAGATCCGACGTTACATAACTTACGGTAAATGGCCCGCCT |
| | CAG- | GGCTGACCGCCCAACGACCCCCGCCCATTGACGTCAATAATGACGTATGTTCC |
| | H2B- | CATAGTAACGCCAATAGGGACTTTCCATTGACGTCAATGGGTGGAGTATTTAC |
| | tdTomato- | GGTAAACTGCCCACTTGGCAGTACATCAAGTGTATCATATGCCAAGTACGCCC |
| | noV5- | CCTATTGACGTCAATGACGGTAAATGGCCCGCCTGGCATTATGCCCAGTACAT |
| | P2A-Fluc- | GACCTTATGGGACTTTCCTACTTGGCAGTACATCTACGTATTAGTCATCGCTAT |
| | WPRE3- | TACCATGGTCGAGGTGAGCCCCACGTTCTGCTTCACTCTCCCCATCTCCCCCCC |
| | DTS | CTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGG |
| | | GCGGGGCGGGGCGAGGCGGAGAGGTGCGGCGGCAGCCAATCAGAGCGGCGCG |
| | | CTCCGAAAGTTTCCTTTTATGGCGAGGCGGCGGCGGCGGCGGCCCTATAAAAA |
| | | GCGAAGCGCGCGGCGGGCGGGAGTCGCTGCGCGCTGCCTTCGCCCCGTGCCCC |
| | | GCTCCGCCGCCGCCTCGCGCCGCCCGCCCCGGCTCTGACTGACCGCGTTACTCC |
| | | CACAGGTGAGCGGGCGGGACGGCCCTTCTCCTCCGGGCTGTAATTAGCGCTTG |
| | | GTTTAATGACGGCTTGTTTCTTTTCTGTGGCTGCGTGAAAGCCTTGAGGGGCTC |
| | | CGGGAGGGCCCTTTGTGCGGGGGGAGCGGCTCGGGGGGTGCGTGCGTGTGTGT |
| | | GTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAACAAAGGCT |
| | | GCGTGCGGGGTGTGTGCGTGGGGGGGTGAGCAGGGGGTGTGGGCGCGTCGGT |
| | | CGGGCTGCAACCCCCCCTGCACCCCCCTCCCCGAGTTGCTGAGCACGGCCCGG |
| | | CTTCGGGTGCGGGGCTCCGTACGGGGCGTGGCGCGGGGCTCGCCGTGCCGGGC |
| | | GGGGGGTGGCGGCAGGTGGGGGTGCCGGGCGGGGCGGGGCCGCCTCGGGCCG |
| | | GGGAGGGCTCGGGGGAGGGGCGCGGCGGCCCCCGGAGCGCCGGCGGCTGTCG |
| | | AGGCGCGGCGAGCCGCAGCCATTGCCTTTTATGGTAATCGTGCGAGAGGGCGC |
| | | AGGGACTTCCTTTGTCCCAAATCTGTGCGGAGCCGAAATCTGGGAGGCGCCGC |
| | | CGCACCCCCTCTAGCGGGCGCGGGGCGAAGCGGTGCGGCGCCGGCAGGAAGG |
| | | AAATGGGCGGGGAGGGCCTTCGTGCGTCGCCGCGCCGCCGTCCCCTTCTCCCTC |
| | | TCCAGCCTCGGGGCTGTCCGCGGGGGGACGGCTGCCTTCGGGGGGGACGGGGC |
| | | AGGGCGGGGTTCGGCTTCTGGCGTGTGACCGGCGGCTCTAGAGCCTCTGCTAA |
| | | CCATGTTCATGCCTTCTTCTTTTTCCTACAGCTCCTGGGCAACGTGCTGGTTATT |
| | | GTGCTGTCTCATCATTTTGGCAAAGAACGCGCGATCGCCACCATGCCAGAGCC |
| | | AGCGAAGTCTGCTCCCGCCCCGAAAAAGGGCTCCAAGAAGGCGGTGACTAAG |
| | | GCGCAGAAGAAAGGCGGCAAGAAGCGCAAGCGCAGCCGCAAGGAGAGCTATT |
| | | CCATCTATGTGTACAAGGTTCTGAAGCAGGTCCACCCTGACACCGGCATTTCGT |
| | | CCAAGGCCATGGGCATCATGAATTCGTTTGTGAACGACATTTTCGAGCGCATC |
| | | GCAGGTGAGGCTTCCCGCCTGGCGCATTACAACAAGCGCTCGACCATCACCTC |
| | | CAGGGAGATCCAGACGGCCGTGCGCCTGCTGCTGCCTGGGGAGTTGGCCAAGC |
| | | ACGCCGTGTCCGAGGGTACTAAGGCCATCACCAAGTACACCAGCGCTAAGGAT |
| | | CCACCGGTCGCCACCATGGTGAGCAAGGGCGAGGAGGTCATCAAAGAGTTCAT |
| | | GCGCTTCAAGGTGCGCATGGAGGGCTCCATGAACGGCCACGAGTTCGAGATCG |
| | | AGGGCGAGGGCGAGGGCCGCCCCTACGAGGGCACCCAGACCGCCAAGCTGAA |
| | | GGTGACCAAGGGCGGCCCCCTGCCCTTCGCCTGGGACATCCTGTCCCCCCAGTT |
| | | CATGTACGGCTCCAAGGCGTACGTGAAGCACCCCGCCGACATCCCCGATTACA |
| | | AGAAGCTGTCCTTCCCCGAGGGCTTCAAGTGGGAGCGCGTGATGAACTTCGAG |
| | | GACGGCGGTCTGGTGACCGTGACCCAGGACTCCTCCCTGCAGGACGGCACGCT |
| | | GATCTACAAGGTGAAGATGCGCGGCACCAACTTCCCCCCCGACGGCCCCGTAA |
| | | TGCAGAAGAAGACCATGGGCTGGGAGGCCTCCACCGAGCGCCTGTACCCCCGC |
| | | GACGGCGTGCTGAAGGGCGAGATCCACCAGGCCCTGAAGCTGAAGGACGGCG |
| | | GCCACTACCTGGTGGAGTTCAAGACCATCTACATGGCCAAGAAGCCCGTGCAA |
| | | CTGCCCGGCTACTACTACGTGGACACCAAGCTGGACATCACCTCCCACAACGA |
| | | GGACTACACCATCGTGGAACAGTACGAGCGCTCCGAGGGCCGCCACCACCTGT |
| | | TCCTGGGGCATGGCACCGGCAGCACCGGCAGCGGCAGCTCCGGCACCGCCTCC |
| | | TCCGAGGACAACAACATGGCCGTCATCAAAGAGTTCATGCGCTTCAAGGTGCG |
| | | CATGGAGGGCTCCATGAACGGCCACGAGTTCGAGATCGAGGGCGAGGGCGAG |
| | | GGCCGCCCCTACGAGGGCACCCAGACCGCCAAGCTGAAGGTGACCAAGGGCG |
| | | GCCCCCTGCCCTTCGCCTGGGACATCCTGTCCCCCCAGTTCATGTACGGCTCCA |
| | | AGGCGTACGTGAAGCACCCCGCCGACATCCCCGATTACAAGAAGCTGTCCTTC |
| | | CCCGAGGGCTTCAAGTGGGAGCGCGTGATGAACTTCGAGGACGGCGGTCTGGT |
| | | GACCGTGACCCAGGACTCCTCCCTGCAGGACGGCACGCTGATCTACAAGGTGA |
| | | AGATGCGCGGCACCAACTTCCCCCCCGACGGCCCCGTAATGCAGAAGAAGACC |
| | | ATGGGCTGGGAGGCCTCCACCGAGCGCCTGTACCCCCGCGACGGCGTGCTGAA |
| | | GGGCGAGATCCACCAGGCCCTGAAGCTGAAGGACGGCGGCCACTACCTGGTG |
| | | GAGTTCAAGACCATCTACATGGCCAAGAAGCCCGTGCAACTGCCCGGCTACTA |
| | | CTACGTGGACACCAAGCTGGACATCACCTCCCACAACGAGGACTACACCATCG |
| | | TGGAACAGTACGAGCGCTCCGAGGGCCGCCACCACCTGTTCCTGTACGGCATG |
| | | GACGAGCTGTACAAGGCTACTAACTTCAGCCTGCTGAAGCAGGCTGGAGACGT |
| | | GGAGGAGAACCCTGGACCTATGGAAGACGCCAAAAACATAAAGAAAGGCCCG |
| | | GCGCCATTCTATCCGCTGGAAGATGGAACCGCTGGAGAGCAACTGCATAAGGC |
| | | TATGAAGAGATACGCCCTGGTTCCTGGAACAATTGCTTTTACAGATGCACATAT |
| | | CGAGGTGGACATCACTTACGCTGAGTACTTCGAAATGTCCGTTCGGTTGGCAG |
| | | AAGCTATGAAACGATATGGGCTGAATACAAATCACAGAATCGTCGTATGCAGT |
| | | GAAAACTCTCTTCAATTCTTTATGCCGGTGTTGGGCGCGTTATTTATCGGAGTT |
| | | GCAGTTGCGCCCGCGAACGACATTTATAATGAACGTGAATTGCTCAACAGTAT |
| | | GGGCATTTCGCAGCCTACCGTGGTGTTCGTTTCCAAAAAGGGGTTGCAAAAAA |
| | | TTTTGAACGTGCAAAAAAAGCTCCCAATCATCCAAAAAATTATTATCATGGATT |
| | | CTAAAACGGATTACCAGGGATTTCAGTCGATGTACACGTTCGTCACATCTCATC |
| | | TACCTCCCGGTTTTAATGAATACGATTTTGTGCCAGAGTCCTTCGATAGGGACA |
| | | AGACAATTGCACTGATCATGAACTCCTCTGGATCTACTGGTCTGCCTAAAGGTG |
| | | TCGCTCTGCCTCATAGAACTGCCTGCGTGAGATTCTCGCATGCCAGAGATCCTA |
| | | TTTTTGGCAATCAAATCATTCCGGATACTGCGATTTTAAGTGTTGTTCCATTCCA |
| | | TCACGGTTTTGGAATGTTTACTACACTCGGATATTTGATATGTGGATTTCGAGT |
| | | CGTCTTAATGTATAGATTTGAAGAAGAGCTGTTTCTGAGGAGCCTTCAGGATTA |
| | | CAAGATTCAAAGTGCGCTGCTGGTGCCAACCCTATTCTCCTTCTTCGCCAAAAG |
| | | CACTCTGATTGACAAATACGATTTATCTAATTTACACGAAATTGCTTCTGGTGG |
| | | CGCTCCCCTCTCTAAGGAAGTCGGGGAAGCGGTTGCCAAGAGGTTCCATCTGC |
| | | CAGGTATCAGGCAAGGATATGGGCTCACTGAGACTACATCAGCTATTCTGATT |
| | | ACACCCGAGGGGGATGATAAACCGGGCGCGGTCGGTAAAGTTGTTCCATTTTT |
| | | TGAAGCGAAGGTTGTGGATCTGGATACCGGGAAAACGCTGGGCGTTAATCAAA |
| | | GAGGCGAACTGTGTGTGAGAGGTCCTATGATTATGTCCGGTTATGTAAACAAT |
| | | CCGGAAGCGACCAACGCCTTGATTGACAAGGATGGATGGCTACATTCTGGAGA |
| | | CATAGCTTACTGGGACGAAGACGAACACTTCTTCATCGTTGACCGCCTGAAGT |
| | | CTCTGATTAAGTACAAAGGCTATCAGGTGGCTCCCGCTGAATTGGAATCCATCT |
| | | TGCTCCAACACCCCAACATCTTCGACGCAGGTGTCGCAGGTCTTCCCGACGATG |
| | | ACGCCGGTGAACTTCCCGCCGCCGTTGTTGTTTTGGAGCACGGAAAGACGATG |
| | | ACGGAAAAAGAGATCGTGGATTACGTCGCCAGTCAAGTAACAACCGCGAAAA |
| | | AGTTGCGCGGAGGAGTTGTGTTTGTGGACGAAGTACCGAAAGGTCTTACCGGA |
| | | AAACTCGACGCAAGAAAAATCAGAGAGATCCTCATAAAGGCCAAGAAGGGCG |
| | | GAAAGATCGCCGTGTAACTCGAGAATCAACCTCTGGATTACAAAATTTGTGAA |
| | | AGATTGACTGGTATTCTTAACTATGTTGCTCCTTTTACGCTATGTGGATACGCTG |
| | | CTTTAATGCCTTTGTATCATGCTATTGCTTCCCGTATGGCTTTCATTTTCTCCTCC |
| | | TTGTATAAATCCTGGTTGCTGTCTCTTTATGAGGAGTTGTGGCCCGTTGTCAGG |
| | | CAACGTGGCGTGGTGTGCACTGTGTTTGCTGACGCAACCCCCACTGGTTGGGG |
| | | CATTGCCACCACCTGTCAGCTCCTTTCCGGGACTTTCGCTTTCCCCCTCCCTATT |
| | | GCCACGGCGGAACTCATCGCCGCCTGCCTTGCCCGCTGCTGGACAGGGGCTCG |
| | | GCTGTTGGGCACTGACAATTCCGTGGGGTGTGCCTTCTAGTTGCCAGCCATCTG |
| | | TTGTTTGCCCCTCCCCCGTGCCTTCCTTGACCCTGGAAGGTGCCACTCCCACTGT |
| | | CCTTTCCTAATAAAATGAGGAAATTGCATCGCATTGTCTGAGTAGGTGTCATTC |
| | | TATTCTGGGGGGTGGGGTGGGGCAGGACAGCAAGGGGGAGGATTGGGAAGAC |
| | | AATAGCAGGCATGCTGGGGATGCGGTGGGCTCTATGGGAAGATGTCTACTGAG |
| | | CTGTGCGATCCCTGCTGGGGACTTTCCGCTGGGGACTTTCCGCTGGGGACTTTC |
| | | CGCCTTCAGCTAAGGAAGCTACCAATATTTAGAGGTACATTTTGTTCTAGAACA |
| | | AAATGTACCGGTACATTTTGTTCTGGTACATTTTGTTCTATTAAGGACAACGTA |
| | | AGTATAGCGCATAGACACGGTCTCGAGGGATAACATGGCCACTCAGGCCATGT |
| | | TATCCCTCGAGACCGTGTCTATGCGCTATACTTACGTTGTCCTTAATAGAACAA |
| | | AATGTACCAGAACAAAATGTACCGGTACATTTTGTTCTAGAACAAAATGTACC |
| | | TCTAAATATTGGTAGCTTCCTTAGCTGAAGGCGGAAAGTCCCCAGCGGAAAGT |
| | | CCCCAGCGGAAAGTCCCCAGCAGGGATCGCACAGCTCAGTAGACATCTTCCCA |
| | | TAGAGCCCACCGCATCCCCAGCATGCCTGCTATTGTCTTCCCAATCCTCCCCCT |
| | | TGCTGTCCTGCCCCACCCCACCCCCCAGAATAGAATGACACCTACTCAGACAA |
| | | TGCGATGCAATTTCCTCATTTTATTAGGAAAGGACAGTGGGAGTGGCACCTTCC |
| | | AGGGTCAAGGAAGGCACGGGGGAGGGGCAAACAACAGATGGCTGGCAACTAG |
| | | AAGGCACACCCCACGGAATTGTCAGTGCCCAACAGCCGAGCCCCTGTCCAGCA |
| | | GCGGGCAAGGCAGGCGGCGATGAGTTCCGCCGTGGCAATAGGGAGGGGGAAA |
| | | GCGAAAGTCCCGGAAAGGAGCTGACAGGTGGTGGCAATGCCCCAACCAGTGG |
| | | GGGTTGCGTCAGCAAACACAGTGCACACCACGCCACGTTGCCTGACAACGGGC |
| | | CACAACTCCTCATAAAGAGACAGCAACCAGGATTTATACAAGGAGGAGAAAA |
| | | TGAAAGCCATACGGGAAGCAATAGCATGATACAAAGGCATTAAAGCAGCGTA |
| | | TCCACATAGCGTAAAAGGAGCAACATAGTTAAGAATACCAGTCAATCTTTCAC |
| | | AAATTTTGTAATCCAGAGGTTGATTCTCGAGTTACACGGCGATCTTTCCGCCCT |
| | | TCTTGGCCTTTATGAGGATCTCTCTGATTTTTCTTGCGTCGAGTTTTCCGGTAAG |
| | | ACCTTTCGGTACTTCGTCCACAAACACAACTCCTCCGCGCAACTTTTTCGCGGT |
| | | TGTTACTTGACTGGCGACGTAATCCACGATCTCTTTTTCCGTCATCGTCTTTCCG |
| | | TGCTCCAAAACAACAACGGCGGCGGGAAGTTCACCGGCGTCATCGTCGGGAAG |
| | | ACCTGCGACACCTGCGTCGAAGATGTTGGGGTGTTGGAGCAAGATGGATTCCA |
| | | ATTCAGCGGGAGCCACCTGATAGCCTTTGTACTTAATCAGAGACTTCAGGCGG |
| | | TCAACGATGAAGAAGTGTTCGTCTTCGTCCCAGTAAGCTATGTCTCCAGAATGT |
| | | AGCCATCCATCCTTGTCAATCAAGGCGTTGGTCGCTTCCGGATTGTTTACATAA |
| | | CCGGACATAATCATAGGACCTCTCACACACAGTTCGCCTCTTTGATTAACGCCC |
| | | AGCGTTTTCCCGGTATCCAGATCCACAACCTTCGCTTCAAAAAATGGAACAACT |
| | | TTACCGACCGCGCCCGGTTTATCATCCCCCTCGGGTGTAATCAGAATAGCTGAT |
| | | GTAGTCTCAGTGAGCCCATATCCTTGCCTGATACCTGGCAGATGGAACCTCTTG |
| | | GCAACCGCTTCCCCGACTTCCTTAGAGAGGGGAGCGCCACCAGAAGCAATTTC |
| | | GTGTAAATTAGATAAATCGTATTTGTCAATCAGAGTGCTTTTGGCGAAGAAGG |
| | | AGAATAGGGTTGGCACCAGCAGCGCACTTTGAATCTTGTAATCCTGAAGGCTC |
| | | CTCAGAAACAGCTCTTCTTCAAATCTATACATTAAGACGACTCGAAATCCACAT |
| | | ATCAAATATCCGAGTGTAGTAAACATTCCAAAACCGTGATGGAATGGAACAAC |
| | | ACTTAAAATCGCAGTATCCGGAATGATTTGATTGCCAAAAATAGGATCTCTGG |
| | | CATGCGAGAATCTCACGCAGGCAGTTCTATGAGGCAGAGCGACACCTTTAGGC |
| | | AGACCAGTAGATCCAGAGGAGTTCATGATCAGTGCAATTGTCTTGTCCCTATCG |
| | | AAGGACTCTGGCACAAAATCGTATTCATTAAAACCGGGAGGTAGATGAGATGT |
| | | GACGAACGTGTACATCGACTGAAATCCCTGGTAATCCGTTTTAGAATCCATGAT |
| | | AATAATTTTTTGGATGATTGGGAGCTTTTTTTGCACGTTCAAAATTTTTTGCAAC |
| | | CCCTTTTTGGAAACGAACACCACGGTAGGCTGCGAAATGCCCATACTGTTGAG |
| | | CAATTCACGTTCATTATAAATGTCGTTCGCGGGCGCAACTGCAACTCCGATAAA |
| | | TAACGCGCCCAACACCGGCATAAAGAATTGAAGAGAGTTTTCACTGCATACGA |
| | | CGATTCTGTGATTTGTATTCAGCCCATATCGTTTCATAGCTTCTGCCAACCGAA |
| | | CGGACATTTCGAAGTACTCAGCGTAAGTGATGTCCACCTCGATATGTGCATCTG |
| | | TAAAAGCAATTGTTCCAGGAACCAGGGCGTATCTCTTCATAGCCTTATGCAGTT |
| | | GCTCTCCAGCGGTTCCATCTTCCAGCGGATAGAATGGCGCCGGGCCTTTCTTTA |
| | | TGTTTTTGGCGTCTTCCATAGGTCCAGGGTTCTCCTCCACGTCTCCAGCCTGCTT |
| | | CAGCAGGCTGAAGTTAGTAGCCTTGTACAGCTCGTCCATGCCGTACAGGAACA |
| | | GGTGGTGGCGGCCCTCGGAGCGCTCGTACTGTTCCACGATGGTGTAGTCCTCGT |
| | | TGTGGGAGGTGATGTCCAGCTTGGTGTCCACGTAGTAGTAGCCGGGCAGTTGC |
| | | ACGGGCTTCTTGGCCATGTAGATGGTCTTGAACTCCACCAGGTAGTGGCCGCC |
| | | GTCCTTCAGCTTCAGGGCCTGGTGGATCTCGCCCTTCAGCACGCCGTCGCGGGG |
| | | GTACAGGCGCTCGGTGGAGGCCTCCCAGCCCATGGTCTTCTTCTGCATTACGGG |
| | | GCCGTCGGGGGGGAAGTTGGTGCCGCGCATCTTCACCTTGTAGATCAGCGTGC |
| | | CGTCCTGCAGGGAGGAGTCCTGGGTCACGGTCACCAGACCGCCGTCCTCGAAG |
| | | TTCATCACGCGCTCCCACTTGAAGCCCTCGGGGAAGGACAGCTTCTTGTAATCG |
| | | GGGATGTCGGCGGGGTGCTTCACGTACGCCTTGGAGCCGTACATGAACTGGGG |
| | | GGACAGGATGTCCCAGGCGAAGGGCAGGGGGCCGCCCTTGGTCACCTTCAGCT |
| | | TGGCGGTCTGGGTGCCCTCGTAGGGGCGGCCCTCGCCCTCGCCCTCGATCTCGA |
| | | ACTCGTGGCCGTTCATGGAGCCCTCCATGCGCACCTTGAAGCGCATGAACTCTT |
| | | TGATGACGGCCATGTTGTTGTCCTCGGAGGAGGCGGTGCCGGAGCTGCCGCTG |
| | | CCGGTGCTGCCGGTGCCATGCCCCAGGAACAGGTGGTGGCGGCCCTCGGAGCG |
| | | CTCGTACTGTTCCACGATGGTGTAGTCCTCGTTGTGGGAGGTGATGTCCAGCTT |
| | | GGTGTCCACGTAGTAGTAGCCGGGCAGTTGCACGGGCTTCTTGGCCATGTAGA |
| | | TGGTCTTGAACTCCACCAGGTAGTGGCCGCCGTCCTTCAGCTTCAGGGCCTGGT |
| | | GGATCTCGCCCTTCAGCACGCCGTCGCGGGGGTACAGGCGCTCGGTGGAGGCC |
| | | TCCCAGCCCATGGTCTTCTTCTGCATTACGGGGCCGTCGGGGGGGAAGTTGGTG |
| | | CCGCGCATCTTCACCTTGTAGATCAGCGTGCCGTCCTGCAGGGAGGAGTCCTG |
| | | GGTCACGGTCACCAGACCGCCGTCCTCGAAGTTCATCACGCGCTCCCACTTGA |
| | | AGCCCTCGGGGAAGGACAGCTTCTTGTAATCGGGGATGTCGGCGGGGTGCTTC |
| | | ACGTACGCCTTGGAGCCGTACATGAACTGGGGGGACAGGATGTCCCAGGCGAA |
| | | GGGCAGGGGGCCGCCCTTGGTCACCTTCAGCTTGGCGGTCTGGGTGCCCTCGT |
| | | AGGGGCGGCCCTCGCCCTCGCCCTCGATCTCGAACTCGTGGCCGTTCATGGAG |
| | | CCCTCCATGCGCACCTTGAAGCGCATGAACTCTTTGATGACCTCCTCGCCCTTG |
| | | CTCACCATGGTGGCGACCGGTGGATCCTTAGCGCTGGTGTACTTGGTGATGGCC |
| | | TTAGTACCCTCGGACACGGCGTGCTTGGCCAACTCCCCAGGCAGCAGCAGGCG |
| | | CACGGCCGTCTGGATCTCCCTGGAGGTGATGGTCGAGCGCTTGTTGTAATGCGC |
| | | CAGGCGGGAAGCCTCACCTGCGATGCGCTCGAAAATGTCGTTCACAAACGAAT |
| | | TCATGATGCCCATGGCCTTGGACGAAATGCCGGTGTCAGGGTGGACCTGCTTC |
| | | AGAACCTTGTACACATAGATGGAATAGCTCTCCTTGCGGCTGCGCTTGCGCTTC |
| | | TTGCCGCCTTTCTTCTGCGCCTTAGTCACCGCCTTCTTGGAGCCCTTTTTCGGGG |
| | | CGGGAGCAGACTTCGCTGGCTCTGGCATGGTGGCGATCGCGCGTTCTTTGCCA |
| | | AAATGATGAGACAGCACAATAACCAGCACGTTGCCCAGGAGCTGTAGGAAAA |
| | | AGAAGAAGGCATGAACATGGTTAGCAGAGGCTCTAGAGCCGCCGGTCACACG |
| | | CCAGAAGCCGAACCCCGCCCTGCCCCGTCCCCCCCGAAGGCAGCCGTCCCCCC |
| | | GCGGACAGCCCCGAGGCTGGAGAGGGAGAAGGGGACGGCGGCGCGGCGACGC |
| | | ACGAAGGCCCTCCCCGCCCATTTCCTTCCTGCCGGCGCCGCACCGCTTCGCCCC |
| | | GCGCCCGCTAGAGGGGGTGCGGCGGCGCCTCCCAGATTTCGGCTCCGCACAGA |
| | | TTTGGGACAAAGGAAGTCCCTGCGCCCTCTCGCACGATTACCATAAAAGGCAA |
| | | TGGCTGCGGCTCGCCGCGCCTCGACAGCCGCCGGCGCTCCGGGGGCCGCCGCG |
| | | CCCCTCCCCCGAGCCCTCCCCGGCCCGAGGCGGCCCCGCCCCGCCCGGCACCC |
| | | CCACCTGCCGCCACCCCCCGCCCGGCACGGCGAGCCCCGCGCCACGCCCCGTA |
| | | CGGAGCCCCGCACCCGAAGCCGGGCCGTGCTCAGCAACTCGGGGAGGGGGGT |
| | | GCAGGGGGGGTTGCAGCCCGACCGACGCGCCCACACCCCCTGCTCACCCCCCC |
| | | ACGCACACACCCCGCACGCAGCCTTTGTTCCCCTCGCAGCCCCCCCCGCACCGC |
| | | GGGGCACCGCCCCCGGCCGCGCTCCCCTCGCGCACACTGCGGAGCGCACAAAG |
| | | CCCCGCGCCGCGCCCGCAGCGCTCACAGCCGCCGGGCAGCGCGGAGCCGCACG |
| | | CGGCGCTCCCCACGCACACACACACGCACGCACCCCCCGAGCCGCTCCCCCCG |
| | | CACAAAGGGCCCTCCCGGAGCCCCTCAAGGCTTTCACGCAGCCACAGAAAAGA |
| | | AACAAGCCGTCATTAAACCAAGCGCTAATTACAGCCCGGAGGAGAAGGGCCG |
| | | TCCCGCCCGCTCACCTGTGGGAGTAACGCGGTCAGTCAGAGCCGGGGGGGGCG |
| | | CCCGCCCGCCGCGCGCTTCGCTTTTTATAGGGCCGCCGCCGCCGCCGCCTCGCC |
| | | GCGCGAGGCGGCGGCGGAGCGGGGCACGGGGCGAAGGCAGCGCGCAGCGACT |
| | | CCCGCCCGCCGCGCGCTTCGCTTTTTATAGGGCCGCCGCCGCCGCCGCCTCGCC |
| | | ATAAAAGGAAACTTTCGGAGCGCGCCGCTCTGATTGGCTGCCGCCGCACCTCT |
| | | CCGCCTCGCCCCGCCCCGCCCCTCGCCCCGCCCCGCCCCGCCTGGCGCGCGCCC |
| | | CCCCCCCCCCCGCCCCCATCGCTGCACAAAATAATTAAAAAATAAATAAATAC |
| | | AAAATTGGGGGTGGGGAGGGGGGGGAGATGGGGAGAGTGAAGCAGAACGTG |
| | | GGGCTCACCTCGACCATGGTAATAGCGATGACTAATACGTAGATGTACTGCCA |
| | | AGTAGGAAAGTCCCATAAGGTCATGTACTGGGCATAATGCCAGGCGGGCCATT |
| | | TACCGTCATTGACGTCAATAGGGGGCGTACTTGGCATATGATACACTTGATGTA |
| | | CTGCCAAGTGGGCAGTTTACCGTAAATACTCCACCCATTGACGTCAATGGAAA |
| | | GTCCCTATTGGCGTTACTATGGGAACATACGTCATTATTGACGTCAATGGGCGG |
| | | GGGTCGTTGGGCGGTCAGCCAGGCGGGCCATTTACCGTAAGTTATGTAACGTC |
| | | GGATCTAGATGCAGGTCTCGAGGGATAACATGGCCACTCAGGCCATGTTATCC |
| | | CT |
| 89. | Telomeric | TTAGGG |
| | motif | |
| 90. | CpG motif | TCCATGACGTTCCTGACGTT |
| 91. | CpG motif | TCGTCGTTTTGTCGTTTTGTCGTT |
| 92. | CpG motif | TCGTCGTTTT |
| 93. | CpG motif | NNCGNN |
| 94. | Adapter- | AGGGTATGGC+A+C+G+G+CCCACGCAGATAGACGCTACTCTACTACATCGCA |
| | ST-1- | GCCAACGCCGTGCCATA |
| | NupApt02 | |
| 95. | Adapter- | AGGGCATGGC+A+C+G+G+CCCACGCAGATAGACGCTACTCTACTACATCGCA |
| | ST-2- | GCCAACGCCGTGCCATG |
| | NupApt02 | |
| 96. | Adapter- | AGGGCATGAC+A+C+G+G+CCCACGCAGATAGACGCTACTCTACTACATCGCA |
| | ST-3- | GCCAACGCCGTGTCATG |
| | NupApt02 | |
| 97. | Adapter- | AGGGCTAACA+T+G+C+G+CCCACGCAGATAGACGCTACTCTACTACATCGCA |
| | ST-4- | GCCAACGCGCATGTTAG |
| | NupApt02 | |
| 98. | Adapter- | AGGGCCCGAA+T+A+T+G+ACCACGCAGATAGACGCTACTCTACTACATCGCA |
| | ST-5- | GCCAACTCATATTCGGG |
| | NupApt02 | |
| 99. | Adapter- | AGGGTCCTGA+C+A+G+A+ACCACGCAGATAGACGCTACTCTACTACATCGCA |
| | ST-6- | GCCAACTTCTGTCAGGA |
| | NupApt02 | |
| 100. | Adapter- | AGGGACCTAG+A+C+G+A+TCCACGCAGATAGACGCTACTCTACTACATCGCA |
| | ST-7- | GCCAACATCGTCTAGGT |
| | NupApt02 | |
| 101. | Adapter- | AGGGTATGGCACGGCMMMMMGCCGTGCCATA |
| | ST-1 | |
| 102. | Adapter- | AGGGCATGGCACGGCMMMMMGCCGTGCCATG |
| | ST-2 | |
| 103. | Adapter- | AGGGCATGACACGGCMMMMMGCCGTGTCATG |
| | ST-3 | |
| 104. | Adapter- | AGGGCTAACATGCGCMMMMMGCGCATGTTAG |
| | ST-4 | |
| 105. | Adapter- | AGGGCCCGAATATGAMMMMMTCATATTCGGG |
| | ST-5 | |
| 106. | Adapter- | AGGGTCCTGACAGAAMMMMMTTCTGTCAGGA |
| | ST-6 | |
| 107. | Adapter- | AGGGACCTAGACGATMMMMMATCGTCTAGGT |
| | ST-7 | |
| 108. | Adapter- | AGGGTATGGC+A+C+G+G+CMMMMMGCCGTGCCATA |
| | ST-1 | |
| 109. | Adapter- | AGGGCATGGC+A+C+G+G+CMMMMMGCCGTGCCATG |
| | ST-2 | |
| 110. | Adapter- | AGGGCATGAC+A+C+G+G+CMMMMMGCCGTGTCATG |
| | ST-3 | |
| 111. | Adapter- | AGGGCTAACA+T+G+C+G+CMMMMMGCGCATGTTAG |
| | ST-4 | |
| 112. | Adapter- | AGGGCCCGAA+T+A+T+G+AMMMMMTCATATTCGGG |
| | ST-5 | |
| 113. | Adapter- | AGGGTCCTGA+C+A+G+A+AMMMMMTTCTGTCAGGA |
| | ST-6 | |
| 114. | Adapter- | AGGGACCTAG+A+C+G+A+TMMMMMATCGTCTAGGT |
| | ST-7 | |
| 115. | Adapter- | AGGGTATGGCACGGCMMMMMGCCGTGCCATA |
| | ST-1 | |
| 116. | Adapter- | AGGGCATGGCACGGCMMMMMGCCGTGCCATG |
| | ST-2 | |
| 117. | Adapter- | AGGGCATGACACGGCMMMMMGCCGTGTCATG |
| | ST-3 | |
| 118. | Adapter- | AGGGCTAACATGCGCMMMMMGCGCATGTTAG |
| | ST-4 | |
| 119. | Adapter- | AGGGCCCGAATATGAMMMMMTCATATTCGGG |
| | ST-5 | |
| 120. | Adapter- | AGGGTCCTGACAGAAMMMMMTTCTGTCAGGA |
| | ST-6 | |
| 121. | Adapter- | AGGGACCTAGACGATMMMMMATCGTCTAGGT |
| | ST-7 | |
| 122. | Adapter- | AGGGTATGGC+A+C+G+G+CMMMMMGCCGTGCCATA |
| | ST-1 | |
| 123. | Adapter- | AGGGCATGGC+A+C+G+G+CMMMMMGCCGTGCCATG |
| | ST-2 | |
| 124. | Adapter- | AGGGCATGAC+A+C+G+G+CMMMMMGCCGTGTCATG |
| | ST-3 | |
| 125. | Adapter- | AGGGCTAACA+T+G+C+G+CMMMMMGCGCATGTTAG |
| | ST-4 | |
| 126. | Adapter- | AGGGCCCGAA+T+A+T+G+AMMMMMTCATATTCGGG |
| | ST-5 | |
| 127. | Adapter- | AGGGTCCTGA+C+A+G+A+AMMMMMTTCTGTCAGGA |
| | ST-6 | |
| 128. | Adapter- | AGGGACCTAG+A+C+G+A+TMMMMMATCGTCTAGGT |
| | ST-7 | |
| 129. | IPO76 | GGATCCTGAGCTACTGACGGGTTATGTGTCAGTCCCCAGGTAATGCTGAGGCTT |
| | Importin | TTGGTTCATTTGGCCGGTTGTGGCACCACTACTGACCATACAC |
| | Aptamer | |
| 130. | IPO72 | GGATCCTGAGCTACTGACTTTGGGTCGAATTGGTTGGCTCGCCCTCTTCTTGGA |
| | Importin | ATTCAGGAGGGCGATTGAACGGACACCACTACTGACCATACAC |
| | Aptamer | |
| 131. | IPO76- | AGGGATAACATGGCCGGATCCTGAGCTACTGACGGGTTATGTGTCAGTCCCCA |
| | with | GGTAATGCTGAGGCTTTTGGTTCATTTGGCCGGTTGTGGCACCACTACTGACCA |
| | adaptor | TACACGGCCATGTTAT |
| 132. | IPO72- | AGGGATAACATGGCCGGATCCTGAGCTACTGACTTTGGGTCGAATTGGTTGGC |
| | with | TCGCCCTCTTCTTGGAATTCAGGAGGGCGATTGAACGGACACCACTACTGACC |
| | adaptor | ATACACGGCCATGTTAT |
| 133. | IPO76- | AGGGATAACA+T+G+G+C+CGGATCCTGAGCTACTGACGGGTTATGTGTCAGT |
| | with | CCCCAGGTAATGCTGAGGCTTTTGGTTCATTTGGCCGGTTGTGGCACCACTACT |
| | adaptor | GACCATACACGGCCATGTTAT |
| 134. | IPO72- | AGGGATAACA+T+G+G+C+CGGATCCTGAGCTACTGACTTTGGGTCGAATTGG |
| | with | TTGGCTCGCCCTCTTCTTGGAATTCAGGAGGGCGATTGAACGGACACCACTACT |
| | adaptor | GACCATACACGGCCATGTTAT |
| 135. | CT1AS1411 | CCCCCCCCCCTCCCCCCCCCGGTGGTGGTGGTTGTGGTGGTGGTGG |
| 136. | CT2AS1411 | CCCCCCTCCCCCCTCCCCCCGGTGGTGGTGGTTGTGGTGGTGGTGG |
| 137. | CT3AS1411 | CCCCTCCCCTCCCCCTCCCCGGTGGTGGTGGTTGTGGTGGT\;.,GGTGG |
| 138. | CT4AS1411 | CCCTCCCTCCCTCCCTCCCCGGTGGTGGTGGTTGTGGTGGTGGTGGGGCCATGT |
| | | TAT |
| 139. | C10AS1411 | CCCCCCCCCCGGTGGTGGTGGTTGTGGTGGTGGTGG |
| 140. | A20AS1411 | AAAAAAAAAAAAAAAAAAAAGGTGGTGGTGGTTGTGGTGGTGGTGG |
| 141. | T20AS1411 | TTTTTTTTTTTTTTTTTTTTGGTGGTGGTGGTTGTGGTGGTGGTGG |
| 142. | CT1AS1411 | CCCCCCCCCCTCCCCCCCCCGGTGGTGGTGGTTGTGGTGGTGGTGG |
| 143. | CT2AS1411 | CCCCCCTCCCCCCTCCCCCCGGTGGTGGTGGTTGTGGTGGTGGTGG |
| 144. | CT3AS1411 | CCCCTCCCCTCCCCCTCCCCGGTGGTGGTGGTTGTGGTGGTGGTGG |
| 145. | CT4AS1411 | CCCTCCCTCCCTCCCTCCCCGGTGGTGGTGGTTGTGGTGGTGGTGGGGCCATGT |
| | | TAT |
| 146. | C10AS1411 | CCCCCCCCCCGGTGGTGGTGGTTGTGGTGGTGGTGG |
| 147. | A20AS1411 | AAAAAAAAAAAAAAAAAAAAGGTGGTGGTGGTTGTGGTGGTGGTGG |
| 148. | T20AS1411 | TTTTTTTTTTTTTTTTTTTTGGTGGTGGTGGTTGTGGTGGTGGTGG |
| 149. | Adapter- | AGGGTATGGCACGGCCCCCCCCCCCCCCCCCCCCCGGTGGTGGTGGTTGTGGT |
| | ST-1- | GGTGGTGGGCCGTGCCATA |
| | C20AS1411 | |
| 150. | Adapter- | AGGGCATGGCACGGCCCCCCCCCCCCCCCCCCCCCGGTGGTGGTGGTTGTGGT |
| | ST-2- | GGTGGTGGGCCGTGCCATG |
| | C20AS1411 | |
| 151. | Adapter- | AGGGCATGACACGGCCCCCCCCCCCCCCCCCCCCCGGTGGTGGTGGTTGTGGT |
| | ST-3- | GGTGGTGGGCCGTGTCATG |
| | C20AS1411 | |
| 152. | Adapter- | AGGGCTAACATGCGCCCCCCCCCCCCCCCCCCCCCGGTGGTGGTGGTTGTGGTG |
| | ST-4- | GTGGTGGGCGCATGTTAG |
| | C20AS1411 | |
| 153. | Adapter- | AGGGCCCGAATATGACCCCCCCCCCCCCCCCCCCCGGTGGTGGTGGTTGTGGT |
| | ST-5- | GGTGGTGGTCATATTCGGG |
| | C20AS1411 | |
| 154. | Adapter- | AGGGTCCTGACAGAACCCCCCCCCCCCCCCCCCCCGGTGGTGGTGGTTGTGGT |
| | ST-6- | GGTGGTGGTTCTGTCAGGA |
| | C20AS1411 | |
| 155. | Adapter- | AGGGACCTAGACGATCCCCCCCCCCCCCCCCCCCCGGTGGTGGTGGTTGTGGT |
| | ST-7- | GGTGGTGGATCGTCTAGGT |
| | C20AS1411 | |
| 156. | Adapter- | AGGGTATGGCACGGCCCTGGATGGGAACTTACCGCTGCAGCCGTGCCATA |
| | ST-1- | |
| | INH-18 | |
| 157. | Adapter- | AGGGCATGGCACGGCCCTGGATGGGAACTTACCGCTGCAGCCGTGCCATG |
| | ST-2- | |
| | INH-18 | |
| 158. | Adapter- | AGGGCATGACACGGCCCTGGATGGGAACTTACCGCTGCAGCCGTGTCATG |
| | ST-3- | |
| | INH-18 | |
| 159. | Adapter- | AGGGCTAACATGCGCCCTGGATGGGAACTTACCGCTGCAGCGCATGTTAG |
| | ST-4- | |
| | INH-18 | |
| 160. | Adapter- | AGGGCCCGAATATGACCTGGATGGGAACTTACCGCTGCATCATATTCGGG |
| | ST-5- | |
| | INH-18 | |
| 161. | Adapter- | AGGGTCCTGACAGAACCTGGATGGGAACTTACCGCTGCATTCTGTCAGGA |
| | ST-6- | |
| | INH-18 | |
| 162. | Adapter- | AGGGACCTAGACGATCCTGGATGGGAACTTACCGCTGCAATCGTCTAGGT |
| | ST-7- | |
| | INH-18 | |
| 163. | Adapter- | AGGGTATGGC+A+C+G+G+CCCCCCCCCCCCCCCCCCCCCGGTGGTGGTGGTT |
| | ST-1- | GTGGTGGTGGTGGGCCGTGCCATA |
| | C20AS1411 | |
| 164. | Adapter- | AGGGCATGGC+A+C+G+G+CCCCCCCCCCCCCCCCCCCCCGGTGGTGGTGGTT |
| | ST-2- | GTGGTGGTGGTGGGCCGTGCCATG |
| | C20AS1411 | |
| 165. | Adapter- | AGGGCATGAC+A+C+G+G+CCCCCCCCCCCCCCCCCCCCCGGTGGTGGTGGTT |
| | ST-3- | GTGGTGGTGGTGGGCCGTGTCATG |
| | C20AS1411 | |
| 166. | Adapter- | AGGGCTAACA+T+G+C+G+CCCCCCCCCCCCCCCCCCCCCGGTGGTGGTGGTT |
| | ST-4- | GTGGTGGTGGTGGGCGCATGTTAG |
| | C20AS1411 | |
| 167. | Adapter- | AGGGCCCGAA+T+A+T+G+ACCCCCCCCCCCCCCCCCCCCGGTGGTGGTGGTT |
| | ST-5- | GTGGTGGTGGTGGTCATATTCGGG |
| | C20AS1411 | |
| 168. | Adapter- | AGGGTCCTGA+C+A+G+A+ACCCCCCCCCCCCCCCCCCCCGGTGGTGGTGGTT |
| | ST-6- | GTGGTGGTGGTGGTTCTGTCAGGA |
| | C20AS1411 | |
| 169. | Adapter- | AGGGACCTAG+A+C+G+A+TCCCCCCCCCCCCCCCCCCCCGGTGGTGGTGGTT |
| | ST-7- | GTGGTGGTGGTGGATCGTCTAGGT |
| | C20AS1411 | |
| 170. | Adapter- | AGGGTATGGC+A+C+G+G+CCCTGGATGGGAACTTACCGCTGCAGCCGTGCC |
| | ST-1- | ATA |
| | INH-18 | |
| 171. | Adapter- | AGGGCATGGC+A+C+G+G+CCCTGGATGGGAACTTACCGCTGCAGCCGTGCC |
| | ST-2- | ATG |
| | INH-18 | |
| 172. | Adapter- | AGGGCATGAC+A+C+G+G+CCCTGGATGGGAACTTACCGCTGCAGCCGTGTC |
| | ST-3- | ATG |
| | INH-18 | |
| 173. | Adapter- | AGGGCTAACA+T+G+C+G+CCCTGGATGGGAACTTACCGCTGCAGCGCATGT |
| | ST-4- | TAG |
| | INH-18 | |
| 174. | Adapter- | AGGGCCCGAA+T+A+T+G+ACCTGGATGGGAACTTACCGCTGCATCATATTCG |
| | ST-5- | GG |
| | INH-18 | |
| 175. | Adapter- | AGGGTCCTGA+C+A+G+A+ACCTGGATGGGAACTTACCGCTGCATTCTGTCAG |
| | ST-6- | GA |
| | INH-18 | |
| 176. | Adapter- | AGGGACCTAG+A+C+G+A+TCCTGGATGGGAACTTACCGCTGCAATCGTCTAG |
| | ST-7- | GT |
| | INH-18 | |
| 177. | Adapter- | AGGGTATGGCACGGCCCCCCCCCCCCCCCCCCCCCGGTGGTGGTGGTTGTGGT |
| | ST-1- | GGTGGTGGGCCGTGCCATA |
| | C20AS1411 | |
| 178. | Adapter- | AGGGCATGGCACGGCCCCCCCCCCCCCCCCCCCCCGGTGGTGGTGGTTGTGGT |
| | ST-2- | GGTGGTGGGCCGTGCCATG |
| | C20AS1411 | |
| 179 | Adapter- | AGGGCATGACACGGCCCCCCCCCCCCCCCCCCCCCGGTGGTGGTGGTTGTGGT |
| | ST-3- | GGTGGTGGGCCGTGTCATG |
| | C20AS1411 | |
| 180. | Adapter- | AGGGCTAACATGCGCCCCCCCCCCCCCCCCCCCCCGGTGGTGGTGGTTGTGGTG |
| | ST-4- | GTGGTGGGCGCATGTTAG |
| | C20AS1411 | |
| 181. | Adapter- | AGGGCCCGAATATGACCCCCCCCCCCCCCCCCCCCGGTGGTGGTGGTTGTGGT |
| | ST-5- | GGTGGTGGTCATATTCGGG |
| | C20AS1411 | |
| 182. | Adapter- | AGGGTCCTGACAGAACCCCCCCCCCCCCCCCCCCCGGTGGTGGTGGTTGTGGT |
| | ST-6- | GGTGGTGGTTCTGTCAGGA |
| | C20AS1411 | |
| 183. | Adapter- | AGGGACCTAGACGATCCCCCCCCCCCCCCCCCCCCGGTGGTGGTGGTTGTGGT |
| | ST-7- | GGTGGTGGATCGTCTAGGT |
| | C20AS1411 | |
| 184. | Adapter- | AGGGTATGGCACGGCCCTGGATGGGAACTTACCGCTGCAGCCGTGCCATA |
| | ST-1- | |
| | INH-18 | |
| 185. | Adapter- | AGGGCATGGCACGGCCCTGGATGGGAACTTACCGCTGCAGCCGTGCCATG |
| | ST-2- | |
| | INH-18 | |
| 186. | Adapter- | AGGGCATGACACGGCCCTGGATGGGAACTTACCGCTGCAGCCGTGTCATG |
| | ST-3- | |
| | INH-18 | |
| 187. | Adapter- | AGGGCTAACATGCGCCCTGGATGGGAACTTACCGCTGCAGCGCATGTTAG |
| | ST-4- | |
| | INH-18 | |
| 188. | Adapter- | AGGGCCCGAATATGACCTGGATGGGAACTTACCGCTGCATCATATTCGGG |
| | ST-5- | |
| | INH-18 | |
| 189. | Adapter- | AGGGTCCTGACAGAACCTGGATGGGAACTTACCGCTGCATTCTGTCAGGA |
| | ST-6- | |
| | INH-18 | |
| 190. | Adapter- | AGGGACCTAGACGATCCTGGATGGGAACTTACCGCTGCAATCGTCTAGGT |
| | ST-7- | |
| | INH-18 | |
| 191. | Adapter- | AGGGTATGGC+A+C+G+G+CCCCCCCCCCCCCCCCCCCCCGGTGGTGGTGGTT |
| | ST-1- | GTGGTGGTGGTGGGCCGTGCCATA |
| | C20AS1411 | |
| 192. | Adapter- | AGGGCATGGC+A+C+G+G+CCCCCCCCCCCCCCCCCCCCCGGTGGTGGTGGTT |
| | ST-2- | GTGGTGGTGGTGGGCCGTGCCATG |
| | C20AS1411 | |
| 193. | Adapter- | AGGGCATGAC+A+C+G+G+CCCCCCCCCCCCCCCCCCCCCGGTGGTGGTGGTT |
| | ST-3- | GTGGTGGTGGTGGGCCGTGTCATG |
| | C20AS1411 | |
| 194. | Adapter- | AGGGCTAACA+T+G+C+G+CCCCCCCCCCCCCCCCCCCCCGGTGGTGGTGGTT |
| | ST-4- | GTGGTGGTGGTGGGCGCATGTTAG |
| | C20AS1411 | |
| 195. | Adapter- | AGGGCCCGAA+T+A+T+G+ACCCCCCCCCCCCCCCCCCCCGGTGGTGGTGGTT |
| | ST-5- | GTGGTGGTGGTGGTCATATTCGGG |
| | C20AS1411 | |
| 196. | Adapter- | AGGGTCCTGA+C+A+G+A+ACCCCCCCCCCCCCCCCCCCCGGTGGTGGTGGTT |
| | ST-6- | GTGGTGGTGGTGGTTCTGTCAGGA |
| | C20AS1411 | |
| 197. | Adapter- | AGGGACCTAG+A+C+G+A+TCCCCCCCCCCCCCCCCCCCCGGTGGTGGTGGTT |
| | ST-7- | GTGGTGGTGGTGGATCGTCTAGGT |
| | C20AS1411 | |
| 198. | Adapter- | AGGGTATGGC+A+C+G+G+CCCTGGATGGGAACTTACCGCTGCAGCCGTGCC |
| | ST-1- | ATA |
| | INH-18 | |
| 199. | Adapter- | AGGGCATGGC+A+C+G+G+CCCTGGATGGGAACTTACCGCTGCAGCCGTGCC |
| | ST-2- | ATG |
| | INH-18 | |
| 200. | Adapter- | AGGGCATGAC+A+C+G+G+CCCTGGATGGGAACTTACCGCTGCAGCCGTGTC |
| | ST-3- | ATG |
| | INH-18 | |
| 201. | Adapter- | AGGGCTAACA+T+G+C+G+CCCTGGATGGGAACTTACCGCTGCAGCGCATGT |
| | ST-4- | TAG |
| | INH-18 | |
| 202. | Adapter- | AGGGCCCGAA+T+A+T+G+ACCTGGATGGGAACTTACCGCTGCATCATATTCG |
| | ST-5- | GG |
| | INH-18 | |
| 203. | Adapter- | AGGGTCCTGA+C+A+G+A+ACCTGGATGGGAACTTACCGCTGCATTCTGTCAG |
| | ST-6- | GA |
| | INH-18 | |
| 204. | Adapter- | AGGGACCTAG+A+C+G+A+TCCTGGATGGGAACTTACCGCTGCAATCGTCTAG |
| | ST-7- | GT |
| | INH-18 | |
| 205. | Adapter- | AGGGTATGGCACGGC |
| | ST-1′ 5′ | |
| | End | |
| 206. | Adapter- | AGGGCATGGCACGGC |
| | ST-2′ 5′ | |
| | End | |
| 207. | Adapter- | AGGGCATGACACGGC |
| | ST-3′ 5′ | |
| | End | |
| 208. | Adapter- | AGGGCTAACATGCGC |
| | ST-4′ 5′ | |
| | End′ | |
| 209. | Adapter- | AGGGCCCGAATATGA |
| | ST-5′ 5′ | |
| | End | |
| 210. | Adapter- | AGGGTCCTGACAGAA |
| | ST-6′ 5′ | |
| | End | |
| 211. | Adapter- | AGGGACCTAGACGAT |
| | ST-7′ 5′ | |
| | End′ | |
| 212. | Adapter- | GCCGTGCCATA |
| | ST-1′ 3′ | |
| | End′ | |
| 213. | Adapter- | GCCGTGCCATG |
| | ST-2′ 3′ | |
| | End | |
| 214. | Adapter- | GCCGTGTCATG |
| | ST-3′ 3′ | |
| | End | |
| 215. | Adapter- | GCGCATGTTAG |
| | ST-4′ 3′ | |
| | End | |
| 216. | Adapter- | TCATATTCGGG |
| | ST-5′ 3′ | |
| | End′ | |
| 217. | Adapter- | TTCTGTCAGGA |
| | ST-6′ 3′ | |
| | End | |
| 218. | Adapter- | ATCGTCTAGGT |
| | ST-7′ 3′ | |
| | End | |
| 219. | Adapter- | AGGGTATGGCACGGC |
| | ST-1′ 5′ | |
| | End | |
| 220. | Adapter- | AGGGCATGGCACGGC |
| | ST-2′ 5′ | |
| | End | |
| 221. | Adapter- | AGGGCATGACACGGC |
| | ST-3′ 5′ | |
| | End | |
| 222. | Adapter- | AGGGCTAACATGCGC |
| | ST-4′ 5 | |
| | End | |
| 223. | Adapter- | AGGGCCCGAATATGA |
| | ST-5′ 5′ | |
| | End′ | |
| 224. | Adapter- | AGGGTCCTGACAGAA |
| | ST-6′ 5′ | |
| | End | |
| 225. | Adapter- | AGGGACCTAGACGAT |
| | ST-7′ 5′ | |
| | End′ | |
| 226. | Adapter- | GCCGTGCCATA |
| | ST-1′ 3′ | |
| | End | |
| 227. | Adapter- | GCCGTGCCATG |
| | ST-2′ 3′ | |
| | End | |
| 228. | Adapter- | GCCGTGTCATG |
| | ST-3′ 3′ | |
| | End | |
| 229. | Adapter- | GCGCATGTTAG |
| | ST-4′ 3′ | |
| | End | |
| 230. | Adapter- | TCATATTCGGG |
| | ST-5′ 3′ | |
| | End | |
| 231. | Adapter- | TTCTGTCAGGA |
| | ST-6′ 3′ | |
| | End | |
| 232. | Adapter- | ATCGTCTAGGT |
| | ST-7′ 3′ | |
| | End | |
| 233. | Adapter- | AGGGTATGGCACGGC |
| | ST-1′ 5′ | |
| | End′ | |
| 234. | Adapter- | AGGGCATGGCACGGC |
| | ST-2′ 5′ | |
| | End′ | |
| 235. | Adapter- | AGGGCATGACACGGC |
| | ST-3′ 5′ | |
| | End′ | |
| 236. | Adapter- | AGGGCTAACATGCGC |
| | ST-4′ 5′ | |
| | End | |
| 237. | Adapter- | AGGGCCCGAATATGA |
| | ST-5′ 5′ | |
| | End | |
| 238. | Adapter- | AGGGTCCTGACAGAA |
| | ST-6′ 5′ | |
| | End | |
| 239. | Adapter- | AGGGACCTAGACGAT |
| | ST-7′ 5′ | |
| | End′ | |
| 240. | Adapter- | GCCGTGCCATA |
| | ST-1′ 3′ | |
| | End′ | |
| 241. | Adapter- | GCCGTGCCATG |
| | ST-2′ 3′ | |
| | End′ | |
| 242. | Adapter- | GCCGTGTCATG |
| | ST-3′ 3′ | |
| | End′ | |
| 243. | Adapter- | GCGCATGTTAG |
| | ST-4′ 3′ | |
| | End′ | |
| 244. | Adapter- | TCATATTCGGG |
| | ST-5′ 3′ | |
| | End′ | |
| 245. | Adapter- | TTCTGTCAGGA |
| | ST-6′ 3′ | |
| | End′ | |
| 246. | Adapter- | ATCGTCTAGGT |
| | ST-7′ 3′ | |
| | End′ | |
| 247. | Adapter- | AGGGTATGGCACGGC |
| | ST-1′ 5′ | |
| | End | |
| 248. | Adapter- | AGGGCATGGCACGGC |
| | ST-2′ 5′ | |
| | End′ | |
| 249. | Adapter- | AGGGCATGACACGGC |
| | ST-3′ 5′ | |
| | End | |
| 250. | Adapter- | AGGGCTAACATGCGC |
| | ST-4′ 5′ | |
| | End′ | |
| 251. | Adapter- | AGGGCCCGAATATGA |
| | ST-5′ 5′ | |
| | End | |
| 252. | Adapter- | AGGGTCCTGACAGAA |
| | ST-6′ 5′ | |
| | End′ | |
| 253. | NP- | CGCGTCTGCAGGCTCAGAGGCACACAGGAGTTTCTGGGCTCACCCTGCCCCCTT |
| | ApoE- | CCAACCCCTCAGTTCCCATCCTCCAGCAGCTGTTTGTGTGCTGCCTCTGAAGTC |
| | AAT- | CACACTGAACAAACTTCAGCCTACTCATGTCCCTAAAATGGGCAAACATTGCA |
| | hBGi- | AGCAGCAAACAGCAAACACACAGCCCTCCCTGCCTGCTGACCTTGGAGCTGGG |
| | FVIII- | GCAGAGGTCAGAGACCTCTCTGGGCCCATGCCACCTCCAACATCCACTCGACC |
| | DM- | CCTTGGAATTTCGGTGGAGAGGAGCAGAGGTTGTCCTGGCGTGGTTTAGGTAG |
| | WPRE3- | TGTGAGAGGGGTCGACTGGACACAGGACGCTGTGGTTTCTGAGCCAGGGGGCG |
| | DTS | ACTCAGATCCCAGCCAGTGGACTTAGCCCCTGTTTGCTCCTCCGATAACTGGGG |
| | | TGACCTTGGTTAATATTCACCAGCAGCCTCCCCCGTTGCCCCTCTGGATCCACT |
| | | GCTTAAATACGGACGAGGACAGGGCCCTGTCTCCTCAGCTTCAGGCACCACCA |
| | | CTGACCTGGGACAGTGAATCGTAAGTACTAGCAGCTACAATCCAGCTACCATT |
| | | CTGCTTTTATTTTATGGTTGGGATAAGGCTGGATTATTCTGAGTCCAAGCTAGG |
| | | CCCTTTTGCTAATCATGTTCATACCTCTTATCTTCCTCCCACAGCTCCTGGGCAA |
| | | CGTGCTGGTCTGTGTGCTGGCCCATCACTTTGGCAAAGAATTGCGATCGCCACC |
| | | ATGCAGATTGAGCTGAGCACCTGCTTCTTCCTGTGCCTGCTGAGGTTCTGCTTC |
| | | TCTGCCACCAGGAGATACTACCTGGGGGCTGTGGAGCTGAGCTGGGACTACAT |
| | | GCAGTCTGACCTGGGGGAGCTGCCTGTGGATGCCAGGTTCCCCCCCAGAGTGC |
| | | CCAAGAGCTTCCCCTTCAACACCTCTGTGGTGTACAAGAAGACCCTGTTTGTGG |
| | | AGTTCACTGACCACCTGTTCAACATTGCCAAGCCCAGGCCCCCCTGGATGGGC |
| | | CTGCTGGGCCCCACCATCCAGGCTGAGGTGTATGACACTGTGGTGATCACCCT |
| | | GAAGAACATGGCCAGCCACCCTGTGAGCCTGCATGCTGTGGGGGTGAGCTACT |
| | | GGAAGGCCTCTGAGGGGGCTGAGTATGATGACCAGACCAGCCAGAGGGAGAA |
| | | GGAGGATGACAAGGTGTTCCCTGGGGGCAGCCACACCTATGTGTGGCAGGTGC |
| | | TGAAGGAGAATGGCCCCATGGCCTCTGACCCCCTGTGCCTGACCTACAGCTAC |
| | | CTGAGCCATGTGGACCTGGTGAAGGACCTGAACTCTGGCCTGATTGGGGCCCT |
| | | GCTGGTGTGCAGGGAGGGCAGCCTGGCCAAGGAGAAGACCCAGACCCTGCAC |
| | | AAGTTCATCCTGCTGTTTGCTGTGTTTGATGAGGGCAAGAGCTGGCACTCTGAA |
| | | ACCAAGAACAGCCTGATGCAGGACAGGGATGCTGCCTCTGCCAGGGCCTGGCC |
| | | CAAGATGCACACTGTGAATGGCTATGTGAACAGGAGCCTGCCTGGCCTGATTG |
| | | GCTGCCACAGGAAGTCTGTGTACTGGCATGTGATTGGCATGGGCACCACCCCT |
| | | GAGGTGCACAGCATCTTCCTGGAGGGCCACACCTTCCTGGTCAGGAACCACAG |
| | | GCAGGCCAGCCTGGAGATCAGCCCCATCACCTTCCTGACTGCCCAGACCCTGC |
| | | TGATGGACCTGGGCCAGTTCCTGCTGTTCTGCCACATCAGCAGCCACCAGCATG |
| | | ATGGCATGGAGGCCTATGTGAAGGTGGACAGCTGCCCTGAGGAGCCCCAGCTG |
| | | AGGATGAAGAACAATGAGGAGGCTGAGGACTATGATGATGACCTGACTGACT |
| | | CTGAGATGGATGTGGTGAGGTTTGATGATGACAACAGCCCCAGCTTCATCCAG |
| | | ATCAGGTCTGTGGCCAAGAAGCACCCCAAGACCTGGGTGCACTACATTGCTGC |
| | | TGAGGAGGAGGACTGGGACTATGCCCCCCTGGTGCTGGCCCCTGATGACAGGA |
| | | GCTACAAGAGCCAGTACCTGAACAATGGCCCCCAGAGGATTGGCAGGAAGTA |
| | | CAAGAAGGTCAGGTTCATGGCCTACACTGATGAAACCTTCAAGACCAGGGAGG |
| | | CCATCCAGCATGAGTCTGGCATCCTGGGCCCCCTGCTGTATGGGGAGGTGGGG |
| | | GACACCCTGCTGATCATCTTCAAGAACCAGGCCAGCAGGCCCTACAACATCTA |
| | | CCCCCATGGCATCACTGATGTGAGGCCCCTGTACAGCAGGAGGCTGCCCAAGG |
| | | GGGTGAAGCACCTGAAGGACTTCCCCATCCTGCCTGGGGAGATCTTCAAGTAC |
| | | AAGTGGACTGTGACTGTGGAGGATGGCCCCACCAAGTCTGACCCCAGGTGCCT |
| | | GACCAGATACTACAGCAGCTTTGTGAACATGGAGAGGGACCTGGCCTCTGGCC |
| | | TGATTGGCCCCCTGCTGATCTGCTACAAGGAGTCTGTGGACCAGAGGGGCAAC |
| | | CAGATCATGTCTGACAAGAGGAATGTGATCCTGTTCTCTGTGTTTGATGAGAAC |
| | | AGGAGCTGGTACCTGACTGAGAACATCCAGAGGTTCCTGCCCAACCCTGCTGG |
| | | GGTGCAGCTGGAGGACCCTGAGTTCCAGGCCAGCAACATCATGCACAGCATCA |
| | | ATGGCTATGTGTTTGACAGCCTGCAGCTGTCTGTGTGCCTGCATGAGGTGGCCT |
| | | ACTGGTACATCCTGAGCATTGGGGCCCAGACTGACTTCCTGTCTGTGTTCTTCT |
| | | CTGGCTACACCTTCAAGCACAAGATGGTGTATGAGGACACCCTGACCCTGTTC |
| | | CCCTTCTCTGGGGAGACTGTGTTCATGAGCATGGAGAACCCTGGCCTGTGGATT |
| | | CTGGGCTGCCACAACTCTGACTTCAGGAACAGGGGCATGACTGCCCTGCTGAA |
| | | AGTCTCCAGCTGTGACAAGAACACTGGGGACTACTATGAGGACAGCTATGAGG |
| | | ACATCTCTGCCTACCTGCTGAGCAAGAACAATGCCATTGAGCCCAGGAGCTTC |
| | | AGCCAGAATGCCACTAATGTGTCTAACAACAGCAACACCAGCAATGACAGCAA |
| | | TGTGTCTCCCCCAGTGCTGAAGAGGCACCAGAGGGAGATCACCAGGACCACCC |
| | | TGCAGTCTGACCAGGAGGAGATTGACTATGATGACACCATCTCTGTGGAGATG |
| | | AAGAAGGAGGACTTTGACATCTACGACGAGGACGAGAACCAGAGCCCCAGGA |
| | | GCTTCCAGAAGAAGACCAGGCACTACTTCATTGCTGCTGTGGAGAGGCTGTGG |
| | | GACTATGGCATGAGCAGCAGCCCCCATGTGCTGAGGAACAGGGCCCAGTCTGG |
| | | CTCTGTGCCCCAGTTCAAGAAGGTGGTGTTCCAGGAGTTCACTGATGGCAGCTT |
| | | CACCCAGCCCCTGTACAGAGGGGAGCTGAATGAGCACCTGGGCCTGCTGGGCC |
| | | CCTACATCAGGGCTGAGGTGGAGGACAACATCATGGTGACCTTCAGGAACCAG |
| | | GCCAGCAGGCCCTACAGCTTCTACAGCAGCCTGATCAGCTATGAGGAGGACCA |
| | | GAGGCAGGGGGCTGAGCCCAGGAAGAACTTTGTGAAGCCCAATGAAACCAAG |
| | | ACCTACTTCTGGAAGGTGCAGCACCACATGGCCCCCACCAAGGATGAGTTTGA |
| | | CTGCAAGGCCTGGGCCTACTTCTCTGATGTGGACCTGGAGAAGGATGTGCACT |
| | | CTGGCCTGATTGGCCCCCTGCTGGTGTGCCACACCAACACCCTGAACCCTGCCC |
| | | ATGGCAGGCAGGTGACTGTGCAGGAGTTTGCCCTGTTCTTCACCATCTTTGATG |
| | | AAACCAAGAGCTGGTACTTCACTGAGAACATGGAGAGGAACTGCAGGGCCCC |
| | | CTGCAACATCCAGATGGAGGACCCCACCTTCAAGGAGAACTACAGGTTCCATG |
| | | CCATCAATGGCTACATCATGGACACCCTGCCTGGCCTGGTGATGGCCCAGGAC |
| | | CAGAGGATCAGGTGGTACCTGCTGAGCATGGGCAGCAATGAGAACATCCACA |
| | | GCATCCACTTCTCTGGCCATGTGTTCACTGTGAGGAAGAAGGAGGAGTACAAG |
| | | ATGGCCCTGTACAACCTGTACCCTGGGGTGTTTGAGACTGTGGAGATGCTGCCC |
| | | AGCAAGGCTGGCATCTGGAGGGTGGAGTGCCTGATTGGGGAGCACCTGCATGC |
| | | TGGCATGAGCACCCTGTTCCTGGTGTACAGCAACAAGTGCCAGACCCCCCTGG |
| | | GCATGGCCTCTGGCCACATCAGGGACTTCCAGATCACTGCCTCTGGCCAGTATG |
| | | GCCAGTGGGCCCCCAAGCTGGCCAGGCTGCACTACTCTGGCAGCATCAATGCC |
| | | TGGAGCACCAAGGAGCCCTTCAGCTGGATCAAGGTGGACCTGCTGGCCCCCAT |
| | | GATCATCCATGGCATCAAGACCCAGGGGGCCAGGCAGAAGTTCAGCAGCCTGT |
| | | ACATCAGCCAGTTCATCATCATGTACAGCCTGGATGGCAAGAAGTGGCAGACC |
| | | TACAGGGGCAACAGCACTGGCACCCTGATGGTGTTCTTTGGCAATGTGGACAG |
| | | CTCTGGCATCAAGCACAACATCTTCAACCCCCCCATCATTGCCAGATACATCAG |
| | | GCTGCACCCCACCCACTACAGCATCAGGAGCACCCTGAGGATGGAGCTGATGG |
| | | GCTGTGACCTGAACAGCTGCAGCATGCCCCTGGGCATGGAGAGCAAGGCCATC |
| | | TCTGATGCCCAGATCACTGCCAGCAGCTACTTCACCAACATGTTTGCCACCTGG |
| | | AGCCCCAGCAAGGCCAGGCTGCACCTGCAGGGCAGGAGCAATGCCTGGAGGC |
| | | CCCAGGTCAACAACCCCAAGGAGTGGCTGCAGGTGGACTTCCAGAAGACCATG |
| | | AAGGTGACTGGGGTGACCACCCAGGGGGTGAAGAGCCTGCTGACCAGCATGT |
| | | ATGTGAAGGAGTTCCTGATCAGCAGCAGCCAGGATGGCCACCAGTGGACCCTG |
| | | TTCTTCCAGAATGGCAAGGTGAAGGTGTTCCAGGGCAACCAGGACAGCTTCAC |
| | | CCCTGTGGTGAACAGCCTGGACCCCCCCCTGCTGACCAGATACCTGAGGATTC |
| | | ACCCCCAGAGCTGGGTGCACCAGATTGCCCTGAGGATGGAGGTGCTGGGCTGT |
| | | GAGGCCCAGGACCTGTACTGATAACTCGAGAATCAACCTCTGGATTACAAAAT |
| | | TTGTGAAAGATTGACTGGTATTCTTAACTATGTTGCTCCTTTTACGCTATGTGGA |
| | | TACGCTGCTTTAATGCCTTTGTATCATGCTATTGCTTCCCGTATGGCTTTCATTT |
| | | TCTCCTCCTTGTATAAATCCTGGTTGCTGTCTCTTTATGAGGAGTTGTGGCCCGT |
| | | TGTCAGGCAACGTGGCGTGGTGTGCACTGTGTTTGCTGACGCAACCCCCACTG |
| | | GTTGGGGCATTGCCACCACCTGTCAGCTCCTTTCCGGGACTTTCGCTTTCCCCCT |
| | | CCCTATTGCCACGGCGGAACTCATCGCCGCCTGCCTTGCCCGCTGCTGGACAGG |
| | | GGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTG |
| | | TCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTA |
| | | CTGAGCTGTGCGATCCCTGCTGGGGACTTTCCGCTGGGGACTTTCCGCTGGGGA |
| | | CTTTCCGCCTTCAGCTAAGGAAGCTACCAATATTTAGAGGTACATTTTGTTCTA |
| | | GAACAAAATGTACCGGTACATTTTGTTCTGGTACATTTTGTTCT |

Underlined portions represent single stranded or partially single stranded portions
Bold portions represent inverted terminal repeat sequences
/I/ inosine nucleotide
Z = 5-(N-naphthylcarboxyamide)-2′-deoxyuridine
+ Locked nucleic acid appears to the right (e.g., TGGCC are LNAs from SEQ ID NO: 55)
N is an unspecified nucleotide (e.g., any one of A, C, T, G, or U)
MMMMM is a sequence of any number of unspecified nucleotides which are positioned within the sequence at the given point between the preceding 5′ end and the following 3′ end

1-3. (canceled)

4. A method for expressing a nucleic acid in a target cell of a subject, the method comprising administering to the subject the nucleic acid, wherein the nucleic acid comprises a cargo polynucleotide and a nuclear localization element, wherein the nuclear localization element increases expression of the cargo polynucleotide in the target cell by at least 1.25 fold as compared to a nucleic acid lacking the nuclear localization element.

5-6. (canceled)

7. The method of claim 4, further comprising administering ultrasonic acoustic energy to the target cell of the subject.

8. The method of claim 7, further comprising administering a sonoactive agent to the subject.

9. (canceled)

10. The method of claim 4, wherein the nuclear localization element increases expression of the cargo polynucleotide in the target cell by at least 1.35, 1.40, at least 1.45, at least 1.50, at least 1.55, at least 1.60, at least 1.65, at least 1.75, at least 1.80, at least 1.85, at least 1.90, at least 1.95, at least 2.0, at least 2.25, at least 2.5, at least 2.75, at least 3, at least 3.5, at least 4, at least 4.5, or at least 5 fold as compared to a nucleic acid lacking the nuclear localization element.

11. (canceled)

12. The method of claim 4, wherein the nuclear localization element comprises an aptamer.

13. The method of claim 12, wherein the aptamer is a DNA aptamer.

14-23. (canceled)

24. The method of claim 12, wherein the aptamer comprises a sequence configured to bind nucleolin.

25-26. (canceled)

27. The method of claim 12, wherein the aptamer comprises a sequence configured to bind an importin protein.

28. The method of claim 27, wherein the sequence configured to bind the importin protein comprises a nucleic acid sequence of SEQ ID NO: 48, 129, OR 130.

29. The method of claim 12, wherein the nuclear localization element or the aptamer comprises a sequence configured to bind a nucleoporin protein.

30. The method of claim 29, wherein the nucleoporin protein is positioned on a cytoplasmic ring or a cytoplasmic filament of the nuclear pore complex.

31. The method of claim 29, wherein the nucleoporin protein comprises NUP 214, NUP 88, or NUP 358.

32. The method of claim 29, wherein the nucleoporin protein comprises NUP 358.

33. The method of claim 29, wherein the sequence configured to bind the nucleoporin protein comprises a nucleic acid sequence of any one of SEQ ID NO: 49-50.

34-79. (canceled)

80. The method of claim 12, wherein the nucleic acid is a closed linear DNA construct comprising a stem region comprising a double stranded DNA sequence covalently closed at both ends by hairpin loops.

81. The method of claim 80, wherein the hairpin loops are single-stranded.

82. The method of claim 80, wherein the hairpin loops form a stem region of the aptamer.

83. The method of claim 80, wherein the hairpin loops comprise the aptamer.

84-120. (canceled)

121. A pharmaceutical composition comprising:
a. a microbubble; and
b. a nucleic acid comprising (1) a cargo polynucleotide comprising an expression cassette that comprises a therapeutic transgene and (2) an aptamer, wherein the aptamer comprises a sequence configured to increase nuclear localization.

122-248. (canceled)

249. An isolated nucleic acid comprising: a cargo polynucleotide comprising an expression cassette that encodes a therapeutic transgene configured for expression in a target cell of a subject, and a nuclear localization element configured to increase expression of the cargo polynucleotide in the target cell.

250-297. (canceled)


SEQUENCE LISTING (253 sequences)

SEQ ID NO: 1 - DNA, 27 bp, synthetic construct
  source 1..27: mol_type = other DNA; note = Nucleolin SELEX Seq.
ggtggtggtggttgtggtggtggtggt

SEQ ID NO: 2 - DNA, 29 bp, synthetic construct
  source 1..29: mol_type = other DNA; note = Hairpin DNA adaptor
  modified_base 15^16: mod_base = OTHER; note = inosine nucleotide
  modified_base 18^19: mod_base = OTHER; note = inosine nucleotide
agggataacatggccctcggccatgttat

SEQ ID NO: 3 - DNA, 14 bp, synthetic construct
  source 1..14: mol_type = other DNA; note = Anti-Nucleolin Aptamer
gttgtttggggtgg

SEQ ID NO: 4 - DNA, 15 bp, synthetic construct
  source 1..15: mol_type = other DNA; note = Anti-Nucleolin Aptamer
gttgtttggggtggt

SEQ ID NO: 5 - DNA, 25 bp, synthetic construct
  source 1..25: mol_type = other DNA; note = Anti-Nucleolin Aptamer
ggttggggtgggtggggtgggtggg

SEQ ID NO: 6 - DNA, 28 bp, synthetic construct
  source 1..28: mol_type = other DNA; note = Anti-Nucleolin Aptamer
tttggtggtggtggttgtggtggtggtg

SEQ ID NO: 7 - DNA, 29 bp, synthetic construct
  source 1..29: mol_type = other DNA; note = Anti-Nucleolin Aptamer
tttggtggtggtggtggtggtggtggtgg

SEQ ID NO: 8 - DNA, 29 bp, synthetic construct
  source 1..29: mol_type = other DNA; note = Anti-Nucleolin Aptamer
tttggtggtggtggtttgggtggtggtgg

SEQ ID NO: 9 - DNA, 13 bp, synthetic construct
  source 1..13: mol_type = other DNA; note = Anti-Nucleolin Aptamer
tggtggtggtggt

SEQ ID NO: 10 - DNA, 14 bp, synthetic construct
  source 1..14: mol_type = other DNA; note = Anti-Nucleolin Aptamer
ggtggttgtggtgg

SEQ ID NO: 11 - DNA, 56 bp, synthetic construct
  source 1..56: mol_type = other DNA; note = Anti-Nucleolin Aptamer
ggtggtggtggttgtggtggtggtggttgtggtggtggtggttgtggtggtggtgg

SEQ ID NO: 12 - DNA, 32 bp, synthetic construct
  source 1..32: mol_type = other DNA; note = Anti-Nucleolin Aptamer
ggtggttgtggtggttgtggtggttgtggtgg

SEQ ID NO: 13 - DNA, 32 bp, synthetic construct
  source 1..32: mol_type = other DNA; note = Anti-Nucleolin Aptamer
tttggtggtggtggttgtggtggtggtggttt

SEQ ID NO: 14 - DNA, 29 bp, synthetic construct
  source 1..29: mol_type = other DNA; note = Anti-Nucleolin Aptamer
ggtggtggtggttgtggtggtggtggttt

SEQ ID NO: 15 - DNA, 10 bp, synthetic construct
  source 1..10: mol_type = other DNA; note = Anti-Nucleolin Aptamer
tggtggtggt

SEQ ID NO: 16 - RNA, 39 nt, synthetic construct
  source 1..39: mol_type = other RNA; note = Anti-Nucleolin Aptamer
ccatctagatctccgtagattcccccggctctttctcgc

SEQ ID NO: 17 - RNA, 41 nt, synthetic construct
  source 1..41: mol_type = other RNA; note = Anti-Nucleolin Aptamer
agccagctttgcataccacgtgcaattcactccacccgtca

SEQ ID NO: 18 - RNA, 40 nt, synthetic construct
  source 1..40: mol_type = other RNA; note = Anti-Nucleolin Aptamer
aagatctgctaagtgcacgcacaatcaccatcgagcgtct

SEQ ID NO: 19 - RNA, 38 nt, synthetic construct
  source 1..38: mol_type = other RNA; note = Anti-Nucleolin Aptamer
cacatggtacgcccaaagcgaggcccgctgcgtagtgc

SEQ ID NO: 20 - RNA, 40 nt, synthetic construct
  source 1..40: mol_type = other RNA; note = Anti-Nucleolin Aptamer
cacggtccagcgctaactgtacctgctgtgccacccaccg

SEQ ID NO: 21 - RNA, 40 nt, synthetic construct
  source 1..40: mol_type = other RNA; note = Anti-Nucleolin Aptamer
accacgcgccaacgtgtcagctacacgccgtgttccccgg

SEQ ID NO: 22 - RNA, 39 nt, synthetic construct
  source 1..39: mol_type = other RNA; note = Anti-Nucleolin Aptamer
aagatcctcgcgcatctgccgagcaatcaccatcggacg

SEQ ID NO: 23 - RNA, 40 nt, synthetic construct
  source 1..40: mol_type = other RNA; note = Anti-Nucleolin Aptamer
ccaaatgccaagccgtagcccggccagtagcccacacgtc

SEQ ID NO: 24 - RNA, 40 nt, synthetic construct
  source 1..40: mol_type = other RNA; note = Anti-Nucleolin Aptamer
tgccaagccgaggcccggccaccatccactgatagtgggc

SEQ ID NO: 25 - RNA, 39 nt, synthetic construct; identical to SEQ ID NO: 16
  source 1..39: mol_type = other RNA; note = Anti-Nucleolin Aptamer

SEQ ID NO: 26 - RNA, 41 nt, synthetic construct; identical to SEQ ID NO: 17
  source 1..41: mol_type = other RNA; note = Anti-Nucleolin Aptamer

SEQ ID NO: 27 - RNA, 40 nt, synthetic construct; identical to SEQ ID NO: 18
  source 1..40: mol_type = other RNA; note = Anti-Nucleolin Aptamer

SEQ ID NO: 28 - RNA, 38 nt, synthetic construct; identical to SEQ ID NO: 19
  source 1..38: mol_type = other RNA; note = Anti-Nucleolin Aptamer

SEQ ID NO: 29 - RNA, 40 nt, synthetic construct; identical to SEQ ID NO: 20
  source 1..40: mol_type = other RNA; note = Anti-Nucleolin Aptamer

SEQ ID NO: 30 - RNA, 40 nt, synthetic construct; identical to SEQ ID NO: 21
  source 1..40: mol_type = other RNA; note = Anti-Nucleolin Aptamer

SEQ ID NO: 31 - RNA, 39 nt, synthetic construct; identical to SEQ ID NO: 22
  source 1..39: mol_type = other RNA; note = Anti-Nucleolin Aptamer

SEQ ID NO: 32 - RNA, 40 nt, synthetic construct; identical to SEQ ID NO: 23
  source 1..40: mol_type = other RNA; note = Anti-Nucleolin Aptamer

SEQ ID NO: 33 - RNA, 40 nt, synthetic construct; identical to SEQ ID NO: 24
  source 1..40: mol_type = other RNA; note = Anti-Nucleolin Aptamer

SEQ ID NO: 34 - RNA, 56 nt, synthetic construct
  source 1..56: mol_type = other RNA; note = Anti-Nucleolin Aptamer
ggaagagggatgggtgccagctttgcataccacgtgcaattcactccacccgtcac

SEQ ID NO: 35 - RNA, 62 nt, synthetic construct
  source 1..62: mol_type = other RNA; note = Anti-Nucleolin Aptamer
gggagagaggaagagggatgggcacggtccagcgctaactgtacctgctgtgccacccaccg

SEQ ID NO: 36 - RNA, 53 nt, synthetic construct
  source 1..53: mol_type = other RNA; note = Anti-Nucleolin Aptamer
ggaagagggatgggcacggtccagcgctaactgtacctgctgtgccacccacc

SEQ ID NO: 37 - RNA, 62 nt, synthetic construct
  source 1..62: mol_type = other RNA; note = Anti-Nucleolin Aptamer
gggagagaggaagagggatgggaccacgcgccaacgtgtcagctacacgccgtgttccccgg

SEQ ID NO: 38 - RNA, 54 nt, synthetic construct
  source 1..54: mol_type = other RNA; note = Anti-Nucleolin Aptamer
gggaggaagagggatgggcacggtccagcgctaactgtacctgctgtgccaccc

SEQ ID NO: 39 - RNA, 52 nt, synthetic construct
  source 1..52: mol_type = other RNA; note = Anti-Nucleolin Aptamer
gggaggaagaggatgggcacggtccagcgctaactgtacctgctgtgccacc

SEQ ID NO: 40 - RNA, 52 nt, synthetic construct
  source 1..52: mol_type = other RNA; note = Anti-Nucleolin Aptamer
gggaggaagagggatgggcacggtccagcgcactgtacctgctgtgccaccc

SEQ ID NO: 41 - RNA, 58 nt, synthetic construct
  source 1..58: mol_type = other RNA; note = Anti-Nucleolin Aptamer
gggagagaggaagagggatgggcacggtccagcgctaactgtacctgctgtgccaccc

SEQ ID NO: 42 - RNA, 60 nt, synthetic construct
  source 1..60: mol_type = other RNA; note = Anti-Nucleolin Aptamer
gggagagaggaagagggatgggcacggtccagcgcactgtacctgctgtgccacccaccg

SEQ ID NO: 43 - DNA, 26 bp, synthetic construct
  source 1..26: mol_type = other DNA; note = Anti-Nucleolin Aptamer AS1411
ggtggtggtggttgtggtggtggtgg

SEQ ID NO: 44 - DNA, 28 bp, synthetic construct
  source 1..28: mol_type = other DNA; note = Anti-Nucleolin Aptamer AT11
tggtggtggttgttgtggtggtggtggt

SEQ ID NO: 45 - DNA, 26 bp, synthetic construct
  source 1..26: mol_type = other DNA; note = Anti-Nucleolin Aptamer AT-11B0
tggtggtggttggtggtggtggtggt

SEQ ID NO: 46 - DNA, 46 bp, synthetic construct
  source 1..46: mol_type = other DNA; note = Anti-Nucleolin Aptamer C20AS1211
ccccccccccccccccccccggtggtggtggttgtggtggtggtgg

SEQ ID NO: 47 - DNA, 24 bp, synthetic construct
  source 1..24: mol_type = other DNA; note = Anti-Nucleolin Aptamer AS1411Z
  modified_base 11..14: mod_base = OTHER; note = 5-(N-naphthylcarboxyamide)-2'-deoxyuridine
ggtggtggtgggtggtggtggtgg

SEQ ID NO: 48 - DNA, 42 bp, synthetic construct
  source 1..42: mol_type = other DNA; note = Anti-Importin Aptamer
tggggactttccgctggggactttccgctggggactttccgc

SEQ ID NO: 49 - DNA, 40 bp, synthetic construct
  source 1..40: mol_type = other DNA; note = Anti-Nucleoporin Aptamer (NUP 358)
tgacagtggcggcagtcactgagaaaaacgaaaccggagc

SEQ ID NO: 50 - DNA, 39 bp, synthetic construct
  source 1..39: mol_type = other DNA; note = Anti-Nucleoporin Aptamer
ccacgcagatagacgctactctactacatcgcagccaac

SEQ ID NO: 51 - DNA, 24 bp, synthetic construct
  source 1..24: mol_type = other DNA; note = Innate Immune Suppressor Aptamer A151
ttagggttagggttagggttaggg

SEQ ID NO: 52 - DNA, 12 bp, synthetic construct
  source 1..12: mol_type = other DNA; note = Innate Immune Suppressor Aptamer 4084-F
cctggatgggaa

SEQ ID NO: 53 - DNA, 24 bp, synthetic construct
  source 1..24: mol_type = other DNA; note = Innate Immune Suppressor Aptamer INH-18
cctggatgggaacttaccgctgca

SEQ ID NO: 54 - DNA, 60 bp, synthetic construct
  source 1..60: mol_type = other DNA; note = Innate Immune Suppressor Aptamer ALL
cctggatgggaattagggttagggttagggttagggcctggatgggaacttaccgctgca

SEQ ID NO: 55 - DNA, 31 bp, synthetic construct
  source 1..31: mol_type = other DNA; note = Hairpin DNA Adaptor
  modified_base 10..15: mod_base = OTHER; note = Locked nucleic acid
agggataacatggccactcaggccatgttat

SEQ ID NO: 56 - DNA, 17 bp, synthetic construct
  source 1..17: mol_type = other DNA; note = Hairpin DNA Adaptor
agggatccactcaggat

SEQ ID NO: 57 - DNA, 17 bp, synthetic construct
  source 1..17: mol_type = other DNA; note = Hairpin DNA Adaptor
  modified_base 8..13: mod_base = OTHER; note = Locked nucleic acid
agggatccactcaggat

SEQ ID NO: 58 - DNA, 21 bp, synthetic construct
  source 1..21: mol_type = other DNA; note = Hairpin DNA Adaptor
agggctaaccactcaggttag

SEQ ID NO: 59 - DNA, 19 bp, synthetic construct
  source 1..19: mol_type = other DNA; note = Hairpin DNA Adaptor
  modified_base 10^11: mod_base = OTHER; note = X can be A, C, T, G, or U
  modified_base 13^14: mod_base = OTHER; note = X can be A, C, T, G, or U
agggctaaccctcggttag

SEQ ID NO: 60 - DNA, 19 bp, synthetic construct
  source 1..19: mol_type = other DNA; note = Hairpin DNA Adaptor
  modified_base 11^12: mod_base = OTHER; note = i5F-dU
  modified_base 12^13: mod_base = OTHER; note = i5F-dU
agggctaaccataggttag

SEQ ID NO: 61 - DNA, 31 bp, synthetic construct; identical to SEQ ID NO: 55
  source 1..31: mol_type = other DNA; note = Hairpin DNA Adaptor

SEQ ID NO: 62 - DNA, 29 bp, synthetic construct
  source 1..29: mol_type = other DNA; note = Hairpin DNA Adaptor
  modified_base 15^16: mod_base = OTHER; note = i8-oxo-dA
  modified_base 18^19: mod_base = OTHER; note = i8-oxo-dA
agggataacatggccctcggccatgttat

SEQ ID NO: 63 - DNA, 18 bp, synthetic construct
  source 1..18: mol_type = other DNA; note = Hairpin DNA Adaptor
  modified_base 10..14: mod_base = OTHER; note = Locked nucleic acid
agggcttacgcgcgtaag

SEQ ID NO: 64 - DNA, 6107 bp, synthetic construct
  source 1..6107: mol_type = other DNA; note = oGOI-CAG-H2B-tdTomato-noV5-P2A-Fluc-WPRE3-DTS-Adapt er-19
agggataacatggccactcaggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccactcaggccatgttatccct

SEQ ID NO: 65 - DNA, 6217 bp, synthetic construct
  source 1..6217: mol_type = other DNA; note = oGOI-CAG-H2B-tdTomato-noV5-P2A-Fluc-WPRE3-DTS-ALL
agggataacatggcccctggatgggaattagggttagggttagggttagggcctggatgggaacttaccgctgcaggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcccctggatgggaattagggttagggttagggttagggcctggatgggaacttaccgctgcaggccatgttatccct

SEQ ID NO: 66 - DNA, 6149 bp, synthetic construct
  source 1..6149: mol_type = other DNA; note = oGOI-CAG-H2B-tdTomato-noV5-P2A-Fluc-WPRE3-DTS-AS141 1
agggataacatggccggtggtggtggttgtggtggtggtggggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccggtggtggtggttgtggtggtggtggggccatgttatccct

SEQ ID NO: 67 - DNA, 6153 bp, synthetic construct
  source 1..6153: mol_type = other DNA; note = oGOI-CAG-H2B-tdTomato-noV5-P2A-Fluc-WPRE3-DTS-AT11
agggataacatggcctggtggtggttgttgtggtggtggtggtggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcctggtggtggttgttgtggtggtggtggtggccatgttatccct

SEQ ID NO: 68 - DNA, 6149 bp, synthetic construct
  source 1..6149: mol_type = other DNA; note = oGOI-CAG-H2B-tdTomato-noV5-P2A-Fluc-WPRE3-DTS-AT-11 B0
agggataacatggcctggtggtggttggtggtggtggtggtggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcctggtggtggttggtggtggtggtggtggccatgttatccct

SEQ ID NO: 69 - DNA, 6189 bp, synthetic construct
  source 1..6189: mol_type = other DNA; note = oGOI-CAG-H2B-tdTomato-noV5-P2A-Fluc-WPRE3-DTS-C20AS 1211
agggataacatggccccccccccccccccccccccggtggtggtggttgtggtggtggtggggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccccccccccccccccccccccggtggtggtggttgtggtggtggtggggccatgttatccct

SEQ ID NO: 70 - DNA, 6145 bp, synthetic construct
  source 1..6145: mol_type = other DNA; note = oGOI-CAG-H2B-tdTomato-noV5-P2A-Fluc-WPRE3-DTS-NH-18
agggataacatggcccctggatgggaacttaccgctgcaggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcccctggatgggaacttaccgctgcaggccatgttatccct

SEQ ID NO: 71 - DNA, 6177 bp, synthetic construct
  source 1..6177: mol_type = other DNA; note = oGOI-CAG-H2B-tdTomato-noV5-P2A-Fluc-WPRE3-DTS-NupAp t01
agggataacatggcctgacagtggcggcagtcactgagaaaaacgaaaccggagcggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcctgacagtggcggcagtcactgagaaaaacgaaaccggagcggccatgttatccct

SEQ ID NO: 72 - DNA, 6175 bp, synthetic construct
  source 1..6175: mol_type = other DNA; note = oGOI-CAG-H2B-tdTomato-noV5-P2A-Fluc-WPRE3-DTS-NupAp t02
agggataacatggccccacgcagatagacgctactctactacatcgcagccaacggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccccacgcagatagacgctactctactacatcgcagccaacggccatgttatccct

SEQ ID NO: 73 - DNA, 6145 bp, synthetic construct
  source 1..6145: mol_type = other DNA; note = oGOI-CAG-H2B-tdTomato-noV5-P2A-Fluc-WPRE3-DTS-A151
agggataacatggccttagggttagggttagggttagggggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccttagggttagggttagggttagggggccatgttatccct

SEQ ID NO: 74 - DNA, 6122 bp, synthetic construct
  source 1..6122: mol_type = other DNA; note = oGOI-CAG-H2B-tdTomato-noV5-P2A-Fluc-WPRE3-DTS-4084- F
gagggataacatggcccctggatgggaaggccatgttatccctcgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggcccctggatgggaaggccatgttatccct

SEQ ID NO: 75 - DNA, 5975 bp, synthetic construct
  source 1..5975: mol_type = other DNA; note = CAG-H2B-tdTomato-noV5-P2A-Fluc-WPRE3-DTS
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 76 - DNA, 15 bp, synthetic construct
  source 1..15: mol_type = other DNA; note = 5 ITR
agggataacatggcc

SEQ ID NO: 77 - DNA, 15 bp, synthetic construct
  source 1..15: mol_type = other DNA; note = 3 ITR
ggccatgttatccct

SEQ ID NO: 78 - DNA, 70 bp, synthetic construct
  source 1..70: mol_type = other DNA; note = 5 ITR-NUP 358 Apt-3 ITR
agggataacatggcctgacagtggcggcagtcactgagaaaaacgaaaccggagcggccatgttatccct

SEQ ID NO: 79 - DNA, 69 bp, synthetic construct
  source 1..69: mol_type = other DNA; note = 5 ITR-NUP Apt-3 ITR
agggataacatggccccacgcagatagacgctactctactacatcgcagccaacggccatgttatccct

SEQ ID NO: 80 - DNA, 65 bp, synthetic construct
  source 1..65: mol_type = other DNA; note = Adapter-ST-1-NupApt02
agggtatggcacggcccacgcagatagacgctactctactacatcgcagccaacgccgtgccata

SEQ ID NO: 81 - DNA, 65 bp, synthetic construct
  source 1..65: mol_type = other DNA; note = Adapter-ST-2-NupApt02
agggcatggcacggcccacgcagatagacgctactctactacatcgcagccaacgccgtgccatg

SEQ ID NO: 82 - DNA, 65 bp, synthetic construct
  source 1..65: mol_type = other DNA; note = Adapter-ST-3-NupApt02
agggcatgacacggcccacgcagatagacgctactctactacatcgcagccaacgccgtgtcatg

SEQ ID NO: 83 - DNA, 65 bp, synthetic construct
  source 1..65: mol_type = other DNA; note = Adapter-ST-4-NupApt02
agggctaacatgcgcccacgcagatagacgctactctactacatcgcagccaacgcgcatgttag

SEQ ID NO: 84 - DNA, 65 bp, synthetic construct
  source 1..65: mol_type = other DNA; note = Adapter-ST-5-NupApt02
agggcccgaatatgaccacgcagatagacgctactctactacatcgcagccaactcatattcggg

SEQ ID NO: 85 - DNA, 65 bp, synthetic construct
  source 1..65: mol_type = other DNA; note = Adapter-ST-6-NupApt02
agggtcctgacagaaccacgcagatagacgctactctactacatcgcagccaacttctgtcagga

SEQ ID NO: 86 - DNA, 7231 bp, synthetic construct
  source 1..7231: mol_type = other DNA; note = PUC57-tdTomato- CAG-H2B-tdTomato-noV5-P2A-Fluc-WPRE3-DTS
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgcggccgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgtcggctagcatggcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcgggtccgtgatcgcagaggatcggtgcgccgaggtcgagtcaaaactaggcctgcactatcctgcctcattcatcccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccaaccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagggatccggagctactaacttcagcctgctgaagcaggctggagacgtggaggagaaccctggacctatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggagggcccgcggttcgaaggtaagcctatccctaaccctctcctcggtctcgattctacgcgtaccggttagtgaatgcatgaattcctgcagccactcgaggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcggccgcatcggatccccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 87 - DNA, 6506 bp, synthetic construct
  source 1..6506: mol_type = other DNA; note = oDNA Adaptor 19- CAG-H2B-tdTomato-noV5-P2A-Fluc-WPRE3-DTS
gtacctcgcgaatgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattag

SEQ ID NO: 88 - DNA, 12144 bp, synthetic construct
  source 1..12144: mol_type = other DNA; note = Np - CAG-H2B-tdTomato-noV5-P2A-Fluc-WPRE3-DTS
cgagacctgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaaggctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacggtctcgagggataacatggccactcaggccatgttatccctcgagaccgtgtctatgcgctatacttacgttgtccttaatagaacaaaatgtaccagaacaaaatgtaccggtacattttgttctagaacaaaatgtacctctaaatattggtagcttccttagctgaaggcggaaagtccccagcggaaagtccccagcggaaagtccccagcagggatcgcacagctcagtagacatcttcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaccccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccataggtccagggttctcctccacgtctccagcctgcttcagcaggctgaagttagtagccttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacggccatgttgttgtcctcggaggaggcggtgccggagctgccgctgccggtgctgccggtgccatgccccaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaccggtggatccttagcgctggtgtacttggtgatggccttagtaccctcggacacggcgtgcttggccaactccccaggcagcagcaggcgcacggccgtctggatctccctggaggtgatggtcgagcgcttgttgtaatgcgccaggcgggaagcctcacctgcgatgcgctcgaaaatgtcgttcacaaacgaattcatgatgcccatggccttggacgaaatgccggtgtcagggtggacctgcttcagaaccttgtacacatagatggaatagctctccttgcggctgcgcttgcgcttcttgccgcctttcttctgcgccttagtcaccgccttcttggagccctttttcggggcgggagcagacttcgctggctctggcatggtggcgatcgcgcgttctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgtcggatctagatgcaggtctcgagggataacatggccactcaggccatgttatccct

SEQ ID NO: 89 - skipped

SEQ ID NO: 90 - DNA, 20 bp, synthetic construct
  source 1..20: mol_type = other DNA; note = CpG motif
tccatgacgttcctgacgtt

SEQ ID NO: 91 - DNA, 24 bp, synthetic construct
  source 1..24: mol_type = other DNA; note = CpG motif
tcgtcgttttgtcgttttgtcgtt

SEQ ID NO: 92 - DNA, 10 bp, synthetic construct
  source 1..10: mol_type = other DNA; note = CpG motif
tcgtcgtttt

SEQ ID NO: 93 - skipped

SEQ ID NO: 94 - DNA, 65 bp, synthetic construct; identical to SEQ ID NO: 80
  source 1..65: mol_type = other DNA; note = Adapter-ST-1-NupApt02
  modified_base 10..15: mod_base = OTHER; note = Locked nucleic acid

SEQ ID NO: 95 - DNA, 65 bp, synthetic construct; identical to SEQ ID NO: 81
  source 1..65: mol_type = other DNA; note = Adapter-ST-2-NupApt02
  modified_base 10..15: mod_base = OTHER; note = Locked nucleic acid

SEQ ID NO: 96 - DNA, 65 bp, synthetic construct; identical to SEQ ID NO: 82
  source 1..65: mol_type = other DNA; note = Adapter-ST-3-NupApt02
  modified_base 10..15: mod_base = OTHER; note = Locked nucleic acid

SEQ ID NO: 97 - DNA, 65 bp, synthetic construct; identical to SEQ ID NO: 83
  source 1..65: mol_type = other DNA; note = Adapter-ST-4-NupApt02
  modified_base 10..15: mod_base = OTHER; note = Locked nucleic acid

SEQ ID NO: 98 - DNA, 65 bp, synthetic construct; identical to SEQ ID NO: 84
  source 1..65: mol_type = other DNA; note = Adapter-ST-5-NupApt02
  modified_base 10..15: mod_base = OTHER; note = Locked nucleic acid

SEQ ID NO: 99 - DNA, 65 bp, synthetic construct; identical to SEQ ID NO: 85
  source 1..65: mol_type = other DNA; note = Adapter-ST-6-NupApt02
  modified_base 10..15: mod_base = OTHER; note = Locked nucleic acid

SEQ ID NO: 100 - DNA, 65 bp, synthetic construct
  source 1..65: mol_type = other DNA; note = Adapter-ST-7-NupApt02
  modified_base 10..15: mod_base = OTHER; note = Locked nucleic acid
agggacctagacgatccacgcagatagacgctactctactacatcgcagccaacatcgtctaggt

SEQ ID NO: 101 - DNA, 31 bp, synthetic construct
  source 1..31: mol_type = other DNA; note = Adapter-ST-1
agggtatggcacggcmmmmmgccgtgccata

SEQ ID NO: 102 - DNA, 31 bp, synthetic construct
  source 1..31: mol_type = other DNA; note = Adapter-ST-2
agggcatggcacggcmmmmmgccgtgccatg

SEQ ID NO: 103 - DNA, 31 bp, synthetic construct
  source 1..31: mol_type = other DNA; note = Adapter-ST-3
agggcatgacacggcmmmmmgccgtgtcatg

SEQ ID NO: 104 - DNA, 31 bp, synthetic construct
  source 1..31: mol_type = other DNA; note = Adapter-ST-4
agggctaacatgcgcmmmmmgcgcatgttag

SEQ ID NO: 105 - DNA, 31 bp, synthetic construct
  source 1..31: mol_type = other DNA; note = Adapter-ST-5
agggcccgaatatgammmmmtcatattcggg

SEQ ID NO: 106 - DNA, 31 bp, synthetic construct
  source 1..31: mol_type = other DNA; note = Adapter-ST-6
agggtcctgacagaammmmmttctgtcagga

SEQ ID NO: 107 - DNA, 31 bp, synthetic construct
  source 1..31: mol_type = other DNA; note = Adapter-ST-7
agggacctagacgatmmmmmatcgtctaggt

SEQ ID NO: 108 - DNA, 31 bp, synthetic construct; identical to SEQ ID NO: 101
  source 1..31: mol_type = other DNA; note = Adapter-ST-1
  modified_base 10..15: mod_base = OTHER; note = Locked nucleic acid

SEQ ID NO: 109 - DNA, 31 bp, synthetic construct; identical to SEQ ID NO: 102
  source 1..31: mol_type = other DNA; note = Adapter-ST-2
  modified_base 10..15: mod_base = OTHER; note = Locked nucleic acid

SEQ ID NO: 110 - DNA, 31 bp, synthetic construct; identical to SEQ ID NO: 103
  source 1..31: mol_type = other DNA; note = Adapter-ST-3
  modified_base 10..15: mod_base = OTHER; note = Locked nucleic acid

SEQ ID NO: 111 - DNA, 31 bp, synthetic construct; identical to SEQ ID NO: 104
  source 1..31: mol_type = other DNA; note = Adapter-ST-4
  modified_base 10..15: mod_base = OTHER; note = Locked nucleic acid

SEQ ID NO: 112 - DNA, 31 bp, synthetic construct; identical to SEQ ID NO: 105
  source 1..31: mol_type = other DNA; note = Adapter-ST-5
  modified_base 10..15: mod_base = OTHER; note = Locked nucleic acid

SEQ ID NO: 113 - DNA, 31 bp, synthetic construct; identical to SEQ ID NO: 106
  source 1..31: mol_type = other DNA; note = Adapter-ST-6
  modified_base 10..15: mod_base = OTHER; note = Locked nucleic acid

SEQ ID NO: 114 - DNA, 31 bp, synthetic construct; identical to SEQ ID NO: 107
  source 1..31: mol_type = other DNA; note = Adapter-ST-7
  modified_base 10..15: mod_base = OTHER; note = Locked nucleic acid

SEQ ID NO: 115 - DNA, 31 bp, synthetic construct; identical to SEQ ID NO: 101
  source 1..31: mol_type = other DNA; note = Adapter-ST-1

SEQ ID NO: 116 - DNA, 31 bp, synthetic construct; identical to SEQ ID NO: 102
  source 1..31: mol_type = other DNA; note = Adapter-ST-2

SEQ ID NO: 117 - DNA, 31 bp, synthetic construct; identical to SEQ ID NO: 103
  source 1..31: mol_type = other DNA; note = Adapter-ST-3

SEQ ID NO: 118 - DNA, 31 bp, synthetic construct; identical to SEQ ID NO: 104
  source 1..31: mol_type = other DNA; note = Adapter-ST-4

SEQ ID NO: 119 - DNA, 31 bp, synthetic construct; identical to SEQ ID NO: 105
  source 1..31: mol_type = other DNA; note = Adapter-ST-5

SEQ ID NO: 120 - DNA, 31 bp, synthetic construct; identical to SEQ ID NO: 106
  source 1..31: mol_type = other DNA; note = Adapter-ST-6

SEQ ID NO: 121 - DNA, 31 bp, synthetic construct; identical to SEQ ID NO: 107
  source 1..31: mol_type = other DNA; note = Adapter-ST-7

SEQ ID NO: 122 - DNA, 31 bp, synthetic construct; identical to SEQ ID NO: 101
  source 1..31: mol_type = other DNA; note = Adapter-ST-1
  modified_base 10..15: mod_base = OTHER; note = Locked nucleic acid

SEQ ID NO: 123 - DNA, 31 bp, synthetic construct; identical to SEQ ID NO: 102
  source 1..31: mol_type = other DNA; note = Adapter-ST-2
  modified_base 10..15: mod_base = OTHER; note = Locked nucleic acid

SEQ ID NO: 124 - DNA, 31 bp, synthetic construct; identical to SEQ ID NO: 103
  source 1..31: mol_type = other DNA; note = Adapter-ST-3
  modified_base 10..15: mod_base = OTHER; note = Locked nucleic acid

SEQ ID NO: 125 - DNA, 31 bp, synthetic construct; identical to SEQ ID NO: 104
  source 1..31: mol_type = other DNA; note = Adapter-ST-4
  modified_base 10..15: mod_base = OTHER; note = Locked nucleic acid

SEQ ID NO: 126 - DNA, 31 bp, synthetic construct; identical to SEQ ID NO: 105
  source 1..31: mol_type = other DNA; note = Adapter-ST-5
  modified_base 10..15: mod_base = OTHER; note = Locked nucleic acid

SEQ ID NO: 127 - DNA, 31 bp, synthetic construct; identical to SEQ ID NO: 106
  source 1..31: mol_type = other DNA; note = Adapter-ST-6
  modified_base 10..15: mod_base = OTHER; note = Locked nucleic acid

SEQ ID NO: 128 - DNA, 31 bp, synthetic construct; identical to SEQ ID NO: 107
  source 1..31: mol_type = other DNA; note = Adapter-ST-7
  modified_base 10..15: mod_base = OTHER; note = Locked nucleic acid

SEQ ID NO: 129 - DNA, 97 bp, unidentified
  source 1..97: mol_type = other DNA; note = IPO76 Importin Aptamer
ggatcctgagctactgacgggttatgtgtcagtccccaggtaatgctgaggcttttggttcatttggccggttgtggcaccactactgaccatacac

SEQ ID NO: 130 - DNA, 97 bp, synthetic construct
  source 1..97: mol_type = other DNA; note = IPO72 Importin Aptamer
ggatcctgagctactgactttgggtcgaattggttggctcgccctcttcttggaattcaggagggcgattgaacggacaccactactgaccatacac

SEQ ID NO: 131 - DNA, 123 bp, synthetic construct
  source 1..123: mol_type = other DNA; note = IPO76-with adaptor
agggataacatggccggatcctgagctactgacgggttatgtgtcagtccccaggtaatgctgaggcttttggttcatttggccggttgtggcaccactactgaccatacacggccatgttat

SEQ ID NO: 132 - DNA, 123 bp, synthetic construct
  source 1..123: mol_type = other DNA; note = IPO72-with adaptor
agggataacatggccggatcctgagctactgactttgggtcgaattggttggctcgccctcttcttggaattcaggagggcgattgaacggacaccactactgaccatacacggccatgttat

SEQ ID NO: 133 - DNA, 123 bp, synthetic construct; identical to SEQ ID NO: 131
  source 1..123: mol_type = other DNA; note = IPO76-with adaptor
  modified_base 10..15: mod_base = OTHER; note = Locked nucleic acid

SEQ ID NO: 134 - DNA, 123 bp, synthetic construct; identical to SEQ ID NO: 132
  source 1..123: mol_type = other DNA; note = IPO72-with adaptor
  modified_base 10..15: mod_base = OTHER; note = Locked nucleic acid

SEQ ID NO: 135 - DNA, 46 bp, synthetic construct
  source 1..46: mol_type = other DNA; note = CT1AS1411
cccccccccctcccccccccggtggtggtggttgtggtggtggtgg

SEQ ID NO: 136 - DNA, 46 bp, synthetic construct
  source 1..46: mol_type = other DNA; note = CT2AS1411
cccccctcccccctccccccggtggtggtggttgtggtggtggtgg

SEQ ID NO: 137 - DNA, 46 bp, synthetic construct
  source 1..46: mol_type = other DNA; note = CT3AS1411
cccctcccctccccctccccggtggtggtggttgtggtggtggtgg

SEQ ID NO: 138 - DNA, 57 bp, synthetic construct
  source 1..57: mol_type = other DNA; note = CT4AS1411
ccctccctccctccctccccggtggtggtggttgtggtggtggtggggccatgttat

SEQ ID NO: 139 - DNA, 36 bp, synthetic construct
  source 1..36: mol_type = other DNA; note = C10AS1411
ccccccccccggtggtggtggttgtggtggtggtgg

SEQ ID NO: 140 - DNA, 46 bp, synthetic construct
  source 1..46: mol_type = other DNA; note = A20AS1411
aaaaaaaaaaaaaaaaaaaaggtggtggtggttgtggtggtggtgg

SEQ ID NO: 141 - DNA, 46 bp, synthetic construct
  source 1..46: mol_type = other DNA; note = T20AS1411
ttttttttttttttttttttggtggtggtggttgtggtggtggtgg

SEQ ID NO: 142 - DNA, 46 bp, synthetic construct; identical to SEQ ID NO: 135
  source 1..46: mol_type = other DNA; note = CT1AS1411

SEQ ID NO: 143 - DNA, 46 bp, synthetic construct; identical to SEQ ID NO: 136
  source 1..46: mol_type = other DNA; note = CT2AS1411

SEQ ID NO: 144 - DNA, 46 bp, synthetic construct; identical to SEQ ID NO: 137
  source 1..46: mol_type = other DNA; note = CT3AS1411

SEQ ID NO: 145 - DNA, 57 bp, synthetic construct; identical to SEQ ID NO: 138
  source 1..57: mol_type = other DNA; note = CT4AS1411

SEQ ID NO: 146 - DNA, 36 bp, synthetic construct; identical to SEQ ID NO: 139
  source 1..36: mol_type = other DNA; note = C10AS1411

SEQ ID NO: 147 - DNA, 46 bp, synthetic construct; identical to SEQ ID NO: 140
  source 1..46: mol_type = other DNA; note = A20AS1411

SEQ ID NO: 148 - DNA, 46 bp, synthetic construct; identical to SEQ ID NO: 141
  source 1..46: mol_type = other DNA; note = T20AS1411

SEQ ID NO: 149 - DNA, 72 bp, synthetic construct
  source 1..72: mol_type = other DNA; note = Adapter-ST-1-C20AS1411
agggtatggcacggcccccccccccccccccccccggtggtggtggttgtggtggtggtgggccgtgccata

SEQ ID NO: 150 - DNA, 72 bp, synthetic construct
  source 1..72: mol_type = other DNA; note = Adapter-ST-2-C20AS1411
agggcatggcacggcccccccccccccccccccccggtggtggtggttgtggtggtggtgggccgtgccatg

SEQ ID NO: 151 - DNA, 72 bp, synthetic construct
  source 1..72: mol_type = other DNA; note = Adapter-ST-3-C20AS1411
agggcatgacacggcccccccccccccccccccccggtggtggtggttgtggtggtggtgggccgtgtcatg

SEQ ID NO: 152 - DNA, 72 bp, synthetic construct
  source 1..72: mol_type = other DNA; note = Adapter-ST-4-C20AS1411
agggctaacatgcgcccccccccccccccccccccggtggtggtggttgtggtggtggtgggcgcatgttag

SEQ ID NO: 153 - DNA, 72 bp, synthetic construct
  source 1..72: mol_type = other DNA; note = Adapter-ST-5-C20AS1411
agggcccgaatatgaccccccccccccccccccccggtggtggtggttgtggtggtggtggtcatattcggg

SEQ ID NO: 154 - DNA, 72 bp, synthetic construct
  source 1..72: mol_type = other DNA; note = Adapter-ST-6-C20AS1411
agggtcctgacagaaccccccccccccccccccccggtggtggtggttgtggtggtggtggttctgtcagga

SEQ ID NO: 155 - DNA, 72 bp, synthetic construct
  source 1..72: mol_type = other DNA; note = Adapter-ST-7-C20AS1411
agggacctagacgatccccccccccccccccccccggtggtggtggttgtggtggtggtggatcgtctaggt

SEQ ID NO: 156 - DNA, 50 bp, synthetic construct
  source 1..50: mol_type = other DNA; note = Adapter-ST-1-INH-18
agggtatggcacggccctggatgggaacttaccgctgcagccgtgccata

SEQ ID NO: 157 - DNA, 50 bp, synthetic construct
  source 1..50: mol_type = other DNA; note = Adapter-ST-2-INH-18
agggcatggcacggccctggatgggaacttaccgctgcagccgtgccatg

SEQ ID NO: 158 - DNA, 50 bp, synthetic construct
  source 1..50: mol_type = other DNA; note = Adapter-ST-3-INH-18
agggcatgacacggccctggatgggaacttaccgctgcagccgtgtcatg

SEQ ID NO: 159 - DNA, 50 bp, synthetic construct
  source 1..50: mol_type = other DNA; note = Adapter-ST-4-INH-18
agggctaacatgcgccctggatgggaacttaccgctgcagcgcatgttag

SEQ ID NO: 160 - DNA, 50 bp, synthetic construct
  source 1..50: mol_type = other DNA; note = Adapter-ST-5-INH-18
agggcccgaatatgacctggatgggaacttaccgctgcatcatattcggg

SEQ ID NO: 161 - DNA, 50 bp, synthetic construct
  source 1..50: mol_type = other DNA; note = Adapter-ST-6-INH-18
agggtcctgacagaacctggatgggaacttaccgctgcattctgtcagga

SEQ ID NO: 162 - DNA, 50 bp, synthetic construct
  source 1..50: mol_type = other DNA; note = Adapter-ST-7-INH-18
agggacctagacgatcctggatgggaacttaccgctgcaatcgtctaggt

SEQ ID NO: 163 - DNA, 72 bp, synthetic construct; identical to SEQ ID NO: 149
  source 1..72: mol_type = other DNA; note = Adapter-ST-1-C20AS1411
  modified_base 10..15: mod_base = OTHER; note = Locked nucleic acid

SEQ ID NO: 164 - DNA, 72 bp, synthetic construct; identical to SEQ ID NO: 150
  source 1..72: mol_type = other DNA; note = Adapter-ST-2-C20AS1411
  modified_base 10..15: mod_base = OTHER; note = Locked nucleic acid

SEQ ID NO: 165 - DNA, 72 bp, synthetic construct; identical to SEQ ID NO: 151
  source 1..72: mol_type = other DNA; note = Adapter-ST-3-C20AS1411
  modified_base 10..15: mod_base = OTHER; note = Locked nucleic acid

SEQ ID NO: 166 - DNA, 72 bp, synthetic construct; identical to SEQ ID NO: 152
  source 1..72: mol_type = other DNA; note = Adapter-ST-4-C20AS1411
  modified_base 10..15: mod_base = OTHER; note = Locked nucleic acid

SEQ ID NO: 167 - DNA, 72 bp, synthetic construct; identical to SEQ ID NO: 153
  source 1..72: mol_type = other DNA; note = Adapter-ST-5-C20AS1411
  modified_base 10..15: mod_base = OTHER; note = Locked nucleic acid

SEQ ID NO: 168 - DNA, 72 bp, synthetic construct; identical to SEQ ID NO: 154
  source 1..72: mol_type = other DNA; note = Adapter-ST-6-C20AS1411
  modified_base 10..15: mod_base = OTHER; note = Locked nucleic acid

SEQ ID NO: 169 - DNA, 72 bp, synthetic construct; identical to SEQ ID NO: 155
  source 1..72: mol_type = other DNA; note = Adapter-ST-7-C20AS1411
  modified_base 10..15: mod_base = OTHER; note = Locked nucleic acid

SEQ ID NO: 170 - DNA, 50 bp, synthetic construct; identical to SEQ ID NO: 156
  source 1..50: mol_type = other DNA; note = Adapter-ST-1-INH-18
  modified_base 10..15: mod_base = OTHER; note = Locked nucleic acid

SEQ ID NO: 171 - DNA, 50 bp, synthetic construct; identical to SEQ ID NO: 157
  source 1..50: mol_type = other DNA; note = Adapter-ST-2-INH-18
  modified_base 10..15: mod_base = OTHER; note = Locked nucleic acid

SEQ ID NO: 172 - DNA, 50 bp, synthetic construct; identical to SEQ ID NO: 158
  source 1..50: mol_type = other DNA; note = Adapter-ST-3-INH-18
  modified_base 10..15: mod_base = OTHER; note = Locked nucleic acid

SEQ ID NO: 173 - DNA, 50 bp, synthetic construct; identical to SEQ ID NO: 159
  source 1..50: mol_type = other DNA; note = Adapter-ST-4-INH-18
  modified_base 10..15: mod_base = OTHER; note = Locked nucleic acid

SEQ ID NO: 174 - DNA, 50 bp, synthetic construct; identical to SEQ ID NO: 160
  source 1..50: mol_type = other DNA; note = Adapter-ST-5-INH-18
  modified_base 10..15: mod_base = OTHER; note = Locked nucleic acid

SEQ ID NO: 175 - DNA, 50 bp, synthetic construct; identical to SEQ ID NO: 161
  source 1..50: mol_type = other DNA; note = Adapter-ST-6-INH-18
  modified_base 10..15: mod_base = OTHER; note = Locked nucleic acid

SEQ ID NO: 176 - DNA, 50 bp, synthetic construct; identical to SEQ ID NO: 162
  source 1..50: mol_type = other DNA; note = Adapter-ST-7-INH-18
  modified_base 10..15: mod_base = OTHER; note = Locked nucleic acid

SEQ ID NO: 177 - DNA, 72 bp, synthetic construct; identical to SEQ ID NO: 149
  source 1..72: mol_type = other DNA; note = Adapter-ST-1-C20AS1411

SEQ ID NO: 178 - DNA, 72 bp, synthetic construct; identical to SEQ ID NO: 150
  source 1..72: mol_type = other DNA; note = Adapter-ST-2-C20AS1411

SEQ ID NO: 179 - DNA, 72 bp, synthetic construct; identical to SEQ ID NO: 151
  source 1..72: mol_type = other DNA; note = Adapter-ST-3-C20AS1411

SEQ ID NO: 180 - DNA, 72 bp, synthetic construct; identical to SEQ ID NO: 152
  source 1..72: mol_type = other DNA; note = Adapter-ST-4-C20AS1411

SEQ ID NO: 181 - DNA, 72 bp, synthetic construct; identical to SEQ ID NO: 153
  source 1..72: mol_type = other DNA; note = Adapter-ST-5-C20AS1411

SEQ ID NO: 182 - DNA, 72 bp, synthetic construct; identical to SEQ ID NO: 154
  source 1..72: mol_type = other DNA; note = Adapter-ST-6-C20AS1411

SEQ ID NO: 183 - DNA, 72 bp, synthetic construct; identical to SEQ ID NO: 155
  source 1..72: mol_type = other DNA; note = Adapter-ST-7-C20AS1411

SEQ ID NO: 184 - DNA, 50 bp, synthetic construct; identical to SEQ ID NO: 156
  source 1..50: mol_type = other DNA; note = Adapter-ST-1-INH-18

SEQ ID NO: 185 - DNA, 50 bp, synthetic construct; identical to SEQ ID NO: 157
  source 1..50: mol_type = other DNA; note = Adapter-ST-2-INH-18

SEQ ID NO: 186 - DNA, 50 bp, synthetic construct; identical to SEQ ID NO: 158
  source 1..50: mol_type = other DNA; note = Adapter-ST-3-INH-18

SEQ ID NO: 187 - DNA, 50 bp, synthetic construct; identical to SEQ ID NO: 159
  source 1..50: mol_type = other DNA; note = Adapter-ST-4-INH-18

SEQ ID NO: 188 - DNA, 50 bp, synthetic construct; identical to SEQ ID NO: 160
  source 1..50: mol_type = other DNA; note = Adapter-ST-5-INH-18

SEQ ID NO: 189 - DNA, 50 bp, synthetic construct; identical to SEQ ID NO: 161
  source 1..50: mol_type = other DNA; note = Adapter-ST-6-INH-18

SEQ ID NO: 190 - DNA, 50 bp, synthetic construct; identical to SEQ ID NO: 162
  source 1..50: mol_type = other DNA; note = Adapter-ST-7-INH-18

SEQ ID NO: 191 - DNA, 72 bp, synthetic construct; identical to SEQ ID NO: 149
  source 1..72: mol_type = other DNA; note = Adapter-ST-1-C20AS1411
  modified_base 10..15: mod_base = OTHER; note = Locked nucleic acid

SEQ ID NO: 192 - DNA, 72 bp, synthetic construct; identical to SEQ ID NO: 150
  source 1..72: mol_type = other DNA; note = Adapter-ST-2-C20AS1411
  modified_base 10..15: mod_base = OTHER; note = Locked nucleic acid

SEQ ID NO: 193 - DNA, 72 bp, synthetic construct; identical to SEQ ID NO: 151
  source 1..72: mol_type = other DNA; note = Adapter-ST-3-C20AS1411
  modified_base 10..15: mod_base = OTHER; note = Locked nucleic acid

SEQ ID NO: 194 - DNA, 72 bp, synthetic construct; identical to SEQ ID NO: 152
  source 1..72: mol_type = other DNA; note = Adapter-ST-4-C20AS1411
  modified_base 10..15: mod_base = OTHER; note = Locked nucleic acid

SEQ ID NO: 195 - DNA, 72 bp, synthetic construct; identical to SEQ ID NO: 153
  source 1..72: mol_type = other DNA; note = Adapter-ST-5-C20AS1411
  modified_base 10..15: mod_base = OTHER; note = Locked nucleic acid

SEQ ID NO: 196 - DNA, 72 bp, synthetic construct; identical to SEQ ID NO: 154
  source 1..72: mol_type = other DNA; note = Adapter-ST-6-C20AS1411
  modified_base 10..15: mod_base = OTHER; note = Locked nucleic acid

SEQ ID NO: 197 - DNA, 72 bp, synthetic construct; identical to SEQ ID NO: 155
  source 1..72: mol_type = other DNA; note = Adapter-ST-7-C20AS1411
  modified_base 10..15: mod_base = OTHER; note = Locked nucleic acid

SEQ ID NO: 198 - DNA, 50 bp, synthetic construct; identical to SEQ ID NO: 156
  source 1..50: mol_type = other DNA; note = Adapter-ST-1-INH-18
  modified_base 10..15: mod_base = OTHER; note = Locked nucleic acid

SEQ ID NO: 199 - DNA, 50 bp, synthetic construct; identical to SEQ ID NO: 157
  source 1..50: mol_type = other DNA; note = Adapter-ST-2-INH-18
  modified_base 10..15: mod_base = OTHER; note = Locked nucleic acid

SEQ ID NO: 200 - DNA, 50 bp, synthetic construct; identical to SEQ ID NO: 158
  source 1..50: mol_type = other DNA; note = Adapter-ST-3-INH-18
  modified_base 10..15: mod_base = OTHER; note = Locked nucleic acid

SEQ ID NO: 201 - DNA, 50 bp, synthetic construct; identical to SEQ ID NO: 159
  source 1..50: mol_type = other DNA; note = Adapter-ST-4-INH-18
  modified_base 10..15: mod_base = OTHER; note = Locked nucleic acid

SEQ ID NO: 202 - DNA, 50 bp, synthetic construct; identical to SEQ ID NO: 160
  source 1..50: mol_type = other DNA; note = Adapter-ST-5-INH-18
  modified_base 10..15: mod_base = OTHER; note = Locked nucleic acid

SEQ ID NO: 203 - DNA, 50 bp, synthetic construct; identical to SEQ ID NO: 161
  source 1..50: mol_type = other DNA; note = Adapter-ST-6-INH-18
  modified_base 10..15: mod_base = OTHER; note = Locked nucleic acid

SEQ ID NO: 204 - DNA, 50 bp, synthetic construct; identical to SEQ ID NO: 162
  source 1..50: mol_type = other DNA; note = Adapter-ST-7-INH-18
  modified_base 10..15: mod_base = OTHER; note = Locked nucleic acid

SEQ ID NO: 205 - DNA, 15 bp, synthetic construct
  source 1..15: mol_type = other DNA; note = Adapter-ST-1 5 End
agggtatggcacggc

SEQ ID NO: 206 - DNA, 15 bp, synthetic construct
  source 1..15: mol_type = other DNA; note = Adapter-ST-2 5 End
agggcatggcacggc

SEQ ID NO: 207 - DNA, 15 bp, synthetic construct
  source 1..15: mol_type = other DNA; note = Adapter-ST-3 5 End
agggcatgacacggc

SEQ ID NO: 208 - DNA, 15 bp, synthetic construct
  source 1..15: mol_type = other DNA; note = Adapter-ST-4 5 End
agggctaacatgcgc

SEQ ID NO: 209 - DNA, 15 bp, synthetic construct
  source 1..15: mol_type = other DNA; note = Adapter-ST-5 5 End
agggcccgaatatga

SEQ ID NO: 210 - DNA, 15 bp, synthetic construct
  source 1..15: mol_type = other DNA; note = Adapter-ST-6 5 End
agggtcctgacagaa

SEQ ID NO: 211 - DNA, 15 bp, synthetic construct
  source 1..15: mol_type = other DNA; note = Adapter-ST-7 5 End
agggacctagacgat

SEQ ID NO: 212 - DNA, 11 bp, synthetic construct
  source 1..11: mol_type = other DNA; note = Adapter-ST-1 3 End
gccgtgccata

SEQ ID NO: 213 - DNA, 11 bp, synthetic construct
  source 1..11: mol_type = other DNA; note = Adapter-ST-2 3 End
gccgtgccatg

SEQ ID NO: 214 - DNA, 11 bp, synthetic construct
  source 1..11: mol_type = other DNA; note = Adapter-ST-3 3 End
gccgtgtcatg

SEQ ID NO: 215 - DNA, 11 bp, synthetic construct
  source 1..11: mol_type = other DNA; note = Adapter-ST-4 3 End
gcgcatgttag

SEQ ID NO: 216 - DNA, 11 bp, synthetic construct
  source 1..11: mol_type = other DNA; note = Adapter-ST-5 3 End
tcatattcggg

SEQ ID NO: 217 - DNA, 11 bp, synthetic construct
  source 1..11: mol_type = other DNA; note = Adapter-ST-6 3 End
ttctgtcagga

SEQ ID NO: 218 - DNA, 11 bp, synthetic construct
  source 1..11: mol_type = other DNA; note = Adapter-ST-7 3 End
atcgtctaggt

SEQ ID NO: 219 - DNA, 15 bp, synthetic construct
  source 1..15: mol_type = other DNA; note = Adapter-ST-1 5 End
agggtatggcacggc

SEQ ID NO: 220 - DNA, 15 bp, synthetic construct
  source 1..15: mol_type = other DNA; note = Adapter-ST-2 5 End
agggcatggcacggc

SEQ ID NO: 221 - DNA, 15 bp, synthetic construct
  source 1..15: mol_type = other DNA; note = Adapter-ST-3 5 End
agggcatgacacggc

SEQ ID NO: 222 - DNA, 15 bp, synthetic construct
  source 1..15: mol_type = other DNA; note = Adapter-ST-4 5 End
agggctaacatgcgc

SEQ ID NO: 223 - DNA, 15 bp, synthetic construct
  source 1..15: mol_type = other DNA; note = Adapter-ST-5 5 End
agggcccgaatatga

SEQ ID NO: 224 - DNA, 15 bp, synthetic construct
  source 1..15: mol_type = other DNA; note = Adapter-ST-6 5 End
agggtcctgacagaa

SEQ ID NO: 225 - DNA, 15 bp, synthetic construct
  source 1..15: mol_type = other DNA; note = Adapter-ST-7 5 End
agggacctagacgat

SEQ ID NO: 226 - DNA, 11 bp, synthetic construct
  source 1..11: mol_type = other DNA; note = Adapter-ST-1 3 End
gccgtgccata

SEQ ID NO: 227 - DNA, 11 bp, synthetic construct
  source 1..11: mol_type = other DNA; note = Adapter-ST-2 3 End
gccgtgccatg

SEQ ID NO: 228 - DNA, 11 bp, synthetic construct
  source 1..11: mol_type = other DNA; note = Adapter-ST-3 3 End
gccgtgtcatg

SEQ ID NO: 229 - DNA, 11 bp, synthetic construct
  source 1..11: mol_type = other DNA; note = Adapter-ST-4 3 End
gcgcatgttag

SEQ ID NO: 230 - DNA, 11 bp, synthetic construct
  source 1..11: mol_type = other DNA; note = Adapter-ST-5 3 End
tcatattcggg

SEQ ID NO: 231 - DNA, 11 bp, synthetic construct
  source 1..11: mol_type = other DNA; note = Adapter-ST-6 3 End
ttctgtcagga

SEQ ID NO: 232 - DNA, 11 bp, synthetic construct
  source 1..11: mol_type = other DNA; note = Adapter-ST-7 3 End
atcgtctaggt

SEQ ID NO: 233 - DNA, 15 bp, synthetic construct
  source 1..15: mol_type = other DNA; note = Adapter-ST-1 5 End
agggtatggcacggc

SEQ ID NO: 234 - DNA, 15 bp, synthetic construct
  source 1..15: mol_type = other DNA; note = Adapter-ST-2 5 End
agggcatggcacggc

SEQ ID NO: 235 - DNA, 15 bp, synthetic construct
  source 1..15: mol_type = other DNA; note = Adapter-ST-3 5 End
agggcatgacacggc

SEQ ID NO: 236 - DNA, 15 bp, synthetic construct
  source 1..15: mol_type = other DNA; note = Adapter-ST-4 5 End
agggctaacatgcgc

SEQ ID NO: 237 - DNA, 15 bp, synthetic construct
  source 1..15: mol_type = other DNA; note = Adapter-ST-5 5 End
agggcccgaatatga

SEQ ID NO: 238 - DNA, 15 bp, synthetic construct
  source 1..15: mol_type = other DNA; note = Adapter-ST-6 5 End
agggtcctgacagaa

SEQ ID NO: 239 - DNA, 15 bp, synthetic construct
  source 1..15: mol_type = other DNA; note = Adapter-ST-7 5 End
agggacctagacgat

SEQ ID NO: 240 - DNA, 11 bp, synthetic construct
  source 1..11: mol_type = other DNA; note = Adapter-ST-1 3 End
gccgtgccata

SEQ ID NO: 241 - DNA, 11 bp, synthetic construct
  source 1..11: mol_type = other DNA; note = Adapter-ST-2 3 End
gccgtgccatg

SEQ ID NO: 242 - DNA, 11 bp, synthetic construct
  source 1..11: mol_type = other DNA; note = Adapter-ST-3 3 End
gccgtgtcatg

SEQ ID NO: 243 - DNA, 11 bp, synthetic construct
  source 1..11: mol_type = other DNA; note = Adapter-ST-4 3 End
gcgcatgttag

SEQ ID NO: 244 - DNA, 11 bp, synthetic construct
  source 1..11: mol_type = other DNA; note = Adapter-ST-5 3 End
tcatattcggg

SEQ ID NO: 245 - DNA, 11 bp, synthetic construct
  source 1..11: mol_type = other DNA; note = Adapter-ST-6 3 End
ttctgtcagga

SEQ ID NO: 246 - DNA, 11 bp, synthetic construct
  source 1..11: mol_type = other DNA; note = Adapter-ST-7 3 End
atcgtctaggt

SEQ ID NO: 247 - DNA, 15 bp, synthetic construct
  source 1..15: mol_type = other DNA; note = Adapter-ST-1 5 End
agggtatggcacggc

SEQ ID NO: 248 - DNA, 15 bp, synthetic construct
  source 1..15: mol_type = other DNA; note = Adapter-ST-2 5 End
agggcatggcacggc

SEQ ID NO: 249 - DNA, 15 bp, synthetic construct
  source 1..15: mol_type = other DNA; note = Adapter-ST-3 5 End
agggcatgacacggc

SEQ ID NO: 250 - DNA, 15 bp, synthetic construct
  source 1..15: mol_type = other DNA; note = Adapter-ST-4 5 End
agggctaacatgcgc

SEQ ID NO: 251 - DNA, 15 bp, synthetic construct
  source 1..15: mol_type = other DNA; note = Adapter-ST-5 5 End
agggcccgaatatga

SEQ ID NO: 252 - DNA, 15 bp, synthetic construct
  source 1..15: mol_type = other DNA; note = Adapter-ST-6 5 End
agggtcctgacagaa

SEQ ID NO: 253 - DNA, 5955 bp, synthetic construct
  source 1..5955: mol_type = other DNA; note = NP-ApoE-AAT-hBGi-FVIII- DM-WPRE3-DTS
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct